(19) United States
(12) Patent Application Publication
(10) Pub. No.: US 2023/0190717 A1
(43) Pub. Date: Jun. 22, 2023

(54) P38 GAMMA INHIBITORS AND METHOD OF USE THEREOF

(71) Applicant: City of Hope, Duarte, CA (US)

(72) Inventors: Xu Hannah Zhang, Duarte, CA (US); Steve Rosen, Duarte, CA (US); David Horne, Duarte, CA (US); Sangkil Nam, Duarte, CA (US); Hongzhi Li, Duarte, CA (US); Jun Xie, Duarte, CA (US)

(21) Appl. No.: 16/604,151

(22) PCT Filed: Apr. 9, 2018
(86) PCT No.: PCT/US18/26720

§ 371 Date: Oct. 9, 2019

Related U.S. Application Data
(60) Provisional application No. 62/483,898, filed on Apr. 10, 2017.

(51) Int. Cl. A61K 31/437; A61K 45/06; A61P 35/00; A61P 7/00; C07D 471/04; A61K 31/167

(52) U.S. Cl. CPC A61K 31/437; A61K 45/06; A61P 35/00; A61P 7/00; C07D 471/04; A61K 31/167

(57) ABSTRACT

Provided herein, inter alia, are methods of treating cancer or cutaneous T-cell lymphoma (CTCL) using compounds of the invention.

### CROSS-REFERENCES TO RELATED APPLICATIONS

This application claims the benefit of U.S. Provisional Patent Application No. 62/483,898, filed Apr. 10, 2017, which is incorporated herein in its entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file 048440-643001WO_ST25.TXT, created on Apr. 5, 2018, 15,050 bytes, machine format IBM-PC, MS Windows operating system, is hereby incorporated by reference.

### BACKGROUND OF THE INVENTION

Cutaneous T cell lymphoma (CTCL) is a severe, disfiguring, and incurable malignancy with poor prognosis for patients with advanced disease. CTCL develops from clonal expansion of effector/central memory CD4+ T cells in a background of chronic inflammation [1]. It most commonly presents on the skin as mycosis fungoides (MF) or the leukemic variant, Sézary syndrome (SS), and may involve the blood, lymph nodes, or other organs [2]. In the US, approximately 3,000 cases are diagnosed each year, and 60,000 patients live with this chronic, relapsing disease. CTCL can affect all ages, although it is typically diagnosed in older adults. If skin-directed therapy is inadequate or disease is advanced, the most effective systemic therapies are biologic response modifiers including interferons, rexinoids/retinoids, and selective histone deacetylase inhibitors (HDACi); chemotherapy may be used for relapsed, progressing, and/or aggressive cases [1,3]. However, current therapies are associated with an abbreviated response and subsequent drug resistance, and prognosis for patients with advanced disease is poor [1,2]. To date, allogeneic stem cell transplantation is the only potential cure

Unlike many cancers, CTCL pathogenesis remains poorly understood. Until recently, no molecular drivers had been identified, prohibiting the development of driver-based targeted therapies. Thus, identifying critical pathways and molecular drivers of CTCL is essential to understanding progression of the disease and developing effective therapies that improve quality of life and outcome for CTCL patients.

Accordingly, additional treatments are needed for curing CTCL. Described herein, inter alia, are solutions to these and other problems in the art.

### BRIEF SUMMARY OF THE INVENTION

Herein is provided, inter alia, a method of treating cutaneous T-cell lymphoma (CTCL) in a subject in need thereof. The method includes administering an effective amount of a p38 gamma (p38γ) kinase inhibitor to the subject.

In an aspect, a p38 gamma (p38γ) kinase inhibitor is a compound represented by Formula (I):

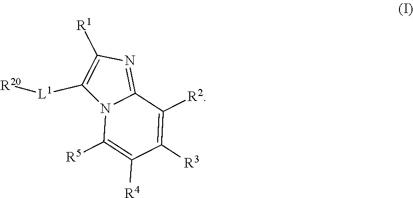

L¹ is a bond, —SOₙ₁₁L^{1A}-, —SOᵥ₁₁NR¹¹L^{1A}-, —NHC(O)NR¹¹L^{1A}-, —NR¹¹L^{1A}-, —C(O)L^{1A}-, —C(O)OL^{1A}-, —C(O)NR¹¹L^{1A}-, —OL^{1A}-, —NR¹¹SO₂L^{1A}-, —NR^{11C}(O)L^{1A}-, —NR¹¹C(O)OL^{1A}-, —NR¹¹OL^{1A}-, —SL^{1A}-, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R¹ is hydrogen, halogen, —CX¹₃, —CHX¹₂, —CH₂X¹, —OCX¹₃, —OCH₂X¹, —OCHX¹₂, —N₃, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R² is hydrogen, halogen, —CX²₃, —CHX²₂, —CH₂X², —OCX²₃, —OCH₂X², —OCHX²₂, —N₃, —CN, —SOₙ₂R^{2D}, —SOᵥ₂NR^{2A}R^{2B}, —NHC(O)NR^{2A}R^{2B}, —N(O)ₘ₂, —NR^{2A}R^{2 B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³ is hydrogen, halogen, —CX³₃, —CHX³₂, —CH₂X³, —OCX³₃, —OCH₂X³, —OCHX³₂, —N₃, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁴ is hydrogen, halogen, —CX⁴₃, —CHX⁴₂, —CH₂X⁴, —OCX⁴₃, —OCH₂X⁴, —OCHX⁴₂, —N₃, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁵ is hydrogen, halogen, —CX⁵₃, —CHX⁵₂, —CH₂X⁵, —OCX⁵₃, —OCH₂X⁵, —OCHX⁵₂, —N₃, —CN, —SOₙ₅R^{5D}, —SOᵥ₅NR^{5A}R^{5B}, —NHC(O)NR^{5A}R^{5B}, —N(O)ₘ₅, —NR^{5A}R^{5B}, —C(O)R^{5C}, —C(O)—OR^{5C}, —C(O)NR^{5A}R^{5B}, —OR^{5D}, —NR^{5A}SO₂R^{5D}, —NR^{5A}C(O)R^{5C}, —NR^{5A}C(O)OR^{5C}, —NR^{5A}OR^{5C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R²⁰ is substituted or unsubstituted cycloalkyl, substituted or unsubstituted heterocycloalkyl, substituted or unsubstituted aryl or substituted or unsubstituted heteroaryl. R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D} and R¹¹ are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L^{1A} is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. n1, n2, n3, n4, n5 and n11 are independently an integer from 0 to 4. m1, m2, m3, m4, m5, v1, v2, v3, v4, v5 and v11 are independently an integer from 1 to 2. X, X¹, X², X³, X⁴, and X⁵ are independently —F, —Cl, —Br, or —I.

In embodiments, the p38γ kinase inhibitor is a compound represented by Formula (II):

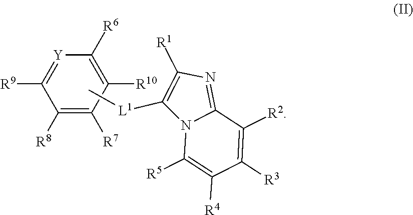

L¹, Y, R¹, R¹, R², R³, R⁴, and R⁵ are described herein. In embodiments, L¹ is —SOₙ₁₁L^{1A}-, —SOᵥ₁₁NR¹¹L^{1A}-, —NHC(O)NR¹¹L^{1A}-, —NR¹¹L^{1A}-, —C(O)L^{1A}-, —C(O)OL^{1A}-, —C(O)NR¹¹L^{1A}-, —OL^{1A}-, —NR¹¹SO₂L^{1A}-, —NR¹¹C(O)L^{1A}-, —NR¹¹C(O)OL^{1A}-, —NR¹¹OL^{1A}-, —SL^{1A}-, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. Y is —N═ or —CR¹²═.

R⁶ is a bond (to L¹), hydrogen, halogen, —CX⁶₃, —CHX⁶₂, —CH₂X⁶, —OCX⁶₃, —OCH₂X⁶, —OCHX⁶₂, —N₃, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁷ is a bond (to L¹), hydrogen, halogen, —CX⁷₃, —CHX⁷₂, —CH₂X⁷, —OCX⁷₃, —OCH₂X⁷, —OCHX⁷₂, —N₃, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁸ is a bond (to L¹), hydrogen, halogen, —CX⁸₃, —CHX⁸₂, —CH₂X⁸, —OCX⁸₃, —OCH₂X⁸, —OCHX⁸₂, —N₃, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —OR^{8D}, —NR^{8A}SO₂R^{8D}, —NR^{8A}C(O)R^{8C}, —NR^{8A}C(O)OR^{8C}, —NR^{8A}OR^{8C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁹ is a bond (to L¹), hydrogen, halogen, —CX⁹₃, —CHX⁹₂, —CH₂X⁹, —OCX⁹₃, —OCH₂X⁹, —OCHX⁹₂, —N₃, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹⁰ is a bond (to L¹), hydrogen, halogen, —CX¹⁰₃, —CHX¹⁰₂, —CH₂X¹⁰, —OCX¹⁰₃, —OCH₂X¹⁰, —OCHX¹⁰₂, —N₃, —CN, —SOₙ₁₀R^{10D}, —SOᵥ₁₀NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹² is hydrogen, halogen, —CX¹²₃, —CHX¹²₂, —CH₂X¹², —OCX¹²₃, —OCH₂X¹², —OCHX¹²₂, —N₃, —CN, —SOₙ₁₂R^{12D}, —SOᵥ₁₂NR^{12A}R^{12B}, —NHC(O)NR^{12A}R^{12B}, —N(O)ₘ₁₂, —NR^{12A}R^{12B}, —C(O)R^{12C}, —C(O)—OR^{12C}, —C(O)NR^{12A}R^{12B}, —OR^{12D}, —NR^{12A}SO₂R^{12D}, —NR^{12A}C(O)R^{12C}, —NR^{12A}C(O)OR^{12C}, —NR^{12A}OR^{12C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

One of R⁶, R⁷, R⁸, R⁹ or R¹⁰ is a bond to L¹. Stated another way, one of R⁶, R⁷, R⁸, R⁹ or R¹⁰ is absent, wherein the carbon to which the absent R⁶, R⁷, R⁸, R⁹ or R¹⁰ is attached serves as the point of attachment to L¹. R^{6A}, R^{6B}, R^{6C}, R^{6D}, R^{7A}, R^{7B}, R^{7C}, R^{7D}, R^{8A}, R^{8B}, R^{8C}, R^{8D}, R^{9A}, R^{9B}, R^{9C}, R^{9D}, R^{10A}, R^{10B}, R^{10C}, R^{10D}, R^{12A}, R^{12B}, R^{12C}, and R^{12D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁷ and R⁸ together with atoms attached thereto may optionally be joined to form substituted or unsubstituted cycloalkyl, substituted or unsubstituted heterocycloalkyl, substituted or unsubstituted aryl, or substituted or unsubstituted heteroaryl. R⁸ and R⁹ together with atoms attached thereto may optionally be joined to form substituted or unsubstituted cycloalkyl, substituted or unsubstituted heterocycloalkyl, substituted or unsubstituted aryl, or substituted or unsubstituted heteroaryl. R⁹ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl. R⁶ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl. R⁶ and R¹⁰ together with atoms attached thereto may optionally be joined to form substituted or unsubstituted cycloalkyl, substituted or unsubstituted heterocycloalkyl, substituted or unsubstituted aryl, or substituted or unsubstituted heteroaryl. R⁷ and R¹⁰ together with atoms attached thereto may optionally be joined to form substituted or unsubstituted cycloalkyl, substituted or unsubstituted heterocycloalkyl, substituted or unsubstituted aryl, or substituted or unsubstituted heteroaryl. n6, n7, n8, n9, n10 and n12 are independently an integer from 0 to 4. m6, m7, m8, m9, m10, m12, v6, v7, v8, v9, v10 and v12 are independently an integer from 1 to 2. X⁶, X⁷, X⁸, X⁹, X¹⁰ and X¹² are independently —F, —Cl, —Br, or —I.

In an aspect is provided is a method of treating a cancer in a subject in need thereof. The method includes administering a combined effective amount of a histone deacetylase (HDAC) inhibitor and a p38 gamma (p38γ) kinase inhibitor as described herein to the subject.

In an aspect is provided is a method of suppressing proliferation of a cutaneous T-cell lymphoma (CTCL) cell. The method includes contacting the cell with an effective amount of a p38 gamma (p38γ) kinase inhibitor as described herein.

In an aspect is provided a compound represented by Formula (I) described herein.

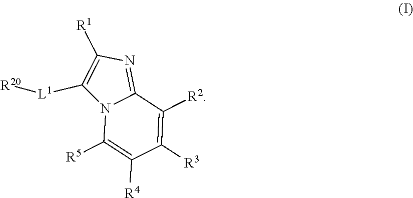

L¹, Y, R¹, R¹, R², R³, R⁴ and R⁵ are described herein. In embodiments when Y is —CH═, L¹ is —SO₂—N(CH₃)N═CH— and R⁴ is —Br, then R²⁰ is not 2-methyl-5-nitrophenyl.

In embodiments, the compound is represented by Formula (II).

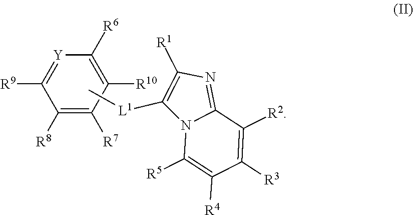

L¹, Y, R¹, R¹, R², R³, R⁴, R⁵, R⁶, R⁷, R⁸, R⁹ and R¹⁰ are described herein. In embodiments, when R¹⁰ is a bond to L¹, Y is —CH═, L¹ is —SO₂—N(CH₃)N═CH—, R⁴ is —Br and R⁶ is unsubstituted methyl, then R⁸ is not —NO₂; or when R⁸ is a bond to L¹, Y is —CH═, L¹ is —SO₂—N(CH₃)N═CH—, R⁴ is —Br and R⁹ is unsubstituted methyl, then R¹⁰ is not —NO₂

In an aspect is provided a pharmaceutical composition including a compound as described herein, or a pharmaceutically acceptable salt thereof.

### BRIEF DESCRIPTION OF THE DRAWINGS

FIG. 1 depicts p38γ as a therapeutic target in CTCL, particularly indicating a target inhibition of p38γ, combined inhibition of p38γ, and HIDAC, or optimized p38γ inhibition.

FIG. 2A depicts a kinase function of p38γ in CTCL; and FIG. 2B depicts a non-kinase function of p38γ in CTCL.

FIG. 3A is a qRT-PCR of mRNA from p38γ gene expression in CD4+ T cells in healthy donors (H1, H2) and SS patient (P1, P2). QRT-PCR analysis was used to determine mRNA expression of indicated p38γ (c), relative to their internal control GAPDH, in CD4+ T cells isolated from healthy donors (n=2) or SS patients (n=2). FIG. 3B is a microarray analysis showing p38γ is elevated in cells from the Sézary Syndrome (GSE17601) and Mycosis Fungoides (GSE12902) patients in comparison to normal T cell (GSE19069). Publically available microarray databases were analyzed for mRNA expression of p38 isoforms in CTCL (GSE17601, n=32 for SS, pink; GSE12902, n=22 for MF, green) and healthy donors/primary CD4⁺ T cell lines (GSE19069, n=8, blue). *p<0.001

FIG. 4A shows reduced proliferation of HH cells (CTCL cells) by expression of Lentiviral shRNA specific against p38γ. FIG. 4B shows inhibition of cell proliferation by p38γ siRNA (SEQ ID NO: 15, SEQ ID NO: 16) in Hut78 cells after 72 hours of treatment. FIG. 4C shows increased cell proliferation by incorporation of p38γ in p38γ depleted Hut78 cells.

FIG. 5 depicts NFATC4 gene expression level increased in SS cells and NFATC1 gene expression level decreased in SS cells, which is computed from microarray analysis of public datasets.

FIG. 6A depicts two p38 pathways, i.e. classical p38 pathways found in both T cells and others and alternative p38 pathway found solely in T cells, showing opposite effects. FIG. 6B shows reduced mRNA expression level of NFATC4 upon shRNA against p38γ but not p38β. FIG. 6C shows reduced mRNA expression level of IL-17A upon shRNA against p38γ but slightly by shRNA against p38β. FIG. 6D shows reduced mRNA expression level of IL-17A upon shRNA against NFATC4. FIG. 6E is an image from con-focal immunofluorescence microscopy showing that alternative p38 pathways activated in Hut78 cells.

FIG. 7 shows that the candidate p38γ inhibitor lead compound (Compound 1 or F7) reduced proliferation of Hut78 cells, in comparison to commercially available p38γ inhibitor Pirfernidone.

FIG. 8A shows viability of Hut 78 cells by inhibition of compound 1 after 72 hours of treatment. FIG. 8B shows comparison effects of compound 1 on Hut78 cells and healthy CD4+ T cells for their cell proliferation. FIG. 8C shows dose dependent inhibition of tumor growth in CTCL xenograft model upon treatment of compound 1.

FIG. 9A shows an enzymatic kinase assay results that was performed in vitro with human recombinant p38 α, β, γ or δ protein (active full-length). FIG. 9B shows an enzymatic kinase assay that was performed in vitro with recombinant p38 γ protein and a synthetic peptide substrate at 10 μM, 100 μM and 250 μM concentrations of ATP. FIG. 9C is determination of ATP-K_{M} value.

FIG. 10A shows reduced cell proliferation (cell number, HH cells) upon treatment with Compound 1 for 4 days at concentrations of 100 nM and 300 nM in comparison to untreated cell. FIG. 10B shows dose-dependent inhibition of tumor growth by p38γ inhibitor (Compound 1 or F7) in a CTCL xenograft model.

FIG. 11A shows mRNA level of p38γ reduced by Compound 1 (F7). FIG. 11B shows protein expression of p38γ reduced by Compound 1 (F7).

FIG. 12 shows combination of p38γ inhibitor Compound 1 and pan-HDAC inhibitor SAHA after 48 hr treatment.

FIG. 13A is a table combination of p38γ inhibitor Compound 1 and pan-HDAC inhibitor (Abexinostat) after 48 hr treatment on H9 cell. FIG. 13B is a table combination of p38γ inhibitor Compound 1 and pan-HDAC inhibitor (Abexinostat) after 48 hr treatment on Hut78 cell.

FIG. 14A shows an exemplary strategy for designing compound of p38γ inhibitor. FIG. 14B shows viability of Hut 78 cells upon treatment of a new analogue F7D3. FIG. 14C shows possible docking sites for analogues on p38γ.

FIG. 15A shows gene expression profiling of Compound 1 treatment (100 nM) shown highly positive correlation to that of the shRNA-p38 gamma (treatments vs control of Hut 78 cells) in Immuno panel of Nanostring RNA analysis. FIG. 15B shows gene expression profiling of Compound 1 treatment (500 nM) shown highly positive correlation to that of the shRNA-p38 gamma (treatments vs control of Hut 78 cells) in Pan-cancer panel Nanostring RNA analysis.

FIG. 16 is an image from con-focal immunofluorescence microscopy showing that Compound 1 (800 nM,10 hr) blocks H3K27 acetylation and the blockage can be released by sorbitol, a p38 gamma inducer/activator.

FIG. 17A show additional analogues, F7D10 and F7D11; FIG. 17B shows viability of Hut 78 cells upon treatment of the analogues F7D10 and F7D11 upon 72 hours of treatment; and FIG. 17C shows inhibition of p38γ activity upon treatment of the analogues F7D10 and F7D11.

FIGS. 18A-18C show that p38γ is elevated in CTCL and is important for viability. FIG. 18A shows that RNA seq database phs000725 was downloaded from dbGAP database with permission. Differential expression analysis of four p38 isoforms between CD4+ T cells of healthy donors (n=5, pink) and that of patients with Sézary syndrome (n=32, blue. *p<0.005) using DESeq 2 package in R, Y-axis indicates log-fold changes of expressed genes RPKM, reads per kilobase of transcript per million mapped reads. FIG. 18B is Western blot used to visualize protein expression of indicated p38 isoforms in peripheral blood mononuclear cells (PBMC) of healthy donors (n=3) and one patient with Sézary syndrome (SS), as well as whole cell lysates of Hut78 and H9 CTCL cell lines (Dotted line indicates that the blotting of H9 sample are from different parts of the same gel with the same exposure). DLGH1 is a downstream target of p38γ kinase activity, indicated by phosphorylation at the 158 residue (p-DLGH1 Ser158); phosphorylation at the unrelated 431 residue (p-DLGH1 Ser431) is shown as a control. GAPDH is a control for protein loading. FIG. 18C shows that Hut78 cells were transduced with lentiviral particles that harbor shRNA against p38γ or control shRNA. (Top) Western blot was used to visualize protein expression of p38γ. GAPDH is a control for protein loading. (Bottom) Cell viability was measured by Trypan blue exclusion and data presented as a percent of control-treated cells. Three replicates were performed for each sample, *p<0.05.

FIGS. 19A-19F are results from screening of a kinase inhibitor library for p38γ inhibitors led to the selection of F7/PIK75. (All experiments are repeated in three independent experiments and data represented are the average of triplicate experiments. CellTiterGlo Cell Viability Assay (promega) was used to measure viability in FIGS. 19B-19C). FIG. 19A shows 260 kinase inhibitor library (EMD Biosciences) screened for p38γ activity using ADP-Glo Max Assay (normalized to DMSO control). The three most potent candidates (A10, A11, and F7) are indicated. 1 μM staurosporine was used as an internal positive control. FIG. 19B shows data normalized to DMSO control, in Hut78 cells treated with varying concentrations of F7/PIK75 or SAHA (an FDA-approved drug for treatment of CTCL, used as a control). Inset table shows calculated IC₅₀ values (μM). FIG. 19C shows cell viability results normalized to DMSO control, in PBMCs isolated from SS patients and treated with varying concentrations of F7/PIK75 or SAHA for 72 h. Inset table shows calculated IC₅₀ values (μM). *p<0.05 FIG. 19D shows western blot used to visualize protein expression of indicated p38 isoforms, phosphorylated DLGH1 Ser 158 and Ser431, and actin (loading control) in Hut78 and H9 CTCL cells treated with 50 nM F7/PIK75, using indicated antibodies. FIG. 19E shows cell viability measured in CD4⁺ T cells from healthy donors (n=2) and SS patient cells (n=2) treated with 100 nM of F7/PIK75 or DMSO control. Data presented as normalized to untreated controls. FIG. 19F is western blot for CD4⁺ T cells from a healthy donor or an SS patient treated with F7/PIK75 (100 nM or 200 nM) or DMSO control to indicate expression of p38 isoforms, and actin as the loading control.

FIGS. 20A-20C show that F7 can target p38γ kinase activity in vitro and in an ATP-dependent manner. FIG. 20A shows ADP-Glo in vitro kinase assay used to calculate IC₅₀ for F7/PIK75 inhibition of kinase activity of the four p38 isoforms, normalized to DMSO control. Calculated IC₅₀ values (μM) are indicated. FIG. 20B shows time-resolved fluorescence energy transfer used to measure in vitro enzyme kinetics of inhibition of p38γ kinase at indicated concentrations of F7. CPM, counts per minute, correspond to product formation level. The error bar of the measurements represents standard deviation of triplicate data. Solid lines represent data fitting to the competitive inhibition model. Calculated Kᵢ and Kₘ values are indicated. FIG. 20C shows mapping of the CSPs induced by F7/PIK75 binding to p38γ on the docked structure of p38γ in complex with F7. F7/PIK75 forms three hydrogen bonds with K56/Y59/R70, which are displayed as blue dots. The ANP molecule X-ray structure is displayed as grey sticks for comparison. The residues with the largest line-broadening effects are indicated in red, and those with significant CSPs (>0.05 ppm) are indicated in green with their sidechains shown in stick. L58 and L170 are within 3 Å distance of F7/PIK75.

FIGS. 21A-21B show that lead compound F7/PIK75 targets p38γ kinase activity in vivo and reduces tumor volume in a dose-dependent manner in Hut78 cell xenograft mice. FIG. 21A shows that tumor volume was measured in tumors excised from Hut78 xenograft mice treated with vehicle control or 2 mg/kg or 10 mg/kg F7 for 8 days; n=7 mice per treatment; 3 tumors were measured per mouse; *p<0.05. FIG. 21B shows western blot used to visualize protein expression of indicated p38 isoforms, phosphorylated DLGH1 Ser158, and GAPDH (loading control) in tumor sections from all Hut78 xenograft mice treated with vehicle control or 2 mg/kg or 10 mg/kg F7/PIK75. FIG. 21C shows immunohistochemistry performed on tumor sections from all Hut78 xenograft mice treated with vehicle control or 10 mg/kg F7/PIK75. Representative images for staining with pDLGH1-Ser158 polyclonal antibody (brown color).

FIGS. 22A-22D show that lead compound F7/PIK75 targets activity of multiple kinases, including pI3K and p38γ. FIG. 22A shows cell viability results measured and normalized to DMSO controls in Hut78 cells treated with varying concentrations of three potent PI3K-specific inhibitors (BEZ235, GDC-0941, or A66) and F7/PIK75. Inset table shows calculated IC₅₀ values. FIG. 22B shows that cell-free-based p38γ kinase assay using ADP Glo was performed with indicated concentrations (1p M and 10 μM) of three PI3K-specific inhibitors (A66, GDC-0941, or BEZ235) and 1 μM of F7/PIK75. Staurosporine (ST; 1 μM), a pan-kinase inhibitor, was used as a positive control. FIG. 22C shows Hut78 cells transduced with shRNA against PI3K 110α or control shRNA. (Top) Western blot was used to visualize protein expression of PI3K 110α. Actin is a control for protein loading. (Bottom) Trypan blue exclusion was used to measure cell viability as a percent of control-treated cells. Three replicates were performed, *p<0.05. FIG. 22D shows western blot used to visualize A66 effects on Hut78 cells, indicated by protein expression level of downstream targets of PI3Kp110α. GAPDH is a control for protein loading.

FIGS. 23A-23D show that p38γ gene expression is limited in human tissues, and is important for viability in CTCL. FIG. 23A shows QRT-PCR analysis was used to determine mRNA expression of indicated p38 isoforms, relative to their internal control GAPDH, in CD4+ T cells isolated from healthy donors (n=2) or SS patients (n=2). FIG. 23B shows Hut78 cells treated with p38γ or control Cell viability presented as a percent of control-treated cells. Two siRNA sequences that targeting p38γ are indicated in the materials and methods. Three replicates performed for each sample, *p<0.05e. FIG. 23C shows western blot was used to visualize protein expression of indicated p38 isoforms or actin (loading control) in Hut78 cells transduced with p38γ shRNA or scrambled control. FIG. 23D shows western blot used to visualize protein expression of indicated p38 isoforms or actin (loading control) in Hut78 cells transduced with p38γ shRNA or scrambled control.

FIGS. 24A-24C show p38γ inhibitors F7/PIK75 and pirfenidone. FIG. 24A shows IC₅₀ determination in CTCL cells by pirfenidone. CELLTITERGLO assay was used to measure viability in Hut78, H9, or HH CTCL cell lines and one SS patient sample treated with DMSO (vehicle control) or Pirfenidone (125 μM). Data are an average of 3 replicates. FIG. 24B shows that p38γ inhibitor Pirfenidone effects on p38γ kinase activity ADP-Glo in vitro kinase assay was used to measure p38γ kinase activity in with varying concentrations of F7 (PIK75) or Pirfenidone, normalized to DMSO control in cell-free-based assays. Data are an average of 3 replicates. FIG. 24C shows that PIK75 interferes p38γ kinase activities. Western blot was used to visualize protein expression of indicated p38 isoforms, phosphorylated DLGH1 Ser158, and actin (loading control) in CD4+ T cells from healthy donors (n=2) or SS patients (n=2) treated with F7/PIK75 (100 nM) or DMSO control for 24 h.

FIG. 25 shows assignments of F7/PIK75 to p38γ residues by NMR experiment. Overlay of the 1H-13C HMQC spectra in the methyl region for p38γ, free (red), and in complex with F7/PIK75 (blue). The peaks that undergo large chemical shift changes (CSP>0.05 ppm) or line broadening are labeled with their corresponding residue number.

FIG. 26 shows A66 effects on cell-based analysis. Western blot was used to visualize A66 effects on Hut78 cells, indicated by protein expression level of downstream targets of PI3Kp110α. GAPDH is a control for protein loading.

FIG. 27 shows RNA seq analysis shows p38γ expression is elevated in 32 SS CTCL patients (right) vs. 5 healthy donors (left).

FIG. 28 shows Cell viability was measured and normalized to DMSO controls in Hut78 cells treated with varying concentrations of the potent PI3K-specific inhibitor A66 or F7. Inset table shows calculated IC₅₀ values.

FIG. 29A-29B show structure-based F7 derivatives design: based on the L¹ length (orange bar). FIG. 29A is adjustment of L1 length of F7 derivatives affects the viability of CTCL cells (bottom left). FIG. 29B is novel designed compounds based on the L1 length (bottom) show various docking scores to p38γ (top).

FIGS. 30A-30C show combined p38γ inhibition and HDACi to target complementary pathways and induce synergistic therapeutic effects. FIG. 30A shows combination of p38γ inhibitor F7 and pan-HDACi Abexinostat (Abex) show synergistic inhibition of CTCL growth at 48 h with indicated concentrations. FIG. 30B shows western blot for downstream targets of single/combined F7 and Abex treatment. FIG. 30C shows gene silencing of p38γ affects cell viability and downstream targets.

FIG. 31 is a scheme of synthetic lethalscreen: To further define the role of p38 and identify targets that increase the antitumor efficacy of p38 inhibition, we performed a synthetic lethal RNA interference (RNAi) screen in Hut78 cells treated with 10 μM of the p38 MAPK inhibitor Ly2228820. We transduced control and Ly2228820-treated Hut78 cells with a pooled retroviral RNAi library consisting of 4290 shRNAs that targeted more than 1000 genes involved in human cancers. If a shRNA from the library is not toxic to the control cells, but causes cell death in Ly2228820-treated cells, the gene targeted by this shRNA would be identified by the screen as synthetically lethal to p38 inhibition. Among many hits identified from the screen, we selected HDAC3 for further analysis.

FIG. 32A shows that docking poses of F7 with p38γ (left) and PI3Kγ (right) in ATP binding site. FIG. 32B shows F7-protein interaction diagram, p38γ (left) and PI3Kγ (right).

FIG. 33 is a schematic outline of medicinal chemistry strategy to optimize lead.

FIGS. 34A-34B show that high-density CRISPR protein scan identifies novel functional elements in Dot1l. FIG. 34A is a schematic outline of the sgRNA library construction and pooled library screen in MLL-AF9-Cas9+ cells. FIG. 34B is PRALINE multiple sequence alignment of Dot1l across evolution (human, mouse, chicken, turtle, frog, zebrafish, fly). Red indicates highly conserved, and blue indicates more diverse between species. Solid black boxes indicate known functional domains. Dot-plots depict changes in representation of each sgRNA construct in the screen before v.s. after 12 days of culture. KMT: lysine methyltransferase. R1: de novo identified functional elements.

FIGS. 35A-35C are identification of novel drugable site in p38γ by virtual ligand screen and high-density CRISPR protein scan. FIG. 35A is docking of the NCI compounds (˜260,000) on p38γ crystal structure identifies three potential drugable sites. FIG. 35B is a structural schema of the three compound docking sites on p38γ. FIG. 35C is PRALINE multiple sequence alignment of human p38 kinase family. Red indicates highly conserved, and blue indicates more diverse between p38 family proteins. Black arrowheads indicate the positions of sgRNA (total 150sgRNA) designed for p38γ high-density CRISPR scan. Dotted box indicates the lipid-binding domains (site 2, and Site 3; total 60 sgRNA targeting this region). The center of Site 2(E195) and Site3(K270), and a critical amino acid known to bind ATP (V41m Site 1) are labeled.

FIG. 36 shows western blot analysis of p38γ MAPK signals in 4 SS patients using the Abcam p38γ MAPK antibody.

### DETAILED DESCRIPTION OF THE INVENTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals, having the number of carbon atoms designated (i.e., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., selected from the group consisting of O, N, P, Si, and S), and wherein the nitrogen and sulfur atoms may optionally be oxidized, and the nitrogen heteroatom may optionally be quaternized. The heteroatom(s) (e.g., O, N, P, S, B, As, and Si) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heteroalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be a —O— bonded to a ring heteroatom nitrogen.

A “fused ring aryl-heterocycloalkyl” is an aryl fused to a heterocycloalkyl. A “fused ring heteroaryl-heterocycloalkyl” is a heteroaryl fused to a heterocycloalkyl. A “fused ring heterocycloalkyl-cycloalkyl” is a heterocycloalkyl fused to a cycloalkyl. A “fused ring heterocycloalkyl-heterocycloalkyl” is a heterocycloalkyl fused to another heterocycloalkyl. Fused ring aryl-heterocycloalkyl, fused ring heteroaryl-heterocycloalkyl, fused ring heterocycloalkyl-cycloalkyl, or fused ring heterocycloalkyl-heterocycloalkyl may each independently be unsubstituted or substituted with one or more of the substituents described herein. Fused ring aryl-heterocycloalkyl, fused ring heteroaryl-heterocycloalkyl, fused ring heterocycloalkyl-cycloalkyl, or fused ring heterocycloalkyl-heterocycloalkyl may each independently be named according to the size of each of the fused rings. Thus, for example, 6,5 aryl-heterocycloalkyl fused ring describes a 6 membered aryl moiety fused to a 5 membered heterocycloalkyl. Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The term “oxo,” as used herein, means an oxygen that is double bonded to a carbon atom.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, —NR′SO₂R″, —NR′C═(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C═(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), Boron (B), Arsenic (As), and silicon (Si).

A “substituent group,” as used herein, means a group selected from the following moieties:

(A) oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
(i) oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl, unsubstituted heteroalkyl, unsubstituted cycloalkyl, unsubstituted heterocycloalkyl, unsubstituted aryl, unsubstituted heteroaryl, and
(ii) alkyl, heteroalkyl, cycloalkyl, heterocycloalkyl, aryl, and heteroaryl, substituted with at least one substituent selected from:
(a) oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl, unsubstituted heteroalkyl, unsubstituted cycloalkyl, unsubstituted heterocycloalkyl, unsubstituted aryl, unsubstituted heteroaryl, and
(b) alkyl, heteroalkyl, cycloalkyl, heterocycloalkyl, aryl, or heteroaryl, substituted with at least one substituent selected from: oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In embodiments, each substituted group described in the compounds herein is substituted with at least one substituent group. More specifically, In embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In embodiments herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those that are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the (R) and (S) configurations for each asymmetric center. Therefore, single stereochemical isomers as well as enantiomeric and diastereomeric mixtures of the present compounds, generally recognized as stable by those skilled in the art, are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, replacement of fluoride by ¹⁸F, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (125I), or carbon-14 (¹⁴C). All isotopic variations of the compounds of the present invention, whether radioactive or not, are encompassed within the scope of the present invention.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

The symbol “ [symbol] ” denotes the point of attachment of a chemical moiety to the remainder of a molecule or chemical formula.

Where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman decimal symbol may be used to distinguish each appearance of that particular R group. For example, where multiple R¹³ substituents are present, each R¹³ substituent may be distinguished as R^{13.1}, R^{13.2}, R^{13.3}, R^{13.4}, etc., wherein each of R^{13.1}, R^{13.2}, R^{13.3}, R^{13.4}, etc. is defined within the scope of the definition of R¹³ and optionally differently. The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Description of compounds of the present invention is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The terms “p38 kinase”, “p38 mitogen-activated protein (MAP) kinase” and/or “p38” are here used interchangeably and according to their common, ordinary meaning and refer to proteins of the same or similar names and functional fragments and homologs thereof. The term includes recombinant or naturally occurring forms of, or variants thereof, that maintain p38 kinase activity (e.g. within at least 30%, 40%, 50%, 60%, 70%, 80%, 90%, 95%, or 100% activity compared to p38 kinase). The p38 kinases have four isoforms such as p38a (MAPK14, SEQ ID NO: 11), p38β (MAPK 11, SEQ ID NO: 12), p38γ (MAPK12, SEQ ID NO: 13), and p38δ (MAPK13, SEQ ID NO: 14).

The terms “p38alpha (p38α)” or “mitogen-activated protein kinase 14 (MAPK14)” (e.g. Protein Data Bank ID: 5ML5 or 5MQV; SEQ ID NO: 11) are here used interchangeably and according to their common, ordinary meaning and refer to proteins of the same or similar names and functional fragments and homologs thereof. The term includes any recombinant or naturally occurring form of, or variants thereof that maintain p38a activity (e.g. within at least 30%, 40%, 50%, 60%, 70%, 80%, 90%, 95%, or 100% activity compared to p38α).

The terms “p38beta (p38p)” or “mitogen-activated protein kinase 11 (MAPK11)” (e.g. Protein Data Bank ID: 3GC7, 3GC8 or 3GC9; SEQ ID NO: 12) are here used interchangeably and according to their common, ordinary meaning and refer to proteins of the same or similar names and functional fragments and homologs thereof. The term includes any recombinant or naturally occurring form of, or variants thereof that maintain p38p activity (e.g. within at least 30%, 40%, 50%, 60%, 70%, 80%, 90%, 95%, or 100% activity compared to p38β).

The terms “p38gamma (p38γ)” or “mitogen-activated protein kinase 12 (MAPK12)” (e.g., Protein Data Bank ID: 4QUM or 4QUN; SEQ ID NO: 13) are here used interchangeably and according to their common, ordinary meaning and refer to proteins of the same or similar names and functional fragments and homologs thereof. The term includes any recombinant or naturally occurring form of, or variants thereof that maintain p38γ activity (e.g. within at least 30%, 40%, 50%, 60%, 70%, 80%, 90%, 95%, or 100% activity compared to p38γ).

The terms “p38delta (p38δ)” or “mitogen-activated protein kinase 13 (MAPK13)” (e.g. Protein Data Bank ID: 4MYG, 5EKN or 5EKO; SEQ ID NO: 14) are here used interchangeably and according to their common, ordinary meaning and refer to proteins of the same or similar names and functional fragments and homologs thereof. The term includes any recombinant or naturally occurring form of, or variants thereof that maintain p38δ activity (e.g. within at least 30%, 40%, 50%, 60%, 70%, 80%, 90%, 95%, or 100% activity compared to p38δ).

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

In some embodiments, the term “inhibition”, “inhibit”, “inhibiting” and the like means negatively affecting (e.g. decreasing or suppressing) the expression of the protein relative to the expression level of the protein in the absence of the inhibitor. In embodiments inhibition means negatively affecting (e.g. decreasing or suppressing) transcription or expression level of mRNA of the protein relative to the transcription or expression level of the mRNA of the protein in the absence of the inhibitor. In embodiments inhibition means negatively affecting (e.g. decreasing or suppressing) expression level of the protein relative to the expression level of the protein in the absence of the inhibitor by elevating or increasing a concentration of a biological molecule which negatively affecting (e.g. decreasing or suppressing) the expression level of the protein.

The terms “p38 inhibitors” or “p38 kinase inhibitors” are agents (e.g. compounds) that reduce the activity, levels and/or expression of p38 relative to the absence of the inhibitor. In embodiments, these p38 kinase inhibitors can sufficiently inhibit the activities of one or more p38 related protein kinases or proteins in p38 related signal transduction cascades. In embodiments, the p38 kinase inhibitors sufficiently suppress or downregulate the expression of p38 kinases, for example, by affecting or suppressing transcription level of mRNA of p38 kinase, protein expression level thereof or other indications for related genes thereof. Non-limiting examples of the p38 inhibitors include small molecules (e.g. synthetic small molecules or natural products and derivatives thereof), antibodies (e.g. monoclonal antibodies), nucleic acids (e.g. siRNA, microRNA and anti-microRNA), and peptides.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

The term “small molecule” or the like as used herein refers, unless indicated otherwise, to a molecule having a molecular weight of less than about 700 Dalton, e.g., less than about 700, 650, 600, 550, 500, 450, 400, 350, 300, 250, 200, 100, or even 50 Dalton.

Antibodies are large, complex molecules (e.g., molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

Accordingly, the term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into a Fab′ monomer. The Fab′ monomer is essentially Fab with part of the hinge region (see FUNDAMENTAL IMMUNOLOGY (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

The term “nucleic acid” refers to deoxyribonucleotides or ribonucleotides and polymers thereof in either single-, double- or multiple-stranded form, or complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (e.g. siRNA and shRNA), and hybrid molecules having mixtures of single and double stranded DNA and RNA. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including nucleic acids with a phosphothioate backbone can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press); and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer. A “fusion protein” refers to a chimeric protein encoding two or more separate protein sequences that is recombinantly expressed as a single moiety. In embodiments, peptides are amino acid polymers of 2-1000, 2-900, 2-800, 2-700, 2-600, 2-500, 2-400, 2-300, 2-100, 2-90, 2-80, 2-70, 2-60, 2-50, 2-40, 2-30, 2-20, 2-10, 2-9, 2-8, 2-7, 2-6, 2-5, 2-4, or 2-3 amino acids. In embodiments, peptides are amino acid polymers of molecule weight of about 120-120000, 120-108000, 120-84000, 120-72000, 120-60000, 120-48000, 120-36000, 120-24000, 12000, 120-10800, 120-9600, 120-8400, 120-7200, 120-6000, 120-4800, 120-3600, 120-2400, 120-1200, 120-1080, 120-960, 120-840, 120-720, 120-600, 120-480, 120-360, or about 120-240 Dalton.

The terms “Histone deacetylase (HDAC) inhibitors (HDACi or HDIs)” are here used to indicate any molecules that sufficiently inhibit the activities (e.g. acetylation) of the histone deacetylases. In addition, these HDAC inhibitors inhibit activities (e.g. acetylation) of the proteins or enzymes included in nonhistone transcription factors and transcriptional co-regulators by increasing or repressing the transcription of genes such as ACTR, cMyb, E2F1, EKLF, FEN 1, GATA, HNF-4, HSP90, Ku70, NF-κB, PCNA, p53, RB, Runx, SF1 Sp3, STAT, TFIIE, TCF, YY1, and the like. Non-limiting examples of the HDAC inhibitors include HDAC5 inhibitor, HDAC6 inhibitor, HDAC10 inhibitor, and HDAC11 inhibitor. Non-limiting examples of the HDAC inhibitors include small molecules (e.g. synthetic small molecules or natural products and derivatives thereof), antibodies (e.g. monoclonal antibodies), nucleic acids (e.g. siRNA, microRNA and anti-microRNA), and peptides. Non-limiting examples of the small molecules as HDACi include HDAC inhibitors include Vorinostat (SAHA), Romidepsin, Abexinostat, CI-994, Belinostat, Panobinostat, Givinostat, Entinostat, Mocetinostat, Trichostatin, SRT501, CUDC-101, JNJ-26481585, Quisinostat, RGFP109 or PCI24781.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Prodrugs of the compounds described herein may be converted in vivo after administration. Additionally, prodrugs can be converted to the compounds of the present invention by chemical or biochemical methods in an ex vivo environment, such as, for example, when contacted with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Patient,” “subject,” “patient in need thereof,” and “subject in need thereof” are herein used interchangeably and refer to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

An “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may also include allowing two species to react, interact, or physically touch, wherein the two species may be a compound as described herein and a protein or enzyme. Contacting may include allowing a compound described herein to interact with a protein or enzyme that is involved in a signaling pathway.

As defined herein, the term “activation,” “activate,” “activating” and the like in reference to a protein-activator interaction means positively affecting (e.g. increasing) the activity or function of the protein relative to the activity or function of the protein in the absence of the activator. Activation may refer to reduction of a disease or symptoms of disease. Activation may refer to an increase in the activity of a particular protein or nucleic acid target. The protein may be cystic fibrosis transmembrane conductance regulator. Thus, activation includes, at least in part, partially or totally increasing stimulation, increasing, promoting, or expediting activation, or activating, sensitizing, or up-regulating signal transduction or enzymatic activity or the amount of a protein.

The term “modulator” refers to a composition that increases or decreases the level of a target molecule or the function of a target molecule or the physical state of the target of the molecule.

The term “modulate” is used in accordance with its plain ordinary meaning and refers to the act of changing or varying one or more properties. “Modulation” refers to the process of changing or varying one or more properties. For example, a modulator of a target protein changes by increasing or decreasing a property or function of the target molecule or the amount of the target molecule. A modulator of a disease decreases a symptom, cause, or characteristic of the targeted disease.

“Selective” or “selectivity” or the like of a compound refers to the compound's ability to discriminate between molecular targets. “Specific”, “specifically”, “specificity”, or the like of a compound refers to the compound's ability to cause a particular action, such as inhibition, to a particular molecular target with minimal or no action to other proteins in the cell.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal) compatible with the preparation. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

“Co-administer” it is meant that a composition described herein is administered at the same time, just prior to, or just after the administration of one or more additional therapies. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered transdermally, by a topical route, or formulated as applicator sticks, solutions, suspensions, emulsions, gels, creams, ointments, pastes, jellies, paints, powders, and aerosols.

The compositions disclosed herein can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions disclosed herein can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions can also be delivered as nanoparticles.

Pharmaceutical compositions may include compositions wherein the active ingredient (e.g. compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule, and/or reducing, eliminating, or slowing the progression of disease symptoms.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

The compounds described herein can be used in combination with one another, with other active drugs known to be useful in treating a disease (e.g. cancer or CTCL) or with adjunctive agents that may not be effective alone, but may contribute to the efficacy of the active agent. Thus, the compounds described herein may be co-administered with one another or with other active drugs known to be useful in treating a disease.

By “co-administer” it is meant that a compound described herein is administered at the same time, just prior to, or just after the administration of one or more additional therapies, for example, an anti-constipation or anti-dry eye agent as described herein. The compounds described herein can be administered alone or can be co-administered to the patient. Co-administration is meant to include simultaneous or sequential administration of the compound individually or in combination (more than one compound or agent). Thus, the preparations can also be combined, when desired, with other active substances (e.g. anti-constipation or anti-dry eye agents).

Co-administration includes administering one active agent (e.g. a complex described herein) within 0.5, 1, 2, 4, 6, 8, 10, 12, 16, 20, or 24 hours of a second active agent (e.g. anti-constipation or anti-dry eye agents). Also contemplated herein, are embodiments, where co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. Co-administration can be accomplished by co-formulation, i.e., preparing a single pharmaceutical composition including both active agents. In other embodiments, the active agents can be formulated separately. The active and/or adjunctive agents may be linked or conjugated to one another. The compounds described herein may be combined with treatments for constipation and dry eye disorders.

The terms “synergy”, “synergism” “synergistic” and “synergistic therapeutic effect” are used herein interchangeably and refer to a measured effect of compounds administered in combination where the measured effect is greater than the sum of the individual effects of each of the compounds administered alone as a single agent.

As used herein, the term “cancer” refers to all types of cancer, neoplasm, or malignant tumors found in mammals, including leukemia, carcinomas and sarcomas. Exemplary cancers include acute myeloid leukemia (“AML”), chronic myelogenous leukemia (“CML”), and cancer of the brain, breast, pancreas, colon, liver, kidney, lung, non-small cell lung, melanoma, ovary, sarcoma, and prostate. Additional examples include, cervix cancers, stomach cancers, head & neck cancers, uterus cancers, mesothelioma, metastatic bone cancer, Medulloblastoma, Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas cancer, prostate cancer, breast cancer including triple negative breast cancer, and cutaneous T-cell lymphoma.

The term “lymphoma” refers to a group of blood cell tumors that develop from cells of the immune system found in lymph, i.e. lymphocytes (e.g. natural killer cells (NK cells), T cells, and B cells). Lymphoma is typically classified into Hodgkin's lymphomas (HL) and the non-Hodgkin lymphomas (NHL) or based on whether it develops in B-lymphocytes (B-cells) or T-lymphocytes (T-cells). In embodiments, lymphoma is developed in B-cells. In embodiments, lymphoma is developed in T-cell.

The term “cutaneous T-cell lymphoma” or CTCL refers to a typical T-cell lymphoma that involves skin, although CTCL also can involve the blood, the lymph nodes, and other internal organs. Non-limiting examples of CTCL include mycosis fungoides and Sézary syndrome. For instance, mycosis fungoides is the most common type of CTCL constituting half cases of all CTCLs, which may cause various skin symptoms such as patches, plaques, or tumors. Meanwhile, Sézary syndrome is an advanced, variant form of mycosis fungoides, which can be characterized by the presence of lymphoma cells (e.g., B-cells or T-cells) in the blood.

Cancer model organism, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g. mice) and from humans.

An “anticancer agent” as used herein refers to a molecule (e.g. compound, peptide, protein, nucleic acid, antibody) used to treat cancer through destruction or inhibition of cancer cells or tissues. Anticancer agents may be selective for certain cancers or certain tissues. In embodiments, anticancer agents herein may include epigenetic inhibitors and multi- or specific kinase inhibitors (e.g. p38γ kinase inhibitor).

An “epigenetic inhibitor” as used herein, refers to an inhibitor of an epigenetic process, such as DNA methylation (a DNA methylation Inhibitor) or modification of histones (a Histone Modification Inhibitor). An epigenetic inhibitor may be a histone-deacetylase (HDAC) inhibitor, a DNA methyltransferase (DNMT) inhibitor, a histone methyltransferase (HMT) inhibitor, a histone demethylase (HDM) inhibitor, or a histone acetyltransferase (HAT). Non-limiting examples of HDAC inhibitors include Vorinostat (SAHA), Romidepsin, Abexinostat, CI-994, Belinostat, Panobinostat, Givinostat, Entinostat, Mocetinostat, Trichostatin, SRT501, CUDC-101, JNJ-26481585, Quisinostat, RGFP109 or PCI24781. Examples of DNMT inhibitors include azacitidine and decitabine. Examples of HMT inhibitors include EPZ-5676. Examples of HDM inhibitors include pargyline and tranylcypromine. Examples of HAT inhibitors include CCT077791 and garcinol.

“Selective” or “selectivity” or the like of a compound refers to the compound's ability to discriminate between molecular targets (e.g. a compound having selectivity toward one or more of p38 kinases (p38α, p38β, p38γ and p38δ) or MAPK (e.g. MAPK 11, MAPK12, MAPK 13 and MAPK14)).

“Specific”, “specifically”, “specificity”, or the like of a compound refers to the compound's ability to cause a particular action, such as inhibition, to a particular molecular target with minimal or no action to other proteins in the cell (e.g. a compound having specificity towards p38 gamma kinase (p38γ) or MAPK12 displays inhibition of the activity of those proteins including suppression of expression thereof as well as inhibition of enzyme properties). Meanwhile, the same compound displays little-to-no inhibition of other p38 kinases such as p38α, p38β and p38δ or MAPK such as MAPK 11, MAPK 13 and MAPK14.

The term “associated” or “associated with” in the context of a substance or substance activity or function associated with a disease means that the disease is caused by (in whole or in part), a symptom of the disease is caused by (in whole or in part) the substance or substance activity or function, or a side-effect of the compound (e.g. toxicity) is caused by (in whole or in part) the substance or substance activity or function.

“Disease” or “condition” refer to a state of being or health status of a patient or subject capable of being treated with the compounds or methods provided herein. Disease as used herein may refer to constipation or dry eye disorders.

Examples of anti-constipation agents include, but are not limited to diphenylmethanes, Lactobacillus paracasei, linaclotide and lubiprostone. Examples of anti-dry eye agents include, but are not limited to, topical cyclosporine, P321 (an ENaC inhibitor) and Diquafosol.

### II. Compounds

Provided herein are compounds having a structure of Formula (I):

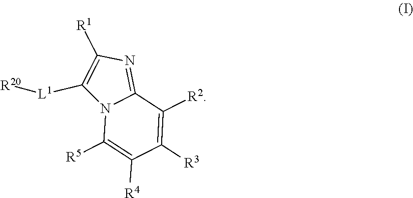

L¹ is a bond, —SOₙ₁₁L^{1A}-, —SOᵥ₁₁NR¹¹L^{1A}-, —NHC(O)NR¹¹L^{1A}-, —NR¹¹L^{1A}-, —C(O)L^{1A}-, —C(O)OL^{1A}-, —C(O)NR¹¹L^{1A}-, —OL^{1A}-, —NR¹¹SO₂L^{1A}-, —NR¹¹C(O)L^{1A}-, —NR¹¹C(O)OL^{1A}-, —NR¹¹OL^{1A}-, —SL^{1A}-, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. R¹ is hydrogen, halogen, —CX¹₃, —CHX¹₂, —CH₂X¹, —OCX¹₃, —OCH₂X¹, —OCHX¹₂, —N₃, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R² is hydrogen, halogen, —CX²₃, —CHX²₂, —CH₂X², —OCX²₃, —OCH₂X², —OCHX²₂, —N₃, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R³ is hydrogen, halogen, —CX³₃, —CHX³₂, —CH₂X³, —OCX³₃, —OCH₂X³, —OCHX³₂, —N₃, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁴ is hydrogen, halogen, —CX⁴₃, —CHX⁴₂, —CH₂X⁴, —OCX⁴₃, —OCH₂X⁴, —OCHX⁴₂, —N₃, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁵ is hydrogen, halogen, —CX⁵₃, —CHX⁵₂, —CH₂X⁵, —OCX⁵₃, —OCH₂X⁵, —OCHX⁵₂, —N₃, —CN, —SOₙ₅R^{5D}, —SOᵥ₅NR^{5A}R^{5B}, —NHC(O)NR^{5A}R^{5B}, —N(O)ₘ₅, —NR^{5A}R^{5B}, —C(O)R^{5C}, —C(O)—OR^{5C}, —C(O)NR^{5A}R^{5B}, —OR^{5D}, —NR^{5A}SO₂R^{5D}, —NR^{5A}C(O)R^{5C}, —NR^{5A}C(O)OR^{5C}, —NR^{5A}OR^{5C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R²⁰ is substituted or unsubstituted cycloalkyl, substituted or unsubstituted heterocycloalkyl, substituted or unsubstituted aryl, or substituted or unsubstituted heteroaryl. R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D} and R¹¹ are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. L^{1A} is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. n1, n2, n3, n4, n5 and n11 are independently an integer from 0 to 4. m1, m2, m3, m4, m5, v1, v2, v3, v4, v5 and v11 are independently an integer from 1 to 2. X, X¹, X², X³, X⁴, and X⁵ are independently —F, —Cl, —Br, or —I.

In embodiments, R²⁰ is substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R²⁰ is substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R²⁰ is substituted or unsubstituted heterocycloalkyl (e.g., 3 to 8 membered, 3 to 6 membered, 4 to 6 membered, 4 to 5 membered, or 5 to 6 membered), or substituted or unsubstituted heteroaryl (e.g., 5 to 10 membered, 5 to 9 membered, or 5 to 6 membered). In embodiments, R²⁰ is substituted or unsubstituted heterocycloalkyl (e.g., 3 to 8 membered, 3 to 6 membered, 4 to 6 membered, 4 to 5 membered, or 5 to 6 membered). In embodiments, R²⁰ is substituted or unsubstituted heteroaryl (e.g., 5 to 10 membered, 5 to 9 membered, or 5 to 6 membered).

In embodiments, R²⁰ is substituted or unsubstituted C₃-C₈ cycloalkyl. In embodiments, R²⁰ is substituted or unsubstituted C₃-C₆ cycloalkyl. In embodiments, R²⁰ is substituted or unsubstituted C₄-C₆ cycloalkyl. In embodiments, R²⁰ is substituted or unsubstituted C₅-C₆ cycloalkyl. In embodiments, R²⁰ is unsubstituted C₃-C₈ cycloalkyl. In embodiments, R²⁰ is unsubstituted C₃-C₆ cycloalkyl. In embodiments, R²⁰ is unsubstituted C₄-C₆ cycloalkyl. In embodiments, R²⁰ is unsubstituted C₅-C₆ cycloalkyl. In embodiments, R²⁰ is substituted or unsubstituted 3 to 8 membered heterocycloalkyl. In embodiments, R²⁰ is substituted or unsubstituted 3 to 6 membered heterocycloalkyl. In embodiments, R²⁰ is substituted or unsubstituted 4 to 6 membered heterocycloalkyl. In embodiments, R²⁰ is substituted or unsubstituted 4 to 5 membered heterocycloalkyl. In embodiments, R²⁰ is substituted or unsubstituted 5 to 6 membered heterocycloalkyl. In embodiments, R²⁰ is unsubstituted 3 to 8 membered heterocycloalkyl. In embodiments, R²⁰ is unsubstituted 3 to 6 membered heterocycloalkyl. In embodiments, R²⁰ is unsubstituted 4 to 6 membered heterocycloalkyl. In embodiments, R²⁰ is unsubstituted 4 to 5 membered heterocycloalkyl. In embodiments, R²⁰ is unsubstituted 5 to 6 membered heterocycloalkyl. In embodiments, R²⁰ is substituted or unsubstituted C₆-C₁₀ aryl. In embodiments, R²⁰ is substituted or unsubstituted phenyl. In embodiments, R²⁰ is unsubstituted C₆-C₁₀ aryl. In embodiments, R²⁰ is unsubstituted phenyl. In embodiments, R²⁰ is substituted or unsubstituted 5 to 10 membered heteroaryl. In embodiments, R²⁰ is substituted or unsubstituted 5 to 9 membered heteroaryl. In embodiments, R²⁰ is substituted or unsubstituted 5 to 6 membered heteroaryl. In embodiments, R²⁰ is substituted or unsubstituted pyridyl. In embodiments, R²⁰ is substituted or unsubstituted piperazinyl. In embodiments, R²⁰ is substituted or unsubstituted pyridazinyl. In embodiments, R²⁰ is substituted or unsubstituted pyrimidyl. In embodiments, R²⁰ is substituted or unsubstituted thienyl. In embodiments, R²⁰ is substituted or unsubstituted furanyl. In embodiments, R²⁰ is substituted or unsubstituted thiazolyl. In embodiments, R²⁰ is substituted or unsubstituted imidazo[1,2-a]pyridine. In embodiments, R²⁰ is substituted or unsubstituted naphthyl. In embodiments, R²⁰ is substituted or unsubstituted indolyl. In embodiments, R²⁰ is substituted or unsubstituted 3H-indolyl.

In embodiments, R²⁰ is R¹⁶-substituted C₃-C₅ cycloalkyl. In embodiments, R²⁰ is R¹⁶-substituted C₃-C₆ cycloalkyl. In embodiments, R²⁰ is R¹⁶-substituted C₄-C₆ cycloalkyl. In embodiments, R²⁰ is R¹⁶-substituted C₅-C₆ cycloalkyl. In embodiments, R²⁰ is unsubstituted C₃-C₈ cycloalkyl. In embodiments, R²⁰ is unsubstituted C₃-C₆ cycloalkyl. In embodiments, R²⁰ is unsubstituted C₄-C₆ cycloalkyl. In embodiments, R²⁰ is unsubstituted C₅-C₆ cycloalkyl.

In embodiments, R²⁰ is R¹⁶-substituted 3 to 8 membered heterocycloalkyl. In embodiments, R²⁰ is R¹⁶-substituted 3 to 6 membered heterocycloalkyl. In embodiments, R²⁰ is R¹⁶-substituted 4 to 6 membered heterocycloalkyl. In embodiments, R²⁰ is R¹⁶-substituted 4 to 5 membered heterocycloalkyl. In embodiments, R²⁰ is R¹⁶-substituted 5 to 6 membered heterocycloalkyl. In embodiments, R²⁰ is unsubstituted 3 to 8 membered heterocycloalkyl. In embodiments, R²⁰ is unsubstituted 3 to 6 membered heterocycloalkyl. In embodiments, R²⁰ is unsubstituted 4 to 6 membered heterocycloalkyl. In embodiments, R²⁰ is unsubstituted 4 to 5 membered heterocycloalkyl. In embodiments, R²⁰ is unsubstituted 5 to 6 membered heterocycloalkyl.

In embodiments, R²⁰ is R¹⁶-substituted C₆-C₁₀ aryl. In embodiments, R²⁰ is R¹⁶-substituted phenyl. In embodiments, R²⁰ is unsubstituted C₆-C₁₀ aryl. In embodiments, R²⁰ is unsubstituted phenyl. In embodiments, R²⁰ is R¹⁶-substituted 5 to 10 membered heteroaryl. In embodiments, R²⁰ is R¹⁶-substituted 5 to 9 membered heteroaryl. In embodiments, R²⁰ is R¹⁶-substituted 5 to 6 membered heteroaryl. In embodiments, R²⁰ is R¹⁶-substituted pyridyl. In embodiments, R²⁰ is R¹⁶-substituted piperazinyl. In embodiments, R²⁰ is R¹⁶-substituted pyridazinyl. In embodiments, R²⁰ is R¹⁶-substituted pyrimidyl. In embodiments, R²⁰ is R¹⁶-substituted thienyl. In embodiments, R²⁰ is R¹⁶-substituted furanyl. In embodiments, R²⁰ is R¹⁶-substituted thiazolyl. In embodiments, R²⁰ is R¹⁶-substituted imidazo[1,2-a]pyridine. In embodiments, R²⁰ is R¹⁶-substituted naphthyl. In embodiments, R²⁰ is R¹⁶-substituted indolyl. In embodiments, R²⁰ is R¹⁶-substituted 3H-indolyl.

In embodiments, R²⁰ is unsubstituted C₆-C₁₀ aryl. In embodiments, R²⁰ is unsubstituted phenyl. In embodiments, R²⁰ is unsubstituted C₆-C₁₀ aryl. In embodiments, R²⁰ is unsubstituted phenyl. In embodiments, R²⁰ is unsubstituted 5 to 10 membered heteroaryl. In embodiments, R²⁰ is unsubstituted 5 to 9 membered heteroaryl. In embodiments, R²⁰ is unsubstituted 5 to 6 membered heteroaryl. In embodiments, R²⁰ is unsubstituted pyridyl. In embodiments, R²⁰ is unsubstituted piperazinyl. In embodiments, R²⁰ is unsubstituted pyridazinyl. In embodiments, R²⁰ is unsubstituted pyrimidyl. In embodiments, R²⁰ is unsubstituted thienyl. In embodiments, R²⁰ is unsubstituted furanyl. In embodiments, R²⁰ is unsubstituted thiazolyl. In embodiments, R²⁰ is unsubstituted imidazo[1,2-a]pyridine. In embodiments, R²⁰ is unsubstituted naphthyl. In embodiments, R²⁰ is unsubstituted indolyl. In embodiments, R²⁰ is unsubstituted 3H-indolyl.

In embodiments, R¹⁶ is independently halogen (e.g., —F, —Cl, Br, —I), —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, R¹⁶ is independently halogen (e.g., —F, —Cl, Br, —I), —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{16E}-substituted or unsubstituted alkyl (e.g., C₁-C₈, C₁-C₆, C₁-C₄, or C₁-C₂), R^{16E}-substituted or unsubstituted heteroalkyl (e.g., 2 to 8 membered, 2 to 6 membered, 4 to 6 membered, 2 to 3 membered, or 4 to 5 membered), R^{16E}-substituted or unsubstituted cycloalkyl (e.g., C₃-C₈, C₃-C₆, C₄-C₆, or C₅-C₆), R^{16E}-substituted or unsubstituted heterocycloalkyl (e.g., 3 to 8 membered, 3 to 6 membered, 4 to 6 membered, 4 to 5 membered, or 5 to 6 membered), R^{16E}-substituted or unsubstituted aryl (e.g., C₆-C₁₂, C₆-C₁₀, or phenyl), or R^{16E}-substituted or unsubstituted heteroaryl (e.g., 5 to 12 membered, 5 to 10 membered, 5 to 9 membered, or 5 to 6 membered). In embodiments, R¹⁶ is independently halogen (e.g., —F, —Cl, Br, —I), —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, R¹⁶ is independently R^{16E}-substituted or unsubstituted C₁-C₆ alkyl, R^{16E}-substituted or unsubstituted 2 to 6 membered heteroalkyl, R^{16E}-substituted or unsubstituted C₃-C₆ cycloalkyl, R^{16E}-substituted or unsubstituted 3 to 6 membered heterocycloalkyl, R^{16E}-substituted or unsubstituted phenyl, or R^{16E}-substituted or unsubstituted 5 to 6 membered heteroaryl. In embodiments, R¹⁶ is independently —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{16E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, R^{16E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{16F}-substituted or unsubstituted alkyl (e.g., C₁-C₈, C₁-C₆, C₁-C₄, or C₁-C₂), R^{16F}-substituted or unsubstituted heteroalkyl (e.g., 2 to 8 membered, 2 to 6 membered, 4 to 6 membered, 2 to 3 membered, or 4 to 5 membered), R^{16F}-substituted or unsubstituted cycloalkyl (e.g., C₃-C₈, C₃-C₆, C₄-C₆, or C₅-C₆), R^{16F}-substituted or unsubstituted heterocycloalkyl (e.g., 3 to 8 membered, 3 to 6 membered, 4 to 6 membered, 4 to 5 membered, or 5 to 6 membered), R^{16F}-substituted or unsubstituted aryl (e.g., C₆-C₁₂, C₆-C₁₀, or phenyl), or R^{16F}-substituted or unsubstituted heteroaryl (e.g., 5 to 12 membered, 5 to 10 membered, 5 to 9 membered, or 5 to 6 membered). In embodiments, R^{16E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R^{16E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{16F}-substituted or unsubstituted C₁-C₆ alkyl, R^{16F}-substituted or unsubstituted 2 to 6 membered heteroalkyl, R^{16F}-substituted or unsubstituted C₃-C₆ cycloalkyl, R^{16F}-substituted or unsubstituted 3 to 6 membered heterocycloalkyl, R^{16F}-substituted or unsubstituted phenyl, or R^{16F}-substituted or unsubstituted 5 to 6 membered heteroaryl. In embodiments, R^{16E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{16F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In embodiments, R^{16F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, C₁-C₆ unsubstituted alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

In embodiments, R¹⁶ is —OH. In embodiments, R¹⁶ is —OCH₃. In embodiments, R¹⁶ is —F. In embodiments, R¹⁶ is —Br. In embodiments, R¹⁶ is —Cl. In embodiments, R¹⁶ is —CH₃. In embodiments, R¹⁶ is —CH₂CH₃. In embodiments, R²⁰ is

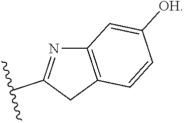 [SMILES: *C1=Nc2cc(O)ccc2C1]

In embodiments, R²⁰ is

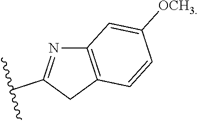 [SMILES: *C1=Nc2cc(OC)ccc2C1]

In embodiments, R²⁰ is

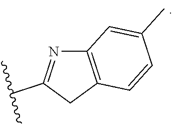 [SMILES: *C1=Nc2cc(C)ccc2C1]

In embodiments, R²⁰ is

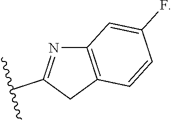 [SMILES: *C1=Nc2cc(F)ccc2C1]

In embodiments, R²⁰ is

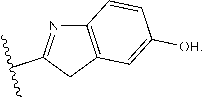 [SMILES: *C1=Nc2ccc(O)cc2C1]

In embodiments, R²⁰ is

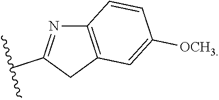 [SMILES: *C1=Nc2ccc(OC)cc2C1]

In embodiments, R²⁰ is

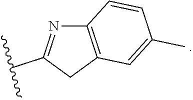 [SMILES: *C1=Nc2ccc(C)cc2C1]

In embodiments, R²⁰ is

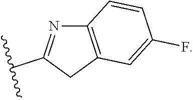 [SMILES: *C1=Nc2ccc(F)cc2C1]

In embodiments, R²⁰ is

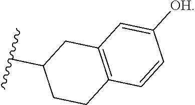 [SMILES: *C1CCc2ccc(O)cc2C1]

In embodiments, R²⁰ is

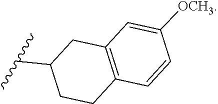 [SMILES: *C1CCc2ccc(OC)cc2C1]

In embodiments, R²⁰ is

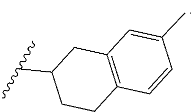 [SMILES: *C1CCc2ccc(C)cc2C1]

In embodiments, R²⁰ is

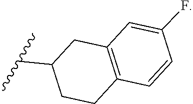 [SMILES: *C1CCc2ccc(F)cc2C1]

In embodiments, R²⁰ is

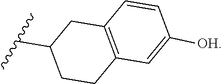 [SMILES: *C1CCc2cc(O)ccc2C1]

In embodiments, R²⁰ is

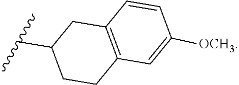 [SMILES: *C1CCc2cc(OC)ccc2C1]

In embodiments, R²⁰ is

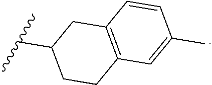 [SMILES: *C1CCc2cc(C)ccc2C1]

In embodiments, R²⁰ is

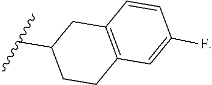 [SMILES: *C1CCc2cc(F)ccc2C1]

In embodiments, R²⁰ is

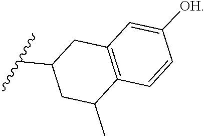 [SMILES: *C1Cc2cc(O)ccc2C(C)C1]

In embodiments, R²⁰ is

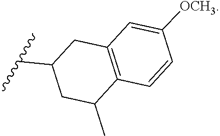 [SMILES: *C1Cc2cc(OC)ccc2C(C)C1]

In embodiments, R²⁰ is

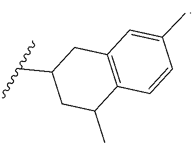 [SMILES: *C1Cc2cc(C)ccc2C(C)C1]

In embodiments, R²⁰ is

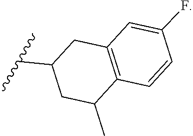 [SMILES: *C1Cc2cc(F)ccc2C(C)C1]

In embodiments, R²⁰ is

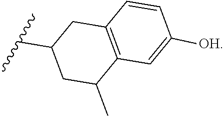 [SMILES: *C1Cc2ccc(O)cc2C(C)C1]

In embodiments, R²⁰ is

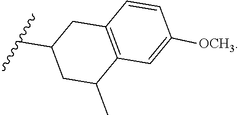 [SMILES: *C1Cc2ccc(OC)cc2C(C)C1]

In embodiments, R²⁰ is

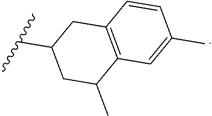 [SMILES: *C1Cc2ccc(C)cc2C(C)C1]

In embodiments, R²⁰ is

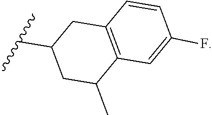 [SMILES: *C1Cc2ccc(F)cc2C(C)C1]

Provided herein are compounds having a structure of formula (II):

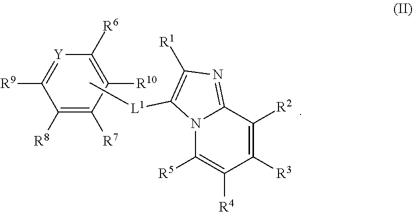

L¹, L^{1A}, R¹, R², R³, R⁴, and R⁵ are as described herein. Y is —N═ or —CR¹²═. R⁶ is a bond (to L¹), hydrogen, halogen, —CX⁶₃, —CHX⁶₂, —CH₂X⁶, —OCX⁶₃, —OCH₂X⁶, —OCHX⁶₂, —N₃, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁷ is a bond (to L¹), hydrogen, halogen, —CX⁷₃, —CHX⁷₂, —CH₂X⁷, —OCX⁷₃, —OCH₂X⁷, —OCHX⁷₂, —N₃, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁷ is a bond (to L¹), hydrogen, halogen, —CX⁸₃, —CHX⁸₂, —CH₂X⁸, —OCX⁸₃, —OCH₂X⁸, —OCHX⁸₂, —N₃, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —OR^{8D}, —NR^{8A}SO₂R^{8D}, —NR^{8A}C(O)R^{8C}, —NR^{8A}C(O)OR^{8C}, —NR^{8A}OR^{8C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁹ is a bond (to L¹), hydrogen, halogen, —CX⁹₃, —CHX⁹₂, —CH₂X⁹, —OCX⁹₃, —OCH₂X⁹, —OCHX⁹₂, —N₃, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹⁰ is a bond (to L¹), hydrogen, halogen, —CX¹⁰₃, —CHX¹⁰₂, —CH₂X¹⁰, —OCX¹⁰₃, —OCH₂X¹⁰, —OCHX¹⁰₂, —N₃, —CN, —SOₙ₁₀R^{10D}, —SOᵥ₁₀NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}AC(O)R^{10C}, —NR^{10A}C(O)OR^{10C}, —NR^{10A}OR^{10C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. One of R⁶, R⁷, R⁸, R⁹ are R¹⁰ is a bond to L¹. R¹² is hydrogen, halogen, —CX¹²₃, —CHX¹²₂, —CH₂X¹², —OCX¹²₃, —OCH₂X¹², —OCHX¹²₂, —N₃, —CN, —SOₙ₁₂R^{12D}, —SOᵥ₁₂NR^{12A}R^{12B}, —NHC(O)NR^{12A}R^{12B}, —N(O)ₘ₁₂, —NR^{12A}R^{12B}, —C(O)R^{12C}, —C(O)—OR^{12C}, —C(O)NR^{12A}R^{12B}, —OR^{12D}, —NR^{12A}SO₂R^{12D}, —NR^{12A}C(O)R^{12C}, —NR^{12A}C(O)OR^{12C}, —NR^{12A}OR^{12C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{6A}, R^{6B}, R^{6C}, R^{6D}, R^{7A}, R^{7B}, R^{7C}, R^{7D}, R^{8A}, R^{8B}, R^{8C}, R^{8D}, R^{9A}, R^{9B}, R^{9C}, R^{9D}, R^{10A}, R^{10B}, R^{10C}, R^{10D}, R^{12A}, R^{12B}, R^{12C}, and R^{12D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁷ and R⁸ together with atoms attached thereto may optionally be joined to form substituted or unsubstituted cycloalkyl, substituted or unsubstituted heterocycloalkyl, substituted or unsubstituted aryl, or substituted or unsubstituted heteroaryl. R⁸ and R⁹ together with atoms attached thereto may optionally be joined to form substituted or unsubstituted cycloalkyl, substituted or unsubstituted heterocycloalkyl, substituted or unsubstituted aryl, or substituted or unsubstituted heteroaryl. R⁹ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl. R⁶ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl. R⁶ and R¹⁰ together with atoms attached thereto may optionally be joined to form substituted or unsubstituted cycloalkyl, substituted or unsubstituted heterocycloalkyl, substituted or unsubstituted aryl, or substituted or unsubstituted heteroaryl. R⁷ and R¹⁰ together with atoms attached thereto may optionally be joined to form substituted or unsubstituted cycloalkyl, substituted or unsubstituted heterocycloalkyl, substituted or unsubstituted aryl, or substituted or unsubstituted heteroaryl. n6, n7, n8, n9, n10 and n12 are independently an integer from 0 to 4. m6, m7, m8, m9, m10, m12, v6, v7, v8, v9, v10 and v12 are independently an integer from 1 to 2. X⁶, X⁷, X⁸, X⁹, X¹⁰ and X¹² are independently —F, —Cl, —Br, or —I.

In embodiments, L¹ is —SOₙ₁₁L^{1A}- (e.g., —SO₂—, —SO₃—, —SO₄—, —SO₂CH₂—, or —SO₂CH₂CH₂—), —SOᵥ₁₁NR¹¹L^{1A}- (e.g., —SO₂NHCH—, —SO₂N(CH₃)CH₂—, —SO₂N(CH₃)—CH═CH—, —SO₂N(CH₃)—N═CH—), —NHC(O)NR¹¹L^{1A}- (e.g., —NHC(O)NHCH₂—, or —NHC(O)N(CH₃)CH₂—)—NR¹¹L^{1A}- (e.g. —NHCH₂— or —NHCH₂CH₂—), —C(O)L^{1A}- (e.g., —C(O)CH₂—, —C(O)CH₂CH₂—, or —C(O)C₆C₄—), —C(O)OL^{1A}- (e.g., —C(O)OCH₂—, —C(O)OCH₂CH₂—, or —C(O)OC₆C₄—), —C(O)NR¹¹L^{1A}- (e.g., —C(O)NHCH₂—, —C(O)NHCH₂CH₂—, or —C(O)NHC₆C₄—), —OL^{1A}- (e.g., —OCH₂— or —OCH₂CH₂—), —NR¹¹SO₂L^{1A}- (e.g., —NHSO₂CH²—, or —N(CH₃)SO₂CH₂—), —NR¹¹C(O)L^{1A}- (e.g., —NHC(O)CH₂—, —NHC(O)—CH═CH—, or —N(CH₃)C(O)CH₂CH₂—), —NR¹¹C(O)OL^{1A}- (e.g., —NHC(O)OCH₂—, —NHC(O)O—CH═CH—, or —N(CH₃)C(O)OCH₂CH₂—), —NR¹¹OL^{1A}- (e.g., —NHOCH₂—, —NHO—CH═CH—, or —N(CH₃)OCH₂CH₂—), —SL^{1A}- (e.g., —SCH₂— or —SCH₂CH₂—), substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered). In embodiments, L¹ is not a bond.

In embodiments, L¹ is —SOₙ₁₁L^{1A}- (e.g., —SO₂—, —SO₃—, —SO₄—, —SO₂CH₂—, or —SO₂CH₂CH₂—). In embodiments, L¹ is —SOᵥ₁₁NR¹¹L^{1A}- (e.g., —SO₂NHCH—, —SO₂N(CH₃)CH₂—, —SO₂N(CH₃)—CH═CH—, —SO₂N(CH₃)—N═CH—). In embodiments, L¹ is —NHC(O)NR¹¹L^{1A}- (e.g., —NHC(O)NHCH₂—, or —NHC(O)N(CH₃)CH₂—). In embodiments, L¹ is —NR¹¹L^{1A}- (e.g. —NHCH₂— or —NHCH₂CH₂—). In embodiments, L¹ is —C(O)L^{1A}- (e.g., —C(O)CH₂—, —C(O)CH₂CH₂—, or —C(O)C₆C₄—). In embodiments, L¹ is —C(O)OL^{1A}- (e.g., —C(O)OCH₂—, —C(O)OCH₂CH₂—, or —C(O)OC₆C₄—). In embodiments, L¹ is —C(O)NR¹¹L^{1A}- (e.g., —C(O)NHCH₂—, —C(O)NHCH₂CH₂—, or —C(O)NHC₆C₄—). In embodiments, L¹ is —OL^{1A}- (e.g., —OCH₂— or —OCH₂CH₂—). In embodiments, L¹ is —NR¹¹SO₂L^{1A}- (e.g., —NHSO₂CH²—, or —N(CH₃)SO₂CH₂—). In embodiments, L¹ is —NR¹¹C(O)L^{1A}- (e.g., —NHC(O)CH₂—, —NHC(O)—CH═CH—, or —N(CH₃)C(O)CH₂CH₂—). In embodiments, L¹ is —NR¹¹C(O)OL^{1A}- (e.g., —NHC(O)OCH₂—, —NHC(O)O—CH═CH—, or —N(CH₃)C(O)OCH₂CH₂—). In embodiments, L¹ is —NR¹¹OL^{1A}- (e.g., —NHOCH₂—, —NHO—CH═CH—, or —N(CH₃)OCH₂CH₂—). In embodiments, L¹ is —SL^{1A}- (e.g., —SCH₂— or —SCH₂CH₂—). In embodiments, L¹ is substituted or unsubstituted alkylene (e.g., C₁-C₈, C₁-C₆, C₁-C₄, or C₁-C₂). In embodiments, L¹ is substituted or unsubstituted heteroalkylene (e.g., 2 to 8 membered, 2 to 6 membered, 4 to 6 membered, 2 to 3 membered, or 4 to 5 membered). In embodiments, L¹ is substituted or unsubstituted cycloalkylene (e.g., C₃-C₈, C₃-C₆, C₄-C₆, or C₅-C₆). In embodiments, L¹ is substituted or unsubstituted heterocycloalkylene (e.g., 3 to 8 membered, 3 to 6 membered, 4 to 6 membered, 4 to 5 membered, or 5 to 6 membered). In embodiments, L¹ is substituted or unsubstituted arylene (e.g., C₆-C₁₀ or phenylene). In embodiments, L¹ is or substituted or unsubstituted heteroarylene (e.g., 5 to 10 membered, 5 to 9 membered, or 5 to 6 membered).

In embodiments, L¹ is —SO₂—. In embodiments, L¹ is —SO₃—. In embodiments, L¹ is —SO₄—. In embodiments, L¹ is —SO₂CH₂—. In embodiments, L¹ is —SO₂CH₂CH₂—. In embodiments, L¹ is —SO₂NHCH—. In embodiments, L¹ is —SO₂N(CH₃)CH₂—. In embodiments, L¹ is —SO₂N(CH₃)—CH═CH—. In embodiments, L¹ is —SO₂N(CH₃)—N═CH—. In embodiments, L¹ is —NHC(O)NHCH₂—. In embodiments, L¹ is —NHC(O)N(CH₃)CH₂—. In embodiments, L¹ is —NHCH₂—, In embodiments, L¹ is —NHCH₂CH₂—. In embodiments, L¹ is —C(O)CH₂—. In embodiments, L¹ is —C(O)CH₂CH₂—. In embodiments, L¹ is —C(O)C₆C₄—. In embodiments, L¹ is —C(O)OCH₂—. In embodiments, L¹ is —C(O)OCH₂CH₂—. In embodiments, L¹ is —C(O)OCH═CH—. In embodiments, L¹ is —C(O)NHCH₂—. In embodiments, L¹ is —C(O)N═CH—. In embodiments, L¹ is —C(O)N═CHCH₂—. In embodiments, L¹ is —C(O)NHCH₂CH₂—. In embodiments, L¹ is —C(O)NHC₆C₄—. In embodiments, L¹ is —OCH₂—. In embodiments, L¹ is —OCH₂CH₂—. In embodiments, L¹ is —NHSO₂CH²—. In embodiments, L¹ is —N(CH₃)SO₂CH₂—. In embodiments, L¹ is —NHC(O)CH₂—. In embodiments, L¹ is —NHC(O)—CH═CH—. In embodiments, L¹ is —N(CH₃)C(O)CH₂CH₂—. In embodiments, L¹ is —NHC(O)OCH₂—. In embodiments, L¹ is —NHC(O)O—CH═CH—. In embodiments, L¹ is —N(CH₃)C(O)OCH₂CH₂—. In embodiments, L¹ is —NHOCH₂—. In embodiments, L¹ is —NHO—CH═CH—. In embodiments, L¹ is —N(CH₃)OCH₂CH₂—. In embodiments, L¹ is —SCH₂—. In embodiments, L¹ is —SCH₂CH₂—.

In embodiments, L¹ is substituted or unsubstituted C₁-C₈ alkylene. In embodiments, L¹ is substituted or unsubstituted C₁-C₆ alkylene. In embodiments, L¹ is substituted or unsubstituted C₁-C₄ alkylene. In embodiments, L¹ is substituted or unsubstituted C₁-C₂ alkylene. In embodiments, L¹ is unsubstituted C₁-C₈ alkylene. In embodiments, L¹ is unsubstituted C₁-C₆ alkylene. In embodiments, L¹ is unsubstituted C₁-C₄ alkylene. In embodiments, L¹ is unsubstituted C₁-C₂ alkylene. In embodiments, L¹ is unsubstituted —CH₂—. In embodiments, L¹ is unsubstituted —CH₂CH₂—. In embodiments, L¹ is unsubstituted —CH₂CH₂CH₂—. In embodiments, L¹ is unsubstituted —CH₂CH₂CH₂CH₂—. In embodiments, L¹ is R¹³-substituted or unsubstituted C₁-C₈ alkylene. In embodiments, L¹ is R¹³-substituted or unsubstituted C₁-C₆ alkylene. In embodiments, L¹ is R¹³-substituted or unsubstituted C₁-C₄ alkylene. In embodiments, L¹ is R¹³-substituted or unsubstituted C₁-C₃ alkylene. In embodiments, L¹ is R¹³-substituted or unsubstituted C₁-C₂ alkylene. In embodiments, L¹ is unsubstituted C₁-C₈ alkylene. In embodiments, L¹ is unsubstituted C₁-C₆ alkylene. In embodiments, L¹ is unsubstituted C₁-C₄ alkylene. In embodiments, L¹ is unsubstituted C₁-C₂ alkylene. In embodiments, L¹ is unsubstituted —CH₂—. In embodiments, L¹ is unsubstituted —CH₂CH₂—. In embodiments, L¹ is unsubstituted —CH₂CH₂CH₂—. In embodiments, L¹ is unsubstituted —CH₂CH₂CH₂CH₂—.

In embodiments, L¹ is substituted or unsubstituted 2 to 8 membered heteroalkylene. In embodiments, L¹ is substituted or unsubstituted 2 to 6 membered heteroalkylene. In embodiments, L¹ is substituted or unsubstituted 4 to 6 membered heteroalkylene. In embodiments, L¹ is substituted or unsubstituted 2 to 3 membered heteroalkylene. In embodiments, L¹ is substituted or unsubstituted 4 to 5 membered heteroalkylene. In embodiments, L¹ is R¹³-substituted or unsubstituted 2 to 8 membered heteroalkylene. In embodiments, L¹ is R¹³-substituted or unsubstituted 2 to 6 membered heteroalkylene. In embodiments, L¹ is R¹³-substituted or unsubstituted 4 to 6 membered heteroalkylene. In embodiments, L¹ is R¹³-substituted or unsubstituted 2 to 3 membered heteroalkylene. In embodiments, L¹ is R¹³-substituted or unsubstituted 4 to 5 membered heteroalkylene. In embodiments, L¹ is unsubstituted 2 to 8 membered heteroalkylene. In embodiments, L¹ is unsubstituted 2 to 6 membered heteroalkylene. In embodiments, L¹ is unsubstituted 4 to 6 membered heteroalkylene. In embodiments, L¹ is unsubstituted 2 to 3 membered heteroalkylene. In embodiments, L¹ is unsubstituted 4 to 5 membered heteroalkylene.

In embodiments, L¹ is substituted or unsubstituted C₃-C₈ cycloalkylene. In embodiments, L¹ is substituted or unsubstituted C₄-C₆ cycloalkylene. In embodiments, L¹ is R¹³-substituted or unsubstituted C₅-C₆ cycloalkylene. In embodiments, L¹ is R¹³-substituted or unsubstituted C₃-C₈ cycloalkylene. In embodiments, L¹ is R¹³-substituted or unsubstituted C₄-C₆ cycloalkylene. In embodiments, L¹ is R¹³-substituted or unsubstituted C₅-C₆ cycloalkylene. In embodiments, L¹ is unsubstituted C₃-C₈ cycloalkylene. In embodiments, L¹ is unsubstituted C₄-C₆ cycloalkylene. In embodiments, L¹ is unsubstituted C₅-C₆ cycloalkylene. In embodiments, L¹ is substituted or unsubstituted 3 to 8 membered heterocycloalkylene. In embodiments, L¹ is substituted or unsubstituted 3 to 6 membered heterocycloalkylene. In embodiments, L¹ is substituted or unsubstituted 4 to 6 membered heterocycloalkylene. In embodiments, L¹ is substituted or unsubstituted 4 to 5 membered heterocycloalkylene. In embodiments, L¹ is substituted or unsubstituted 5 to 6 membered heterocycloalkylene. In embodiments, L¹ is R¹³-substituted or unsubstituted 3 to 8 membered heterocycloalkylene. In embodiments, L¹ is R¹³-substituted or unsubstituted 3 to 6 membered heterocycloalkylene. In embodiments, L¹ is R¹³-substituted or unsubstituted 4 to 6 membered heterocycloalkylene. In embodiments, L¹ is R¹³-substituted or unsubstituted 4 to 5 membered heterocycloalkylene. In embodiments, L¹ is R¹³-substituted or unsubstituted 5 to 6 membered heterocycloalkylene. In embodiments, L¹ is unsubstituted 3 to 8 membered heterocycloalkylene. In embodiments, L¹ is unsubstituted 3 to 6 membered heterocycloalkylene. In embodiments, L¹ is unsubstituted 4 to 6 membered heterocycloalkylene. In embodiments, L¹ is unsubstituted 4 to 5 membered heterocycloalkylene. In embodiments, L¹ is unsubstituted 5 to 6 membered heterocycloalkylene.

In embodiments, L¹ is substituted or unsubstituted C₆-C₁₀ arylene. In embodiments, L¹ is substituted or unsubstituted phenylene. In embodiments, L¹ is R¹³-substituted or unsubstituted C₆-C₁₀ arylene. In embodiments, L¹ is R¹³-substituted or unsubstituted phenylene. In embodiments, L¹ is unsubstituted C₆-C₁₀ arylene. In embodiments, L¹ is unsubstituted phenylene. In embodiments, L¹ is substituted or unsubstituted 5 to 10 membered heteroarylene. In embodiments, L¹ is substituted or unsubstituted 5 to 9 membered heteroarylene. In embodiments, L¹ is substituted or unsubstituted 5 to 6 membered heteroarylene. In embodiments, L¹ is R¹³-substituted or unsubstituted 5 to 10 membered heteroarylene. In embodiments, L¹ is R¹³-substituted or unsubstituted 5 to 9 membered heteroarylene. In embodiments, L¹ is R¹³-substituted or unsubstituted 5 to 6 membered heteroarylene. In embodiments, L¹ is unsubstituted 5 to 10 membered heteroarylene. In embodiments, L¹ is unsubstituted 5 to 9 membered heteroarylene. In embodiments, L¹ is unsubstituted 5 to 6 membered heteroarylene.

In embodiments, L¹ is

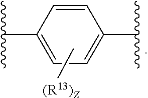

In embodiments, L¹ is

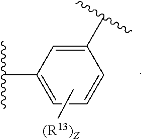

In embodiments, L¹ is

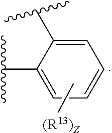

In embodiments, L¹ is

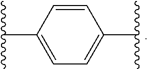 [SMILES: *c1ccc(*)cc1]

In embodiments, L¹ is

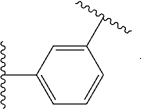 [SMILES: *c1cccc(*)c1]

In embodiments, L¹ is

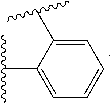 [SMILES: *c1ccccc1*]

In embodiments, z is 0. In embodiments, z is 1. In embodiments, z is 2. In embodiments, z is 3. In embodiments, z is 4.

In embodiments, R¹³ is halogen (e.g., —F, —Cl, Br, or —I). In embodiments, R¹³ is —CX¹³₃ (e.g., —CF₃, —CCl₃, —CBr₃, or —CI₃). In embodiments, R¹³ is —CHX¹³₂ (e.g., —CHF₂, —CHCl₂, —CHBr₂ or —CHI₂). In embodiments, R¹³ is —CH₂X¹³ (e.g., —CH₂F, —CH₂Cl, —CH₂Br, or —CH₂I). In embodiments, R¹³ is —OCX¹³₃ (e.g., —OCF₃, —OCCl₃, —OCBr₃, —OC₃). In embodiments, R¹³ is —OCH₂X¹³ (e.g., —OCH₂F, —OCH₂Cl, —OCH₂Br, or —OCH₂I). In embodiments, R¹³ is —OCHX¹³₂ (e.g., —OCHF₂, —OCHCl₂, —OCHBr₂, or —OCHI₂). In embodiments, R¹³ is —N₃. In embodiments, R¹³ is —CN. In embodiments, R¹³ is —SOₙ₁₃R^{13D} (e.g., —SH, —SCH₃, —SO₂H, —SO₂CH₃, —SO₃H, or —SO₄CH₃). In embodiments, R¹³ is —SOᵥ₁₃NR^{13A}R^{13B} (e.g., —SO₂NH₂, or —SO₂NHCH₃). In embodiments, R¹³ is —NHC(O)NR^{13A}R^{13B} (e.g., —NHC(O)NH₂, or —NHC(O)NHCH₃). In embodiments, R¹³ is —N(O)ₘ₁₃ (e.g. —NO₂). In embodiments, R¹³ is —NR^{13A}R^{13B} (e.g., —NH₂, or —NHCH₃). In embodiments, R¹³ is —C(O)R^{13C} (e.g., —C(O)H or —C(O)CH₃). In embodiments, R¹³ is —C(O)—OR^{13C} (e.g., —C(O)OH, or —C(O)OCH₃). In embodiments, R¹³ is —C(O)NR^{13A}R^{13B} (e.g., —C(O)NH₂ or —C(O)NHCH₃). In embodiments, R¹³ is —OR^{13D} (e.g., —OH, or —OCH₃). In embodiments, R¹³ is —NR^{13A}SO₂R^{13D} (e.g., —NHSO₂H or —NHSO₂CH₃). In embodiments, R¹³ is —NR^{13A}C(O)R^{13C} (e.g., —NHC(O)H or —NCH₃C(O)H). In embodiments, R¹³ is —NR^{13A}C(O)OR^{13C} (e.g., —NHC(O)OH or —NCH₃C(O)OH). In embodiments, R¹³ is —NR^{13A}OR^{13C} (e.g., —NHOH, —NCH₃OH or —NCH₃OCH₃). X¹³ is independently —F, —Cl, —Br, or —I.

In embodiments, R¹³ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, R¹³ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), or substituted or unsubstituted heterocycloalkyl (e.g., 3 to 8 membered, 3 to 6 membered, 4 to 6 membered, 4 to 5 membered, or 5 to 6 membered). In embodiments, R¹³ is substituted or unsubstituted alkyl (e.g., C₁-C₈, C₁-C₆, C₁-C₄, or C₁-C₂) or substituted or unsubstituted heteroalkyl (e.g., 2 to 8 membered, 2 to 6 membered, 4 to 6 membered, 2 to 3 membered, or 4 to 5 membered). In embodiments, R¹³ is unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R¹³ is unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), or unsubstituted heterocycloalkyl (e.g., 3 to 8 membered, 3 to 6 membered, 4 to 6 membered, 4 to 5 membered, or 5 to 6 membered). In embodiments, R¹³ is unsubstituted alkyl (e.g., C₁-C₈, C₁-C₆, C₁-C₄, or C₁-C₂) or unsubstituted heteroalkyl (e.g., 2 to 8 membered, 2 to 6 membered, 4 to 6 membered, 2 to 3 membered, or 4 to 5 membered).

In embodiments, R¹³ is substituted or unsubstituted C₁-C₈ alkyl. In embodiments, R¹³ is substituted or unsubstituted C₁-C₆ alkyl. In embodiments, R¹ is substituted or unsubstituted C₁-C₄ alkyl. In embodiments, R¹³ is substituted or unsubstituted C₁-C₂ alkyl. In embodiments, R¹³ is unsubstituted C₁-C₈ alkyl. In embodiments, R¹ is unsubstituted C₁-C₆ alkyl. In embodiments, R¹³ is unsubstituted C₁-C₄ alkyl. In embodiments, R¹³ is unsubstituted C₁-C₂ alkyl. In embodiments, R¹³ is unsubstituted propyl. In embodiments, R¹³ is unsubstituted isopropyl. In embodiments, R¹³ is unsubstituted ethyl. In embodiments, R¹³ is unsubstituted methyl. In embodiments, R¹³ is unsubstituted butyl. In embodiments, R¹³ is unsubstituted tert-butyl. In embodiments, R¹³ is unsubstituted iso-butyl. R¹³ is unsubstituted sec-butyl.

In embodiments, R¹³ is substituted or unsubstituted 2 to 8 membered heteroalkyl. In embodiments, R¹³ is substituted or unsubstituted 2 to 6 membered heteroalkyl. In embodiments, R¹³ is substituted or unsubstituted 4 to 6 membered heteroalkyl. In embodiments, R¹³ is substituted or unsubstituted 2 to 3 membered heteroalkyl. In embodiments, R¹³ is substituted or unsubstituted 4 to 5 membered heteroalkyl. In embodiments, R¹³ is unsubstituted 2 to 8 membered heteroalkyl. In embodiments, R¹³ is unsubstituted 2 to 6 membered heteroalkyl. In embodiments, R¹³ is unsubstituted 4 to 6 membered heteroalkyl. In embodiments, R¹³ is unsubstituted 2 to 3 membered heteroalkyl. In embodiments, R¹³ is unsubstituted 4 to 5 membered heteroalkyl.

In embodiments, R¹³ is halogen, or substituted or unsubstituted alkyl. In embodiments, R¹³ is or substituted or unsubstituted C₁-C₄ alkyl. In embodiments, R¹³ is hydrogen, or halogen. In embodiments, R¹³ is —F. In embodiments, R¹³ is —Cl. In embodiments, R¹³ is —Br. In embodiments, R¹³ is —I. In embodiments, R¹³ is substituted or unsubstituted C₁-C₃alkyl. In embodiments, R¹³ is substituted or unsubstituted methyl. In embodiments, R¹³ is substituted or unsubstituted ethyl. In embodiments, R¹³ is substituted or unsubstituted propyl. In embodiments, R¹³ is substituted or unsubstituted isopropyl. In embodiments, R¹³ is unsubstituted C₁-C₃alkyl. In embodiments, R¹³ is unsubstituted methyl. In embodiments, R¹³ is unsubstituted ethyl. In embodiments, R¹³ is unsubstituted propyl. In embodiments, R¹³ is unsubstituted isopropyl.

In embodiments, L^{1A} is independently a bond. In embodiments, L^{1A} is substituted or unsubstituted C₁-C₄ alkylene. In embodiments, L^{1A} is substituted or unsubstituted 2 to 6 membered heteroalkylene. In embodiments, L^{1A} is substituted or unsubstituted C₃-C₆ cycloalkylene. In embodiments, L^{1A} is substituted or unsubstituted 3 to 6 membered heterocycloalkylene. In embodiments, L^{1A} is substituted or unsubstituted phenylene. In embodiments, L^{1A} is substituted or unsubstituted 5 to 6 membered heteroarylene. In embodiments, L^{1A} is unsubstituted C₁-C₄ alkylene. In embodiments, L^{1A} is unsubstituted 2 to 6 membered heteroalkylene. In embodiments, L^{1A} is unsubstituted C₃-C₆ cycloalkylene. In embodiments, L^{1A} is unsubstituted 3 to 6 membered heterocycloalkylene. In embodiments, L^{1A} is unsubstituted phenylene. In embodiments, L^{1A} is substituted or unsubstituted 5 to 6 membered heteroarylene. In embodiments, L^{1A} is unsubstituted methylene. In embodiments, L^{1A} is unsubstituted ethylene. In embodiments, L^{1A} is unsubstituted propylene. In embodiments, L^{1A} is —OCH²—. In embodiments, L^{1A} is —SCH²—. In embodiments, L^{1A} is —N(CH₃)—. In embodiments, L^{1A} is —NH—. In embodiments, L^{1A} is —OCH₂CH₂—. In embodiments, L^{1A} is —OCH═CH—. In embodiments, L^{1A} is —SCH═CH—. In embodiments, L^{1A} is unsubstituted cyclopropylene. In embodiments, L^{1A} is unsubstituted cyclobutylene. In embodiments, L^{1A} is unsubstituted cyclopeptylene. In embodiments, L^{1A} is unsubstituted cyclohexylene. In In embodiments, L^{1A} is unsubstituted phenylene. In embodiments, L^{1A} is unsubstituted pyridylene. In embodiments, L^{1A} is unsubstituted pyrimidinylene. In embodiments, L^{1A} is unsubstituted morpholinylene. In embodiments, L^{1A} is —N(CH₃)—N═CH—.

In embodiments, L^{1A} is independently a bond, unsubstituted C₁-C₄ alkylene, unsubstituted 2 to 6 membered heteroalkylene, unsubstituted C₃-C₆ cycloalkylene, unsubstituted 3 to 6 membered heterocycloalkylene, unsubstituted phenylene, or substituted or unsubstituted 5 to 6 membered heteroarylene.

In embodiments, R⁶ is a bond to L¹ and R⁷, R⁸, R⁹ and R¹⁰ are not bonds. In embodiments, R⁷ is a bond to L¹ and R⁶, R⁸, R⁹ and R¹⁰ are not a bonds. In embodiments, R⁷ is a bond to L¹ and R⁶, R⁷, R⁹ and R¹⁰ are not bonds. In embodiments, R⁹ is a bond to L¹ and R⁶, R⁷, R⁸ and R¹⁰ are not bonds. In embodiments, R¹⁰ is a bond to L¹ and R⁶, R⁷, R⁸ and R⁹ are not bonds. In embodiments, R⁶ is not a bond. In embodiments, R⁷ is not a bond. In embodiments, R⁸ is not a bond. In embodiments, R⁹ is not a bond. In embodiments, R¹⁰ is not a bond.

In embodiments, R¹⁰ is a bond to L¹. In embodiments, the compound has a structure of formula (III):

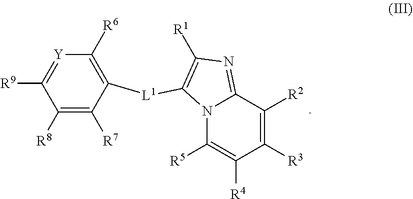

L¹, Y, R¹, R², R³, R⁴, R⁵, R⁶, R⁷, R⁸, and R⁹ are as described herein. In embodiments, R⁸ is a bond to L¹. In embodiments, the compound has a structure of formula (III′)

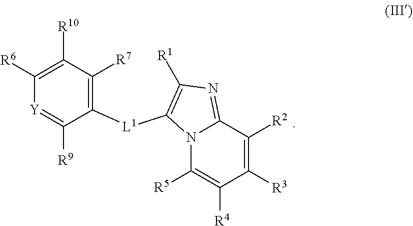

L¹, Y, R¹, R², R³, R⁴, R⁵, R⁶, R⁷, R⁹, and R¹⁰ are as described herein.

In embodiments, R⁶ is a bond to L¹. In embodiments, the compound has a structure of formula (III-A)

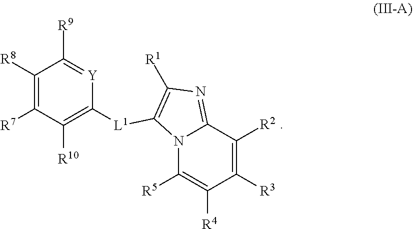

L¹, Y, R¹, R², R³, R⁴, R⁵, R⁷, R⁸, R⁹, and R¹⁰ are as described herein. In embodiments, R⁹ is a bond to L¹. In embodiments, the compound has a structure of formula (III-A′)

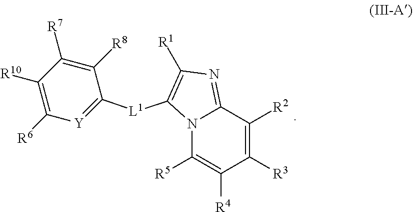

L¹, Y, R¹, R², R³, R⁴, R⁵, R⁶, R⁷, R⁸, and R¹⁰ are as described herein.

In embodiment, R⁷ is a bond to L¹. In embodiments, the compound has a structure of formula (III-B)

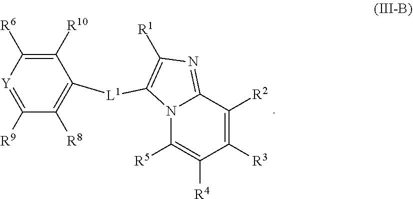

L¹, Y, R¹, R², R³, R⁴, R⁵, R⁶, R⁸, R⁹, and R¹⁰ are as described herein.

In embodiments, R¹ is hydrogen. In embodiments, R¹ is halogen (e.g., —F, —Cl, Br, or —I). In embodiments, R¹ is —CX¹₃ (e.g., —CF₃, —CCl₃, —CBr₃, or —CI₃). In embodiments, R¹ is —CHX¹₂ (e.g., —CHF₂, —CHCl₂, —CHBr₂ or —CHI₂). In embodiments, R¹ is —CH₂X¹ (e.g., —CH₂F, —CH₂Cl, —CH₂Br, or —CH₂I). In embodiments, R¹ is —OCX¹₃ (e.g., —OCF₃, —OCCl₃, —OCBr₃, —OC₃). In embodiments, R¹ is —OCH₂X¹ (e.g., —OCH₂F, —OCH₂Cl, —OCH₂Br, or —OCH₂I). In embodiments, R¹ is —OCHX¹₂ (e.g., —OCHF₂, —OCHCl₂, —OCHBr₂, or —OCHI₂). In embodiments, R¹ is —N₃. In embodiments, R¹ is —CN. In embodiments, R¹ is —SOₙ₁R^{1D} (e.g., —SH, —SCH₃, —SO₂H, —SO₂CH₃, —SO₃H, or —SO₄CH₃). In embodiments, R¹ is —SOᵥ₁NR^{1A}R^{1B} (e.g., —SO₂NH₂, or —SO₂NHCH₃). In embodiments, R¹ is —NHC(O)NR^{1A}R^{1B} (e.g., —NHC(O)NH₂, or —NHC(O)NHCH₃). In embodiments, R¹ is —N(O)ₘ₁ (e.g. —NO₂). In embodiments, R¹ is —NR^{1A}R^{1B} (e.g., —NH₂, or —NHCH₃). In embodiments, R¹ is —C(O)R^{1C} (e.g., —C(O)H or —C(O)CH₃). In embodiments, R¹ is —C(O)—OR^{1C} (e.g., —C(O)OH, or —C(O)OCH₃). In embodiments, R¹ is —C(O)NR^{1A}R^{1B} (e.g., —C(O)NH₂ or —C(O)NHCH₃). In embodiments, R¹ is —OR^{1D} (e.g., —OH, or —OCH₃). In embodiments, R¹ is —NR^{1A}SO₂R^{1D} (e.g., —NHSO₂H or —NHSO₂CH₃). In embodiments, R¹ is —NR^{1A}C(O)R^{1C} (e.g., —NHC(O)H or —NCH₃C(O)H). In embodiments, R¹ is —NR^{1A}C(O)OR^{1C} (e.g., —NHC(O)OH or —NCH₃C(O)OH). In embodiments, R¹ is —NR^{1A}OR^{1C} (e.g., —NHOH, —NCH₃OH or —NCH₃OCH₃).

In embodiments, R¹ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, R¹ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), or substituted or unsubstituted heterocycloalkyl (e.g., 3 to 8 membered, 3 to 6 membered, 4 to 6 membered, 4 to 5 membered, or 5 to 6 membered). In embodiments, R¹ is substituted or unsubstituted alkyl (e.g., C₁-C₈, C₁-C₆, C₁-C₄, or C₁-C₂) or substituted or unsubstituted heteroalkyl (e.g., 2 to 8 membered, 2 to 6 membered, 4 to 6 membered, 2 to 3 membered, or 4 to 5 membered). In embodiments, R¹ is unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R¹ is unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), or unsubstituted heterocycloalkyl (e.g., 3 to 8 membered, 3 to 6 membered, 4 to 6 membered, 4 to 5 membered, or 5 to 6 membered). In embodiments, R¹ is unsubstituted alkyl (e.g., C₁-C₈, C₁-C₆, C₁-C₄, or C₁-C₂) or unsubstituted heteroalkyl (e.g., 2 to 8 membered, 2 to 6 membered, 4 to 6 membered, 2 to 3 membered, or 4 to 5 membered).

In embodiments, R¹ is substituted or unsubstituted C₁-C₈ alkyl. In embodiments, R¹ is substituted or unsubstituted C₁-C₆ alkyl. In embodiments, R¹ is substituted or unsubstituted C₁-C₄ alkyl. In embodiments, R¹ is substituted or unsubstituted C₁-C₂ alkyl. In embodiments, R¹ is unsubstituted C₁-C₈ alkyl. In embodiments, R¹ is unsubstituted C₁-C₆ alkyl. In embodiments, R¹ is unsubstituted C₁-C₄ alkyl. In embodiments, R¹ is unsubstituted C₁-C₂ alkyl. In embodiments, R¹ is unsubstituted propyl. In embodiments, R¹ is unsubstituted isopropyl. In embodiments, R¹ is unsubstituted ethyl. In embodiments, R¹ is unsubstituted methyl. In embodiments, R¹ is unsubstituted butyl. In embodiments, R¹ is unsubstituted tert-butyl. In embodiments, R¹ is unsubstituted iso-butyl. R¹ is unsubstituted sec-butyl.

In embodiments, R¹ is substituted or unsubstituted 2 to 8 membered heteroalkyl. In embodiments, R¹ is substituted or unsubstituted 2 to 6 membered heteroalkyl. In embodiments, R¹ is substituted or unsubstituted 4 to 6 membered heteroalkyl. In embodiments, R¹ is substituted or unsubstituted 2 to 3 membered heteroalkyl. In embodiments, R¹ is substituted or unsubstituted 4 to 5 membered heteroalkyl. In embodiments, R¹ is unsubstituted 2 to 8 membered heteroalkyl. In embodiments, R¹ is unsubstituted 2 to 6 membered heteroalkyl. In embodiments, R¹ is unsubstituted 4 to 6 membered heteroalkyl. In embodiments, R¹ is unsubstituted 2 to 3 membered heteroalkyl. In embodiments, R¹ is unsubstituted 4 to 5 membered heteroalkyl.

In embodiments, R¹ is hydrogen, halogen, or unsubstituted C₁-C₄ alkyl. In embodiments, R¹ is hydrogen, or unsubstituted C₁-C₄ alkyl. In embodiments, R¹ is hydrogen, or halogen. In embodiments, R¹ is —F. In embodiments, R¹ is —Cl. In embodiments, R¹ is —Br. In embodiments, R¹ is —I. In embodiments, R¹ is substituted or unsubstituted methyl. In embodiments, R¹ is substituted or unsubstituted ethyl. In embodiments, R¹ is substituted or unsubstituted propyl. In embodiments, R¹ is substituted or unsubstituted isopropyl. In embodiments, R¹ is substituted or unsubstituted n-butyl. R¹ is substituted or unsubstituted sec-butyl. In embodiments, R¹ is substituted or unsubstituted iso-butyl. In embodiments, R¹ is substituted or unsubstituted tert-butyl.

In embodiments, the compound has a structure of formula (III-C):

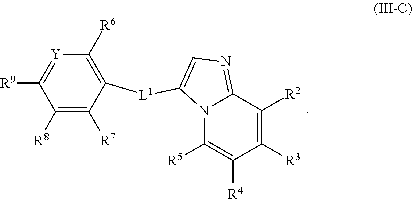

L¹, Y, R², R³, R⁴, R⁵, R⁶, R⁷, R⁸, and R⁹ are as described herein.

In embodiments, Y is —CR¹²═. In embodiments, the compound has a structure of formula (III-D):

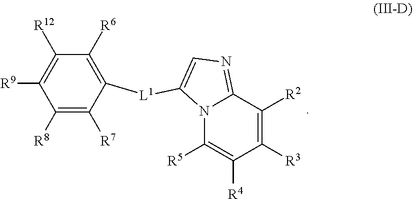

L¹, R², R³, R⁴, R⁵, R⁶, R⁷, R, and R⁹ are as described herein.

In embodiments, R¹² is hydrogen. In embodiments, R¹² is halogen (e.g., —F, —Cl, Br, or —I). In embodiments, R¹² is —CX¹²₃ (e.g., —CF₃, —CCl₃, —CBr₃, or —CI₃). In embodiments, R¹² is —CHX¹²₂ (e.g., —CHF₂, —CHCl₂, —CHBr₂ or —CHI₂). In embodiments, R¹² is —CH₂X¹² (e.g., —CH₂F, —CH₂Cl, —CH₂Br, or —CH₂I). In embodiments, R¹² is —OCX¹²₃ (e.g., —OCF₃, —OCCl₃, —OCBr₃, or —OCI₃). In embodiments, R¹² is —OCH₂X¹² (e.g., —OCH₂F, —OCH₂Cl, —OCH₂Br, or —OCH₂I). In embodiments, R¹² is —OCHX¹²₂ (e.g., —OCHF₂, —OCHCl₂, —OCHBr₂, or —OCHI₂). In embodiments, R¹² is —N₃. In embodiments, R¹² is —CN. In embodiments, R¹² is —SOₙ₁₂R^{12D} (e.g., —SH, —SCH₃, —SO₂H, —SO₂CH₃, —SO₃H, or —SO₄CH₃). In embodiments, R¹² is —SOᵥ₁₂NR^{12A}R^{12B} (e.g., —SO₂NH₂, or —SO₂NHCH₃). In embodiments, R¹² is —NHC(O)NR^{12A}R^{12B} (e.g., —NHC(O)NH₂, or —NHC(O)NHCH₃). In embodiments, R¹² is —N(O)ₘ₁₂ (e.g. —NO₂). In embodiments, R¹² is —NR^{12A}R^{12B} (e.g., —NH₂, or —NHCH₃). In embodiments, R¹² is —C(O)R^{12C} (e.g., —C(O)H or —C(O)CH₃). In embodiments, R¹² is —C(O)—OR^{12C} (e.g., —C(O)OH, or —C(O)OCH₃). In embodiments, R¹² is —C(O)NR^{12A}R^{12B} (e.g., —C(O)NH₂ or —C(O)NHCH₃). In embodiments, R¹² is —OR^{12D} (e.g., —OH, or —OCH₃). In embodiments, R¹² is —NR^{12A}SO₂R^{12D} (e.g., —NHSO₂H or —NHSO₂CH₃). In embodiments, R¹² is —NR^{12A}C(O)R^{12C} (e.g., —NHC(O)H or —N(CH₃)C(O)H). In embodiments, R¹² is —NR^{12A}C(O)OR^{12C} (e.g., —NHC(O)OH or —N(CH₃)C(O)OH). In embodiments, R¹² is —NR^{12A}OR^{12C} (e.g., —NHOH, —N(CH₃)OH or —N(CH₃)OCH₃).

In embodiments, R¹² is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, R¹² is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), or substituted or unsubstituted heterocycloalkyl (e.g., 3 to 8 membered, 3 to 6 membered, 4 to 6 membered, 4 to 5 membered, or 5 to 6 membered). In embodiments, R¹² is substituted or unsubstituted alkyl (e.g., C₁-C₈, C₁-C₆, C₁-C₄, or C₁-C₂) or substituted or unsubstituted heteroalkyl (e.g., 2 to 8 membered, 2 to 6 membered, 4 to 6 membered, 2 to 3 membered, or 4 to 5 membered). In embodiments, R¹² is unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R¹² is unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), or unsubstituted heterocycloalkyl (e.g., 3 to 8 membered, 3 to 6 membered, 4 to 6 membered, 4 to 5 membered, or 5 to 6 membered). In embodiments, R¹² is unsubstituted alkyl (e.g., C₁-C₈, C₁-C₆, C₁-C₄, or C₁-C₂) or unsubstituted heteroalkyl (e.g., 2 to 8 membered, 2 to 6 membered, 4 to 6 membered, 2 to 3 membered, or 4 to 5 membered).

In embodiments, R¹² is substituted or unsubstituted C₁-C₈ alkyl. In embodiments, R¹² is substituted or unsubstituted C₁-C₆ alkyl. In embodiments, R¹ is substituted or unsubstituted C₁-C₄ alkyl. In embodiments, R¹² is substituted or unsubstituted C₁-C₂ alkyl. In embodiments, R¹² is unsubstituted C₁-C₈ alkyl. In embodiments, R¹ is unsubstituted C₁-C₆ alkyl. In embodiments, R¹² is unsubstituted C₁-C₄ alkyl. In embodiments, R¹² is unsubstituted C₁-C₂ alkyl. In embodiments, R¹² is unsubstituted propyl. In embodiments, R¹² is unsubstituted isopropyl. In embodiments, R¹² is unsubstituted ethyl. In embodiments, R¹² is unsubstituted methyl. In embodiments, R¹² is unsubstituted butyl. In embodiments, R¹² is unsubstituted tert-butyl. In embodiments, R¹² is unsubstituted iso-butyl. In embodiments, R¹² is unsubstituted sec-butyl.

In embodiments, R¹² is substituted or unsubstituted 2 to 8 membered heteroalkyl. In embodiments, R¹² is substituted or unsubstituted 2 to 6 membered heteroalkyl. In embodiments, R¹² is substituted or unsubstituted 4 to 6 membered heteroalkyl. In embodiments, R¹² is substituted or unsubstituted 2 to 3 membered heteroalkyl. In embodiments, R¹² is substituted or unsubstituted 4 to 5 membered heteroalkyl. In embodiments, R¹² is unsubstituted 2 to 8 membered heteroalkyl. In embodiments, R¹² is unsubstituted 2 to 6 membered heteroalkyl. In embodiments, R¹² is unsubstituted 4 to 6 membered heteroalkyl. In embodiments, R¹² is unsubstituted 2 to 3 membered heteroalkyl. In embodiments, R¹² is unsubstituted 4 to 5 membered heteroalkyl.

In embodiments, R¹² is hydrogen, halogen, or unsubstituted C₁-C₄ alkyl. In embodiments, R¹² is hydrogen, or unsubstituted C₁-C₄ alkyl. In embodiments, R¹² is hydrogen, or halogen. In embodiments, R¹² is —F. In embodiments, R¹² is —Cl. In embodiments, R¹² is —Br. In embodiments, R¹² is —I. In embodiments, R¹² is substituted or unsubstituted methyl. In embodiments, R¹² is substituted or unsubstituted ethyl. In embodiments, R¹² is substituted or unsubstituted propyl. In embodiments, R¹² is substituted or unsubstituted isopropyl. In embodiments, R¹² is substituted or unsubstituted n-butyl. R¹² is substituted or unsubstituted sec-butyl. In embodiments, R¹² is substituted or unsubstituted iso-butyl. In embodiments, R¹² is substituted or unsubstituted tert-butyl.

In embodiments, Y is —CH═. In embodiments, Y is —CF═. In embodiments, Y is —CCl═. In embodiments, Y is —CBr—. In embodiments, Y is —CI═. In embodiments, Y is —C(CH₃)═. In embodiments, Y is —C(CH₂CH₃)═. In embodiments, Y is —C(CH₂CH₂CH₃)═. In embodiments, Y is —C(CH(CH₃)₂)═. In embodiments, Y is —C(CH₂CH₂ CH₂CH₃)═. In embodiments, Y is —C(CH₂CH(CH₃)₂)═. In embodiments, Y is —C(CH(CH₃)CH₂CH₃)═. In embodiments, Y is —C(CH₂C(CH₃)₃)═.

In embodiments, Y is —N═. In embodiments, R¹ is hydrogen. In embodiments, the compound has a structure of formula (III-E):

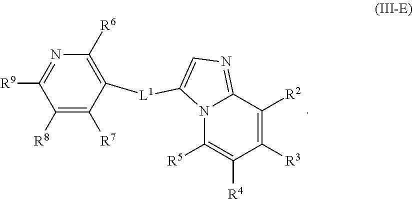

L¹, R², R³, R⁴, R⁵, R⁶, R⁷, R⁸, and R⁹ are as described herein.

In embodiments, L¹ is R¹³-substituted or unsubstituted alkylene (e.g., C₁-C₈, C₁-C₆, C₁-C₄, or C₁-C₂), R¹³-substituted or unsubstituted heteroalkylene (e.g., 2 to 8 membered, 2 to 6 membered, 4 to 6 membered, 2 to 3 membered, or 4 to 5 membered), R¹³-substituted or unsubstituted cycloalkylene (e.g., C₃-C₈, C₃-C₆, C₄-C₆, or C₅-C₆), R¹³-substituted or unsubstituted heterocycloalkylene (e.g., 3 to 8 membered, 3 to 6 membered, 4 to 6 membered, 4 to 5 membered, or 5 to 6 membered), R¹³-substituted or unsubstituted arylene (e.g., C₆-C₁₀ or phenylene), or R¹³-substituted or unsubstituted heteroarylene (e.g., 5 to 10 membered, 5 to 9 membered, or 5 to 6 membered). In embodiments, L¹ is R¹³-substituted or unsubstituted C₁-C₆ alkylene. In embodiments, L¹ is R¹³-substituted or unsubstituted 2 to 6 membered heteroalkylene. In embodiments, L¹ is R¹³-substituted or unsubstituted C₃-C₆ cycloalkylene. In embodiments, L¹ is R¹³-substituted or unsubstituted 3 to 6 membered heterocycloalkylene. In embodiments, L¹ is R¹³-substituted or unsubstituted phenylene. In embodiments, L¹ is R¹³-substituted or unsubstituted 5 to 6 membered heteroarylene. In embodiments, L¹ is unsubstituted C₁-C₆ alkylene. In embodiments, L¹ is unsubstituted 2 to 6 membered heteroalkylene. In embodiments, L¹ is unsubstituted C₃-C₆ cycloalkylene. In embodiments, L¹ is unsubstituted 3 to 6 membered heterocycloalkylene. In embodiments, L¹ is unsubstituted phenylene. In embodiments, L¹ is unsubstituted 5 to 6 membered heteroarylene. R¹³ is as described herein.

In embodiments, the compound has a structure of formula (IV):

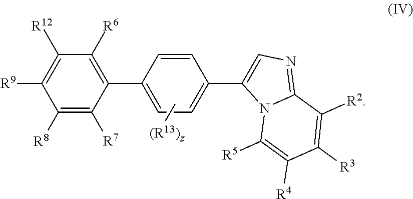

R², R³, R⁴, R⁵, R⁶, R⁷, R⁸, R⁹, R¹² and R¹³ are as described herein. z is an integer from 0 to 4.

In embodiments, the compound has a structure of formula (IV-A):

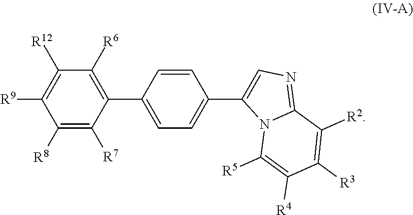

In embodiments, the compound has a structure of formula (IV-A′):

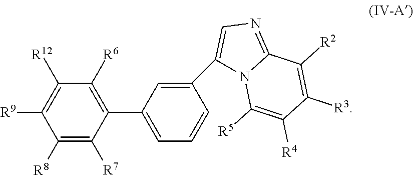

In embodiments, the compound has a structure of formula (IV-A″):

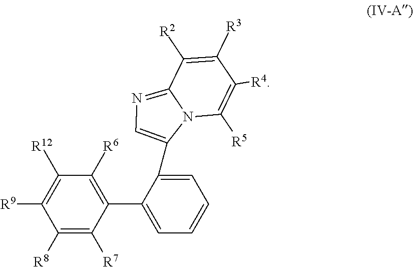

R², R³, R⁴, R⁵, R⁶, R⁷, R⁸, R⁹, and R¹² are as described herein.

In embodiments, R¹² is hydrogen. In embodiment, R¹² is —OH. In embodiment, R¹² is —OCH³. In embodiment, R¹² is —NH₂. In embodiment, R¹² is —NHCH₃.

In embodiments, L¹ is R¹³-substituted or unsubstituted heteroalkylene (e.g., 2 to 8 membered, 2 to 6 membered, 4 to 6 membered, 2 to 3 membered, or 4 to 5 membered). In embodiments, L¹ is R¹³-substituted or unsubstituted 2 to 8 membered heteroalkylene. In embodiments, L¹ is R¹³-substituted or unsubstituted 2 to 6 membered heteroalkylene. L¹ is R¹³-substituted or unsubstituted 4 to 6 membered heteroalkylene. In embodiments, L¹ is R¹³-substituted or unsubstituted 2 to 3 membered heteroalkylene. In embodiments, L¹ is R¹³-substituted or unsubstituted 5 to 6 membered heteroalkylene. In embodiments, L¹ is substituted or unsubstituted 2 to 6 membered heteroalkyl. In embodiments, L¹ includes one or more of heteroatoms selected from O, N, P, Si, and S. In embodiments, L¹ includes one or more of heteroatoms selected from O, N, P, and S. In embodiments, L¹ includes one or more of heteroatoms selected from O, N, and S. In embodiments, L¹ includes one or more of O. In embodiments, L¹ includes one or more of N. In embodiments, L¹ includes one or more of S. In embodiments, L¹ is —SO₂—N(R¹⁴)N═CH—. In embodiments, L¹ is —CH₂N(R¹⁴)N═CH—. In embodiments, L¹ is —C(O)N(R¹⁴)CH₂—. In embodiments, L¹ is —C(O)N═CHCH₂—.

In embodiments, R¹⁴ is hydrogen. In embodiments, R¹⁴ is —CX¹⁴₃ (e.g., —CF₃, —CCl₃, —CBr₃, or —CI₃). In embodiments, R¹⁴ is —CHX¹⁴₂ (e.g., —CHF₂, —CHCl₂, —CHBr₂ or —CHI₂). In embodiments, R¹⁴ is —CH₂X¹⁴ (e.g., —CH₂F, —CH₂Cl, —CH₂Br, or —CH₂I). In embodiments, R¹⁴ is —C(O)OH. In embodiments, R¹⁴ is —C(O)NH₂.

In embodiments, R¹⁴ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, R¹⁴ is substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), or substituted or unsubstituted heterocycloalkyl (e.g., 3 to 8 membered, 3 to 6 membered, 4 to 6 membered, 4 to 5 membered, or 5 to 6 membered). In embodiments, R¹⁴ is substituted or unsubstituted alkyl (e.g., C₁-C₈, C₁-C₆, C₁-C₄, or C₁-C₂) or substituted or unsubstituted heteroalkyl (e.g., 2 to 8 membered, 2 to 6 membered, 4 to 6 membered, 2 to 3 membered, or 4 to 5 membered). In embodiments, R¹⁴ is unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R¹⁴ is unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), or unsubstituted heterocycloalkyl (e.g., 3 to 8 membered, 3 to 6 membered, 4 to 6 membered, 4 to 5 membered, or 5 to 6 membered). In embodiments, R¹⁴ is unsubstituted alkyl (e.g., C₁-C₈, C₁-C₆, C₁-C₄, or C₁-C₂) or unsubstituted heteroalkyl (e.g., 2 to 8 membered, 2 to 6 membered, 4 to 6 membered, 2 to 3 membered, or 4 to 5 membered).

In embodiments, R¹⁴ is substituted or unsubstituted C₁-C₈ alkyl. In embodiments, R¹⁴ is substituted or unsubstituted C₁-C₆ alkyl. In embodiments, R¹ is substituted or unsubstituted C₁-C₄ alkyl. In embodiments, R¹⁴ is substituted or unsubstituted C₁-C₃alkyl. In embodiments, R¹⁴ is substituted or unsubstituted C₁-C₂ alkyl. In embodiments, R¹⁴ is unsubstituted C₁-C₈ alkyl. In embodiments, R¹ is unsubstituted C₁-C₆ alkyl. In embodiments, R¹⁴ is unsubstituted C₁-C₄ alkyl. In embodiments, R¹⁴ is unsubstituted C₁-C₃ alkyl. In embodiments, R¹⁴ is unsubstituted C₁-C₂ alkyl. In embodiments, R¹⁴ is unsubstituted propyl. In embodiments, R¹⁴ is unsubstituted isopropyl. In embodiments, R¹⁴ is unsubstituted ethyl. In embodiments, R¹⁴ is unsubstituted methyl.

In embodiments, R¹⁴ is substituted or unsubstituted 2 to 8 membered heteroalkyl. In embodiments, R¹⁴ is substituted or unsubstituted 2 to 6 membered heteroalkyl. In embodiments, R¹⁴ is substituted or unsubstituted 4 to 6 membered heteroalkyl. In embodiments, R¹⁴ is substituted or unsubstituted 2 to 3 membered heteroalkyl. In embodiments, R¹⁴ is substituted or unsubstituted 4 to 5 membered heteroalkyl. In embodiments, R¹⁴ is unsubstituted 2 to 8 membered heteroalkyl. In embodiments, R¹⁴ is unsubstituted 2 to 6 membered heteroalkyl. In embodiments, R¹⁴ is unsubstituted 4 to 6 membered heteroalkyl. In embodiments, R¹⁴ is unsubstituted 2 to 3 membered heteroalkyl. In embodiments, R¹⁴ is unsubstituted 4 to 5 membered heteroalkyl.

In embodiments, the compound has a structure of formula (V-A):

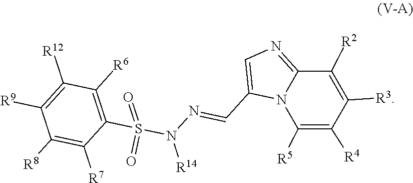

R², R³, R⁴, R⁵, R⁶, R⁷, R⁸, R⁹, R¹² and R¹⁴ are described herein.

In embodiments, the compound has a structure of formula (V-B)

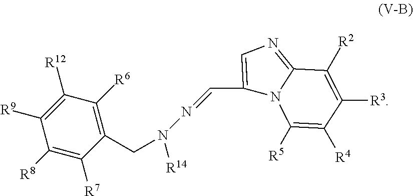

R², R³, R⁴, R⁵, R⁶, R⁷, R⁸, R⁹, R¹² and R¹⁴ are described herein.

In embodiments, the compounds has a structure of formula (V-C):

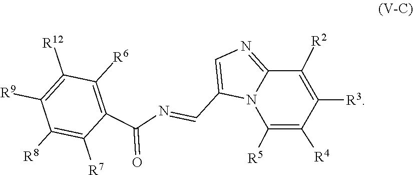

R², R³, R⁴, R⁵, R⁶, R⁷, R⁸, R⁹, and R¹² are as described herein.

In embodiments, the compounds has a structure of formula (V-D):

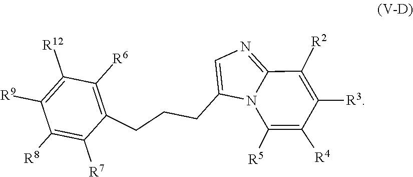

R², R³, R⁴, R⁵, R⁶, R⁷, R⁸, R⁹, and R¹² are as described herein.

In embodiments, R⁶ is hydrogen. In embodiment, R⁶ is —OH. In embodiment, R⁶ is —OCH³. In embodiment, R⁶ is —NH₂. In embodiment, R⁶ is —NHCH₃. In embodiments, R⁷ is hydrogen. In embodiment, R⁷ is —OH. In embodiment, R⁷ is —OCH³. In embodiment, R⁷ is —NH₂. In embodiment, R⁷ is —NHCH₃. In embodiments, R⁸ is hydrogen. In embodiment, R⁸ is —OH. In embodiment, R⁸ is —OCH³. In embodiment, R⁸ is —NH₂. In embodiment, R⁸ is —NHCH₃. In embodiments, R⁹ is hydrogen. In embodiment, R⁹ is —OH. In embodiment, R⁹ is —OCH³. In embodiment, R⁹ is —NH₂. In embodiment, R⁹ is —NHCH₃. In embodiments, R¹² is hydrogen. In embodiment, R¹² is —OH. In embodiment, R¹² is —OCH³. In embodiment, R¹² is —NH₂. In embodiment, R¹² is —NHCH₃.

In embodiments, R⁴ is —Br. In embodiments, R⁴ is —Cl. In embodiments, R⁴ is —F. In embodiments, R⁴ is —I. In embodiments, R⁴ is hydrogen. In embodiments, R⁴ is —

In embodiments, L¹ is —SO₂—N(CH₃)N═CH—. In embodiments, L¹ is —CH₂N(CH₃)N═CH—. In embodiments, L¹ is —C(O)N═CH—. In embodiments, L¹ is —C(O)N═CHCH₂—.

In embodiments, the compounds has a structure of formula (VI-A):

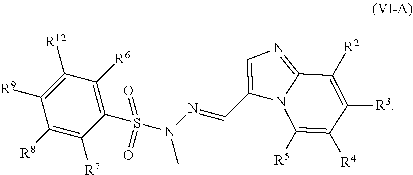

R², R³, R⁴, R⁵, R⁶, R⁷, R⁸, R⁹, and R¹² are as described herein.

In embodiments, the compound has a structure of formula (VI-B),

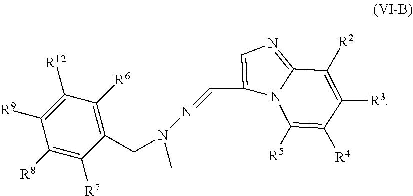

R², R³, R⁴, R⁵, R⁶, R⁷, R⁸, R⁹, and R¹² are described herein.

In embodiments, at least one of R², R³, R⁴ and R⁵ is halogen. In embodiments, at least two of R², R³, R⁴ and R⁵ are halogen. In embodiments, at least three of R², R³, R⁴ and R⁵ are halogen. In embodiments, R², R³, R⁴ and R⁵ are halogen. In embodiments, R² is halogen. In embodiments, R³ is halogen. In embodiments, R⁴ is halogen. In embodiments, R⁵ is halogen. In embodiments, R² and R³ are halogen. In embodiments, R² and R⁴ are halogen. In embodiments, R² and R⁵ are halogen. In embodiments, R³ and R⁴ are halogen. In embodiments, R³ and R⁵ are halogen. In embodiments, R⁴ and R⁵ are halogen. In embodiments, R², R³ and R⁵ are halogen. In embodiments, R², R⁴ and R⁵ are halogen. In embodiments, R³, R⁴ and R⁵ are halogen. In embodiments, R², R³, R⁴ and R⁵ are halogen. In embodiments, R² is —F. In embodiments, R² is —Cl. In embodiments, R² is —Br. In embodiments, R² is —I. In embodiments, R³ is —F. In embodiments, R³ is —Cl. In embodiments, R³ is —Br. In embodiments, R³ is —I. In embodiments, R⁴ is —F. In embodiments, R⁴ is —Cl. In embodiments, R⁴ is —Br. In embodiments, R⁴ is —I. In embodiments, R⁵ is —F. In embodiments, R⁵ is —Cl. In embodiments, R⁵ is —Br. In embodiments, R⁵ is —I.

In embodiments, at least one of R², R³, R⁴ and R⁵ is hydrogen. In embodiments, at least two of R², R³, R⁴ and R⁵ is hydrogen. In embodiments, at least three of R², R³, R⁴ and R⁵ is hydrogen. In embodiments, R² is hydrogen. In embodiments, R³ is hydrogen. In embodiments, R⁴ is hydrogen. In embodiments, R⁵ is hydrogen. In embodiments, R² and R³ are hydrogen. In embodiments, R² and R⁴ are hydrogen. In embodiments, R² and R⁵ are hydrogen. In embodiments, R³ and R⁴ are hydrogen. In embodiments, R³ and R⁵ are hydrogen. In embodiments, R⁴ and R⁵ are hydrogen. In embodiments, R², R³ and R⁵ are hydrogen. In embodiments, R², R⁴ and R⁵ are hydrogen. In embodiments, R³, R⁴ and R⁵ are hydrogen. In embodiments, R², R³, R⁴ and R⁵ are hydrogen.

In embodiments, R² is hydrogen. In embodiments, R³ is hydrogen. In embodiments, R⁵ is hydrogen. In embodiments, R⁴ is —F. In embodiments, R⁴ is —Cl. In embodiments, R⁴ is —Br. In embodiments, R² and R³ are hydrogen and R⁴ is —F. In embodiments, R² and R⁵ are hydrogen and R⁴ is —F. In embodiments, R³ and R⁵ are hydrogen and R⁴ is —F. In embodiments, R² and R³ are hydrogen and R⁴ is —Cl. In embodiments, R² and R⁵ are hydrogen and R⁴ is —Cl. In embodiments, R³ and R⁵ are hydrogen and R⁴ is —Cl. In embodiments, R² and R³ are hydrogen and R⁴ is —Br. In embodiments, R² and R⁵ are hydrogen and R⁴ is —Br. In embodiments, R³ and R⁵ are hydrogen and R⁴ is —Br. In embodiments, R², R³ and R⁵ are hydrogen and R⁴ is —F. In embodiments, R², R³ and R⁵ are hydrogen and R⁴ is —Cl. In embodiments, R², R³ and R⁵ are hydrogen and R⁴ is —Br.

In embodiments, at least one of R², R³, R⁴ and R⁵ is substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, at least one of R², R³, R⁴ and R⁵ is unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, at least one of R², R³, R⁴ and R⁵ is substituted or unsubstituted phenyl, or substituted or unsubstituted 5 to 6 membered heteroaryl. In embodiments, at least one of R², R³, R⁴ and R⁵ is unsubstituted phenyl, or unsubstituted 5 to 6 membered heteroaryl. In embodiments, at least one of R², R³, R⁴ and R⁵ is substituted or unsubstituted phenyl. In embodiments, at least one of R², R³, R⁴ and R⁵ is unsubstituted phenyl. In embodiments, at least one of R², R³, R⁴ and R⁵ is unsubstituted 5 to 6 membered heteroaryl. In embodiments, at least one of R², R³, R⁴ and R⁵ is substituted or unsubstituted pyridyl. In embodiments, at least one of R², R³, R⁴ and R⁵ is unsubstituted pyridyl. In embodiments, at least two of R², R³, R⁴ and R⁵ are substituted or unsubstituted pyridyl. In embodiments, at least two of R², R³, R⁴ and R⁵ are unsubstituted pyridyl. In embodiments, R² is substituted or unsubstituted pyridyl. In embodiments, R² is R^{2E}-substituted or unsubstituted pyridyl. In embodiments, R² is unsubstituted pyridyl. In embodiments, R³ is substituted or unsubstituted pyridyl. In embodiments, R³ is R^{3E}-substituted or unsubstituted pyridyl. In embodiments, R³ is unsubstituted pyridyl. In embodiments, R⁴ is substituted or unsubstituted pyridyl. In embodiments, R⁴ is R^{4E}-substituted or unsubstituted pyridyl. In embodiments, R⁴ is unsubstituted pyridyl. In embodiments, R⁵ is substituted or unsubstituted pyridyl. In embodiments, R⁵ is R^{5E}-substituted or unsubstituted pyridyl. In embodiments, R⁵ is unsubstituted pyridyl.

In embodiments, R^{2E} is independently halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, or —OCH₂I. In embodiments, R^{2E} is independently halogen. In embodiments, R^{2E} is independently —CF₃. In embodiments, R^{2E} is independently —CCl₃. In embodiments, R^{2E} is independently —CBr₃. In embodiments, R^{2E} is independently —CI₃. In embodiments, R^{2E} is independently —CHF₂. In embodiments, R^{2E} is independently —CHCl₂. In embodiments, R^{2E} is independently —CHBr₂. In embodiments, R^{2E} is independently —CHI₂. In embodiments, R^{2E} is independently —CHF. In embodiments, R^{2E} is independently —CH₂Cl. In embodiments, R^{2E} is independently —CH₂Br. In embodiments, R^{2E} is independently —CHI. In embodiments, R^{2E} is independently —CN. In embodiments, R^{2E} is independently —OH. In embodiments, R^{2E} is independently —NH₂. In embodiments, R^{2E} is independently —COOH. In embodiments, R^{2E} is independently —CONH₂. In embodiments, R^{2E} is independently —NO₂. In embodiments, R^{2E} is independently —SH. In embodiments, R^{2E} is independently —SO₃H. In embodiments, R^{2E} is independently —SO₄H. In embodiments, R^{2E} is independently —NHC(O)NHNH₂. In embodiments, R^{2E} is independently —NHC(O)NH₂. In embodiments, R^{2E} is independently —NHOH. In embodiments, R^{2E} is independently —OCF₃. In embodiments, R^{2E} is independently —OCCl₃. In embodiments, R^{2E} is independently —OCBr₃. In embodiments, R^{2E} is independently —OC₃. In embodiments, R^{2E} is independently —OCHF₂. In embodiments, R^{2E} is independently —OCHCl₂. In embodiments, R^{2E} is independently —OCHBr₂. In embodiments, R^{2E} is independently or —OCHI₂. In embodiments, R^{2E} is independently substituted or unsubstituted C₁-C₄ alkyl. In embodiments, R^{2E} is independently unsubstituted C₁-C₄ alkyl. In embodiments, R^{2E} is independently unsubstituted methyl. In embodiments, R^{2E} is independently unsubstituted ethyl. In embodiments, R^{2E} is independently unsubstituted propyl. In embodiments, R^{2E} is independently substituted or unsubstituted 2 to 6 membered heteroalkyl. In embodiments, R^{2E} is independently unsubstituted 2 to 6 membered heteroalkyl. In embodiments, R^{2E} is independently substituted or unsubstituted C₃-C₆ cycloalkyl. In embodiments, R^{2E} is independently unsubstituted C₃-C₆ cycloalkyl. In embodiments, R^{2E} is independently substituted or unsubstituted 5 to 6 membered heterocycloalkyl. In embodiments, R^{2E} is independently unsubstituted 5 to 6 membered heterocycloalkyl. In embodiments, R^{2E} is independently substituted or unsubstituted phenyl. In embodiments, R^{2E} is independently unsubstituted phenyl. In embodiments, R^{2E} is independently substituted or unsubstituted 5 to 6 membered heteroaryl. In embodiments, R^{2E} is independently unsubstituted 5 to 6 membered heteroaryl.

In embodiments, R^{3E} is independently halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, or —OCH₂I. In embodiments, R^{3E} is independently halogen. In embodiments, R^{3E} is independently —CF₃. In embodiments, R^{3E} is independently —CCl₃. In embodiments, R^{3E} is independently —CBr₃. In embodiments, R^{3E} is independently —CI₃. In embodiments, R^{3E} is independently —CHF₂. In embodiments, R^{3E} is independently —CHCl₂. In embodiments, R^{3E} is independently —CHBr₂. In embodiments, R^{3E} is independently —CHI₂. In embodiments, R^{3E} is independently —CH₂F. In embodiments, R^{3E} is independently —CH₂C₁. In embodiments, R^{3E} is independently —CH₂Br. In embodiments, R^{3E} is independently —CH₂I. In embodiments, R^{3E} is independently —CN. In embodiments, R^{3E} is independently —OH. In embodiments, R^{3E} is independently —NH₂. In embodiments, R^{3E} is independently —COOH. In embodiments, R^{3E} is independently —CONH₂. In embodiments, R^{3E} is independently —NO₂. In embodiments, R^{3E} is independently —SH. In embodiments, R^{3E} is independently —SO₃H. In embodiments, R^{3E} is independently —SO₄H. In embodiments, R^{3E} is independently —NHC(O)NHNH₂. In embodiments, R^{3E} is independently —NHC(O)NH₂. In embodiments, R^{3E} is independently —NHOH. In embodiments, R^{3E} is independently —OCF₃. In embodiments, R^{3E} is independently —OCCl₃. In embodiments, R^{3E} is independently —OCBr₃. In embodiments, R^{3E} is independently —OCI₃. In embodiments, R^{3E} is independently —OCHF₂. In embodiments, R^{3E} is independently —OCHCl₂. In embodiments, R^{3E} is independently —OCHBr₂. In embodiments, R^{3E} is independently or —OCHI₂. In embodiments, R^{3E} is independently substituted or unsubstituted C₁-C₄ alkyl. In embodiments, R^{3E} is independently unsubstituted C₁-C₄ alkyl. In embodiments, R^{3E} is independently unsubstituted methyl. In embodiments, R^{3E} is independently unsubstituted ethyl. In embodiments, R^{3E} is independently unsubstituted propyl. In embodiments, R^{3E} is independently substituted or unsubstituted 2 to 6 membered heteroalkyl. In embodiments, R^{3E} is independently unsubstituted 2 to 6 membered heteroalkyl. In embodiments, R^{3E} is independently substituted or unsubstituted C₃-C₆ cycloalkyl. In embodiments, R^{3E} is independently unsubstituted C₃-C₆ cycloalkyl. In embodiments, R^{3E} is independently substituted or unsubstituted 5 to 6 membered heterocycloalkyl. In embodiments, R^{3E} is independently unsubstituted 5 to 6 membered heterocycloalkyl. In embodiments, R^{3E} is independently substituted or unsubstituted phenyl. In embodiments, R^{3E} is independently unsubstituted phenyl. In embodiments, R^{3E} is independently substituted or unsubstituted 5 to 6 membered heteroaryl. In embodiments, R^{3E} is independently unsubstituted 5 to 6 membered heteroaryl.

In embodiments, R^{4E} is independently halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, or —OCH₂I. In embodiments, R^{4E} is independently halogen. In embodiments, R^{4E} is independently —CF₃. In embodiments, R^{4E} is independently —CCl₃. In embodiments, R^{4E} is independently —CBr₃. In embodiments, R^{4E} is independently —CI₃. In embodiments, R^{4E} is independently —CHF₂. In embodiments, R^{4E} is independently —CHCl₂. In embodiments, R^{4E} is independently —CHBr₂. In embodiments, R^{4E} is independently —CHI₂. In embodiments, R^{4E} is independently —CH₂F. In embodiments, R^{4E} is independently —CH₂C₁. In embodiments, R^{4E} is independently —CH₂Br. In embodiments, R^{4E} is independently —CHI. In embodiments, R^{4E} is independently —CN. In embodiments, R^{4E} is independently —OH. In embodiments, R^{4E} is independently —NH₂. In embodiments, R^{4E} is independently —COOH. In embodiments, R^{4E} is independently —CONH₂. In embodiments, R^{4E} is independently —NO₂. In embodiments, R^{4E} is independently —SH. In embodiments, R^{4E} is independently —SO₃H. In embodiments, R^{4E} is independently —SO₄H. In embodiments, R^{4E} is independently —NHC(O)NHNH₂. In embodiments, R^{4E} is independently —NHC(O)NH₂. In embodiments, R^{4E} is independently —NHOH. In embodiments, R^{4E} is independently —OCF₃. In embodiments, R^{4E} is independently —OCCl₃. In embodiments, R^{4E} is independently —OCBr₃. In embodiments, R^{4E} is independently —OCI₃. In embodiments, R^{4E} is independently —OCHF₂. In embodiments, R^{4E} is independently —OCHCl₂. In embodiments, R^{4E} is independently —OCHBr₂. In embodiments, R^{4E} is independently or —OCHI₂. In embodiments, R^{4E} is independently substituted or unsubstituted C₁-C₄ alkyl. In embodiments, R^{4E} is independently unsubstituted C₁-C₄ alkyl. In embodiments, R^{4E} is independently unsubstituted methyl. In embodiments, R^{4E} is independently unsubstituted ethyl. In embodiments, R^{4E} is independently unsubstituted propyl. In embodiments, R^{4E} is independently substituted or unsubstituted 2 to 6 membered heteroalkyl. In embodiments, R^{4E} is independently unsubstituted 2 to 6 membered heteroalkyl. In embodiments, R^{4E} is independently substituted or unsubstituted C₃-C₆ cycloalkyl. In embodiments, R^{4E} is independently unsubstituted C₃-C₆ cycloalkyl. In embodiments, R^{4E} is independently substituted or unsubstituted 5 to 6 membered heterocycloalkyl. In embodiments, R^{4E} is independently unsubstituted 5 to 6 membered heterocycloalkyl. In embodiments, R^{4E} is independently substituted or unsubstituted phenyl. In embodiments, R^{4E} is independently unsubstituted phenyl. In embodiments, R^{4E} is independently substituted or unsubstituted 5 to 6 membered heteroaryl. In embodiments, R^{4E} is independently unsubstituted 5 to 6 membered heteroaryl.

In embodiments, R^{5E} is independently halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, or —OCH₂I. In embodiments, R^{5E} is independently halogen. In embodiments, R^{5E} is independently —CF₃. In embodiments, R^{5E} is independently —CCl₃. In embodiments, R^{5E} is independently —CBr₃. In embodiments, R^{5E} is independently —CI₃. In embodiments, R^{5E} is independently —CHF₂. In embodiments, R^{5E} is independently —CHCl₂. In embodiments, R^{5E} is independently —CHBr₂. In embodiments, R^{5E} is independently —CHI₂. In embodiments, R^{5E} is independently —CH₂F. In embodiments, R^{5E} is independently —CH₂Cl. In embodiments, R^{5E} is independently —CH₂Br. In embodiments, R^{5E} is independently —CH₂I. In embodiments, R^{5E} is independently —CN. In embodiments, R^{5E} is independently —OH. In embodiments, R^{5E} is independently —NH₂. In embodiments, R^{5E} is independently —COOH. In embodiments, R^{5E} is independently —CONH₂. In embodiments, R^{5E} is independently —NO₂. In embodiments, R^{5E} is independently —SH. In embodiments, R^{5E} is independently —SO₃H. In embodiments, R^{5E} is independently —SO₄H. In embodiments, R^{5E} is independently —NHC(O)NHNH₂. In embodiments, R^{5E} is independently —NHC(O)NH₂. In embodiments, R^{5E} is independently —NHOH. In embodiments, R^{5E} is independently —OCF₃. In embodiments, R^{5E} is independently —OCCl₃. In embodiments, R^{5E} is independently —OCBr₃. In embodiments, R^{5E} is independently —OCI₃. In embodiments, R^{5E} is independently —OCHF₂. In embodiments, R^{5E} is independently —OCHCl₂. In embodiments, R^{5E} is independently —OCHBr₂. In embodiments, R^{5E} is independently or —OCHI₂. In embodiments, R^{5E} is independently substituted or unsubstituted C₁-C₄ alkyl. In embodiments, R^{5E} is independently unsubstituted C₁-C₄ alkyl. In embodiments, R^{5E} is independently unsubstituted methyl. In embodiments, R^{5E} is independently unsubstituted ethyl. In embodiments, R^{5E} is independently unsubstituted propyl. In embodiments, R^{5E} is independently substituted or unsubstituted 2 to 6 membered heteroalkyl. In embodiments, R^{5E} is independently unsubstituted 2 to 6 membered heteroalkyl. In embodiments, R^{5E} is independently substituted or unsubstituted C₃-C₆ cycloalkyl. In embodiments, R^{5E} is independently unsubstituted C₃-C₆ cycloalkyl. In embodiments, R^{5E} is independently substituted or unsubstituted 5 to 6 membered heterocycloalkyl. In embodiments, R^{5E} is independently unsubstituted 5 to 6 membered heterocycloalkyl. In embodiments, R^{5E} is independently substituted or unsubstituted phenyl. In embodiments, R^{5E} is independently unsubstituted phenyl. In embodiments, R^{5E} is independently substituted or unsubstituted 5 to 6 membered heteroaryl. In embodiments, R^{5E} is independently unsubstituted 5 to 6 membered heteroaryl.

In embodiment, R², R³ and R⁴ are hydrogen, and R⁵ is substituted or unsubstituted pyridyl. In embodiment, R², R³ and R⁴ are hydrogen, and R⁵ is R^{5E}-substituted pyridyl. In embodiment, R², R³ and R⁴ are hydrogen, and R⁵ is halogen-substituted pyridyl. In embodiment, R², R³ and R⁴ are hydrogen, and R⁵ is unsubstituted pyridyl. In embodiment, R², R³ and R⁵ are hydrogen, and R⁴ is substituted or unsubstituted pyridyl. In embodiment, R², R³ and R⁵ are hydrogen, and R⁴ is R^{4E}-substituted pyridyl. In embodiment, R², R³ and R⁵ are hydrogen, and R⁴ is halogen-substituted pyridyl. In embodiment, R², R³ and R⁵ are hydrogen, and R⁴ is unsubstituted pyridyl. In embodiment, R², R⁴ and R⁵ are hydrogen, and R³ is substituted or unsubstituted pyridyl. In embodiment, R², R⁴ and R⁵ are hydrogen, and R³ is R^{3E}-substituted pyridyl. In embodiment, R², R⁴ and R⁵ are hydrogen, and R³ is halogen-substituted pyridyl. In embodiment, R², R⁴ and R⁵ are hydrogen, and R³ is unsubstituted pyridyl. In embodiment, R³, R⁴ and R⁵ are hydrogen, and R² is substituted or unsubstituted pyridyl. In embodiment, R³, R⁴ and R⁵ are hydrogen, and R² is R^{2E}-substituted pyridyl. In embodiment, R³, R⁴ and R⁵ are hydrogen, and R² is halogen-substituted pyridyl. In embodiment, R³, R⁴ and R⁵ are hydrogen, and R² is unsubstituted pyridyl.

In embodiments, the compound has a structure of formula (VII-A)

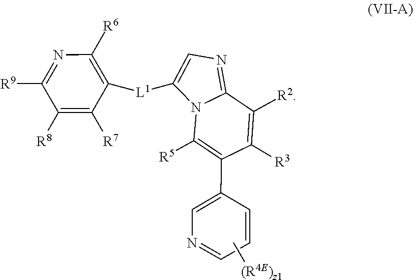

R², R³, R⁵, R⁶, R⁷, R⁸, R⁹, and R^{4E} are described herein. z1 is an integer from 0 to 4. In embodiments, z1 is 0. In embodiments, z1 is 1. In embodiments, z1 is 2. In embodiments, z1 is 3. In embodiments, z1 is 4. In embodiments, R^{4E} is halogen. In embodiments, R^{4E} is —Cl. In embodiments, R^{4E} is —Br. In embodiments, R^{4E} is —F. In embodiments, R^{4E} is —I. In embodiments, z1 is 1 and R^{4E} is —Cl. In embodiments, z1 is 1 and R^{4E} is —Br. In embodiments, z1 is 1 and R^{4E} is —F. In embodiments, z1 is 1 and R^{4E} is —I.

In embodiments, the compound has a structure of formula (VII-B)

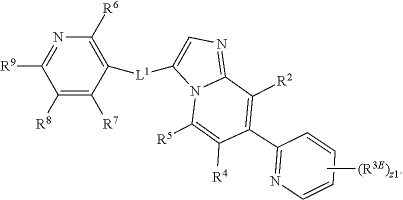

R², R^{3E}, R⁴, R⁵, R⁶, R⁷, R⁸, R⁹, and z1 are described herein. In embodiments, R^{3E} is halogen. In embodiments, R^{3E} is —Cl. In embodiments, R^{3E} is —Br. In embodiments, R^{3E} is —F. In embodiments, R^{3E} is —I. In embodiments, z1 is 1 and R^{3E} is —Cl. In embodiments, z1 is 1 and R^{3E} is —Br. In embodiments, z1 is 1 and R^{3E} is —F. In embodiments, z1 is 1 and R^{3E} is —I.

In embodiments, the compound has a structure of formula (VII-C)

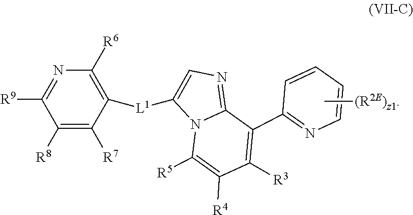

R^{2E}, R³, R⁴, R⁵, R⁶, R⁷, R⁸, R⁹ and z1 are described herein. In embodiments, R^{2E} is halogen. In embodiments, R^{2E} is —Cl. In embodiments, R^{2E} is —Br. In embodiments, R^{2E} is —F. In embodiments, R^{2E} is —I. In embodiments, z1 is 1 and R^{2E} is —Cl. In embodiments, z1 is 1 and R^{2E} is —Br. In embodiments, z1 is 1 and R^{2E} is —F. In embodiments, z1 is 1 and R^{2E} is —I.

In embodiments, the compound has a structure of formula (VII-D)

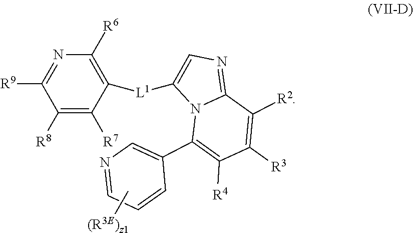

R², R³, R⁴, R^{5E}, R⁶, R⁷, R⁸, R⁹ and z1 are described herein. In embodiments, R^{3E} is halogen. In embodiments, R^{3E} is —Cl. In embodiments, R^{3E} is —Br. In embodiments, R^{3E} is —F. In embodiments, R^{3E} is —I. In embodiments, z1 is 1 and R^{3E} is —Cl. In embodiments, z1 is 1 and R^{3E} is —Br. In embodiments, z1 is 1 and R^{3E} is —F. In embodiments, z1 is 1 and R^{3E} is —I.

In embodiments, R⁶ is hydrogen. In embodiments, R⁶ is halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, or —OCH₂I. In embodiments, R⁶ is substituted or unsubstituted C₁-C₈ alkyl. In embodiments, R⁶ is substituted or unsubstituted C₁-C₆ alkyl. In embodiments, R¹ is substituted or unsubstituted C₁-C₄ alkyl. In embodiments, R⁶ is substituted or unsubstituted C₁-C₃alkyl. In embodiments, R⁶ is substituted or unsubstituted C₁-C₂ alkyl. In embodiments, R⁶ is unsubstituted C₁-C₈ alkyl. In embodiments, R¹ is unsubstituted C₁-C₆ alkyl. In embodiments, R⁶ is unsubstituted C₁-C₄ alkyl. In embodiments, R⁶ is unsubstituted C₁-C₃ alkyl. In embodiments, R⁶ is unsubstituted C₁-C₂ alkyl. In embodiments, R⁶ is unsubstituted propyl. In embodiments, R⁶ is unsubstituted isopropyl. In embodiments, R⁶ is unsubstituted ethyl. In embodiments, R⁶ is unsubstituted methyl. In embodiments, R⁶ is substituted or unsubstituted 2 to 8 membered heteroalkyl. In embodiments, R⁶ is substituted or unsubstituted 2 to 6 membered heteroalkyl. In embodiments, R⁶ is substituted or unsubstituted 4 to 6 membered heteroalkyl. In embodiments, R⁶ is substituted or unsubstituted 2 to 3 membered heteroalkyl. In embodiments, R⁶ is substituted or unsubstituted 4 to 5 membered heteroalkyl. In embodiments, R⁶ is unsubstituted 2 to 8 membered heteroalkyl. In embodiments, R⁶ is unsubstituted 2 to 6 membered heteroalkyl. In embodiments, R⁶ is unsubstituted 4 to 6 membered heteroalkyl. In embodiments, R⁶ is unsubstituted 2 to 3 membered heteroalkyl. In embodiments, R⁶ is unsubstituted 4 to 5 membered heteroalkyl. In embodiments, R⁶ is hydrogen, halogen, —NO₂, or substituted or unsubstituted C₁-C₄ alkyl. In embodiments, R⁶ is hydrogen, halogen, —NO₂, or unsubstituted C₁-C₄ alkyl. In embodiments, R⁶ is hydrogen, halogen, or —NO₂. In embodiments, R⁶ is hydrogen, —NO₂, or unsubstituted C₁-C₄ alkyl. In embodiments, R⁶ is halogen, —NO₂, or unsubstituted C₁-C₄ alkyl. In embodiments, R⁶ is hydrogen, or unsubstituted C₁-C₄ alkyl. In embodiments, R⁶ is hydrogen, halogen, —NO₂, unsubstituted methyl or unsubstituted ethyl.

In embodiments, R⁷ is hydrogen. In embodiments, R⁷ is halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, or —OCH₂I. In embodiments, R⁷ is substituted or unsubstituted C₁-C₈ alkyl. In embodiments, R⁷ is substituted or unsubstituted C₁-C₆ alkyl. In embodiments, R¹ is substituted or unsubstituted C₁-C₄ alkyl. In embodiments, R⁷ is substituted or unsubstituted C₁-C₃alkyl. In embodiments, R⁷ is substituted or unsubstituted C₁-C₂ alkyl. In embodiments, R⁷ is unsubstituted C₁-C₈ alkyl. In embodiments, R¹ is unsubstituted C₁-C₆ alkyl. In embodiments, R⁷ is unsubstituted C₁-C₄ alkyl. In embodiments, R⁷ is unsubstituted C₁-C₃ alkyl. In embodiments, R⁷ is unsubstituted C₁-C₂ alkyl. In embodiments, R⁷ is unsubstituted propyl. In embodiments, R⁷ is unsubstituted isopropyl. In embodiments, R⁷ is unsubstituted ethyl. In embodiments, R⁷ is unsubstituted methyl. In embodiments, R⁷ is substituted or unsubstituted 2 to 8 membered heteroalkyl. In embodiments, R⁷ is substituted or unsubstituted 2 to 6 membered heteroalkyl. In embodiments, R⁷ is substituted or unsubstituted 4 to 6 membered heteroalkyl. In embodiments, R⁷ is substituted or unsubstituted 2 to 3 membered heteroalkyl. In embodiments, R⁷ is substituted or unsubstituted 4 to 5 membered heteroalkyl. In embodiments, R⁷ is unsubstituted 2 to 8 membered heteroalkyl. In embodiments, R⁷ is unsubstituted 2 to 6 membered heteroalkyl. In embodiments, R⁷ is unsubstituted 4 to 6 membered heteroalkyl. In embodiments, R⁷ is unsubstituted 2 to 3 membered heteroalkyl. In embodiments, R⁷ is unsubstituted 4 to 5 membered heteroalkyl. In embodiments, R⁷ is hydrogen, halogen, —NO₂, or substituted or unsubstituted C₁-C₄ alkyl. In embodiments, R⁷ is hydrogen, halogen, —NO₂, or unsubstituted C₁-C₄ alkyl. In embodiments, R⁷ is hydrogen, halogen, or —NO₂. In embodiments, R⁷ is hydrogen, —NO₂, or unsubstituted C₁-C₄ alkyl. In embodiments, R⁷ is halogen, —NO₂, or unsubstituted C₁-C₄ alkyl. In embodiments, R⁷ is hydrogen, or unsubstituted C₁-C₄ alkyl. In embodiments, R⁷ is hydrogen, halogen, —NO₂, unsubstituted methyl or unsubstituted ethyl.

In embodiments, R⁸ is hydrogen. In embodiments, R⁸ is halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, or —OCH₂I. In embodiments, R⁸ is substituted or unsubstituted C₁-C₈ alkyl. In embodiments, R⁸ is substituted or unsubstituted C₁-C₆ alkyl. In embodiments, R¹ is substituted or unsubstituted C₁-C₄ alkyl. In embodiments, R⁸ is substituted or unsubstituted C₁-C₃alkyl. In embodiments, R⁸ is substituted or unsubstituted C₁-C₂ alkyl. In embodiments, R⁸ is unsubstituted C₁-C₈ alkyl. In embodiments, R¹ is unsubstituted C₁-C₆ alkyl. In embodiments, R⁸ is unsubstituted C₁-C₄ alkyl. In embodiments, R⁸ is unsubstituted C₁-C₃ alkyl. In embodiments, R⁸ is unsubstituted C₁-C₂ alkyl. In embodiments, R⁸ is unsubstituted propyl. In embodiments, R⁸ is unsubstituted isopropyl. In embodiments, R⁸ is unsubstituted ethyl. In embodiments, R⁸ is unsubstituted methyl. In embodiments, R⁸ is substituted or unsubstituted 2 to 8 membered heteroalkyl. In embodiments, R⁸ is substituted or unsubstituted 2 to 6 membered heteroalkyl. In embodiments, R⁸ is substituted or unsubstituted 4 to 6 membered heteroalkyl. In embodiments, R⁸ is substituted or unsubstituted 2 to 3 membered heteroalkyl. In embodiments, R⁸ is substituted or unsubstituted 4 to 5 membered heteroalkyl. In embodiments, R⁸ is unsubstituted 2 to 8 membered heteroalkyl. In embodiments, R⁸ is unsubstituted 2 to 6 membered heteroalkyl. In embodiments, R⁸ is unsubstituted 4 to 6 membered heteroalkyl. In embodiments, R⁸ is unsubstituted 2 to 3 membered heteroalkyl. In embodiments, R⁸ is unsubstituted 4 to 5 membered heteroalkyl. In embodiments, R⁸ is hydrogen, halogen, —NO₂, or substituted or unsubstituted C₁-C₄ alkyl. In embodiments, R⁸ is hydrogen, halogen, —NO₂, or unsubstituted C₁-C₄ alkyl. In embodiments, R⁸ is hydrogen, halogen, or —NO₂. In embodiments, R⁸ is hydrogen, —NO₂, or unsubstituted C₁-C₄ alkyl. In embodiments, R⁸ is halogen, —NO₂, or unsubstituted C₁-C₄ alkyl. In embodiments, R⁸ is hydrogen, or unsubstituted C₁-C₄ alkyl. In embodiments, R⁸ is hydrogen, halogen, —NO₂, unsubstituted methyl or unsubstituted ethyl.

In embodiments, R⁹ is hydrogen. In embodiments, R⁹ is halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, or —OCH₂I. In embodiments, R⁹ is substituted or unsubstituted C₁-C₈ alkyl. In embodiments, R⁹ is substituted or unsubstituted C₁-C₆ alkyl. In embodiments, R¹ is substituted or unsubstituted C₁-C₄ alkyl. In embodiments, R⁹ is substituted or unsubstituted C₁-C₃ alkyl. In embodiments, R⁹ is substituted or unsubstituted C₁-C₂ alkyl. In embodiments, R⁹ is unsubstituted C₁-C₈ alkyl. In embodiments, R¹ is unsubstituted C₁-C₆ alkyl. In embodiments, R⁹ is unsubstituted C₁-C₄ alkyl. In embodiments, R⁹ is unsubstituted C₁-C₃ alkyl. In embodiments, R⁹ is unsubstituted C₁-C₂ alkyl. In embodiments, R⁹ is unsubstituted propyl. In embodiments, R⁹ is unsubstituted isopropyl. In embodiments, R⁹ is unsubstituted ethyl. In embodiments, R⁹ is unsubstituted methyl. In embodiments, R⁹ is substituted or unsubstituted 2 to 8 membered heteroalkyl. In embodiments, R⁹ is substituted or unsubstituted 2 to 6 membered heteroalkyl. In embodiments, R⁹ is substituted or unsubstituted 4 to 6 membered heteroalkyl. In embodiments, R⁹ is substituted or unsubstituted 2 to 3 membered heteroalkyl. In embodiments, R⁹ is substituted or unsubstituted 4 to 5 membered heteroalkyl. In embodiments, R⁹ is unsubstituted 2 to 8 membered heteroalkyl. In embodiments, R⁹ is unsubstituted 2 to 6 membered heteroalkyl. In embodiments, R⁹ is unsubstituted 4 to 6 membered heteroalkyl. In embodiments, R⁹ is unsubstituted 2 to 3 membered heteroalkyl. In embodiments, R⁹ is unsubstituted 4 to 5 membered heteroalkyl. In embodiments, R⁹ is hydrogen, halogen, —NO₂, or substituted or unsubstituted C₁-C₄ alkyl. In embodiments, R⁹ is hydrogen, halogen, —NO₂, or unsubstituted C₁-C₄ alkyl. In embodiments, R⁹ is hydrogen, halogen, or —NO₂. In embodiments, R⁹ is hydrogen, —NO₂, or unsubstituted C₁-C₄ alkyl. In embodiments, R⁹ is halogen, —NO₂, or unsubstituted C₁-C₄ alkyl. In embodiments, R⁹ is hydrogen, or unsubstituted C₁-C₄ alkyl. In embodiments, R⁹ is hydrogen, halogen, —NO₂, unsubstituted methyl or unsubstituted ethyl.

In embodiments, R⁶, R⁷, R⁸ and R⁹ are independently hydrogen, halogen, —NO₂, or substituted or unsubstituted C₁-C₃ alkyl. In embodiments, R⁶, R⁷, R⁸ and R⁹ are independently hydrogen, halogen, —NO₂, or unsubstituted C₁-C₃ alkyl. In embodiments, R⁶, R⁷, R⁸ and R⁹ are independently hydrogen, halogen, or unsubstituted C₁-C₃ alkyl. In embodiments, R⁶, R⁷, R⁸ and R⁹ are independently hydrogen, —NO₂, or unsubstituted C₁-C₃ alkyl. In embodiments, R⁶, R⁷, R⁸ and R⁹ are independently hydrogen, halogen, or unsubstituted C₁-C₃ alkyl. In embodiments, R⁶, R⁷, R⁸ and R⁹ are independently hydrogen, or —NO₂. In embodiments, R⁶, R⁷, R⁸ and R⁹ are independently hydrogen, or halogen.

In embodiments, L¹ is unsubstituted phenylene. In embodiments, R¹, R², R³ and R⁵ are hydrogen. In embodiments, R⁴ is —F, —Cl or —Br. In embodiments, R⁶, R⁷, R⁸ and R⁹ are independently hydrogen, halogen, —NO₂, or unsubstituted C₁-C₃ alkyl. In embodiments, L¹ is unsubstituted phenylene; and R¹, R², R³ and R⁵ are hydrogen. In embodiments, L¹ is unsubstituted phenylene; and R⁴ is —F, —Cl or —Br. In embodiments, L¹ is unsubstituted phenylene; and R⁶, R⁷, R⁸ and R⁹ are independently hydrogen, halogen, —NO₂, or unsubstituted C₁-C₃ alkyl. In embodiments, L¹ is unsubstituted phenylene and R¹, R², R³ and R⁵ are hydrogen. In embodiments, L¹ is unsubstituted phenylene; R¹, R², R³ and R⁵ are hydrogen; and R⁴ is —F, —Cl or —Br. In embodiments, L¹ is unsubstituted phenylene; R¹, R², R³ and R⁵ are hydrogen; R⁴ is —F, —Cl or —Br; and R⁶, R⁷, R⁸ and R⁹ are independently hydrogen, halogen, —NO₂, or unsubstituted C₁-C₃ alkyl.

In embodiments, L¹ is substituted or unsubstituted C₄-C₆ alkylene, or substituted or unsubstituted 4 to 6 membered heteroalkylene. In embodiments, R¹, R², R³ and R⁵ are hydrogen. In embodiments, R⁴ is —Cl or —Br. In embodiments, R⁶, R⁷, R⁸ and R⁹ are independently hydrogen, halogen, —NO₂, or unsubstituted C₁-C₃ alkyl. In embodiments, L¹ is substituted or unsubstituted C₄-C₆ alkylene, or substituted or unsubstituted 4 to 6 membered heteroalkylene; and R¹, R², R³ and R⁵ are hydrogen. In embodiments, L¹ is substituted or unsubstituted C₄-C₆ alkylene, or substituted or unsubstituted 4 to 6 membered heteroalkylene; and R⁴ is —Cl or —Br. In embodiments, L¹ is substituted or unsubstituted C₄-C₆ alkylene, or substituted or unsubstituted 4 to 6 membered heteroalkylene; and R⁶, R⁷, R⁸ and R⁹ are independently hydrogen, halogen, —NO₂, or unsubstituted C₁-C₃ alkyl. In embodiments, L¹ is substituted or unsubstituted C₄-C₆ alkylene, or substituted or unsubstituted 4 to 6 membered heteroalkylene; R⁴ is —Cl or —Br; and R⁶, R⁷, R⁸ and R⁹ are independently hydrogen, halogen, —NO₂, or unsubstituted C₁-C₃ alkyl. In embodiments, L¹ is substituted or unsubstituted C₄-C₆ alkylene, or substituted or unsubstituted 4 to 6 membered heteroalkylene; R¹, R², R³ and R⁵ are hydrogen; and R⁴ is —Cl or —Br. In embodiments, L¹ is substituted or unsubstituted C₄-C₆ alkylene, or substituted or unsubstituted 4 to 6 membered heteroalkylene; R⁴ is —Cl or —Br; and R⁶, R⁷, R⁸ and R⁹ are independently hydrogen, halogen, —NO₂, or unsubstituted C₁-C₃ alkyl. In embodiments, L¹ is substituted or unsubstituted C₄-C₆ alkylene, or substituted or unsubstituted 4 to 6 membered heteroalkylene; R¹, R², R³ and R⁵ are hydrogen; and R⁶, R⁷, R⁸ and R⁹ are independently hydrogen, halogen, —NO₂, or unsubstituted C₁-C₃ alkyl.

In embodiments, L¹ is —SO₂—N(CH₃)N═CH—. In embodiments, R², R³ and R⁵ is hydrogen. In embodiments, R⁴ is halogen, or substituted or unsubstituted pyridyl. In embodiments, R⁴ is halogen-substituted pyridyl or unsubstituted pyridyl. In embodiments, R⁶ is unsubstituted methyl. In embodiments, R⁷ is hydrogen. In embodiments, R⁸ is —NO₂. In embodiments, R⁹ is hydrogen. In embodiments, L¹ is —SO₂—N(CH₃)N═CH—; and R², R³ and R⁵ is hydrogen. In embodiments, L¹ is —SO₂—N(CH₃)N═CH—; and R⁴ is halogen, or substituted or unsubstituted pyridyl. In embodiments, L¹ is —SO₂—N(CH₃)N═CH—; and R⁴ is halogen-substituted pyridyl or unsubstituted pyridyl. In embodiments, L¹ is —SO₂—N(CH₃)N═CH—; and R⁶ is unsubstituted methyl. In embodiments, L¹ is —SO₂—N(CH₃)N═CH—; and R⁷ is hydrogen. In embodiments, L¹ is —SO₂—N(CH₃)N═CH—; and R⁸ is —NO₂. In embodiments, L¹ is —SO₂—N(CH₃)N═CH—; and R⁹ is hydrogen. In embodiments, L¹ is —SO₂—N(CH₃)N═CH—; R², R³ and R⁵ is hydrogen; and R⁴ is halogen, or substituted or unsubstituted pyridyl. In embodiments, L¹ is —SO₂—N(CH₃)N═CH—; R², R³ and R⁵ is hydrogen; and R⁴ is halogen-substituted pyridyl or unsubstituted pyridyl. In embodiments, L¹ is —SO₂—N(CH₃)N═CH—; R², R³ and R⁸ is hydrogen; and R⁶ is unsubstituted methyl. In embodiments, L¹ is —SO₂—N(CH₃)N═CH—; R², R³ and R⁵ is hydrogen; and R⁷ is hydrogen. In embodiments, L¹ is —SO₂—N(CH₃)N═CH—; R², R³ and R⁵ is hydrogen; and R⁸ is —NO₂. In embodiments, L¹ is —SO₂—N(CH₃)N═CH—; R², R³ and R⁵ is hydrogen; and R⁹ is hydrogen. In embodiments, L¹ is —SO₂—N(CH₃)N═CH—; R², R³ and R⁵ is hydrogen; R⁴ is halogen, or substituted or unsubstituted pyridyl; and R⁶ is unsubstituted methyl. In embodiments, L¹ is —SO₂—N(CH₃)N═CH—; R², R³ and R⁵ is hydrogen; R⁴ is halogen, or substituted or unsubstituted pyridyl; and R⁷ is hydrogen. In embodiments, L¹ is —SO₂—N(CH₃)N═CH—; R², R³ and R⁵ is hydrogen; R⁴ is halogen, or substituted or unsubstituted pyridyl; and R⁸ is —NO₂. In embodiments, L¹ is —SO₂—N(CH₃)N═CH—; R², R³ and R⁵ is hydrogen; R⁴ is halogen, or substituted or unsubstituted pyridyl; and R⁹ is hydrogen. In embodiments, L¹ is —SO₂—N(CH₃)N═CH—; R², R³ and R⁵ is hydrogen; R⁴ is halogen, or substituted or unsubstituted pyridyl; R⁴ is halogen-substituted pyridyl or unsubstituted pyridyl; R⁶ is unsubstituted methyl; and R⁷ is hydrogen. In embodiments, L¹ is —SO₂—N(CH₃)N═CH—; R², R³ and R⁵ is hydrogen; R⁴ is halogen, or substituted or unsubstituted pyridyl; R⁸ is —NO₂; and R⁹ is hydrogen. In embodiments, L¹ is —SO₂—N(CH₃)N═CH—; R², R³ and R⁵ is hydrogen; R⁴ is halogen, or substituted or unsubstituted pyridyl; R⁴ is halogen-substituted pyridyl or unsubstituted pyridyl; R⁶ is unsubstituted methyl; R⁷ is hydrogen R⁸ is —NO₂; and R⁹ is hydrogen. In embodiments, L¹ is —SO₂—N(CH₃)N═CH—; R², R³ and R⁵ is hydrogen; R⁴ is halogen-substituted pyridyl or unsubstituted pyridyl; R⁶ is unsubstituted methyl; R⁷ is hydrogen R⁸ is —NO₂; and R⁹ is hydrogen.

In embodiments, R⁴ is Ring A. In embodiments, the compound has a structure of formula (VIII-A):

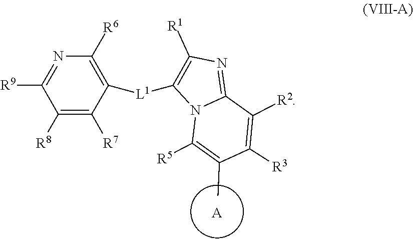

R¹, R², R³, R⁵, R⁶, R⁸, R⁹, and R¹⁰ are as described herein. Ring A is independently substituted or unsubstituted cycloalkyl, substituted or unsubstituted heterocycloalkyl, substituted or unsubstituted aryl, or substituted or unsubstituted heteroaryl.

In embodiments, Ring A is substituted or unsubstituted cycloalkyl. In embodiments, Ring A is substituted or unsubstituted heterocycloalkyl. In embodiments, Ring A is substituted or unsubstituted aryl. In embodiments, Ring A is substituted or unsubstituted heteroaryl. In embodiments, Ring A is substituted or unsubstituted (C₃-C₁₀) cycloalkyl, substituted or unsubstituted 3 to 10 membered heterocycloalkyl, substituted or unsubstituted (C₆-C₁₀) aryl, or substituted or unsubstituted 5 to 10 membered heteroaryl. In embodiments, Ring A is substituted or unsubstituted (C₃-C₁₀) cycloalkyl. In embodiments, Ring A is substituted or unsubstituted 3 to 10 membered heterocycloalkyl. In embodiments, Ring A is substituted or unsubstituted (C₆-C₁₀) aryl. In embodiments, Ring A is substituted or unsubstituted 5 to 10 membered heteroaryl. In embodiments, Ring A is substituted or unsubstituted (C₃-C₆) cycloalkyl. In embodiments, Ring A is substituted or unsubstituted 3 to 6 membered heterocycloalkyl. In embodiments, Ring A is substituted or unsubstituted phenyl. In embodiments, Ring A is substituted or unsubstituted naphthyl. In embodiments, Ring A is substituted or unsubstituted 5 to 9 membered heteroaryl. In embodiments, Ring A is substituted or unsubstituted 5 to 6 membered heteroaryl. In embodiments, Ring A is an unsubstituted 5 to 6 membered heteroaryl. In embodiments, Ring A is substituted or unsubstituted 5 membered heteroaryl. In embodiments, Ring A is a substituted 5 membered heteroaryl. In embodiments, Ring A is an unsubstituted 5 membered heteroaryl.

In embodiments, Ring A is substituted cycloalkyl. In embodiments, Ring A is substituted heterocycloalkyl. In embodiments, Ring A is substituted aryl. In embodiments, Ring A is substituted heteroaryl. In embodiments, Ring A is substituted (C₃-C₁₀) cycloalkyl, substituted 3 to 10 membered heterocycloalkyl, substituted (C₆-C₁₀) aryl, or substituted 5 to 10 membered heteroaryl. In embodiments, Ring A is substituted (C₃-C₁₀) cycloalkyl. In embodiments, Ring A is substituted 3 to 10 membered heterocycloalkyl. In embodiments, Ring A is substituted (C₆-C₁₀) aryl. In embodiments, Ring A is substituted 5 to 10 membered heteroaryl. In embodiments, Ring A is substituted (C₃-C₆) cycloalkyl. In embodiments, Ring A is substituted 3 to 6 membered heterocycloalkyl. In embodiments, Ring A is substituted phenyl. In embodiments, Ring A is substituted naphthyl. In embodiments, Ring A is substituted 5 to 9 membered heteroaryl. In embodiments, Ring A is substituted 5 to 6 membered heteroaryl.

In embodiments, Ring A is R¹⁶-substituted cycloalkyl. In embodiments, Ring A is R¹⁶-substituted heterocycloalkyl. In embodiments, Ring A is R¹⁶-substituted aryl. In embodiments, Ring A is R¹⁶-substituted heteroaryl. In embodiments, Ring A is R¹⁶-substituted (C₃-C₁₀) cycloalkyl, R¹⁶-substituted 3 to 10 membered heterocycloalkyl, R¹⁶-substituted (C₆-C₁₀) aryl, or R¹⁶-substituted 5 to 10 membered heteroaryl. In embodiments, Ring A is R¹⁶-substituted (C₃-C₁₀) cycloalkyl. In embodiments, Ring A is R¹⁶-substituted 3 to 10 membered heterocycloalkyl. In embodiments, Ring A is R¹⁶-substituted (C₆-C₁₀) aryl. In embodiments, Ring A is R¹⁶-substituted 5 to 10 membered heteroaryl. In embodiments, Ring A is R¹⁶-substituted (C₃-C₆) cycloalkyl. In embodiments, Ring A is R¹⁶-substituted 3 to 6 membered heterocycloalkyl. In embodiments, Ring A is R¹⁶-substituted phenyl. In embodiments, Ring A is R¹⁶-substituted naphthyl. In embodiments, Ring A is R¹⁶-substituted 5 to 9 membered heteroaryl. In embodiments, Ring A is R¹⁶-substituted 5 to 6 membered heteroaryl.

In embodiments, Ring A is unsubstituted cycloalkyl. In embodiments, Ring A is unsubstituted heterocycloalkyl. In embodiments, Ring A is unsubstituted aryl. In embodiments, Ring A is unsubstituted heteroaryl. In embodiments, Ring A is unsubstituted (C₃-C₁₀) cycloalkyl, unsubstituted 3 to 10 membered heterocycloalkyl, unsubstituted (C₆-C₁₀) aryl, or unsubstituted 5 to 10 membered heteroaryl. In embodiments, Ring A is unsubstituted (C₃-C₁₀) cycloalkyl. In embodiments, Ring A is unsubstituted 3 to 10 membered heterocycloalkyl. In embodiments, Ring A is unsubstituted (C₆-C₁₀) aryl. In embodiments, Ring A is unsubstituted 5 to 10 membered heteroaryl. In embodiments, Ring A is unsubstituted (C₃-C₆) cycloalkyl. In embodiments, Ring A is unsubstituted 3 to 6 membered heterocycloalkyl. In embodiments, Ring A is unsubstituted phenyl. In embodiments, Ring A is unsubstituted naphthyl. In embodiments, Ring A is unsubstituted 5 to 9 membered heteroaryl. In embodiments, Ring A is unsubstituted 5 to 6 membered heteroaryl.

In embodiments, Ring A is substituted or unsubstituted (C₆-C₁₀) aryl or substituted or unsubstituted 5 to 10 membered heteroaryl. In embodiments, Ring A is substituted or unsubstituted (C₆-C₁₀) aryl or substituted or unsubstituted 5 to 10 membered heteroaryl.

In embodiments, Ring A is substituted or unsubstituted (C₆-C₁₀) aryl. Ring A is substituted or unsubstituted phenyl. In embodiments, Ring A is substituted or unsubstituted napthyl. In embodiments, Ring A is substituted or unsubstituted 5 to 10 membered heteroaryl. In embodiments, Ring A is substituted or unsubstituted 5 to 6 membered heteroaryl. In embodiments, Ring A is substituted or unsubstituted thienyl. In embodiments, Ring A is substituted or unsubstituted furanyl. In embodiments, Ring A is substituted or unsubstituted pyrrolyl. In embodiments, Ring A is substituted or unsubstituted imidazolyl. In embodiments, Ring A is substituted or unsubstituted pyrazolyl. In embodiments, Ring A is substituted or unsubstituted oxazolyl. In embodiments, Ring A is substituted or unsubstituted isoxazolyl. In embodiments, Ring A is substituted or unsubstituted pyridinyl. In embodiments, Ring A is substituted or unsubstituted pyridyl. In embodiments, Ring A is substituted or unsubstituted pyrazinyl. Ring A is substituted or unsubstituted pyrimidinyl. In embodiments, Ring A is substituted or unsubstituted pyridazinyl. In embodiments, Ring A is substituted or unsubstituted 1,2,3-triazinyl. In embodiments, Ring A is substituted or unsubstituted 1,2,4-triazinyl. In embodiments, Ring A is substituted or unsubstituted 1,3,5-triazinyl.

In embodiments, Ring A is R¹⁶-substituted (C₆-C₁₀) aryl or R¹⁶-substituted 5 to 10 membered heteroaryl. In embodiments, Ring A is R¹⁶-substituted (C₆-C₁₀) aryl or R¹⁶-substituted 5 to 10 membered heteroaryl. In embodiments, Ring A is R¹⁶-substituted (C₆-C₁₀) aryl. In embodiments, Ring A is R¹⁶-substituted phenyl. In embodiments, Ring A is R¹⁶-substituted napthyl. In embodiments, Ring A is R¹⁶-substituted 5 to 10 membered heteroaryl. In embodiments, Ring A is R¹⁶-substituted 5 to 6 membered heteroaryl. In embodiments, Ring A is R¹⁶-substituted thienyl. In embodiments, Ring A is R¹⁶-substituted furanyl. In embodiments, Ring A is R¹⁶-substituted pyrrolyl. In embodiments, Ring A is R¹⁶-substituted imidazolyl. In embodiments, Ring A is R¹⁶-substituted pyrazolyl. In embodiments, Ring A is R¹⁶-substituted oxazolyl. In embodiments, Ring A is R¹⁶-substituted isoxazolyl. In embodiments, Ring A is R¹⁶-substituted pyridinyl. In embodiments, Ring A is R¹⁶-substituted pyridyl. In embodiments, Ring A is R¹⁶-substituted pyrazinyl. In embodiments, Ring A is R¹⁶-substituted pyrimidinyl. In embodiments, Ring A is R¹⁶-substituted pyridazinyl. In embodiments, Ring A is R¹⁶-substituted 1,2,3-triazinyl. In embodiments, Ring A is R¹⁶-substituted 1,2,4-triazinyl. In embodiments, Ring A is R¹⁶-substituted 1,3,5-triazinyl.

In embodiments, Ring A is unsubstituted (C₆-C₁₀) aryl or unsubstituted 5 to 10 membered heteroaryl. In embodiments, Ring A is unsubstituted (C₆-C₁₀) aryl or unsubstituted 5 to 10 membered heteroaryl. In embodiments, Ring A is unsubstituted (C₆-C₁₀) aryl. Ring A is unsubstituted phenyl. In embodiments, Ring A is unsubstituted napthyl. In embodiments, Ring A is unsubstituted 5 to 10 membered heteroaryl. In embodiments, Ring A is unsubstituted 5 to 6 membered heteroaryl. In embodiments, Ring A is unsubstituted thienyl. In embodiments, Ring A is unsubstituted furanyl. In embodiments, Ring A is unsubstituted pyrrolyl. In embodiments, Ring A is unsubstituted imidazolyl. In embodiments, Ring A is unsubstituted pyrazolyl. In embodiments, Ring A is unsubstituted oxazolyl. In embodiments, Ring A is unsubstituted isoxazolyl. In embodiments, Ring A is unsubstituted pyridinyl. In embodiments, Ring A is unsubstituted pyridyl. In embodiments, Ring A is unsubstituted pyrazinyl. In embodiments, Ring A is unsubstituted pyrimidinyl. In embodiments, Ring A is unsubstituted pyridazinyl. In embodiments, Ring A is unsubstituted 1,2,3-triazinyl. In embodiments, Ring A is unsubstituted 1,2,4-triazinyl. In embodiments, Ring A is unsubstituted 1,3,5-triazinyl.

In embodiments, Ring A is substituted or unsubstituted 2-pyridyl. In embodiments, Ring A is substituted or unsubstituted 3-pyridyl. In embodiments, Ring A is substituted or unsubstituted 4-pyridyl. In embodiments, Ring A is halogen-substituted 2-pyridyl. In embodiments, Ring A is halogen-substituted 3-pyridyl. In embodiments, Ring A is halogen-substituted 4-pyridyl. In embodiments, Ring A is unsubstituted 2-pyridyl. In embodiments, Ring A is unsubstituted 3-pyridyl. In embodiments, Ring A is unsubstituted 4-pyridyl.

In embodiments, Ring A is R¹⁶-substituted 2-pyridyl. In embodiments, Ring A is R¹⁶-substituted 3-pyridyl. In embodiments, Ring A is R¹⁶-substituted 4-pyridyl. In embodiments, Ring A is halogen-substituted 2-pyridyl. In embodiments, Ring A is halogen-substituted 3-pyridyl. In embodiments, Ring A is halogen-substituted 4-pyridyl. In embodiments, Ring A is unsubstituted 2-pyridyl. In embodiments, Ring A is unsubstituted 3-pyridyl. In embodiments, Ring A is unsubstituted 4-pyridyl.

In embodiments, L¹ has a length of about 4 to 12 Å. In embodiments, L¹ has a length of about 4 to 11 Å. In embodiments, L¹ has a length of about 4 to 10 Å. In embodiments, L¹ has a length of about 4 to 9 Å. In embodiments, L¹ has a length of about 4 to 8 Å. In embodiments, L¹ has a length of about 4 to 7 Å. In embodiments, L¹ has a length of about 4 to 6 Å. In embodiments, L¹ has a length of about 4 to 5.5 Å. In embodiments, L¹ has a length of about 4.5 to 5.5 Å.

In embodiments, the compound has an IC₅₀ value of about 50 μM or less against p38γ kinase activity. In embodiments, the compound has an IC₅₀ value of about 40 μM or less against p38γ kinase activity. In embodiments, the compound has an IC₅₀ value of about 30 μM or less against p38γ kinase activity. In embodiments, the compound has an IC₅₀ value of about 25 μM or less against p38γ kinase activity. In embodiments, the compound has an IC₅₀ value of about 20 μM or less against p38γ kinase activity. In embodiments, the compound has an IC₅₀ value of about 15 μM or less against p38γ kinase activity. In embodiments, the compound has an IC₅₀ value of about 10 μM or less against p38γ kinase activity. In embodiments, the compound has an IC₅₀ value of about 9 μM or less against p38γ kinase activity. In embodiments, the compound has an IC₅₀ value of about 8 μM or less against p38γ kinase activity. In embodiments, the compound has an IC₅₀ value of about 7 μM or less against p38γ kinase activity. In embodiments, the compound has an IC₅₀ value of about 6 μM or less against p38γ kinase activity. In embodiments, the compound has an IC₅₀ value of about 5 μM or less against p38γ kinase activity. In embodiments, the compound has an IC₅₀ value of about 4 μM or less against p38γ kinase activity. In embodiments, the compound has an IC₅₀ value of about 3 μM or less against p38γ kinase activity. In embodiments, the compound has an IC₅₀ value of about 2 μM or less against p38γ kinase activity. In embodiments, the compound has an IC₅₀ value of about 1 μM or less against p38γ kinase activity. In embodiments, the compound has an IC₅₀ value of about 900 nM or less against p38γ kinase activity. In embodiments, the compound has an IC₅₀ value of about 800 nM or less against p38γ kinase activity. In embodiments, the compound has an IC₅₀ value of about 700 nM or less against p38γ kinase activity. In embodiments, the compound has an IC₅₀ value of about 600 nM or less against p38γ kinase activity. In embodiments, the compound has an IC₅₀ value of about 500 nM or less against p38γ kinase activity. In embodiments, the compound has an IC₅₀ value of about 400 nM or less against p38γ kinase activity. In embodiments, the compound has an IC₅₀ value of about 300 nM or less against p38γ kinase activity. In embodiments, the compound has an IC₅₀ value of about 200 nM or less against p38γ kinase activity. In embodiments, the compound has an IC₅₀ value of about 100 nM or less against p38γ kinase activity. In embodiments, the compound has an IC₅₀ value of about 50 nM or less against p38γ kinase activity. In embodiments, the compound has an IC₅₀ value of about 40 nM or less against p38γ kinase activity. In embodiments, the compound has an IC₅₀ value of about 30 nM or less against p38γ kinase activity. In embodiments, the compound has an IC₅₀ value of about 20 nM or less against p38γ kinase activity. In embodiments, the compound has an IC₅₀ value of about 10 nM or less against p38γ kinase activity.

In embodiments, the compound has an IC₅₀ value 50 μM or less against p38γ kinase activity. In embodiments, the compound has an IC₅₀ value 40 μM or less against p38γ kinase activity. In embodiments, the compound has an IC₅₀ value 30 μM or less against p38γ kinase activity. In embodiments, the compound has an IC₅₀ value 25 μM or less against p38γ kinase activity. In embodiments, the compound has an IC₅₀ value 20 μM or less against p38γ kinase activity. In embodiments, the compound has an IC₅₀ value 15 μM or less against p38γ kinase activity. In embodiments, the compound has an IC₅₀ value 10 μM or less against p38γ kinase activity. In embodiments, the compound has an IC₅₀ value 9 μM or less against p38γ kinase activity. In embodiments, the compound has an IC₅₀ value 8 μM or less against p38γ kinase activity. In embodiments, the compound has an IC₅₀ value 7 μM or less against p38γ kinase activity. In embodiments, the compound has an IC₅₀ value 6 μM or less against p38γ kinase activity. In embodiments, the compound has an IC₅₀ value 5 μM or less against p38γ kinase activity. In embodiments, the compound has an IC₅₀ value 4 μM or less against p38γ kinase activity. In embodiments, the compound has an IC₅₀ value 3 μM or less against p38γ kinase activity. In embodiments, the compound has an IC₅₀ value 2 μM or less against p38γ kinase activity. In embodiments, the compound has an IC₅₀ value 1 μM or less against p38γ kinase activity. In embodiments, the compound has an IC₅₀ value 900 nM or less against p38γ kinase activity. In embodiments, the compound has an IC₅₀ value 800 nM or less against p38γ kinase activity. In embodiments, the compound has an IC₅₀ value 700 nM or less against p38γ kinase activity. In embodiments, the compound has an IC₅₀ value 600 nM or less against p38γ kinase activity. In embodiments, the compound has an IC₅₀ value 500 nM or less against p38γ kinase activity. In embodiments, the compound has an IC₅₀ value 400 nM or less against p38γ kinase activity. In embodiments, the compound has an IC₅₀ value 300 nM or less against p38γ kinase activity. In embodiments, the compound has an IC₅₀ value 200 nM or less against p38γ kinase activity. In embodiments, the compound has an IC₅₀ value 100 nM or less against p38γ kinase activity. In embodiments, the compound has an IC₅₀ value 50 nM or less against p38γ kinase activity. In embodiments, the compound has an IC₅₀ value 40 nM or less against p38γ kinase activity. In embodiments, the compound has an IC₅₀ value 30 nM or less against p38γ kinase activity. In embodiments, the compound has an IC₅₀ value 20 nM or less against p38γ kinase activity. In embodiments, the compound has an IC₅₀ value 10 nM or less against p38γ kinase activity.

R¹ is hydrogen, halogen (e.g., —F, —Cl, Br, —I), —CX¹₃, —CHX¹₂, —CH₂X¹, —OCX¹₃, —OCH₂X¹, —OCHX¹₂, —N₃, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). X¹ is independently —F, —Cl, —Br, or —I. In embodiments, R¹ is hydrogen, halogen (e.g., —F, —Cl, Br, —I), —CX¹₃, —CHX¹₂, —CH₂X¹, —OCX¹₃, —OCH₂X¹, —OCHX¹₂, —N₃, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}S O₂R^{1D}, —NR^{1A}C(O)R^{1C}, —NR^{1A}C(O)OR^{1C}, —NR^{1A}OR^{1C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), R^{1E}-substituted or unsubstituted alkyl (e.g., C₁-C₈, C₁-C₆, C₁-C₄, or C₁-C₂), R^{1E}-substituted or unsubstituted heteroalkyl (e.g., 2 to 8 membered, 2 to 6 membered, 4 to 6 membered, 2 to 3 membered, or 4 to 5 membered), R^{1E}-substituted or unsubstituted cycloalkyl (e.g., C₃-C₈, C₃-C₆, C₄-C₆, or C₅-C₆), R^{1E}-substituted or unsubstituted heterocycloalkyl (e.g., 3 to 8 membered, 3 to 6 membered, 4 to 6 membered, 4 to 5 membered, or 5 to 6 membered), R^{1E}-substituted or unsubstituted aryl (e.g., C₆-C₁₂, C₆-C₁₀, or phenyl), or R^{1E}-substituted or unsubstituted heteroaryl (e.g., 5 to 12 membered, 5 to 10 membered, 5 to 9 membered, or 5 to 6 membered). In embodiments, R¹ is hydrogen, halogen (e.g., —F, —Cl, Br, —I), —CX¹₃, —CHX¹₂, —CH₂X¹, —OCX¹₃, —OCH₂X¹, —OCHX¹₂, —N₃, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, R¹ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R¹ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{1E}-substituted or unsubstituted C₁-C₆ alkyl, R^{1E}-substituted or unsubstituted 2 to 6 membered heteroalkyl, R^{1E}-substituted or unsubstituted C₃-C₆ cycloalkyl, R^{1E}-substituted or unsubstituted 3 to 6 membered heterocycloalkyl, R^{1E}-substituted or unsubstituted phenyl, or R^{1E}-substituted or unsubstituted 5 to 6 membered heteroaryl. In embodiments, R¹ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{1E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, RE is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{1F}-substituted or unsubstituted alkyl (e.g., C₁-C₈, C₁-C₆, C₁-C₄, or C₁-C₂), R^{1F}-substituted or unsubstituted heteroalkyl (e.g., 2 to 8 membered, 2 to 6 membered, 4 to 6 membered, 2 to 3 membered, or 4 to 5 membered), R^{1F}-substituted or unsubstituted cycloalkyl (e.g., C₃-C₈, C₃-C₆, C₄-C₆, or C₅-C₆), R^{1F}-substituted or unsubstituted heterocycloalkyl (e.g., 3 to 8 membered, 3 to 6 membered, 4 to 6 membered, 4 to 5 membered, or 5 to 6 membered), R^{1F}-substituted or unsubstituted aryl (e.g., C₆-C₁₂, C₆-C₁₀, or phenyl), or R^{1F}-substituted or unsubstituted heteroaryl (e.g., 5 to 12 membered, 5 to 10 membered, 5 to 9 membered, or 5 to 6 membered). In embodiments, R^{1E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R^{1E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{1F}-substituted or unsubstituted C₁-C₆ alkyl, R^{1F}-substituted or unsubstituted 2 to 6 membered heteroalkyl, R^{1F}-substituted or unsubstituted C₃-C₆ cycloalkyl, R^{1F}-substituted or unsubstituted 3 to 6 membered heterocycloalkyl, R^{1F}-substituted or unsubstituted phenyl, or R^{1F}-substituted or unsubstituted 5 to 6 membered heteroaryl. In embodiments, R^{1E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{1F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In embodiments, R^{1F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, C₁-C₆ unsubstituted alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

In embodiments, R² is hydrogen, halogen (e.g., —F, —Cl, Br, —I), —CX²₃, —CHX²₂, —CH₂X², —OCX²₃, —OCH₂X², —OCHX²₂, —N₃, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}S O₂R^{2D}, —NR^{2A}C(O)R^{2C}, —NR^{2A}C(O)OR^{2C}, —NR^{2A}OR^{2C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). X² is independently —F, —Cl, —Br, or —I. In embodiments, R² is hydrogen, halogen (e.g., —F, —Cl, Br, —I), —CX²₃, —CHX²₂, —CH₂X², —OCX²₃, —OCH₂X², —OCHX²₂, —N₃, —CN, —SOₙ₂R^{2D}, —SO₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}S O₂R^{2D}, —NR^{2A}C(O)R^{2C}, —NR^{2A}C(O)OR^{2C}, —NR^{2A}OR^{2C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), R^{2E}-substituted or unsubstituted alkyl (e.g., C₁-C₈, C₁-C₆, C₁-C₄, or C₁-C₂), R^{2E}-substituted or unsubstituted heteroalkyl (e.g., 2 to 8 membered, 2 to 6 membered, 4 to 6 membered, 2 to 3 membered, or 4 to 5 membered), R^{2E}-substituted or unsubstituted cycloalkyl (e.g., C₃-C₈, C₃-C₆, C₄-C₆, or C₅-C₆), R^{2E}-substituted or unsubstituted heterocycloalkyl (e.g., 3 to 8 membered, 3 to 6 membered, 4 to 6 membered, 4 to 5 membered, or 5 to 6 membered), R^{2E}-substituted or unsubstituted aryl (e.g., C₆-C₁₂, C₆-C₁₀, or phenyl), or R^{2E}-substituted or unsubstituted heteroaryl (e.g., 5 to 12 membered, 5 to 10 membered, 5 to 9 membered, or 5 to 6 membered). In embodiments, R² is hydrogen, halogen (e.g., —F, —Cl, Br, —I), —CX²₃, —CHX²₂, —CH₂X², —OCX²₃, —OCH₂X², —OCHX²₂, —N₃, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A} SO₂R^{2D}, —NR^{2A}C(O)R^{2C}, —NR^{2A}C(O)OR^{2C}, —NR^{2A}OR^{2C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, R² is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R² is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{2E}-substituted or unsubstituted C₁-C₆ alkyl, R^{2E}-substituted or unsubstituted 2 to 6 membered heteroalkyl, R^{2E}-substituted or unsubstituted C₃-C₆ cycloalkyl, R^{2E}-substituted or unsubstituted 3 to 6 membered heterocycloalkyl, R^{2E}-substituted or unsubstituted phenyl, or R^{2E}-substituted or unsubstituted 5 to 6 membered heteroaryl. In embodiments, R² is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{2E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, R^{2E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{2F}-substituted or unsubstituted alkyl (e.g., C₁-C₈, C₁-C₆, C₁-C₄, or C₁-C₂), R^{2F}-substituted or unsubstituted heteroalkyl (e.g., 2 to 8 membered, 2 to 6 membered, 4 to 6 membered, 2 to 3 membered, or 4 to 5 membered), R^{2F}-substituted or unsubstituted cycloalkyl (e.g., C₃-C₈, C₃-C₆, C₄-C₆, or C₅-C₆), R^{2F}-substituted or unsubstituted heterocycloalkyl (e.g., 3 to 8 membered, 3 to 6 membered, 4 to 6 membered, 4 to 5 membered, or 5 to 6 membered), R^{2F}-substituted or unsubstituted aryl (e.g., C₆-C₁₂, C₆-C₁₀, or phenyl), or R^{2F}-substituted or unsubstituted heteroaryl (e.g., 5 to 12 membered, 5 to 10 membered, 5 to 9 membered, or 5 to 6 membered). In embodiments, R^{2E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R^{2E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{2F}-substituted or unsubstituted C₁-C₆ alkyl, R^{2F}-substituted or unsubstituted 2 to 6 membered heteroalkyl, R^{2F}-substituted or unsubstituted C₃-C₆ cycloalkyl, R^{2F}-substituted or unsubstituted 3 to 6 membered heterocycloalkyl, R^{2F}-substituted or unsubstituted phenyl, or R^{2F}-substituted or unsubstituted 5 to 6 membered heteroaryl. In embodiments, R^{2E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{2F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In embodiments, R^{2F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, C₁-C₆ unsubstituted alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

In embodiments, R³ is hydrogen, halogen (e.g., —F, —Cl, Br, —I), —CX³₃, —CHX³₂, —CH₂X³, —OCX³₃, —OCH₂X³, —OCHX³₂, —N₃, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}S O₂R^{3D}, —NR^{3A}C(O)R^{3C}, —NR^{3A}C(O)OR^{3C}, —NR^{3A}OR^{3C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). X³ is independently —F, —Cl, —Br, or —I. In embodiments, R³ is hydrogen, halogen (e.g., —F, —Cl, Br, —I), —CX³₃, —CHX³₂, —CH₂X³, —OCX³₃, —OCH₂X³, —OCHX³₂, —N₃, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}S O₂R^{3D}, —NR^{3A}C(O)R^{3C}, —NR^{3A}C(O)OR^{3C}, —NR^{3A}OR^{3C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), R^{3E}-substituted or unsubstituted alkyl (e.g., C₁-C₈, C₁-C₆, C₁-C₄, or C₁-C₂), R^{3E}-substituted or unsubstituted heteroalkyl (e.g., 2 to 8 membered, 2 to 6 membered, 4 to 6 membered, 2 to 3 membered, or 4 to 5 membered), R^{3E}-substituted or unsubstituted cycloalkyl (e.g., C₃-C₈, C₃-C₆, C₄-C₆, or C₅-C₆), R^{3E}-substituted or unsubstituted heterocycloalkyl (e.g., 3 to 8 membered, 3 to 6 membered, 4 to 6 membered, 4 to 5 membered, or 5 to 6 membered), R^{3E}-substituted or unsubstituted aryl (e.g., C₆-C₁₂, C₆-C₁₀, or phenyl), or R^{3E}-substituted or unsubstituted heteroaryl (e.g., 5 to 12 membered, 5 to 10 membered, 5 to 9 membered, or 5 to 6 membered). In embodiments, R³ is hydrogen, halogen (e.g., —F, —Cl, Br, —I), —CX³₃, —CHX³₂, —CH₂X³, —OCX³₃, —OCH₂X³, —OCHX³₂, —N₃, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}S O₂R^{3D}, —NR^{3A}C(O)R^{3C}, —NR^{3A}C(O)OR^{3C}, —NR^{3A}OR^{3C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, R³ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R³ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{3E}-substituted or unsubstituted C₁-C₆ alkyl, R^{3E}-substituted or unsubstituted 2 to 6 membered heteroalkyl, R^{3E}-substituted or unsubstituted C₃-C₆ cycloalkyl, R^{3E}-substituted or unsubstituted 3 to 6 membered heterocycloalkyl, R^{3E}-substituted or unsubstituted phenyl, or R^{3E}-substituted or unsubstituted 5 to 6 membered heteroaryl. In embodiments, R³ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{3E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, R^{3E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{3F}-substituted or unsubstituted alkyl (e.g., C₁-C₈, C₁-C₆, C₁-C₄, or C₁-C₂), R^{3F}-substituted or unsubstituted heteroalkyl (e.g., 2 to 8 membered, 2 to 6 membered, 4 to 6 membered, 2 to 3 membered, or 4 to 5 membered), R^{3F}-substituted or unsubstituted cycloalkyl (e.g., C₃-C₈, C₃-C₆, C₄-C₆, or C₅-06), R^{3F}-substituted or unsubstituted heterocycloalkyl (e.g., 3 to 8 membered, 3 to 6 membered, 4 to 6 membered, 4 to 5 membered, or 5 to 6 membered), R^{3F}-substituted or unsubstituted aryl (e.g., C₆-C₁₂, C₆-C₁₀, or phenyl), or R^{3F}-substituted or unsubstituted heteroaryl (e.g., 5 to 12 membered, 5 to 10 membered, 5 to 9 membered, or 5 to 6 membered). In embodiments, R^{3E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH2I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R^{3E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{3F}-substituted or unsubstituted C₁-C₆ alkyl, R^{3F}-substituted or unsubstituted 2 to 6 membered heteroalkyl, R^{3F}-substituted or unsubstituted C₃-C₆ cycloalkyl, R^{3F}-substituted or unsubstituted 3 to 6 membered heterocycloalkyl, R^{3F}-substituted or unsubstituted phenyl, or R^{3F}-substituted or unsubstituted 5 to 6 membered heteroaryl. In embodiments, R^{3E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{3F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In embodiments, R^{3F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, C₁-C₆ unsubstituted alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

In embodiments, R⁴ is hydrogen, halogen (e.g., —F, —Cl, Br, —I), —CX⁴₃, —CHX⁴₂, —CH₂X⁴, —OCX⁴₃, —OCH₂X⁴, —OCHX⁴₂, —N₃, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). X⁴ is independently —F, —Cl, —Br, or —I. In embodiments, R⁴ is hydrogen, halogen (e.g., —F, —Cl, Br, —I), —CX⁴₃, —CHX⁴₂, —CH₂X⁴, —OCX⁴₃, —OCH₂X⁴, —OCHX⁴₂, —N₃, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), R^{4E}-substituted or unsubstituted alkyl (e.g., C₁-C₈, C₁-C₆, C₁-C₄, or C₁-C₂), R^{4E}-substituted or unsubstituted heteroalkyl (e.g., 2 to 8 membered, 2 to 6 membered, 4 to 6 membered, 2 to 3 membered, or 4 to 5 membered), R^{4E}-substituted or unsubstituted cycloalkyl (e.g., C₃-C₈, C₃-C₆, C₄-C₆, or C₅-C₆), R^{4E}-substituted or unsubstituted heterocycloalkyl (e.g., 3 to 8 membered, 3 to 6 membered, 4 to 6 membered, 4 to 5 membered, or 5 to 6 membered), R^{4E}-substituted or unsubstituted aryl (e.g., C₆-C₁₂, C₆-C₁₀, or phenyl), or R^{4E}-substituted or unsubstituted heteroaryl (e.g., 5 to 12 membered, 5 to 10 membered, 5 to 9 membered, or 5 to 6 membered). In embodiments, R⁴ is hydrogen, halogen (e.g., —F, —Cl, Br, —I), —CX⁴₃, —CHX⁴₂, —CH₂X⁴, —OCX⁴₃, —OCH₂X⁴, —OCHX⁴₂, —N₃, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —C(O)—OR^{4C}, —C(O)NR^{4A}R^{4B}, —OR^{4D}, —NR^{4A}S O₂R^{4D}, —NR^{4A}C(O)R^{4C}, —NR^{4A}C(O)OR^{4C}, —NR^{4A}OR^{4C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, R⁴ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁴ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{4E}-substituted or unsubstituted C₁-C₆ alkyl, R^{4E}-substituted or unsubstituted 2 to 6 membered heteroalkyl, R^{4E}-substituted or unsubstituted C₃-C₆ cycloalkyl, R^{4E}-substituted or unsubstituted 3 to 6 membered heterocycloalkyl, R^{4E}-substituted or unsubstituted phenyl, or R^{4E}-substituted or unsubstituted 5 to 6 membered heteroaryl. In embodiments, R⁴ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{4E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, R^{4E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{4F}-substituted or unsubstituted alkyl (e.g., C₁-C₈, C₁-C₆, C₁-C₄, or C₁-C₂), R^{4F}-substituted or unsubstituted heteroalkyl (e.g., 2 to 8 membered, 2 to 6 membered, 4 to 6 membered, 2 to 3 membered, or 4 to 5 membered), R^{4F}-substituted or unsubstituted cycloalkyl (e.g., C₃-C₈, C₃-C₆, C₄-C₆, or C₅-C₆), R^{4F}-substituted or unsubstituted heterocycloalkyl (e.g., 3 to 8 membered, 3 to 6 membered, 4 to 6 membered, 4 to 5 membered, or 5 to 6 membered), R^{4F}-substituted or unsubstituted aryl (e.g., C₆-C₁₂, C₆-C₁₀, or phenyl), or R^{4F}-substituted or unsubstituted heteroaryl (e.g., 5 to 12 membered, 5 to 10 membered, 5 to 9 membered, or 5 to 6 membered). In embodiments, R^{4E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R^{4E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{4F}-substituted or unsubstituted C₁-C₆ alkyl, R^{4F}-substituted or unsubstituted 2 to 6 membered heteroalkyl, R^{4F}-substituted or unsubstituted C₃-C₆ cycloalkyl, R^{4F}-substituted or unsubstituted 3 to 6 membered heterocycloalkyl, R^{4F}-substituted or unsubstituted phenyl, or R^{4F}-substituted or unsubstituted 5 to 6 membered heteroaryl. In embodiments, R^{4E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{4F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In embodiments, R^{4F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, C₁-C₆ unsubstituted alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

In embodiments, R⁵ is hydrogen, halogen (e.g., —F, —Cl, Br, —I), —CX⁵₃, —CHX⁵₂, —CH₂X⁵, —OCX⁵₃, —OCH₂X⁵, —OCHX⁵₂, —N₃, —CN, —SOₙ₅R_{5D}, —SOᵥ₅NR^{5A}R^{5B}, —NHC(O)NR^{5A}R^{5B}, —N(O)ₘ₅, —NR^{5A}R^{5B}, —C(O)R^{5C}, —C(O)—OR^{5C}, —C(O)NR^{5A}R^{5B}, —OR^{5D}, —NR^{5A}S O₂R^{5D}, —NR^{5A}C(O)R^{5C}, —NR^{5A}C(O)OR^{5C}, —NR^{5A}OR^{5C}, (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). X⁵ is independently —F, —Cl, —Br, or —I. In embodiments, R⁵ is hydrogen, halogen (e.g., —F, —Cl, Br, —I), —CX⁵₃, —CHX⁵₂, —CH₂X⁵, —OCX⁵₃, —OCH₂X⁵, —OCHX⁵₂, —N₃, —CN, —SOₙ₅R^{5D}, —SOᵥ₅NR^{5A}R^{5B}, —NHC(O)NR^{5A}R^{5B}, —N(O)ₘ₅, —NR^{5A}R^{5B}, —C(O)R^{5C}, —C(O)—OR^{5C}, —C(O)NR^{5A}R^{5B}, —OR^{5D}, —NR^{5A}S O₂R^{5D}, —NR^{5A}C(O)R^{5C}, —NR^{5A}C(O)OR^{5C}, —NR^{5A}OR^{5C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), R^{5E}-substituted or unsubstituted alkyl (e.g., C₁-C₈, C₁-C₆, C₁-C₄, or C₁-C₂), R^{5E}-substituted or unsubstituted heteroalkyl (e.g., 2 to 8 membered, 2 to 6 membered, 4 to 6 membered, 2 to 3 membered, or 4 to 5 membered), R^{5E}-substituted or unsubstituted cycloalkyl (e.g., C₃-C₈, C₃-C₆, C₄-C₆, or C₅-C₆), R^{5E}-substituted or unsubstituted heterocycloalkyl (e.g., 3 to 8 membered, 3 to 6 membered, 4 to 6 membered, 4 to 5 membered, or 5 to 6 membered), R^{5E}-substituted or unsubstituted aryl (e.g., C₆-C₁₂, C₆-C₁₀, or phenyl), or R^{5E}-substituted or unsubstituted heteroaryl (e.g., 5 to 12 membered, 5 to 10 membered, 5 to 9 membered, or 5 to 6 membered). In embodiments, R⁵ is hydrogen, halogen (e.g., —F, —Cl, Br, —I), —CX⁵₃, —CHX⁵₂, —CH₂X⁵, —OCX⁵₃, —OCH₂X⁵, —OCHX⁵₂, —N₃, —CN, —SOₙ₅R^{5D}, —SOᵥ₅NR^{5A}R^{5B}, —NHC(O)NR^{5A}R^{5B}, —N(O)ₘ₅, —NR^{5A}R^{5B}, —C(O)R^{5C}, —C(O)—OR^{5C}, —C(O)NR^{5A}R^{5B}, —OR^{5D}, —NR^{5A}S O₂R^{5D}, —NR^{5A}C(O)R^{5C}, —NR^{5A}C(O)OR^{5C}, —NR^{5A}OR^{5C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, R⁵ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁵ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{5E}-substituted or unsubstituted C₁-C₆ alkyl, R^{5E}-substituted or unsubstituted 2 to 6 membered heteroalkyl, R^{5E}-substituted or unsubstituted C₃-C₆ cycloalkyl, R^{5E}-substituted or unsubstituted 3 to 6 membered heterocycloalkyl, R^{5E}-substituted or unsubstituted phenyl, or R^{5E}-substituted or unsubstituted 5 to 6 membered heteroaryl. In embodiments, R⁵ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{5E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, R^{5E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{5F}-substituted or unsubstituted alkyl (e.g., C₁-C₈, C₁-C₆, C₁-C₄, or C₁-C₂), R^{5F}-substituted or unsubstituted heteroalkyl (e.g., 2 to 8 membered, 2 to 6 membered, 4 to 6 membered, 2 to 3 membered, or 4 to 5 membered), R^{5F}-substituted or unsubstituted cycloalkyl (e.g., C₃-C₈, C₃-C₆, C₄-C₆, or C₅-C₆), R^{5F}-substituted or unsubstituted heterocycloalkyl (e.g., 3 to 8 membered, 3 to 6 membered, 4 to 6 membered, 4 to 5 membered, or 5 to 6 membered), R^{5F}-substituted or unsubstituted aryl (e.g., C₆-C₁₂, C₆-C₁₀, or phenyl), or R^{5F}-substituted or unsubstituted heteroaryl (e.g., 5 to 12 membered, 5 to 10 membered, 5 to 9 membered, or 5 to 6 membered). In embodiments, R^{5E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R^{5E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{5F}-substituted or unsubstituted C₁-C₆ alkyl, R^{5F}-substituted or unsubstituted 2 to 6 membered heteroalkyl, R^{5F}-substituted or unsubstituted C₃-C₆ cycloalkyl, R^{5F}-substituted or unsubstituted 3 to 6 membered heterocycloalkyl, R^{5F}-substituted or unsubstituted phenyl, or R^{5F}-substituted or unsubstituted 5 to 6 membered heteroaryl. In embodiments, R^{5E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{5F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In embodiments, R^{5F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, C₁-C₆ unsubstituted alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

In embodiments, R⁶ is a bond (to L¹), hydrogen, halogen (e.g., —F, —Cl, Br, —I), —CX⁶₃, —CHX⁶₂, —CH₂X⁶, —OCX⁶₃, —OCH₂X⁶, —OCHX⁶₂, —N₃, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}S O₂R^{6D}, —NR^{6A}C(O)R^{6C}, —NR^{6A}C(O)OR^{6C}, —NR^{6A}OR^{6C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). X⁶ is independently —F, —Cl, —Br, or —I. In embodiments, R⁶ is a bond (to L¹), hydrogen, halogen (e.g., —F, —Cl, Br, —I), —CX⁶₃, —CHX⁶₂, —CH₂X⁶, —OCX⁶₃, —OCH₂X⁶, —OCHX⁶₂, —N₃, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), R^{6E}-substituted or unsubstituted alkyl (e.g., C₁-C₈, C₁-C₆, C₁-C₄, or C₁-C₂), R^{6E}-substituted or unsubstituted heteroalkyl (e.g., 2 to 8 membered, 2 to 6 membered, 4 to 6 membered, 2 to 3 membered, or 4 to 5 membered), R^{6E}-substituted or unsubstituted cycloalkyl (e.g., C₃-C₈, C₃-C₆, C₄-C₆, or C₅-C₆), R^{6E}-substituted or unsubstituted heterocycloalkyl (e.g., 3 to 8 membered, 3 to 6 membered, 4 to 6 membered, 4 to 5 membered, or 5 to 6 membered), R^{6E}-substituted or unsubstituted aryl (e.g., C₆-C₁₂, C₆-C₁₀, or phenyl), or R^{6E}-substituted or unsubstituted heteroaryl (e.g., 5 to 12 membered, 5 to 10 membered, 5 to 9 membered, or 5 to 6 membered). In embodiments, R⁶ is a bond (to L¹), hydrogen, halogen (e.g., —F, —Cl, Br, —I), —CX⁶₃, —CHX⁶₂, —CH₂X⁶, —OCX⁶₃, —OCH₂X⁶, —OCHX⁶₂, —N₃, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}S O₂R^{6D}, —NR^{6A}C(O)R^{6C}, —NR^{6A}C(O)OR^{6C}, —NR^{6A}OR^{6C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, R⁶ is a bond (to L¹), hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁶ is a bond (to L¹), hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{6E}-substituted or unsubstituted C₁-C₆ alkyl, R^{6E}-substituted or unsubstituted 2 to 6 membered heteroalkyl, R^{6E}-substituted or unsubstituted C₃-C₆ cycloalkyl, R^{6E}-substituted or unsubstituted 3 to 6 membered heterocycloalkyl, R^{6E}-substituted or unsubstituted phenyl, or R^{6E}-substituted or unsubstituted 5 to 6 membered heteroaryl. In embodiments, R⁶ is a bond (to L¹), hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

In embodiments, R⁶ is hydrogen, halogen (e.g., —F, —Cl, Br, —I), —CX⁶₃, —CHX⁶₂, —CH₂X⁶, —OCX⁶₃, —OCH₂X⁶, —OCHX⁶₂, —N₃, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}S O₂R^{6D}, —NR^{6A}C(O)R^{6C}, —NR^{6A}C(O)OR^{6C}, —NR^{6A}OR^{6C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). X⁶ is independently —F, —Cl, —Br, or —I. In embodiments, R⁶ is hydrogen, halogen (e.g., —F, —Cl, Br, —I), —CX⁶₃, —CHX⁶₂, —CH₂X⁶, —OCX⁶₃, —OCH₂X⁶, —OCHX⁶₂, —N₃, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), R^{6E}-substituted or unsubstituted alkyl (e.g., C₁-C₈, C₁-C₆, C₁-C₄, or C₁-C₂), R^{6E}-substituted or unsubstituted heteroalkyl (e.g., 2 to 8 membered, 2 to 6 membered, 4 to 6 membered, 2 to 3 membered, or 4 to 5 membered), R^{6E}-substituted or unsubstituted cycloalkyl (e.g., C₃-C₈, C₃-C₆, C₄-C₆, or C₅-C₆), R^{6E}-substituted or unsubstituted heterocycloalkyl (e.g., 3 to 8 membered, 3 to 6 membered, 4 to 6 membered, 4 to 5 membered, or 5 to 6 membered), R^{6E}-substituted or unsubstituted aryl (e.g., C₆-C₁₂, C₆-C₁₀, or phenyl), or R^{6E}-substituted or unsubstituted heteroaryl (e.g., 5 to 12 membered, 5 to 10 membered, 5 to 9 membered, or 5 to 6 membered). In embodiments, R⁶ is hydrogen, halogen (e.g., —F, —Cl, Br, —I), —CX⁶₃, —CHX⁶₂, —CH₂X⁶, —OCX⁶₃, —OCH₂X⁶, —OCHX⁶₂, —N₃, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}S O₂R^{6D}, —NR^{6A}C(O)R^{6C}, —NR^{6A}C(O)OR^{6C}, —NR^{6A}OR^{6C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, R⁶ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁶ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{6E}-substituted or unsubstituted C₁-C₆ alkyl, R^{6E}-substituted or unsubstituted 2 to 6 membered heteroalkyl, R^{6E}-substituted or unsubstituted C₃-C₆ cycloalkyl, R^{6E}-substituted or unsubstituted 3 to 6 membered heterocycloalkyl, R^{6E}-substituted or unsubstituted phenyl, or R^{6E}-substituted or unsubstituted 5 to 6 membered heteroaryl. In embodiments, R⁶ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{6E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, R^{6E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{6F}-substituted or unsubstituted alkyl (e.g., C₁-C₈, C₁-C₆, C₁-C₄, or C₁-C₂), R^{6F}-substituted or unsubstituted heteroalkyl (e.g., 2 to 8 membered, 2 to 6 membered, 4 to 6 membered, 2 to 3 membered, or 4 to 5 membered), R^{6F}-substituted or unsubstituted cycloalkyl (e.g., C₃-C₈, C₃-C₆, C₄-C₆, or C₅-C₆), R^{6F}-substituted or unsubstituted heterocycloalkyl (e.g., 3 to 8 membered, 3 to 6 membered, 4 to 6 membered, 4 to 5 membered, or 5 to 6 membered), R^{6F}-substituted or unsubstituted aryl (e.g., C₆-C₁₂, C₆-C₁₀, or phenyl), or R^{6F}-substituted or unsubstituted heteroaryl (e.g., 5 to 12 membered, 5 to 10 membered, 5 to 9 membered, or 5 to 6 membered). In embodiments, R^{6E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R^{6E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{6F}-substituted or unsubstituted C₁-C₆ alkyl, R^{6F}-substituted or unsubstituted 2 to 6 membered heteroalkyl, R^{6F}-substituted or unsubstituted C₃-C₆ cycloalkyl, R^{6F}-substituted or unsubstituted 3 to 6 membered heterocycloalkyl, R^{6F}-substituted or unsubstituted phenyl, or R^{6F}-substituted or unsubstituted 5 to 6 membered heteroaryl. In embodiments, R^{6E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{6F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In embodiments, R^{6F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, C₁-C₆ unsubstituted alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

In embodiments, R⁷ is a bond (to L¹), hydrogen, halogen (e.g., —F, —Cl, Br, —I), —CX⁷₃, —CHX⁷₂, —CH₂X⁷, —OCX⁷₃, —OCH₂X⁷, —OCHX⁷₂, —N₃, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}S O₂R^{7D}, —NR^{7A}C(O)R^{7C}, —NR^{7A}C(O)OR^{7C}, —NR^{7A}OR^{7C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). X⁷ is independently —F, —Cl, —Br, or —I. In embodiments, R⁷ is a bond (to L¹), hydrogen, halogen (e.g., —F, —Cl, Br, —I), —CX⁷₃, —CHX⁷₂, —CH₂X⁷, —OCX⁷₃, —OCH₂X⁷, —OCHX⁷₂, —N₃, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), R^{7E}-substituted or unsubstituted alkyl (e.g., C₁-C₈, C₁-C₆, C₁-C₄, or C₁-C₂), R^{7E}-substituted or unsubstituted heteroalkyl (e.g., 2 to 8 membered, 2 to 6 membered, 4 to 6 membered, 2 to 3 membered, or 4 to 5 membered), R^{7E}-substituted or unsubstituted cycloalkyl (e.g., C₃-C₈, C₃-C₆, C₄-C₆, or C₅-C₆), R^{7E}-substituted or unsubstituted heterocycloalkyl (e.g., 3 to 8 membered, 3 to 6 membered, 4 to 6 membered, 4 to 5 membered, or 5 to 6 membered), R^{7E}-substituted or unsubstituted aryl (e.g., C₆-C₁₂, C₆-C₁₀, or phenyl), or R^{7E}-substituted or unsubstituted heteroaryl (e.g., 5 to 12 membered, 5 to 10 membered, 5 to 9 membered, or 5 to 6 membered). In embodiments, R⁷ is a bond (to L¹), hydrogen, halogen (e.g., —F, —Cl, Br, —I), —CX⁷₃, —CHX⁷₂, —CH₂X⁷, —OCX⁷₃, —OCH₂X⁷, —OCHX⁷₂, —N₃, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A} SO₂R^{7D}, —NR^{7A}C(O)R^{7C}, —NR^{7A}C(O)OR^{7C}, —NR^{7A}OR^{7C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, R⁷ is a bond (to L¹), hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁷ is a bond (to L¹), hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —CH₃C(O)OH, —NHOH, —NCH₃OH, —NCH₃OCH₃, R^{7E}-substituted or unsubstituted C₁-C₆ alkyl, R^{7E}-substituted or unsubstituted 2 to 6 membered heteroalkyl, R^{7E}-substituted or unsubstituted C₃-C₆ cycloalkyl, R^{7E}-substituted or unsubstituted 3 to 6 membered heterocycloalkyl, R^{7E}-substituted or unsubstituted phenyl, or R^{7E}-substituted or unsubstituted 5 to 6 membered heteroaryl. In embodiments, R⁷ is a bond (to L¹), hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

In embodiments, R⁷ is hydrogen, halogen (e.g., —F, —Cl, Br, —I), —CX⁷₃, —CHX⁷₂, —CH₂X⁷, —OCX⁷₃, —OCH₂X⁷, —OCHX⁷₂, —N₃, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}S O₂R^{7D}, —NR^{7A}C(O)R^{7C}, —NR^{7A}C(O)OR^{7C}, —NR^{7A}OR^{7C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). X⁷ is independently —F, —Cl, —Br, or —I. In embodiments, R⁷ is hydrogen, halogen (e.g., —F, —Cl, Br, —I), —CX⁷₃, —CHX⁷₂, —CH₂X⁷, —OCX⁷₃, —OCH₂X⁷, —OCHX⁷₂, —N₃, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), R^{7E}-substituted or unsubstituted alkyl (e.g., C₁-C₈, C₁-C₆, C₁-C₄, or C₁-C₂), R^{7E}-substituted or unsubstituted heteroalkyl (e.g., 2 to 8 membered, 2 to 6 membered, 4 to 6 membered, 2 to 3 membered, or 4 to 5 membered), R^{7E}-substituted or unsubstituted cycloalkyl (e.g., C₃-C₈, C₃-C₆, C₄-C₆, or C₅-C₆), R^{7E}-substituted or unsubstituted heterocycloalkyl (e.g., 3 to 8 membered, 3 to 6 membered, 4 to 6 membered, 4 to 5 membered, or 5 to 6 membered), R^{7E}-substituted or unsubstituted aryl (e.g., C₆-C₁₂, C₆-C₁₀, or phenyl), or R^{7E}-substituted or unsubstituted heteroaryl (e.g., 5 to 12 membered, 5 to 10 membered, 5 to 9 membered, or 5 to 6 membered). In embodiments, R⁷ is hydrogen, halogen (e.g., —F, —Cl, Br, —I), —CX⁷₃, —CHX⁷₂, —CH₂X⁷, —OCX⁷₃, —OCH₂X⁷, —OCHX⁷₂, —N₃, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A} SO₂R^{7D}, —NR^{7A}C(O)R^{7C}, —NR^{7A}C(O)OR^{7C}, —NR^{7A}OR^{7C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, R⁷ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁷ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —CH₃C(O)OH, —NHOH, —NCH₃OH, —NCH₃OCH₃, R^{7E}-substituted or unsubstituted C₁-C₆ alkyl, R^{7E}-substituted or unsubstituted 2 to 6 membered heteroalkyl, R^{7E}-substituted or unsubstituted C₃-C₆ cycloalkyl, R^{7E}-substituted or unsubstituted 3 to 6 membered heterocycloalkyl, R^{7E}-substituted or unsubstituted phenyl, or R^{7E}-substituted or unsubstituted 5 to 6 membered heteroaryl. In embodiments, R⁷ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{7E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, R^{7E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{7F}-substituted or unsubstituted alkyl (e.g., C₁-C₈, C₁-C₆, C₁-C₄, or C₁-C₂), R^{7F}-substituted or unsubstituted heteroalkyl (e.g., 2 to 8 membered, 2 to 6 membered, 4 to 6 membered, 2 to 3 membered, or 4 to 5 membered), R^{7F}-substituted or unsubstituted cycloalkyl (e.g., C₃-C₈, C₃-C₆, C₄-C₆, or C₅-C₆), R^{7F}-substituted or unsubstituted heterocycloalkyl (e.g., 3 to 8 membered, 3 to 6 membered, 4 to 6 membered, 4 to 5 membered, or 5 to 6 membered), R^{7F}-substituted or unsubstituted aryl (e.g., C₆-C₁₂, C₆-C₁₀, or phenyl), or R^{7F}-substituted or unsubstituted heteroaryl (e.g., 5 to 12 membered, 5 to 10 membered, 5 to 9 membered, or 5 to 6 membered). In embodiments, R^{7E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R^{7E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{7F}-substituted or unsubstituted C₁-C₆ alkyl, R^{7F}-substituted or unsubstituted 2 to 6 membered heteroalkyl, R^{7F}-substituted or unsubstituted C₃-C₆ cycloalkyl, R^{7F}-substituted or unsubstituted 3 to 6 membered heterocycloalkyl, R^{7F}-substituted or unsubstituted phenyl, or R^{7F}-substituted or unsubstituted 5 to 6 membered heteroaryl. In embodiments, R^{7E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{7F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In embodiments, R^{7F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, C₁-C₆ unsubstituted alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

In embodiments, R⁸ is a bond (to L¹), hydrogen, halogen (e.g., —F, —Cl, Br, —I), —CX⁸₃, —CHX⁸₂, —CH₂X⁸, —OCX⁸₃, —OCH₂X⁸, —OCHX⁸₂, —N₃, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —OR^{8D}, —NR^{8A}SO₂R^{8D}, —NR^{8A}C(O)R^{8C}, —NR^{8A}C(O)OR^{8C}, —NR^{8A}OR^{8C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). X⁸ is independently —F, —Cl, —Br, or —I. In embodiments, R⁸ is a bond (to L¹), hydrogen, halogen (e.g., —F, —Cl, Br, —I), —CX⁸₃, —CHX⁸₂, —CH₂X⁸, —OCX⁸₃, —OCH₂X⁸, —OCHX⁸₂, —N₃, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —OR^{8D}, —NR^{8A}SO₂R^{8D}, —NR^{8A}C(O)R^{8C}, —NR^{8A}C(O)OR^{8C}, —NR^{8A}OR^{8C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), R^{8E}-substituted or unsubstituted alkyl (e.g., C₁-C₈, C₁-C₆, C₁-C₄, or C₁-C₂), R^{8E}-substituted or unsubstituted heteroalkyl (e.g., 2 to 8 membered, 2 to 6 membered, 4 to 6 membered, 2 to 3 membered, or 4 to 5 membered), R^{8E}-substituted or unsubstituted cycloalkyl (e.g., C₃-C₈, C₃-C₆, C₄-C₆, or C₅-C₆), R^{8E}-substituted or unsubstituted heterocycloalkyl (e.g., 3 to 8 membered, 3 to 6 membered, 4 to 6 membered, 4 to 5 membered, or 5 to 6 membered), R^{8E}-substituted or unsubstituted aryl (e.g., C₆-C₁₂, C₆-C₁₀, or phenyl), or R^{8E}-substituted or unsubstituted heteroaryl (e.g., 5 to 12 membered, 5 to 10 membered, 5 to 9 membered, or 5 to 6 membered). In embodiments, R⁸ is a bond (to L¹), hydrogen, halogen (e.g., —F, —Cl, Br, —I), —CX⁸₃, —CHX⁸₂, —CH₂X⁸, —OCX⁸₃, —OCH₂X⁸, —OCHX⁸₂, —N₃, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —OR^{8D}, —NR^{8A}S O₂R^{8D}, —NR^{8A}C(O)R^{8C}, —NR^{8A}C(O)OR^{8C}, —NR^{8A}OR^{8C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, R⁸ is a bond (to L¹), hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁸ is a bond (to L¹), hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{8E}-substituted or unsubstituted C₁-C₆ alkyl, R^{8E}-substituted or unsubstituted 2 to 6 membered heteroalkyl, R^{8E}-substituted or unsubstituted C₃-C₆ cycloalkyl, R^{8E}-substituted or unsubstituted 3 to 6 membered heterocycloalkyl, R^{8E}-substituted or unsubstituted phenyl, or R^{8E}-substituted or unsubstituted 5 to 6 membered heteroaryl. In embodiments, R⁸ is a bond (to L¹), hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

In embodiments, R⁸ is hydrogen, halogen (e.g., —F, —Cl, Br, —I), —CX⁸₃, —CHX⁸₂, —CH₂X⁸, —OCX⁸₃, —OCH₂X⁸, —OCHX⁸₂, —N₃, —CN, —SOₘ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —OR^{8D}, —NR^{8A}S O₂R^{8D}, —NR^{8A}C(O)R^{8C}, —NR^{8A}C(O)OR^{8C}, —NR^{8A}OR^{8C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). X⁸ is independently —F, —Cl, —Br, or —I. In embodiments, R⁸ is hydrogen, halogen (e.g., —F, —Cl, Br, —I), —CX⁸₃, —CHX⁸₂, —CH₂X⁸, —OCX⁸₃, —OCH₂X⁸, —OCHX⁸₂, —N₃, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —OR^{8D}, —NR^{8A}SO₂R^{8D}, —NR^{8A}C(O)R^{8C}, —NR^{8A}C(O)OR^{8C}, —NR^{8A}OR^{8C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), R^{8E}-substituted or unsubstituted alkyl (e.g., C₁-C₈, C₁-C₆, C₁-C₄, or C₁-C₂), R^{8E}-substituted or unsubstituted heteroalkyl (e.g., 2 to 8 membered, 2 to 6 membered, 4 to 6 membered, 2 to 3 membered, or 4 to 5 membered), R^{8E}-substituted or unsubstituted cycloalkyl (e.g., C₃-C₈, C₃-C₆, C₄-C₆, or C₅-C₆), R^{8E}-substituted or unsubstituted heterocycloalkyl (e.g., 3 to 8 membered, 3 to 6 membered, 4 to 6 membered, 4 to 5 membered, or 5 to 6 membered), R^{8E}-substituted or unsubstituted aryl (e.g., C₆-C₁₂, C₆-C₁₀, or phenyl), or R^{8E}-substituted or unsubstituted heteroaryl (e.g., 5 to 12 membered, 5 to 10 membered, 5 to 9 membered, or 5 to 6 membered). In embodiments, R⁸ is hydrogen, halogen (e.g., —F, —Cl, Br, —I), —CX⁸₃, —CHX⁸₂, —CH₂X⁸, —OCX⁸₃, —OCH₂X⁸, —OCHX⁸₂, —N₃, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —OR^{8D}, —NR^{8A}S O₂R^{8D}, —NR^{8A}C(O)R^{8C}, —NR^{8A}C(O)OR^{8C}, —NR^{8A}OR^{8C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, R⁸ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁸ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{8E}-substituted or unsubstituted C₁-C₆ alkyl, R^{8E}-substituted or unsubstituted 2 to 6 membered heteroalkyl, R^{8E}-substituted or unsubstituted C₃-C₆ cycloalkyl, R^{8E}-substituted or unsubstituted 3 to 6 membered heterocycloalkyl, R^{8E}-substituted or unsubstituted phenyl, or R^{8E}-substituted or unsubstituted 5 to 6 membered heteroaryl. In embodiments, R⁸ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{8E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, R^{8E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{8F}-substituted or unsubstituted alkyl (e.g., C₁-C₈, C₁-C₆, C₁-C₄, or C₁-C₂), R^{8F}-substituted or unsubstituted heteroalkyl (e.g., 2 to 8 membered, 2 to 6 membered, 4 to 6 membered, 2 to 3 membered, or 4 to 5 membered), R^{8F}-substituted or unsubstituted cycloalkyl (e.g., C₃-C₈, C₃-C₆, C₄-C₆, or C₅-C₆), R^{8F}-substituted or unsubstituted heterocycloalkyl (e.g., 3 to 8 membered, 3 to 6 membered, 4 to 6 membered, 4 to 5 membered, or 5 to 6 membered), R^{8F}-substituted or unsubstituted aryl (e.g., C₆-C₁₂, C₆-C₁₀, or phenyl), or R^{8F}-substituted or unsubstituted heteroaryl (e.g., 5 to 12 membered, 5 to 10 membered, 5 to 9 membered, or 5 to 6 membered). In embodiments, R^{8E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R^{8E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{8F}-substituted or unsubstituted C₁-C₆ alkyl, R^{8F}-substituted or unsubstituted 2 to 6 membered heteroalkyl, R^{8F}-substituted or unsubstituted C₃-C₆ cycloalkyl, R^{8F}-substituted or unsubstituted 3 to 6 membered heterocycloalkyl, R^{8F}-substituted or unsubstituted phenyl, or R^{8F}-substituted or unsubstituted 5 to 6 membered heteroaryl. In embodiments, R^{8E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{8F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In embodiments, R^{8F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, C₁-C₆ unsubstituted alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

In embodiments, R⁹ is a bond (to L¹), hydrogen, halogen (e.g., —F, —Cl, Br, —I), —CX⁹₃, —CHX⁹₂, —CH₂X⁹, —OCX⁹₃, —OCH₂X⁹, —OCHX⁹₂, —N₃, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). X⁹ is independently —F, —Cl, —Br, or —I. In embodiments, R⁹ is a bond (to L¹), hydrogen, halogen (e.g., —F, —Cl, Br, —I), —CX⁹₃, —CHX⁹₂, —CH₂X⁹, —OCX⁹₃, —OCH₂X⁹, —OCHX⁹₂, —N₃, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), R^{9E}-substituted or unsubstituted alkyl (e.g., C₁-C₈, C₁-C₆, C₁-C₄, or C₁-C₂), R^{9E}-substituted or unsubstituted heteroalkyl (e.g., 2 to 8 membered, 2 to 6 membered, 4 to 6 membered, 2 to 3 membered, or 4 to 5 membered), R^{9E}-substituted or unsubstituted cycloalkyl (e.g., C₃-C₈, C₃-C₆, C₄-C₆, or C₅-C₆), R^{9E}-substituted or unsubstituted heterocycloalkyl (e.g., 3 to 8 membered, 3 to 6 membered, 4 to 6 membered, 4 to 5 membered, or 5 to 6 membered), R^{9E}-substituted or unsubstituted aryl (e.g., C₆-C₁₂, C₆-C₁₀, or phenyl), or R^{9E}-substituted or unsubstituted heteroaryl (e.g., 5 to 12 membered, 5 to 10 membered, 5 to 9 membered, or 5 to 6 membered). In embodiments, R⁹ is a bond (to L¹), hydrogen, halogen (e.g., —F, —Cl, Br, —I), —CX⁹₃, —CHX⁹₂, —CH₂X⁹, —OCX⁹₃, —OCH₂X⁹, —OCHX⁹₂, —N₃, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, R⁹ is a bond (to L¹), hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁹ is a bond (to L¹), hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{9E}-substituted or unsubstituted C₁-C₆ alkyl, R^{9E}-substituted or unsubstituted 2 to 6 membered heteroalkyl, R^{9E}-substituted or unsubstituted C₃-C₆ cycloalkyl, R^{9E}-substituted or unsubstituted 3 to 6 membered heterocycloalkyl, R^{9E}-substituted or unsubstituted phenyl, or R^{9E}-substituted or unsubstituted 5 to 6 membered heteroaryl. In embodiments, R⁹ is a bond (to L¹), hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

In embodiments, R⁹ is hydrogen, halogen (e.g., —F, —Cl, Br, —I), —CX⁹₃, —CHX⁹₂, —CH₂X⁹, —OCX⁹₃, —OCH₂X⁹, —OCHX⁹₂, —N₃, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}S O₂R^{9D}, —NR^{9A}C(O)R^{9C}, —NR^{9A}C(O)OR^{9C}, —NR^{9A}OR^{9C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). X⁹ is independently —F, —Cl, —Br, or —I. In embodiments, R⁹ is hydrogen, halogen (e.g., —F, —Cl, Br, —I), —CX⁹₃, —CHX⁹₂, —CH₂X⁹, —OCX⁹₃, —OCH₂X⁹, —OCHX⁹₂, —N₃, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), R^{9E}-substituted or unsubstituted alkyl (e.g., C₁-C₈, C₁-C₆, C₁-C₄, or C₁-C₂), R^{9E}-substituted or unsubstituted heteroalkyl (e.g., 2 to 8 membered, 2 to 6 membered, 4 to 6 membered, 2 to 3 membered, or 4 to 5 membered), R^{9E}-substituted or unsubstituted cycloalkyl (e.g., C₃-C₈, C₃-C₆, C₄-C₆, or C₅-C₆), R^{9E}-substituted or unsubstituted heterocycloalkyl (e.g., 3 to 8 membered, 3 to 6 membered, 4 to 6 membered, 4 to 5 membered, or 5 to 6 membered), R^{9E}-substituted or unsubstituted aryl (e.g., C₆-C₁₂, C₆-C₁₀, or phenyl), or R^{9E}-substituted or unsubstituted heteroaryl (e.g., 5 to 12 membered, 5 to 10 membered, 5 to 9 membered, or 5 to 6 membered). In embodiments, R⁹ is hydrogen, halogen (e.g., —F, —Cl, Br, —I), —CX⁹₃, —CHX⁹₂, —CH₂X⁹, —OCX⁹₃, —OCH₂X⁹, —OCHX⁹₂, —N₃, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, R⁹ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁹ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{9E}-substituted or unsubstituted C₁-C₆ alkyl, R^{9E}-substituted or unsubstituted 2 to 6 membered heteroalkyl, R^{9E}-substituted or unsubstituted C₃-C₆ cycloalkyl, R^{9E}-substituted or unsubstituted 3 to 6 membered heterocycloalkyl, R^{9E}-substituted or unsubstituted phenyl, or R^{9E}-substituted or unsubstituted 5 to 6 membered heteroaryl. In embodiments, R⁹ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{9E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, R^{9E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{9F}-substituted or unsubstituted alkyl (e.g., C₁-C₈, C₁-C₆, C₁-C₄, or C₁-C₂), R^{9F}-substituted or unsubstituted heteroalkyl (e.g., 2 to 8 membered, 2 to 6 membered, 4 to 6 membered, 2 to 3 membered, or 4 to 5 membered), R^{9F}-substituted or unsubstituted cycloalkyl (e.g., C₃-C₈, C₃-C₆, C₄-C₆, or C₅-C₆), R^{9F}-substituted or unsubstituted heterocycloalkyl (e.g., 3 to 8 membered, 3 to 6 membered, 4 to 6 membered, 4 to 5 membered, or 5 to 6 membered), R^{9F}-substituted or unsubstituted aryl (e.g., C₆-C₁₂, C₆-C₁₀, or phenyl), or R^{9F}-substituted or unsubstituted heteroaryl (e.g., 5 to 12 membered, 5 to 10 membered, 5 to 9 membered, or 5 to 6 membered). In embodiments, R^{9E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R^{9E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{9F}-substituted or unsubstituted C₁-C₆ alkyl, R^{9F}-substituted or unsubstituted 2 to 6 membered heteroalkyl, R^{9F}-substituted or unsubstituted C₃-C₆ cycloalkyl, R^{9F}-substituted or unsubstituted 3 to 6 membered heterocycloalkyl, R^{9F}-substituted or unsubstituted phenyl, or R^{9F}-substituted or unsubstituted 5 to 6 membered heteroaryl. In embodiments, R^{9E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{9F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In embodiments, R^{9F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, C₁-C₆ unsubstituted alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

In embodiments, R¹⁰ is a bond (to L¹), hydrogen, halogen (e.g., —F, —Cl, Br, —I), —CX¹⁰₃, —CHX¹⁰₂, —CH₂X¹⁰, —OCX¹⁰₃, —OCH₂X¹⁰, —OCHX¹⁰₂, —N₃, —CN, —SOₙ₁₀R^{10D}, —SOᵥ₁₀NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O) NR^{10A}R^{10B}, —OR^{10D}, —NR^{10A}SO₂R^{10D}, —NR^{10A}C(O)R^{10C}, —NR^{10A}AC(O)OR^{10C}, —NR^{10A}OR^{10C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). X¹⁰ is independently —F, —Cl, —Br, or —I. In embodiments, R¹⁰ is a bond (to L¹), hydrogen, halogen (e.g., —F, —Cl, Br, —I), —CX¹⁰₃, —CHX¹⁰₂, —CH₂X¹⁰, —OCX¹⁰₃, —OCH₂X¹⁰, —OCHX¹⁰₂, —N₃, —CN, —SOₙ₁₀R^{10D}, —SOᵥ₁₀NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), R^{10E}-substituted or unsubstituted alkyl (e.g., C₁-C₈, C₁-C₆, C₁-C₄, or C₁-C₂), R^{10E}-substituted or unsubstituted heteroalkyl (e.g., 2 to 8 membered, 2 to 6 membered, 4 to 6 membered, 2 to 3 membered, or 4 to 5 membered), R^{10E}-substituted or unsubstituted cycloalkyl (e.g., C₃-C₈, C₃-C₆, C₄-C₆, or C₅-C₆), R^{10E}-substituted or unsubstituted heterocycloalkyl (e.g., 3 to 8 membered, 3 to 6 membered, 4 to 6 membered, 4 to 5 membered, or 5 to 6 membered), R^{10E}-substituted or unsubstituted aryl (e.g., C₆-C₁₂, C₆-C₁₀, or phenyl), or R^{10E}-substituted or unsubstituted heteroaryl (e.g., 5 to 12 membered, 5 to 10 membered, 5 to 9 membered, or 5 to 6 membered). In embodiments, R¹⁰ is a bond (to L¹), hydrogen, halogen (e.g., —F, —Cl, Br, —I), —CX¹⁰₃, —CHX¹⁰₂, —CH₂X¹⁰, —OCX¹⁰₃, —OCH₂X¹⁰, —OCHX¹⁰₂, —N₃, —CN, —SOₙ₁₀R^{10D}, —SOᵥ₁₀NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}—NR^{10A}C(O)OR^{10C}, —NR^{10A}OR^{10C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, R¹⁰ is a bond (to L¹), hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R¹⁰ is a bond (to L¹), hydrogen, —F, —Cl Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{10E}-substituted or unsubstituted C₁-C₆ alkyl, R^{10E}-substituted or unsubstituted 2 to 6 membered heteroalkyl, R^{10E}-substituted or unsubstituted C₃-C₆ cycloalkyl, R^{10E}-substituted or unsubstituted 3 to 6 membered heterocycloalkyl, R^{10E}-substituted or unsubstituted phenyl, or R^{10E}-substituted or unsubstituted 5 to 6 membered heteroaryl. In embodiments, R¹⁰ is a bond (to L¹), hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

In embodiments, R¹⁰ is hydrogen, halogen (e.g., —F, —Cl, Br, —I), —CX¹⁰₃, —CHX¹⁰₂, —CH₂X¹⁰, —OCX¹⁰₃, —OCH₂X¹⁰, —OCHX¹⁰₂, —N₃, —CN, —SOₙ₁₀R^{10D}, —SOᵥ₁₀NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). X¹⁰ is independently —F, —Cl, —Br, or —I. In embodiments, R¹⁰ is hydrogen, halogen (e.g., —F, —Cl, Br, —I), —CX¹⁰₃, —CHX¹⁰₂, —CH₂X¹⁰, —OCX¹⁰₃, —OCH₂X¹⁰, —OCHX¹⁰₂, —N₃, —CN, —SOₙ₁₀R^{10D}, —SOᵥ₁₀NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), R^{10E}-substituted or unsubstituted alkyl (e.g., C₁-C₈, C₁-C₆, C₁-C₄, or C₁-C₂), R^{10E}-substituted or unsubstituted heteroalkyl (e.g., 2 to 8 membered, 2 to 6 membered, 4 to 6 membered, 2 to 3 membered, or 4 to 5 membered), R^{10E}-substituted or unsubstituted cycloalkyl (e.g., C₃-C₈, C₃-C₆, C₄-C₆, or C₅-C₆), R^{10E}-substituted or unsubstituted heterocycloalkyl (e.g., 3 to 8 membered, 3 to 6 membered, 4 to 6 membered, 4 to 5 membered, or 5 to 6 membered), R^{10E}-substituted or unsubstituted aryl (e.g., C₆-C₁₂, C₆-C₁₀, or phenyl), or R^{10E}-substituted or unsubstituted heteroaryl (e.g., 5 to 12 membered, 5 to 10 membered, 5 to 9 membered, or 5 to 6 membered). In embodiments, R¹⁰ is hydrogen, halogen (e.g., —F, —Cl, Br, —I), —CX¹⁰₃, —CHX¹⁰₂, —CH₂X¹⁰, —OCX¹⁰₃, —OCH₂X¹⁰, —OCHX¹⁰₂, —N₃, —CN, —SOₙ₁₀R^{10D}D, —SOᵥ₁₀NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, R¹⁰ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R¹⁰ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{10E}-substituted or unsubstituted C₁-C₆ alkyl, R^{10E}-substituted or unsubstituted 2 to 6 membered heteroalkyl, R^{10E}-substituted or unsubstituted C₃-C₆ cycloalkyl, R^{10E}-substituted or unsubstituted 3 to 6 membered heterocycloalkyl, R^{10E}-substituted or unsubstituted phenyl, or R^{10E}-substituted or unsubstituted 5 to 6 membered heteroaryl. In embodiments, R¹⁰ is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{10E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, R^{10E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{10F}-substituted or unsubstituted alkyl (e.g., C₁-C₈, C₁-C₆, C₁-C₄, or C₁-C₂), R^{10F}-substituted or unsubstituted heteroalkyl (e.g., 2 to 8 membered, 2 to 6 membered, 4 to 6 membered, 2 to 3 membered, or 4 to 5 membered), R^{10F}-substituted or unsubstituted cycloalkyl (e.g., C₃-C₈, C₃-C₆, C₄-C₆, or C₅-C₆), R^{10F}-substituted or unsubstituted heterocycloalkyl (e.g., 3 to 8 membered, 3 to 6 membered, 4 to 6 membered, 4 to 5 membered, or 5 to 6 membered), R^{10F}-substituted or unsubstituted aryl (e.g., C₆-C₁₂, C₆-C₁₀, or phenyl), or R^{10F}-substituted or unsubstituted heteroaryl (e.g., 5 to 12 membered, 5 to 10 membered, 5 to 9 membered, or 5 to 6 membered). In embodiments, R^{10E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R^{10E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{10F}-substituted or unsubstituted C₁-C₆ alkyl, R^{10F}-substituted or unsubstituted 2 to 6 membered heteroalkyl, R^{10F}-substituted or unsubstituted C₃-C₆ cycloalkyl, R^{10F}-substituted or unsubstituted 3 to 6 membered heterocycloalkyl, R^{10F}-substituted or unsubstituted phenyl, or R^{10F}-substituted or unsubstituted 5 to 6 membered heteroaryl. In embodiments, R^{10E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{10F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In embodiments, R^{10F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, C₁-C₆ unsubstituted alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

In embodiments, R¹² is hydrogen, halogen (e.g., —F, —Cl, Br, —I), —CX¹²₃, —CHX¹²₂, —CH₂X¹², —OCX¹²₃, —OCH₂X¹², —OCHX¹²₂, —CN, —SOₙ₁₂R^{12D}, —SOᵥ₁₂NR^{12A}R^{12B}, —NHC(O)NR^{12A}R^{12B}, —N(O)ₘ₁₂, —NR^{12A}R^{12B}, —C(O)R^{12C}, —C(O)—OR^{12C}, —C(O)NR^{12A}R^{12B}, —OR^{12D}, —NR^{12A}SO₂R^{12D}, —NR^{12A}C(O)R^{12C}, —NR^{12A}C(O)OR^{12C}, —NR^{12A}OR^{12C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). X¹² is independently —F, —Cl, —Br, or —I. In embodiments, R¹² is hydrogen, halogen (e.g., —F, —Cl, Br, —I), —CX¹²₃, —CHX¹²₂, —CH₂X¹², —OCX¹²₃, —OCH₂X¹², —OCHX¹²₂, —CN, —SOₙ₁₂R^{12D}, —SOᵥ₁₂NR^{12A}R^{12B}, —NHC(O)NR^{12A}R^{12B}, —N(O)ₘ₁₂, —NR^{12A}R^{12B}, —C(O)R^{12C}, —C(O)—OR^{12C}, —C(O)NR^{12A}R^{12B}, —OR^{12D}, —NR^{12A}SO₂R^{12D}, —NR^{12A}C(O)R^{12C}, —NR^{12A}C(O)OR^{12C}, —NR^{12A}O R^{12C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), R^{12E}-substituted or unsubstituted alkyl (e.g., C₁-C₈, C₁-C₆, C₁-C₄, or C₁-C₂), R^{12E}-substituted or unsubstituted heteroalkyl (e.g., 2 to 8 membered, 2 to 6 membered, 4 to 6 membered, 2 to 3 membered, or 4 to 5 membered), R^{12E}-substituted or unsubstituted cycloalkyl (e.g., C₃-C₈, C₃-C₆, C₄-C₆, or C₅-C₆), R^{12E}-substituted or unsubstituted heterocycloalkyl (e.g., 3 to 8 membered, 3 to 6 membered, 4 to 6 membered, 4 to 5 membered, or 5 to 6 membered), R^{12E}-substituted or unsubstituted aryl (e.g., C₆-C₁₂, C₆-C₁₀, or phenyl), or R^{12E}-substituted or unsubstituted heteroaryl (e.g., 5 to 12 membered, 5 to 10 membered, 5 to 9 membered, or 5 to 6 membered). In embodiments, R¹² is hydrogen, halogen (e.g., —F, —Cl, Br, —I), —CX¹²₃, —CHX¹²₂, —CH₂X¹², —OCX¹²₃, —OCH₂X¹², —OCHX¹²₂, —CN, —SOₙ₁₂R^{12D}, —SOᵥ₁₂NR^{12A}R^{12B}, —NHC(O)NR^{12A}R^{12B}, —N(O)ₘ₁₂, —NR^{12A}R^{12B}, —C(O)R^{12C}, —C(O)—OR^{12C}, —C(O)NR^{12A}R^{12B}, —OR^{12D}, —NR^{12A}SO₂R^{12D}, —NR^{12A}C(O)R^{12C}, —NR^{12A}C(O)OR^{12C}, —NR^{12A}OR^{12C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, R¹² is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R¹² is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{12E}-substituted or unsubstituted C₁-C₆ alkyl, R^{12E}-substituted or unsubstituted 2 to 6 membered heteroalkyl, R^{12E}-substituted or unsubstituted C₃-C₆ cycloalkyl, R^{12E}-substituted or unsubstituted 3 to 6 membered heterocycloalkyl, R^{12E}-substituted or unsubstituted phenyl, or R^{12E}-substituted or unsubstituted 5 to 6 membered heteroaryl. In embodiments, R¹² is hydrogen, —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{12E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, R^{12E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{12F}-substituted or unsubstituted alkyl (e.g., C₁-C₈, C₁-C₆, C₁-C₄, or C₁-C₂), R^{12F}-substituted or unsubstituted heteroalkyl (e.g., 2 to 8 membered, 2 to 6 membered, 4 to 6 membered, 2 to 3 membered, or 4 to 5 membered), R^{12F}-substituted or unsubstituted cycloalkyl (e.g., C₃-C₈, C₃-C₆, C₄-C₆, or C₅-C₆), R^{12F}-substituted or unsubstituted heterocycloalkyl (e.g., 3 to 8 membered, 3 to 6 membered, 4 to 6 membered, 4 to 5 membered, or 5 to 6 membered), R^{12F}-substituted or unsubstituted aryl (e.g., C₆-C₁₂, C₆-C₁₀, or phenyl), or R^{12F}-substituted or unsubstituted heteroaryl (e.g., 5 to 12 membered, 5 to 10 membered, 5 to 9 membered, or 5 to 6 membered). In embodiments, R^{12E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R^{12E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{12F}-substituted or unsubstituted C₁-C₆ alkyl, R^{12F}-substituted or unsubstituted 2 to 6 membered heteroalkyl, R^{12F}-substituted or unsubstituted C₃-C₆ cycloalkyl, R^{12F}-substituted or unsubstituted 3 to 6 membered heterocycloalkyl, R^{12F}-substituted or unsubstituted phenyl, or R^{12F}-substituted or unsubstituted 5 to 6 membered heteroaryl. In embodiments, R^{12E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{12F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In embodiments, R^{12F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, C₁-C₆ unsubstituted alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

In embodiments, R¹³ is halogen (e.g., —F, —Cl, Br, —I), —CX¹³₃, —CHX¹³₂, —CH₂X¹³, —OCX¹³₃, —OCH₂X¹³, —OCHX¹³₂, —N₃, —CN, —SOₙ₁₃R^{13D}, —SOᵥ₁₃NR^{13A}R^{13B}, —NHC(O)NR^{13A}R^{13B}, —N(O)ₘ₁₃, —NR^{13A}R^{13B}, —C(O)R^{13C}, —C(O)—OR^{13C}, —C(O)NR^{13A}R^{13B}, —OR^{13D}, —NR^{13A}SO₂R^{13D}, —NR^{13A}C(O)R^{13C}, —NR^{13A}C(O)OR^{13C}, —NR^{13A}OR^{13C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). X¹³ is independently —F, —Cl, —Br, or —I. In embodiments, R¹³ is halogen (e.g., —F, —Cl, Br, —I), —CX¹³₃, —CHX¹³₂, —CH₂X¹³, —OCX¹³₃, —OCH₂X¹³, —OCHX¹³₂, —N₃, —CN, —SOₙ₁₃R^{13D}, —SOᵥ₁₃NR^{13A}R^{13B}, —NHC(O)NR^{13A}R^{13B}, —N(O)ₘ₁₃, —NR^{13A}R^{13B}, —C(O)R^{13C}, —C(O)—OR^{13C}, —C(O)NR^{13A}R^{13B}, —OR^{13D}, —NR^{13A}SO₂R^{13D}, —NR^{13A}C(O)R^{13C}, —NR^{13A}C(O)OR^{13C}, —NR^{13A}OR^{13C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), R^{13E}-substituted or unsubstituted alkyl (e.g., C₁-C₈, C₁-C₆, C₁-C₄, or C₁-C₂), R^{13E}-substituted or unsubstituted heteroalkyl (e.g., 2 to 8 membered, 2 to 6 membered, 4 to 6 membered, 2 to 3 membered, or 4 to 5 membered), R^{13E}-substituted or unsubstituted cycloalkyl (e.g., C₃-C₈, C₃-C₆, C₄-C₆, or C₅-C₆), R^{13E}-substituted or unsubstituted heterocycloalkyl (e.g., 3 to 8 membered, 3 to 6 membered, 4 to 6 membered, 4 to 5 membered, or 5 to 6 membered), R^{13E}-substituted or unsubstituted aryl (e.g., C₆-C₁₂, C₆-C₁₀, or phenyl), or R^{13E}-substituted or unsubstituted heteroaryl (e.g., 5 to 12 membered, 5 to 10 membered, 5 to 9 membered, or 5 to 6 membered). In embodiments, R¹³ is halogen (e.g., —F, —Cl, Br, —I), —CX¹³₃, —CHX¹³₂, —CH₂X¹³, —OCX¹³₃, —OCH₂X¹³, —OCHX¹³₂, —N₃, —CN, —SOₙ₁₃R^{13D}, δOᵥ₁₃NR^{13A}R^{13B}, —NHC(O)NR^{13A}R^{13B}, —N(O)ₘ₁₃, —NR^{13A}R^{13B}, —C(O)R^{13C}, —C(O)—OR^{13C}, —C(O)NR^{13A}R^{13B}, —OR^{13D}, —NR^{13A}SO₂R^{13D}, —NR^{13A}C(O)R^{13C}, —NR^{13A}C(O)OR^{13C}, —NR^{13A}OR^{13C} (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, or —NCH₃OCH₃), unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, R¹³ is —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R¹³ is —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{13E}-substituted or unsubstituted C₁-C₆ alkyl, R^{13E}-substituted or unsubstituted 2 to 6 membered heteroalkyl, R^{13E}-substituted or unsubstituted C₃-C₆ cycloalkyl, R^{13E}-substituted or unsubstituted 3 to 6 membered heterocycloalkyl, R^{13E}-substituted or unsubstituted phenyl, or R^{13E}-substituted or unsubstituted 5 to 6 membered heteroaryl. In embodiments, R¹³ is —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{13E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, R^{13E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{13F}-substituted or unsubstituted alkyl (e.g., C₁-C₈, C₁-C₆, C₁-C₄, or C₁-C₂), R^{13F}-substituted or unsubstituted heteroalkyl (e.g., 2 to 8 membered, 2 to 6 membered, 4 to 6 membered, 2 to 3 membered, or 4 to 5 membered), R^{13F}-substituted or unsubstituted cycloalkyl (e.g., C₃-C₈, C₃-C₆, C₄-C₆, or C₅-C₆), R^{13F}-substituted or unsubstituted heterocycloalkyl (e.g., 3 to 8 membered, 3 to 6 membered, 4 to 6 membered, 4 to 5 membered, or 5 to 6 membered), R^{13F}-substituted or unsubstituted aryl (e.g., C₆-C₁₂, C₆-C₁₀, or phenyl), or R^{13F}-substituted or unsubstituted heteroaryl (e.g., 5 to 12 membered, 5 to 10 membered, 5 to 9 membered, or 5 to 6 membered). In embodiments, R^{13E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R^{13E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{13F}-substituted or unsubstituted C₁-C₆ alkyl, R^{13F}-substituted or unsubstituted 2 to 6 membered heteroalkyl, R^{13F}-substituted or unsubstituted C₃-C₆ cycloalkyl, R^{13F}-substituted or unsubstituted 3 to 6 membered heterocycloalkyl, R^{13F}-substituted or unsubstituted phenyl, or R^{13F}-substituted or unsubstituted 5 to 6 membered heteroaryl. In embodiments, R^{13E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{13F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In embodiments, R^{13F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, C₁-C₆ unsubstituted alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

In embodiments, R¹⁴ is hydrogen, —CX¹⁴₃, —CHX¹⁴₂, —CH₂X¹⁴ (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I), —COOH, —CONH₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). X¹⁴ is independently —F, —Cl, —Br, or —I. In embodiments, R¹⁴ is hydrogen, —CX¹⁴₃, —CHX¹⁴₂, —CH₂X¹⁴ (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I), —COOH, —CONH₂, R^{14E}-substituted or unsubstituted alkyl (e.g., C₁-C₈, C₁-C₆, C₁-C₄, or C₁-C₂), R^{14E}-substituted or unsubstituted heteroalkyl (e.g., 2 to 8 membered, 2 to 6 membered, 4 to 6 membered, 2 to 3 membered, or 4 to 5 membered), R^{14E}-substituted or unsubstituted cycloalkyl (e.g., C₃-C₈, C₃-C₆, C₄-C₆, or C₅-C₆), R^{14E}-substituted or unsubstituted heterocycloalkyl (e.g., 3 to 8 membered, 3 to 6 membered, 4 to 6 membered, 4 to 5 membered, or 5 to 6 membered), R^{14E}-substituted or unsubstituted aryl (e.g., C₆-C₁₂, C₆-C₁₀, or phenyl), or R^{14E}-substituted or unsubstituted heteroaryl (e.g., 5 to 12 membered, 5 to 10 membered, 5 to 9 membered, or 5 to 6 membered). In embodiments, R¹⁴ is hydrogen, —CX¹⁴₃, —CHX¹⁴₂, —CH₂X¹⁴ (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I), —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, R¹⁴ is hydrogen, —CF₃, —CHF₂, —CH₂F, —CCl₃, —CHCl₂, —CH₂Cl, —CBr₃, —CHBr₂, —CH₂Br, —CI₃, —CHI₂, —CH₂I, —C(O)OH, —C(O)NH₂, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R¹⁴ is hydrogen, —CF₃, —CHF₂, —CH₂F, —CCl₃, —CHCl₂, —CH₂Cl, —CBr₃, —CHBr₂, —CH₂Br, —CI₃, —CHI₂, —CH₂I, —C(O)OH, —C(O)NH₂, R^{14E}-substituted or unsubstituted C₁-C₆ alkyl, R^{14E}-substituted or unsubstituted 2 to 6 membered heteroalkyl, R^{14E}-substituted or unsubstituted C₃-C₆ cycloalkyl, R^{14E}-substituted or unsubstituted 3 to 6 membered heterocycloalkyl, R^{14E}-substituted or unsubstituted phenyl, or R^{14E}-substituted or unsubstituted 5 to 6 membered heteroaryl. In embodiments, R¹⁴ is hydrogen, —CF₃, —CHF₂, —CH₂F, —CCl₃, —CHCl₂, —CH₂Cl, —CBr₃, —CHBr₂, —CH₂Br, —CI₃, —CHI₂, —CH₂I, —C(O)OH, —C(O)NH₂, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{14E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, R^{14E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{14F}-substituted or unsubstituted alkyl (e.g., C₁-C₈, C₁-C₆, C₁-C₄, or C₁-C₂), R^{14F}-substituted or unsubstituted heteroalkyl (e.g., 2 to 8 membered, 2 to 6 membered, 4 to 6 membered, 2 to 3 membered, or 4 to 5 membered), R^{14F}-substituted or unsubstituted cycloalkyl (e.g., C₃-C₈, C₃-C₆, C₄-C₆, or C₅-C₆), R^{14F}-substituted or unsubstituted heterocycloalkyl (e.g., 3 to 8 membered, 3 to 6 membered, 4 to 6 membered, 4 to 5 membered, or 5 to 6 membered), R^{14F}-substituted or unsubstituted aryl (e.g., C₆-C₁₂, C₆-C₁₀, or phenyl), or R^{14F}-substituted or unsubstituted heteroaryl (e.g., 5 to 12 membered, 5 to 10 membered, 5 to 9 membered, or 5 to 6 membered). In embodiments, R^{14E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R^{14E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{14F}-substituted or unsubstituted C₁-C₆ alkyl, R^{14F}-substituted or unsubstituted 2 to 6 membered heteroalkyl, R^{14F}-substituted or unsubstituted C₃-C₆ cycloalkyl, R^{14F}-substituted or unsubstituted 3 to 6 membered heterocycloalkyl, R^{14F}-substituted or unsubstituted phenyl, or R^{14F}-substituted or unsubstituted 5 to 6 membered heteroaryl. In embodiments, R^{14E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{14F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In embodiments, R^{14F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, C₁-C₆ unsubstituted alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

In embodiments, R¹⁵ is independently halogen (e.g., —F, —Cl, Br, —I), —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, R¹⁵ is independently halogen (e.g., —F, —Cl, Br, —I), —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, R^{15E}-substituted or unsubstituted alkyl (e.g., C₁-C₈, C₁-C₆, C₁-C₄, or C₁-C₂), R^{15E}-substituted or unsubstituted heteroalkyl (e.g., 2 to 8 membered, 2 to 6 membered, 4 to 6 membered, 2 to 3 membered, or 4 to 5 membered), R^{15E}-substituted or unsubstituted cycloalkyl (e.g., C₃-C₈, C₃-C₆, C₄-C₆, or C₅-C₆), R^{15E}-substituted or unsubstituted heterocycloalkyl (e.g., 3 to 8 membered, 3 to 6 membered, 4 to 6 membered, 4 to 5 membered, or 5 to 6 membered), R^{15E}-substituted or unsubstituted aryl (e.g., C₆-C₁₂, C₆-C₁₀, or phenyl), or R^{15E}-substituted or unsubstituted heteroaryl (e.g., 5 to 12 membered, 5 to 10 membered, 5 to 9 membered, or 5 to 6 membered). In embodiments, R¹⁵ is independently halogen (e.g., —F, —Cl, Br, —I), —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered).

In embodiments, R¹⁵ is independently R^{15E}-substituted or unsubstituted C₁-C₆ alkyl, R^{15E}-substituted or unsubstituted 2 to 6 membered heteroalkyl, R^{15E}-substituted or unsubstituted C₃-C₆ cycloalkyl, R^{15E}-substituted or unsubstituted 3 to 6 membered heterocycloalkyl, R^{15E}-substituted or unsubstituted phenyl, or R^{15E}-substituted or unsubstituted 5 to 6 membered heteroaryl. In embodiments, R¹⁵ is independently —F, —Cl, Br, —I, —CF₃, —CHF₂, —CH₂F, —CCl₃, —CHCl₂, —CH₂Cl, —CBr₃, —CHBr₂, —CH₂Br, —CI₃, —CHI₂, —CH₂I, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, —N₃, —CN, —SH, —SCH₃, —SO₂H, —SO₂CH₃, —SO₂NH₂, —SO₂NHCH₃, —NHC(O)NH₂, —NHC(O)NHCH₃, —NO₂, —NH₂, —NHCH₃, —C(O)H, —C(O)CH₃, —C(O)OH, —C(O)OCH₃, —C(O)NH₂, —C(O)NHCH₃, —OH, —OCH₃, —NHSO₂H, —NHSO₂CH₃, —NHC(O)H, —NCH₃C(O)H, —NHC(O)OH, —NCH₃C(O)OH, —NHOH, —NCH₃OH, —NCH₃OCH₃, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{15E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₂, C₆-C₁₀, or phenyl), or substituted or unsubstituted heteroaryl (e.g., 5 to 12 membered, 5 to 10 membered, 5 to 9 membered, or 5 to 6 membered). In embodiments, R^{15E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{15F}-substituted or unsubstituted alkyl (e.g., C₁-C₈, C₁-C₆, C₁-C₄, or C₁-C₂), R^{15F}-substituted or unsubstituted heteroalkyl (e.g., 2 to 8 membered, 2 to 6 membered, 4 to 6 membered, 2 to 3 membered, or 4 to 5 membered), R^{15F}-substituted or unsubstituted cycloalkyl (e.g., C₃-C₈, C₃-C₆, C₄-C₆, or C₅-C₆), R^{15F}-substituted or unsubstituted heterocycloalkyl (e.g., 3 to 8 membered, 3 to 6 membered, 4 to 6 membered, 4 to 5 membered, or 5 to 6 membered), R^{15F}-substituted or unsubstituted aryl (e.g., C₆-C₁₂, C₆-C₁₀, or phenyl), or R^{15F}-substituted or unsubstituted heteroaryl (e.g., 5 to 12 membered, 5 to 10 membered, 5 to 9 membered, or 5 to 6 membered). In embodiments, R^{15E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). In embodiments, R^{15E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, R^{15F}-substituted or unsubstituted C₁-C₆ alkyl, R^{15F}-substituted or unsubstituted 2 to 6 membered heteroalkyl, R^{15F}-substituted or unsubstituted C₃-C₆ cycloalkyl, R^{15F}-substituted or unsubstituted 3 to 6 membered heterocycloalkyl, R^{15F}-substituted or unsubstituted phenyl, or R^{15F}-substituted or unsubstituted 5 to 6 membered heteroaryl. In embodiments, R^{15E} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

R^{15F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In embodiments, R^{15F} is independently oxo, —F, —Cl, —Br, —I, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, C₁-C₆ unsubstituted alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

L^{1A} is independently a bond, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenyl), or substituted or unsubstituted heteroarylene (e.g., 5 to 10 membered, 5 to 9 membered, or 5 to 6 membered). In embodiments, L^{1A} is independently a bond, R¹⁵-substituted or unsubstituted alkylene (e.g., C₁-C₈, C₁-C₆, C₁-C₄, or C₁-C₂), R¹⁵-substituted or unsubstituted heteroalkylene (e.g., 2 to 8 membered, 2 to 6 membered, 4 to 6 membered, 2 to 3 membered, or 4 to 5 membered), R¹⁵-substituted or unsubstituted cycloalkylene (e.g., C₃-C₈, C₃-C₆, C₄-C₆, or C₅-C₆), R¹⁵-substituted or unsubstituted heterocycloalkylene (e.g., 3 to 8 membered, 3 to 6 membered, 4 to 6 membered, 4 to 5 membered, or 5 to 6 membered), R¹⁵-substituted or unsubstituted arylene (e.g., C₆-C₁₀ or phenyl), or R¹⁵-substituted or unsubstituted heteroarylene (e.g., 5 to 10 membered, 5 to 9 membered, or 5 to 6 membered). In embodiments, L^{1A} is independently a bond, unsubstituted alkylene (e.g., C₁-C₈, C₁-C₆, C₁-C₄, or C₁-C₂), unsubstituted heteroalkylene (e.g., 2 to 8 membered, 2 to 6 membered, 4 to 6 membered, 2 to 3 membered, or 4 to 5 membered), unsubstituted cycloalkylene (e.g., C₃-C₈, C₃-C₆, C₄-C₆, or C₅-C₆), unsubstituted heterocycloalkylene (e.g., 3 to 8 membered, 3 to 6 membered, 4 to 6 membered, 4 to 5 membered, or 5 to 6 membered), unsubstituted arylene (e.g., C₆-C₁₀ or phenyl), or unsubstituted heteroarylene (e.g., 5 to 10 membered, 5 to 9 membered, or 5 to 6 membered). In embodiments, L^{1A} is independently a bond, substituted or unsubstituted C₁-C₆ alkylene, substituted or unsubstituted 2 to 6 membered heteroalkylene, substituted or unsubstituted C₃-C₆ cycloalkylene, substituted or unsubstituted 3 to 6 membered heterocycloalkylene, substituted or unsubstituted phenylene, or substituted or unsubstituted 5 to 6 membered heteroarylene. In embodiments, L^{1A} is independently a bond, R¹⁵-substituted or unsubstituted C₁-C₆ alkylene, R¹⁵-substituted or unsubstituted 2 to 6 membered heteroalkylene, R¹⁵-substituted or unsubstituted C₃-C₆ cycloalkylene, R¹⁵-substituted or unsubstituted 3 to 6 membered heterocycloalkylene, R¹⁵-substituted or unsubstituted phenylene, or R¹⁵-substituted or unsubstituted 5 to 6 membered heteroarylene. In embodiments, L^{1A} is independently a bond, unsubstituted C₁-C₆ alkylene, unsubstituted 2 to 6 membered heteroalkylene, unsubstituted C₃-C₆ cycloalkylene, unsubstituted 3 to 6 membered heterocycloalkylene, unsubstituted phenylene, or unsubstituted 5 to 6 membered heteroarylene.

Each R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}, R^{6B}, R^{6C}, R^{6D}, R^{7A}, R^{7B}, R^{7C}, R^{7D}, R^{8A}, R^{8B}, R^{8C}, R^{8D}, R^{9A}, R^{9B}, R^{9C}, R^{9D}, R^{10A}, R^{10B}, R^{10C}, R^{10D}, R¹¹, R^{12A}, R^{12B}, R^{12C}, R^{12D}, R^{13A}, R^{13B}, R^{13C}, R^{13D}, R^{14A}, R^{14B}, R^{14C}, and R^{14D} are independently hydrogen, —CX₃, —CHX₂, —CH₂X (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I), —COOH, —CONH₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). X is independently —F, —Cl, —Br, or —I. In embodiments, each R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}, R^{6B}, R^{6C}, R^{6D}, R^{7A}, R^{7B}, R^{7C}, R^{7D}, R^{8A}, R^{8B}, R^{8C}, R^{8D}, R^{9A}, R^{9B}, R^{9C}, R^{9D}, R^{10A}, R^{10B}, R^{10C}, R^{10D}, R¹¹, R^{12A}, R^{12B}, R^{12C}, R^{12D}, R^{13A}, R^{13B}, R^{13C}, R^{13D}, R^{14A}, R^{14B}, R^{14C}, and R^{14D} are independently hydrogen, —CX₃, —CHX₂, —CH₂X (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I), —COOH, —CONH₂, R¹⁵-substituted or unsubstituted alkyl (e.g., C₁-C₈, C₁-C₆, C₁-C₄, or C₁-C₂), R¹⁵-substituted or unsubstituted heteroalkyl (e.g., 2 to 8 membered, 2 to 6 membered, 4 to 6 membered, 2 to 3 membered, or 4 to 5 membered), R¹⁵-substituted or unsubstituted cycloalkyl (e.g., C₃-C₈, C₃-C₆, C₄-C₆, or C₅-C₆), R¹⁵-substituted or unsubstituted heterocycloalkyl (e.g., 3 to 8 membered, 3 to 6 membered, 4 to 6 membered, 4 to 5 membered, or 5 to 6 membered), R¹⁵-substituted or unsubstituted aryl (e.g., C₆-C₁₀ or phenyl), or R¹⁵-substituted or unsubstituted heteroaryl (e.g., 5 to 10 membered, 5 to 9 membered, or 5 to 6 membered). In embodiments, each R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}, R^{6B}, R^{6C}, R^{6D}, R^{7A}, R^{7B}, R^{7C}, R^{7D}, R^{8A}, R^{8B}, R^{8C}, R^{8D}, R^{9A}, R^{9B}, R^{9C}, R^{9D}, R^{10A}, R^{10B}, R^{10C}, R^{10D}, R¹¹, R^{12A}, R^{12B}, R^{12C}, R^{12D}, R^{13A}, R^{13B}, R^{13C}, R^{13D}, R^{14A}, R^{14B}, R^{14C}, and R^{14D} are independently hydrogen, —CX₃, —CHX₂, —CH₂X (e.g., —CF₃, —CHF₂, —CH₂F, —CCl₃, —CHCl₂, —CH₂Cl, —CBr₃, —CHBr₂, —CH₂Br, —CI₃, —CHI₂, —CH₂I), —COOH, —CONH₂, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In embodiments, each R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}, R^{6B}, R^{6C}, R^{6D}, R^{7A}, R^{7B}, R^{7C}, R^{7D}, R^{8A}, R^{8B}, R^{8C}, R^{8D}, R^{9A}, R^{9B}, R^{9C}, R^{9D}, R^{10A}, R^{10B}, R^{10C}, R^{10D}, R¹¹, R^{12A}, R^{12B}, R^{12C}, R^{12D}, R^{13A}, R^{13B}, R^{13C}, R^{13D}, R^{14A}, R^{14B}, R^{14C}, and R^{14D} are independently hydrogen, —CF₃, —CHF₂, —CH₂F, —CCl₃, —CHCl₂, —CH₂Cl, —CBr₃, —CHBr₂, —CH₂Br, —CI₃, —CHI₂, —CH₂I, —COOH, —CONH₂, substituted or unsubstituted C₁-C₆ alkyl, substituted or unsubstituted 2 to 6 membered heteroalkyl, substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, each R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}, R^{6B}, R^{6C}, R^{6D}, R^{7A}, R^{7B}, R^{7C}, R^{7D}, R^{8A}, R^{8B}, R^{8C}, R^{8D}, R^{9A}, R^{9B}, R^{9C}, R^{9D}, R^{10A}, R^{10B}, R^{10C}, R^{10D}, R¹¹, R^{12A}, R^{12B}, R^{12C}, R^{12D}, R^{13A}, R^{13B}, R^{13C}, R^{13D}, R^{14A}, R^{14B}, R^{14C}, and R^{14D} are independently hydrogen, —CF₃, —CHF₂, —CH₂F, —CCl₃, —CHCl₂, —CH₂Cl, —CBr₃, —CHBr₂, —CH₂Br, —CI₃, —CHI₂, —CH₂I, —COOH, —CONH₂, R¹⁵-substituted or unsubstituted C₁-C₆ alkyl, R¹⁵-substituted or unsubstituted 2 to 6 membered heteroalkyl, R¹⁵-substituted or unsubstituted C₃-C₆ cycloalkyl, R¹⁵-substituted or unsubstituted 3 to 6 membered heterocycloalkyl, R¹⁵-substituted or unsubstituted phenyl, or R¹⁵-substituted or unsubstituted 5 to 6 membered heteroaryl. In embodiments, each R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D}, R^{6A}, R^{6B}, R^{6C}, R^{6D}, R^{7A}, R^{7B}, R^{7C}, R^{7D}, R^{8A}, R^{8B}, R^{8C}, R^{8D}, R^{9A}, R^{9B}, R^{9C}, R^{9D}, R^{10A}, R^{10B}, R^{10C}, R^{10D}, R¹¹, R^{12A}, R^{12B}, R^{12C}, R^{12D}, R^{13A}, R^{13B}, R^{13C}, R^{13D}, R^{14A}, R^{14B}, R^{14C}, and R^{14D} are independently hydrogen, —CF₃, —CHF₂, —CH₂F, —CCl₃, —CHCl₂, —CH₂Cl, —CBr₃, —CHBr₂, —CH₂Br, —CI₃, —CHI₂, —CH₂I, —COOH, —CONH₂, unsubstituted C₁-C₆ alkyl, unsubstituted 2 to 6 membered heteroalkyl, unsubstituted C₃-C₆ cycloalkyl, unsubstituted 3 to 6 membered heterocycloalkyl, unsubstituted phenyl, or unsubstituted 5 to 6 membered heteroaryl.

In embodiments, R⁷ and R⁸ together with atoms attached thereto are joined to form substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R⁷ and R⁸ together with atoms attached thereto are joined to form R^{7E}-substituted or unsubstituted cycloalkyl (e.g., C₃-C₈, C₃-C₆, C₄-C₆, or C₅-C₆), R^{7E}-substituted or unsubstituted heterocycloalkyl (e.g., 3 to 8 membered, 3 to 6 membered, 4 to 6 membered, 4 to 5 membered, or 5 to 6 membered), R^{7E}-substituted or unsubstituted aryl (e.g., C₆-C₁₀ or phenyl), or R^{7E}-substituted or unsubstituted heteroaryl (e.g., 5 to 10 membered, 5 to 9 membered, or 5 to 6 membered). In embodiments, R⁷ and R⁸ together with atoms attached thereto are joined to form unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In embodiments, R⁷ and R⁸ together with atoms attached thereto are joined to form substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁷ and R⁸ together with atoms attached thereto are joined to form R^{7E}-substituted or unsubstituted C₃-C₆ cycloalkyl, R^{7E}-substituted or unsubstituted 3 to 6 membered heterocycloalkyl, R^{7E}-substituted or unsubstituted phenyl, or R^{7E}-substituted or unsubstituted 5 to 6 membered heteroaryl. In embodiments, R⁷ and R⁸ together with atoms attached thereto are joined to form unsubstituted C₃-C₆ cycloalkyl, unsubstituted 3 to 6 membered heterocycloalkyl, unsubstituted phenyl, or unsubstituted 5 to 6 membered heteroaryl.

In embodiments, R⁸ and R⁹ together with atoms attached thereto are joined to form substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R⁸ and R⁹ together with atoms attached thereto are joined to form R^{8E}-substituted or unsubstituted cycloalkyl (e.g., C₃-C₈, C₃-C₆, C₄-C₆, or C₅-C₆), R^{8E}-substituted or unsubstituted heterocycloalkyl (e.g., 3 to 8 membered, 3 to 6 membered, 4 to 6 membered, 4 to 5 membered, or 5 to 6 membered), R^{8E}-substituted or unsubstituted aryl (e.g., C₆-C₁₀ or phenyl), or R^{8E}-substituted or unsubstituted heteroaryl (e.g., 5 to 10 membered, 5 to 9 membered, or 5 to 6 membered). In embodiments, R⁸ and R⁹ together with atoms attached thereto are joined to form unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In embodiments, R⁸ and R⁹ together with atoms attached thereto are joined to form substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁸ and R⁹ together with atoms attached thereto are joined to form R^{8E}-substituted or unsubstituted C₃-C₆ cycloalkyl, R^{8E}-substituted or unsubstituted 3 to 6 membered heterocycloalkyl, R^{8E}-substituted or unsubstituted phenyl, or R^{8E}-substituted or unsubstituted 5 to 6 membered heteroaryl. In embodiments, R⁸ and R⁹ together with atoms attached thereto are joined to form unsubstituted C₃-C₆ cycloalkyl, unsubstituted 3 to 6 membered heterocycloalkyl, unsubstituted phenyl, or unsubstituted 5 to 6 membered heteroaryl.

In embodiments, R⁹ and R¹² together with atoms attached thereto are joined to form substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R⁹ and R¹² together with atoms attached thereto are joined to form R^{9E}-substituted or unsubstituted cycloalkyl (e.g., C₃-C₈, C₃-C₆, C₄-C₆, or C₅-C₆), R^{9E}-substituted or unsubstituted heterocycloalkyl (e.g., 3 to 8 membered, 3 to 6 membered, 4 to 6 membered, 4 to 5 membered, or 5 to 6 membered), R^{9E}-substituted or unsubstituted aryl (e.g., C₆-C₁₀ or phenyl), or R^{9E}-substituted or unsubstituted heteroaryl (e.g., 5 to 10 membered, 5 to 9 membered, or 5 to 6 membered). In embodiments, R⁹ and R¹² together with atoms attached thereto are joined to form unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In embodiments, R⁹ and R¹² together with atoms attached thereto are joined to form substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁹ and R¹² together with atoms attached thereto are joined to form R^{9E}-substituted or unsubstituted C₃-C₆ cycloalkyl, R^{9E}-substituted or unsubstituted 3 to 6 membered heterocycloalkyl, R^{9E}-substituted or unsubstituted phenyl, or R^{9E}-substituted or unsubstituted 5 to 6 membered heteroaryl. In embodiments, R⁹ and R¹² together with atoms attached thereto are joined to form unsubstituted C₃-C₆ cycloalkyl, unsubstituted 3 to 6 membered heterocycloalkyl, unsubstituted phenyl, or unsubstituted 5 to 6 membered heteroaryl.

In embodiments, R⁶ and R¹² together with atoms attached thereto are joined to form substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R⁶ and R¹² together with atoms attached thereto are joined to form R^{12E}-substituted or unsubstituted cycloalkyl (e.g., C₃-C₈, C₃-C₆, C₄-C₆, or C₅-C₆), R^{12E}-substituted or unsubstituted heterocycloalkyl (e.g., 3 to 8 membered, 3 to 6 membered, 4 to 6 membered, 4 to 5 membered, or 5 to 6 membered), R^{12E}-substituted or unsubstituted aryl (e.g., C₆-C₁₀ or phenyl), or R^{12E}-substituted or unsubstituted heteroaryl (e.g., 5 to 10 membered, 5 to 9 membered, or 5 to 6 membered). In embodiments, R⁶ and R¹² together with atoms attached thereto are joined to form unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In embodiments, R⁶ and R¹² together with atoms attached thereto are joined to form substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁶ and R¹² together with atoms attached thereto are joined to form R^{12E}-substituted or unsubstituted C₃-C₆ cycloalkyl, R^{12E}-substituted or unsubstituted 3 to 6 membered heterocycloalkyl, R^{12E}-substituted or unsubstituted phenyl, or R^{12E}-substituted or unsubstituted 5 to 6 membered heteroaryl. In embodiments, R⁶ and R¹² together with atoms attached thereto are joined to form unsubstituted C₃-C₆ cycloalkyl, unsubstituted 3 to 6 membered heterocycloalkyl, unsubstituted phenyl, or unsubstituted 5 to 6 membered heteroaryl.

In embodiments, R⁶ and R¹⁰ together with atoms attached thereto are joined to form substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R⁶ and R¹⁰ together with atoms attached thereto are joined to form R^{6E}-substituted or unsubstituted cycloalkyl (e.g., C₃-C₈, C₃-C₆, C₄-C₆, or C₅-C₆), R^{6E}-substituted or unsubstituted heterocycloalkyl (e.g., 3 to 8 membered, 3 to 6 membered, 4 to 6 membered, 4 to 5 membered, or 5 to 6 membered), R^{6E}-substituted or unsubstituted aryl (e.g., C₆-C₁₀ or phenyl), or R^{6E}-substituted or unsubstituted heteroaryl (e.g., 5 to 10 membered, 5 to 9 membered, or 5 to 6 membered). In embodiments, R⁶ and R¹⁰ together with atoms attached thereto are joined to form unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In embodiments, R⁶ and R¹⁰ together with atoms attached thereto are joined to form substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁶ and R¹⁰ together with atoms attached thereto are joined to form R^{6E}-substituted or unsubstituted C₃-C₆ cycloalkyl, R^{6E}-substituted or unsubstituted 3 to 6 membered heterocycloalkyl, R^{6E}-substituted or unsubstituted phenyl, or R^{6E}-substituted or unsubstituted 5 to 6 membered heteroaryl. In embodiments, R⁶ and R¹⁰ together with atoms attached thereto are joined to form unsubstituted C₃-C₆ cycloalkyl, unsubstituted 3 to 6 membered heterocycloalkyl, unsubstituted phenyl, or unsubstituted 5 to 6 membered heteroaryl.

In embodiments, R⁷ and R¹⁰ together with atoms attached thereto are joined to form substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In embodiments, R⁷ and R¹⁰ together with atoms attached thereto are joined to form R^{10E}-substituted or unsubstituted cycloalkyl (e.g., C₃-C₈, C₃-C₆, C₄-C₆, or C₅-C₆), R^{10E}-substituted or unsubstituted heterocycloalkyl (e.g., 3 to 8 membered, 3 to 6 membered, 4 to 6 membered, 4 to 5 membered, or 5 to 6 membered), R^{10E}-substituted or unsubstituted aryl (e.g., C₆-C₁₀ or phenyl), or R^{10E}-substituted or unsubstituted heteroaryl (e.g., 5 to 10 membered, 5 to 9 membered, or 5 to 6 membered). In embodiments, R⁷ and R¹⁰ together with atoms attached thereto are joined to form unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₀ or phenyl), or unsubstituted heteroaryl (e.g., 5 to 10 membered, 5 to 9 membered, or 5 to 6 membered). In embodiments, R⁷ and R¹⁰ together with atoms attached thereto are joined to form substituted or unsubstituted C₃-C₆ cycloalkyl, substituted or unsubstituted 3 to 6 membered heterocycloalkyl, substituted or unsubstituted phenyl, or substituted or unsubstituted 5 to 6 membered heteroaryl. In embodiments, R⁷ and R¹⁰ together with atoms attached thereto are joined to form R^{10E}-substituted or unsubstituted C₃-C₆ cycloalkyl, R^{10E}-substituted or unsubstituted 3 to 6 membered heterocycloalkyl, R^{10E}-substituted or unsubstituted phenyl, or R^{10E}-substituted or unsubstituted 5 to 6 membered heteroaryl. In embodiments, R⁷ and R¹⁰ together with atoms attached thereto are joined to form unsubstituted C₃-C₆ cycloalkyl, unsubstituted 3 to 6 membered heterocycloalkyl, unsubstituted phenyl, or unsubstituted 5 to 6 membered heteroaryl.

X, X¹, X², X³, X⁴, X⁵, X⁶, X⁷, X⁸, X⁹, X¹⁰, X¹², X¹³, X¹⁴ and X¹⁵ are independently —F, —Cl, —Br, or —I. In embodiments, X is —F. In embodiments, X is —Cl. In embodiments, X is —Br. In embodiments, X is —I. In embodiments, X¹ is —F. In embodiments, X¹ is —Cl. In embodiments, X¹ is —Br. In embodiments, X¹ is —I. In embodiments, X² is —F. In embodiments, X² is —Cl. In embodiments, X² is —Br. In embodiments, X² is —I. In embodiments, X³ is —F. In embodiments, X³ is —Cl. In embodiments, X³ is —Br. In embodiments, X³ is —I. In embodiments, X⁴ is —F. In embodiments, X⁴ is —Cl. In embodiments, X⁴ is —Br. In embodiments, X⁴ is —I. In embodiments, X⁵ is —F. In embodiments, X⁵ is —Cl. In embodiments, X⁵ is —Br. In embodiments, X⁵ is —I. In embodiments, X⁶ is —F. In embodiments, X⁶ is —Cl. In embodiments, X⁶ is —Br. In embodiments, X⁶ is —I. In embodiments, X⁷ is —F. In embodiments, X⁷ is —Cl. In embodiments, X⁷ is —Br. In embodiments, X⁷ is —I. In embodiments, X⁸ is —F. In embodiments, X⁸ is —Cl. In embodiments, X⁸ is —Br. In embodiments, X⁸ is —I. In embodiments, X⁹ is —F. In embodiments, X⁹ is —Cl. In embodiments, X⁹ is —Br. In embodiments, X⁹ is —I. In embodiments, X¹⁰ is —F. In embodiments, X¹⁰ is —Cl. In embodiments, X¹⁰ is —Br. In embodiments, X¹⁰ is —I. In embodiments, X¹² is —F. In embodiments, X¹² is —Cl. In embodiments, X¹² is —Br. In embodiments, X¹² is —I. In embodiments, X¹³ is —F. In embodiments, X¹³ is —Cl. In embodiments, X¹³ is —Br. In embodiments, X¹³ is —I. In embodiments, X¹⁴ is —F. In embodiments, X¹⁴ is —Cl. In embodiments, X¹⁴ is —Br. In embodiments, X¹⁴ is —I. In embodiments, X¹⁵ is —F. In embodiments, X¹⁵ is —Cl. In embodiments, X¹⁵ is —Br. In embodiments, X¹⁵ is —I.

n1, n2, n3, n4, n5, n6, n7, n8, n9, n10, n11, n12, and n13 are independently an integer from 0 to 4. In embodiments, n1 is 0. In embodiments, n1 is 1. In embodiments, n1 is 2. In embodiments, n1 is 3. In embodiments, n1 is 4. In embodiments, n2 is 0. In embodiments, n2 is 1. In embodiments, n2 is 2. In embodiments, n2 is 3. In embodiments, n2 is 4. In embodiments, n3 is 0. In embodiments, n3 is 1. In embodiments, n3 is 2. In embodiments, n3 is 3. In embodiments, n3 is 4. In embodiments, n4 is 0. In embodiments, n4 is 1. In embodiments, n4 is 2. In embodiments, n4 is 3. In embodiments, n4 is 4. In embodiments, n5 is 0. In embodiments, n5 is 1. In embodiments, n5 is 2. In embodiments, n5 is 3. In embodiments, n5 is 4. In embodiments, n6 is 0. In embodiments, n6 is 1. In embodiments, n6 is 2. In embodiments, n6 is 3. In embodiments, n6 is 4. In embodiments, n7 is 0. In embodiments, n7 is 1. In embodiments, n7 is 2. In embodiments, n7 is 3. In embodiments, n7 is 4. In embodiments, n8 is 0. In embodiments, n8 is 1. In embodiments, n8 is 2. In embodiments, n8 is 3. In embodiments, n8 is 4. In embodiments, n9 is 0. In embodiments, n9 is 1. In embodiments, n9 is 2. In embodiments, n9 is 3. In embodiments, n9 is 4. In embodiments, n10 is 0. In embodiments, n10 is 1. In embodiments, n10 is 2. In embodiments, n10 is 3. In embodiments, n10 is 4. In embodiments, n11 is 0. In embodiments, n11 is 1. In embodiments, n11 is 2. In embodiments, n11 is 3. In embodiments, n11 is 4. In embodiments, n12 is 0. In embodiments, n12 is 1. In embodiments, n12 is 2. In embodiments, n12 is 3. In embodiments, n12 is 4. In embodiments, n13 is 0. In embodiments, n13 is 1. In embodiments, n13 is 2. In embodiments, n13 is 3. In embodiments, n13 is 4.

m1, m2, m3, m4, m5, m6, m7, m8, m9, m10, m12, and m13 are independently an integer from 1 to 2. In embodiments, m1 is 1. In embodiment, m1 is 2. In embodiments, m2 is 1. In embodiment, m2 is 2. In embodiments, m3 is 1. In embodiment, m3 is 2. In embodiments, m4 is 1. In embodiment, m4 is 2. In embodiments, m5 is 1. In embodiment, m5 is 2. In embodiments, m6 is 1. In embodiment, m6 is 2. In embodiments, m7 is 1. In embodiment, m7 is 2. In embodiments, m8 is 1. In embodiment, m8 is 2. In embodiments, m9 is 1. In embodiment, m9 is 2. In embodiments, m10 is 1. In embodiment, m10 is 2. In embodiments, m12 is 1. In embodiment, m12 is 2. In embodiments, m13 is 1. In embodiment, m13 is 2.

v1, v2, v3, v4, v5, v6, v7, v8, v9, v10, v12, and v13 are independently an integer from 1 to 2. In embodiments, v1 is 1. In embodiment, v1 is 2. In embodiments, v2 is 1. In embodiment, v2 is 2. In embodiments, v3 is 1. In embodiment, v3 is 2. In embodiments, v4 is 1. In embodiment, v4 is 2. In embodiments, v5 is 1. In embodiment, v5 is 2. In embodiments, v6 is 1. In embodiment, v6 is 2. In embodiments, v7 is 1. In embodiment, v7 is 2. In embodiments, v8 is 1. In embodiment, v8 is 2. In embodiments, v9 is 1. In embodiment, v9 is 2. In embodiments, v10 is 1. In embodiment, v10 is 2. In embodiments, v11 is 1. In embodiment, v11 is 2. In embodiments, v12 is 1. In embodiment, v12 is 2. In embodiments, v13 is 1. In embodiment, v13 is 2.

In embodiments, when Y is —CH═, L¹ is —SO₂—N(CH₃)N═CH— and R⁴ is —Br, then R²⁰ is not 2-methyl-5-nitrophenyl. In embodiments, when Y is —CH═, L¹ is —SO₂—N(CH₃)N═CH— and R⁴ is —Br, then R²⁰ is not 5-methyl-2-nitrophenyl. In embodiments, when Y is —CH═, L¹ is —SO₂—N(H)N═CH— and R⁴ is —Br, then R²⁰ is not 2-methyl-5-nitrophenyl. In embodiments, when Y is —CH═, L¹ is —SO₂—N(H)N═CH— and R⁴ is —Br, then R²⁰ is not 5-methyl-2-nitrophenyl. In embodiments, when Y is —CH═, L¹ is —SO₂—N(CH₃)N═CH— and R²⁰ is 2-methyl-5-nitrophenyl, then R⁴ is not —Br. In embodiments, when Y is —CH═, L¹ is —SO₂—N(CH₃)N═CH— and R²⁰ is 5-methyl-2-nitrophenyl, then R⁴ is not —Br. In embodiments, R²⁰ is not 2-methyl-5-nitrophenyl. In embodiments, R²⁰ is not 5-methyl-2-nitrophenyl.

In embodiments, when R¹⁰ is a bond to L¹, Y is —CH═, L¹ is —SO₂—N(CH₃)N═CH—, R⁴ is —Br and R⁶ is unsubstituted methyl, then R⁸ is not —NO₂. In embodiments, when R¹⁰ is a bond to L¹, Y is —CH═, L¹ is —SO₂—N(CH₃)N═CH—, R⁸ is —NO₂ and R⁶ is unsubstituted methyl, then R⁴ is not —Br. In embodiments, when R¹⁰ is a bond to L¹, Y is —CH═, L¹ is —SO₂—N(CH₃)N═CH—, R⁸ is —NO₂ and R⁴ is —Br, then R⁶ is not unsubstituted methyl. In embodiments, when R¹⁰ is a bond to L¹, Y is —CH═, L¹ is —SO₂—N(CH₃)N═CH— and R⁴ is —Br, R⁶ is not substituted or unsubstituted C₁-C₃ alkyl. In embodiments, when R¹⁰ is a bond to L¹, Y is —CH═, L¹ is —SO₂—N(CH₃)N═CH— and R⁴ is —Br, R⁶ is not unsubstituted C₁-C₃ alkyl. In embodiments, when R¹⁰ is a bond to L¹, Y is —CH═, L¹ is —SO₂—N(CH₃)N═CH— and R⁴ is —Br, R⁶ is not unsubstituted methyl. In embodiments, when R¹⁰ is a bond to L¹, Y is —CH═, L¹ is —SO₂—N(CH₃)N═CH— and R⁴ is —Br, R⁶ is not unsubstituted ethyl. In embodiments, when R¹⁰ is a bond to L¹, Y is —CH═, L¹ is —SO₂—N(CH₃)N═CH— and R⁴ is —Br, R⁶ is not unsubstituted propyl. In embodiments, when R¹⁰ is a bond to L¹, Y is —CH═, L¹ is —SO₂—N(R¹⁴)N═CH—, R⁴ is —Br and R¹⁴ is substituted or unsubstituted C₁-C₃ alkyl, R⁶ is not unsubstituted C₁-C₃ alkyl. In embodiments, when R¹⁰ is a bond to L¹, Y is —CH═, L¹ is —SO₂—N(R¹⁴)N═CH—, R⁴ is —Br and R¹⁴ is unsubstituted C₁-C₃ alkyl, R⁶ is not unsubstituted methyl. In embodiments, when R¹⁰ is a bond to L¹, Y is —CH═, L¹ is —SO₂—N(R¹⁴)N═CH—, R⁴ is —Br and R¹⁴ is unsubstituted methyl, R⁶ is not unsubstituted methyl. In embodiments, when R¹⁰ is a bond to L¹, Y is —CH═, L¹ is —SO₂—N(R¹⁴)N═CH—, R⁴ is —Br and R¹⁴ is unsubstituted ethyl, R⁶ is not unsubstituted methyl. In embodiments, when R¹⁰ is a bond to L¹, Y is —CH═, L¹ is —SO₂—N(R¹⁴)N═CH—, R⁴ is —Br and R¹⁴ is unsubstituted propyl, R⁶ is not unsubstituted methyl. In embodiments, when R¹⁰ is a bond to L¹, Y is —CH═, L¹ is —SO₂—N(R¹⁴)N═CH—, R⁴ is —Br and R¹⁴ is unsubstituted C₁-C₃ alkyl, R⁶ is not unsubstituted ethyl. In embodiments, when R¹⁰ is a bond to L¹, Y is —CH═, L¹ is —SO₂—N(R¹⁴)N═CH—, R⁴ is —Br and R¹⁴ is unsubstituted methyl, R⁶ is not unsubstituted ethyl. In embodiments, when R¹⁰ is a bond to L¹, Y is —CH═, L¹ is —SO₂—N(R¹⁴)N═CH—, R⁴ is —Br and R¹⁴ is unsubstituted propyl, R⁶ is not unsubstituted ethyl. In embodiments, when R¹⁰ is a bond to L¹, Y is —CH═, L¹ is —SO₂—N(R¹⁴)N═CH—, R⁴ is —Br and R¹⁴ is unsubstituted C₁-C₃ alkyl, R⁶ is not unsubstituted propyl. In embodiments, when R¹⁰ is a bond to L¹, Y is —CH═, L¹ is —SO₂—N(R¹⁴)N═CH—, R⁴ is —Br and R¹⁴ is unsubstituted methyl, R⁶ is not unsubstituted propyl. In embodiments, when R¹⁰ is a bond to L¹, Y is —CH═, L¹ is —SO₂—N(R¹⁴)N═CH—, R⁴ is —Br and R¹⁴ is unsubstituted propyl, R⁶ is not unsubstituted propyl. In embodiments, when R¹⁰ is a bond to L¹, Y is —CH═, L¹ is —SO₂—N(R¹⁴)N═CH—, R⁴ is —Br and R¹⁴ is substituted or unsubstituted C₁-C₃ alkyl, R⁸ is not —NO₂. In embodiments, when R¹⁰ is a bond to L¹, Y is —CH═, L¹ is —SO₂—N(R¹⁴)N═CH—, R⁴ is —Br and R¹⁴ is unsubstituted C₁-C₃ alkyl, R⁸ is not —NO₂. In embodiments, when R¹⁰ is a bond to L¹, Y is —CH═, L¹ is —SO₂—N(R¹⁴)N═CH—, R⁴ is —Br and R¹⁴ is unsubstituted methyl, R⁸ is not —NO₂. In embodiments, when R¹⁰ is a bond to L¹, Y is —CH═, L¹ is —SO₂—N(R¹⁴)N═CH—, R⁴ is —Br and R¹⁴ is unsubstituted ethyl, R⁸ is not —NO₂. In embodiments, when R¹⁰ is a bond to L¹, Y is —CH═, L¹ is —SO₂—N(R¹⁴)N═CH—, R⁴ is —Br and R¹⁴ is unsubstituted propyl, R⁸ is not —NO₂.

In embodiments, when R⁸ is a bond to L¹, Y is —CH═, L¹ is —SO₂—N(CH₃)N═CH—, R⁴ is —B and R⁹ is not unsubstituted methyl, then R¹⁰ is not —NO₂. In embodiments, when R⁸ is a bond to L¹, Y is —CH═, L¹ is —SO₂—N(CH₃)N═CH—, R¹⁰ is —NO₂ and R⁹ is unsubstituted methyl, then R⁴ is not —Br. In embodiments, when R⁸ is a bond to L¹, Y is —CH═, L¹ is —SO₂—N(CH₃)N═CH—, R¹⁰ is —NO₂ and R⁴ is —Br, then R⁹ is not unsubstituted methyl. In embodiments, when R⁸ is a bond to L¹, Y is —CH═, L¹ is —SO₂—N(CH₃)N═CH— and R⁴ is —Br, R⁹ is not substituted or unsubstituted C₁-C₃ alkyl. In embodiments, when R⁸ is a bond to L¹, Y is —CH═, L¹ is —SO₂—N(CH₃)N═CH— and R⁴ is —Br, R⁹ is not unsubstituted C₁-C₃ alkyl. In embodiments, when R⁸ is a bond to L¹, Y is —CH═, L¹ is —SO₂—N(CH₃)N═CH— and R⁴ is —Br, R⁹ is not unsubstituted methyl. In embodiments, when R⁸ is a bond to L¹, Y is —CH═, L¹ is —SO₂—N(CH₃)N═CH— and R⁴ is —Br, R⁹ is not unsubstituted ethyl. In embodiments, when R⁸ is a bond to L¹, Y is —CH═, L¹ is —SO₂—N(CH₃)N═CH— and R⁴ is —Br, R⁹ is not unsubstituted propyl. In embodiments, when R⁸ is a bond to L¹, Y is —CH═, L¹ is —SO₂—N(R¹⁴)N═CH—, R⁴ is —Br and R¹⁴ is substituted or unsubstituted C₁-C₃ alkyl, R⁹ is not unsubstituted C₁-C₃ alkyl. In embodiments, when R⁸ is a bond to L¹, Y is —CH═, L¹ is —SO₂—N(R¹⁴)N═CH—, R⁴ is —Br and R¹⁴ is unsubstituted C₁-C₃ alkyl, R⁹ is not unsubstituted methyl. In embodiments, when R⁸ is a bond to L¹, Y is —CH═, L¹ is —SO₂—N(R¹⁴)N═CH—, R⁴ is —Br and R¹⁴ is unsubstituted methyl, R⁹ is not unsubstituted methyl. In embodiments, when R⁸ is a bond to L¹, Y is —CH═, L¹ is —SO₂—N(R¹⁴)N═CH—, R⁴ is —Br and R¹⁴ is unsubstituted ethyl, R⁹ is not unsubstituted methyl. In embodiments, when R⁸ is a bond to L¹, Y is —CH═, L¹ is —SO₂—N(R¹⁴)N═CH—, R⁴ is —Br and R¹⁴ is unsubstituted propyl, R⁹ is not unsubstituted methyl. In embodiments, when R⁸ is a bond to L¹, Y is —CH═, L¹ is —SO₂—N(R¹⁴)N═CH—, R⁴ is —Br and R¹⁴ is unsubstituted C₁-C₃ alkyl, R⁹ is not unsubstituted ethyl. In embodiments, when R⁸ is a bond to L¹, Y is —CH═, L¹ is —SO₂—N(R¹⁴)N═CH—, R⁴ is —Br and R¹⁴ is unsubstituted methyl, R⁹ is not unsubstituted ethyl. In embodiments, when R⁸ is a bond to L¹, Y is —CH═, L¹ is —SO₂—N(R¹⁴)N═CH—, R⁴ is —Br and R¹⁴ is unsubstituted propyl, R⁹ is not unsubstituted ethyl. In embodiments, when R⁸ is a bond to L¹, Y is —CH═, L¹ is —SO₂—N(R¹⁴)N═CH—, R⁴ is —Br and R¹⁴ is unsubstituted C₁-C₃ alkyl, R⁹ is not unsubstituted propyl. In embodiments, when R⁸ is a bond to L¹, Y is —CH═, L¹ is —SO₂—N(R¹⁴)N═CH—, R⁴ is —Br and R¹⁴ is unsubstituted methyl, R⁹ is not unsubstituted propyl. In embodiments, when R⁸ is a bond to L¹, Y is —CH═, L¹ is —SO₂—N(R¹⁴)N═CH—, R⁴ is —Br and R¹⁴ is unsubstituted propyl, R⁹ is not unsubstituted propyl. In embodiments, when R⁸ is a bond to L¹, Y is —CH═, L¹ is —SO₂—N(R¹⁴)N═CH—, R⁴ is —Br and R¹⁴ is substituted or unsubstituted C₁-C₃ alkyl, R¹⁰ is not —NO₂. In embodiments, when R⁸ is a bond to L¹, Y is —CH═, L¹ is —SO₂—N(R¹⁴)N═CH—, R⁴ is —Br and R¹⁴ is unsubstituted C₁-C₃ alkyl, R¹⁰ is not —NO₂. In embodiments, when R⁸ is a bond to L¹, Y is —CH═, L¹ is —SO₂—N(R¹⁴)N═CH—, R⁴ is —Br and R¹⁴ is unsubstituted methyl, R¹⁰ is not —NO₂. In embodiments, when R⁸ is a bond to L¹, Y is —CH═, L¹ is —SO₂—N(R¹⁴)N═CH—, R⁴ is —Br and R¹⁴ is unsubstituted ethyl, R¹⁰ is not —NO₂. In embodiments, when R⁸ is a bond to L¹, Y is —CH═, L¹ is —SO₂—N(R¹⁴)N═CH—, R⁴ is —Br and R¹⁴ is unsubstituted propyl, R¹⁰ is not —NO₂.

In embodiments, R⁴ is not halogen. In embodiments, R⁴ is not Br. In embodiments, R⁶ is not unsubstituted methyl. In embodiments, R⁶ is not unsubstituted ethyl. In embodiments, R⁶ is not unsubstituted propyl. In embodiments, R⁷ is not unsubstituted methyl. In embodiments, R⁷ is not unsubstituted ethyl. In embodiments, R⁷ is not unsubstituted propyl. In embodiments, R⁸ is not unsubstituted methyl. In embodiments, R⁸ is not unsubstituted ethyl. In embodiments, R⁸ is not unsubstituted propyl. In embodiments, R⁹ is not unsubstituted methyl. In embodiments, R⁹ is not unsubstituted ethyl. In embodiments, R⁹ is not unsubstituted propyl. In embodiments, R¹⁰ is not unsubstituted methyl. In embodiments, R¹⁰ is not unsubstituted ethyl. In embodiments, R¹⁰ is not unsubstituted propyl. In embodiments, R⁶ is not —NO₂. In embodiments, R⁷ is not —NO₂. In embodiments, R⁸ is not —NO₂. In embodiments, R⁹ is not —NO₂. In embodiments, R¹⁰ is not —NO₂. In embodiments, R⁶ is unsubstituted methyl and R⁸ is not —NO₂. In embodiments, R⁶ is not unsubstituted methyl and R⁸ is —NO₂. In embodiments, R⁴ is not —Br and R⁸ is not —NO₂. In embodiments, R⁶ is unsubstituted methyl and R⁴ is not —Br. In embodiments, R⁶ is not unsubstituted methyl and R⁴ is —Br. In embodiments, R⁶ is not unsubstituted methyl and R⁴ is not —Br. In embodiments, R⁴ is —Br and R⁸ is not —NO₂. In embodiments, R⁴ is not —Br and R⁸ is —NO₂. In embodiments, R⁴ is not —Br and R⁸ is not —NO₂. In embodiments, R⁹ is unsubstituted methyl and R¹⁰ is not —NO₂. In embodiments, R⁹ is not unsubstituted methyl and R¹⁰ is —NO₂. In embodiments, R⁴ is not —Br and R¹⁰ is not —NO₂. In embodiments, R⁹ is unsubstituted methyl and R⁴ is not —Br. In embodiments, R⁹ is not unsubstituted methyl and R⁴ is —Br. In embodiments, R⁹ is not unsubstituted methyl and R⁴ is not —Br. In embodiments, R⁴ is —Br and R¹⁰ is not —NO₂. In embodiments, R⁴ is not —Br and R¹⁰ is —NO₂. In embodiments, R⁴ is not —Br and R¹⁰ is not —NO₂.

In embodiments, the compound is:

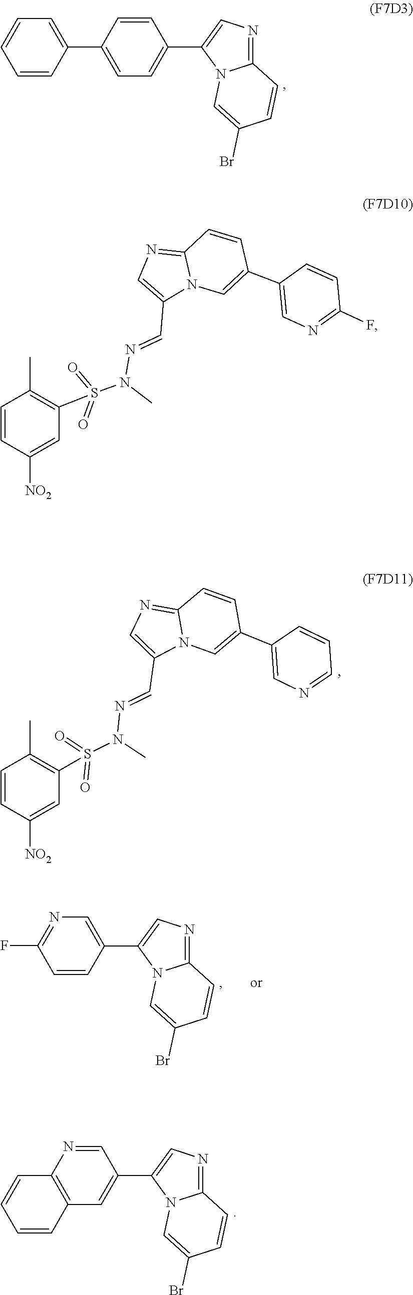 [SMILES: Brc1ccc2ncc(-c3ccc(-c4ccccc4)cc3)n2c1 ; Cc1ccc([N+](=O)[O-])cc1S(=O)(=O)N(C)/N=C/c1cnc2ccc(-c3ccc(F)nc3)cn12 ; Cc1ccc([N+](=O)[O-])cc1S(=O)(=O)N(C)/N=C/c1cnc2ccc(-c3cccnc3)cn12 ; Fc1ccc(-c2cnc3ccc(Br)cn23)cn1 ; Brc1ccc2ncc(-c3cnc4ccccc4c3)n2c1]

In embodiments, the compound is:

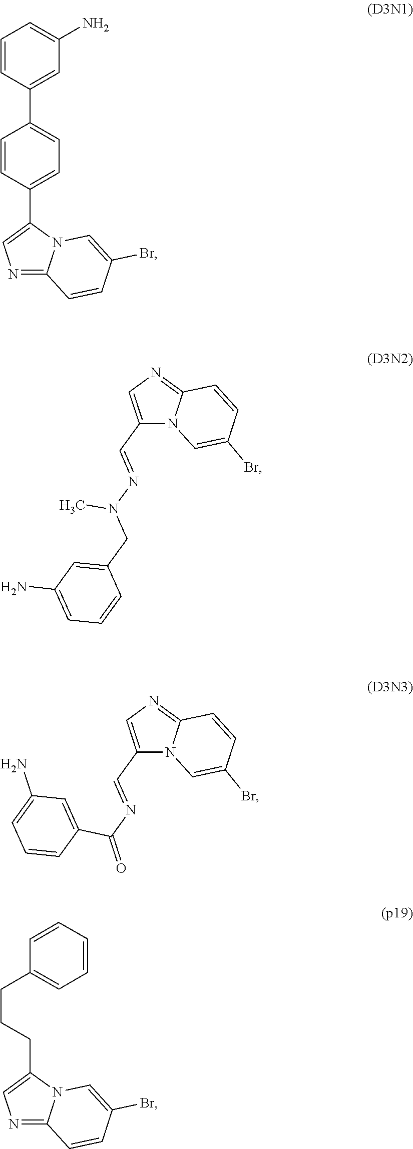 [SMILES: Nc1cccc(-c2ccc(-c3cnc4ccc(Br)cn34)cc2)c1 ; CN(Cc1cccc(N)c1)/N=C/c1cnc2ccc(Br)cn12 ; Nc1cccc(C(=O)/N=C/c2cnc3ccc(Br)cn23)c1 ; Brc1ccc2ncc(CCCc3ccccc3)n2c1]

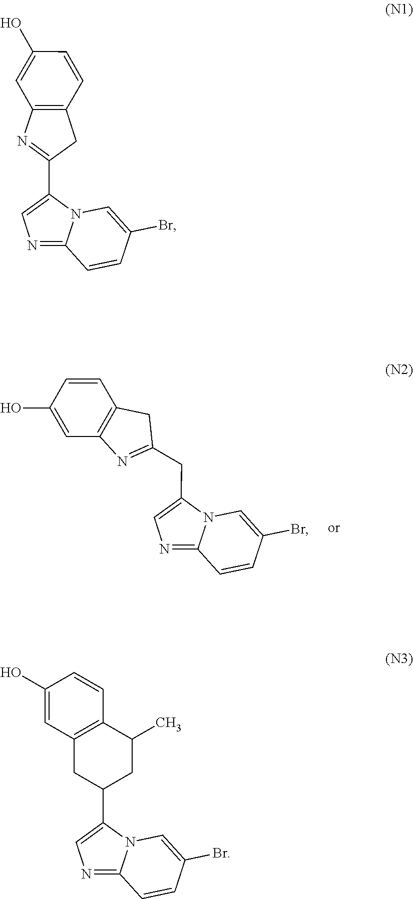 [SMILES: Oc1ccc2c(c1)N=C(c1cnc3ccc(Br)cn13)C2 ; Oc1ccc2c(c1)N=C(Cc1cnc3ccc(Br)cn13)C2 ; "or" ; CC1CC(c2cnc3ccc(Br)cn23)Cc2cc(O)ccc21]

In embodiments, the compound is a compound described herein (e.g., in an aspect, embodiment, example, table, figure, scheme, appendix, or claim).

### III. Pharmaceutical Compositions

Also provided herein are pharmaceutical formulations. In embodiments, the pharmaceutical formulation includes a compound (e.g. formulae (I), (II), (III), (III′), (III-A), (III-A′), (III-B), (III-C), (III-D), (III-E), (IV), (IV-A), (IV-A′), (IV-A″), (V-A), (V-B), (V-C), (V-D), (VI-A), (VI-B), (VII-A), (VII-B), (VII-C), (VII-D) or (VIII-A)) described above (including all embodiments thereof) and a pharmaceutically acceptable excipient.

In one aspect is a pharmaceutical formulation that includes a histone deacetylase (HDAC) inhibitor, a p38 gamma (p38γ) kinase inhibitor and a pharmaceutically acceptable excipient.

In embodiments of the pharmaceutical compositions, the histone deacetylase (HDAC) inhibitor and the p38 gamma (p38γ) kinase inhibitor, or pharmaceutically acceptable salts thereof, is included in a therapeutically effective amount.

The pharmaceutical composition may contain a dosage of the compound in a therapeutically effective amount.

In embodiments, the pharmaceutical composition includes

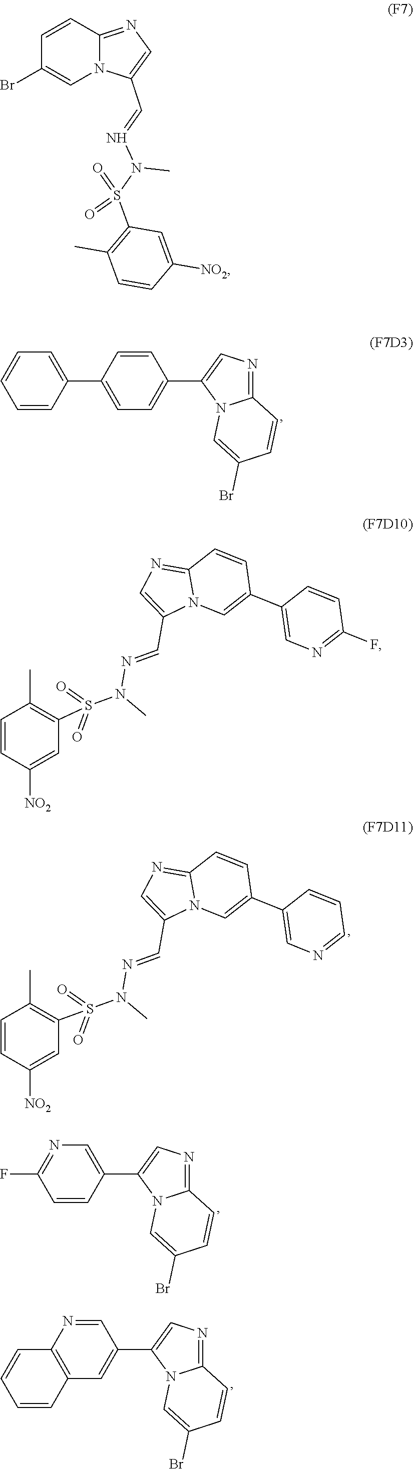

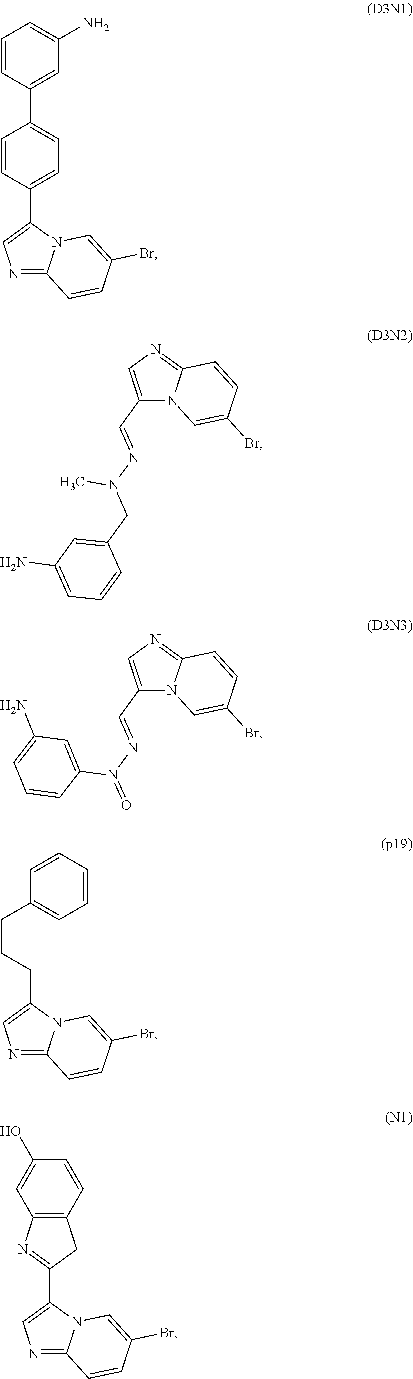

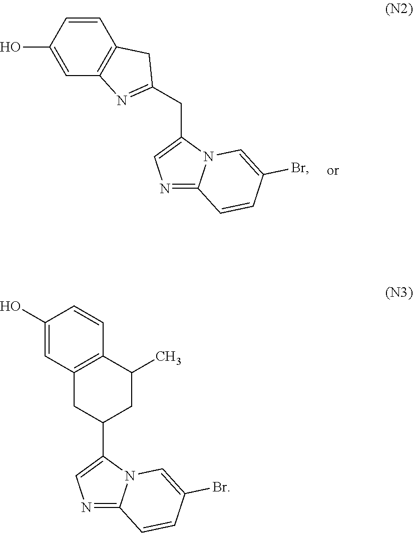 [SMILES: Oc1ccc2c(c1)N=C(Cc1cnc3ccc(Br)cn13)C2 ; CC1CC(c2cnc3ccc(Br)cn23)Cc2cc(O)ccc21]

In embodiments, the pharmaceutical composition includes a compound described herein (e.g., in an aspect, embodiment, example, table, figure, scheme, appendix, or claim).

### 1. Formulations

The pharmaceutical composition may be prepared and administered in a wide variety of dosage formulations. Compounds described may be administered orally, rectally, or by injection (e.g. intravenously, intramuscularly, intracutaneously, subcutaneously, intraduodenally, or intraperitoneally).

For preparing pharmaceutical compositions from compounds described herein, pharmaceutically acceptable carriers can be either solid or liquid. Solid form preparations include powders, tablets, pills, capsules, cachets, suppositories, and dispersible granules. A solid carrier may be one or more substance that may also act as diluents, flavoring agents, binders, preservatives, tablet disintegrating agents, or an encapsulating material.

In powders, the carrier may be a finely divided solid in a mixture with the finely divided active component. In tablets, the active component may be mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight. Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The pharmaceutical compositions may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

The pharmaceutical composition may be intended for intravenous use. The pharmaceutically acceptable excipient can include buffers to adjust the pH to a desirable range for intravenous use. Many buffers including salts of inorganic acids such as phosphate, borate, and sulfate are known.

### 2. Effective Dosages

The pharmaceutical composition may include compositions wherein the active ingredient is contained in a therapeutically effective amount, i.e., in an amount effective to achieve its intended purpose. The actual amount effective for a particular application will depend, inter alia, on the condition being treated.

The dosage and frequency (single or multiple doses) of compounds administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds disclosed herein.

Therapeutically effective amounts for use in humans may be determined from animal models. For example, a dose for humans can be formulated to achieve a concentration that has been found to be effective in animals. The dosage in humans can be adjusted by monitoring response of the constipation or dry eye to the treatment and adjusting the dosage upwards or downwards, as described above.

Dosages may be varied depending upon the requirements of the subject and the compound being employed. The dose administered to a subject, in the context of the pharmaceutical compositions presented herein, should be sufficient to effect a beneficial therapeutic response in the subject over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached.

Dosage amounts and intervals can be adjusted individually to provide levels of the administered compounds effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

### 3. Toxicity

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch. 1, p. 1, 1975. The exact formulation, route of administration, and dosage can be chosen by the individual physician in view of the patient's condition and the particular method in which the compound is used.

When parenteral application is needed or desired, particularly suitable admixtures for the compounds included in the pharmaceutical composition may be injectable, sterile solutions, oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. Pharmaceutical admixtures suitable for use in the pharmaceutical compositions presented herein may include those described, for example, in Pharmaceutical Sciences (17th Ed., Mack Pub. Co., Easton, Pa.) and WO 96/05309, the teachings of both of which are hereby incorporated by reference.

### IV. Methods of Modulating

Provided herein are methods of modulating p38 gamma (p38γ) kinase in a cell. In embodiments, is a method of reducing or suppressing expression of p38 gamma (p38γ) kinase in the cell. In embodiments, is a method of reducing or suppressing mRNA of p38 gamma (p38γ) kinase in the cell. In embodiments, is a method of reducing or suppressing mRNA of p38 gamma (p38γ) kinase in the cell using a compound (e.g. formulae (I), (II), (III), (III′), (III-A), (III-A′), (III-B), (III-C), (III-D), (III-E), (IV), (IV-A), (IV-A′), (IV-A″), (V-A), (V-B), (V-C), (V-D), (VI-A), (VI-B), (VII-A), (VII-B), (VII-C), (VII-D) or (VIII-A)) described above (including all embodiments thereof). In embodiments, is a method of reducing or suppressing expression of p38 gamma (p38γ) kinase in the cell by contacting the cell with a compound. In embodiments, is a method of reducing or suppressing mRNA of p38 gamma (p38γ) kinase in the cell by contacting the cell with a compound (e.g. formulae (I), (II), (III), (III′), (III-A), (III-A′), (III-B), (III-C), (III-D), (III-E), (IV), (IV-A), (IV-A′), (IV-A″), (V-A), (V-B), (V-C), (V-D), (VI-A), (VI-B), (VII-A), (VII-B), (VII-C), (VII-D) or (VIII-A)). The contacting may be performed in vitro. The contacting may be performed in vivo.

In embodiments, is a method of suppressing or inhibiting activity p38 gamma (p38γ) kinase in the cell. In embodiments, is a method of suppressing or inhibiting activity p38 gamma (p38γ) kinase in a cell using at least the compounds described herein. In embodiments, the methods comprise contacting the cell with at least a compound (e.g. formulae (I), (II), (III), (III′), (III-A), (III-A′), (III-B), (III-C), (III-D), (III-E), (IV), (IV-A), (IV-A′), (IV-A″), (V-A), (V-B), (V-C), (V-D), (VI-A), (VI-B), (VII-A), (VII-B), (VII-C), (VII-D) or (VIII-A)) described above (including all embodiments thereof). The contacting may be performed in vitro. The contacting may be performed in vivo.

In embodiments, the compound has an IC₅₀ value of about 50 μM or less against p38γ kinase activity. In embodiments, the compound has an IC₅₀ value of about 40 μM or less against p38γ kinase activity. In embodiments, the compound has an IC₅₀ value of about 30 μM or less against p38γ kinase activity. In embodiments, the compound has an IC₅₀ value of about 25 μM or less against p38γ kinase activity. In embodiments, the compound has an IC₅₀ value of about 20 μM or less against p38γ kinase activity. In embodiments, the compound has an IC₅₀ value of about 15 μM or less against p38γ kinase activity. In embodiments, the compound has an IC₅₀ value of about 10 μM or less against p38γ kinase activity. In embodiments, the compound has an IC₅₀ value of about 9 μM or less against p38γ kinase activity. In embodiments, the compound has an IC₅₀ value of about 8 μM or less against p38γ kinase activity. In embodiments, the compound has an IC₅₀ value of about 7 μM or less against p38γ kinase activity. In embodiments, the compound has an IC₅₀ value of about 6 μM or less against p38γ kinase activity. In embodiments, the compound has an IC₅₀ value of about 5 μM or less against p38γ kinase activity. In embodiments, the compound has an IC₅₀ value of about 4 μM or less against p38γ kinase activity. In embodiments, the compound has an IC₅₀ value of about 3 μM or less against p38γ kinase activity. In embodiments, the compound has an IC₅₀ value of about 2 μM or less against p38γ kinase activity. In embodiments, the compound has an IC₅₀ value of about 1 μM or less against p38γ kinase activity. In embodiments, the compound has an IC₅₀ value of about 900 nM or less against p38γ kinase activity. In embodiments, the compound has an IC₅₀ value of about 800 nM or less against p38γ kinase activity. In embodiments, the compound has an IC₅₀ value of about 700 nM or less against p38γ kinase activity. In embodiments, the compound has an IC₅₀ value of about 600 nM or less against p38γ kinase activity. In embodiments, the compound has an IC₅₀ value of about 500 nM or less against p38γ kinase activity. In embodiments, the compound has an IC₅₀ value of about 400 nM or less against p38γ kinase activity. In embodiments, the compound has an IC₅₀ value of about 300 nM or less against p38γ kinase activity. In embodiments, the compound has an IC₅₀ value of about 200 nM or less against p38γ kinase activity. In embodiments, the compound has an IC₅₀ value of about 100 nM or less against p38γ kinase activity. In embodiments, the compound has an IC₅₀ value of about 50 nM or less against p38γ kinase activity. In embodiments, the compound has an IC₅₀ value of about 40 nM or less against p38γ kinase activity. In embodiments, the compound has an IC₅₀ value of about 30 nM or less against p38γ kinase activity. In embodiments, the compound has an IC₅₀ value of about 20 nM or less against p38γ kinase activity. In embodiments, the compound has an IC₅₀ value of about 10 nM or less against p38γ kinase activity.

In embodiments, the compound has an IC₅₀ value 50 μM or less against p38γ kinase activity. In embodiments, the compound has an IC₅₀ value 40 μM or less against p38γ kinase activity. In embodiments, the compound has an IC₅₀ value 30 μM or less against p38γ kinase activity. In embodiments, the compound has an IC₅₀ value 25 μM or less against p38γ kinase activity. In embodiments, the compound has an IC₅₀ value 20 μM or less against p38γ kinase activity. In embodiments, the compound has an IC₅₀ value 15 μM or less against p38γ kinase activity. In embodiments, the compound has an IC₅₀ value 10 μM or less against p38γ kinase activity. In embodiments, the compound has an IC₅₀ value 9 μM or less against p38γ kinase activity. In embodiments, the compound has an IC₅₀ value 8 μM or less against p38γ kinase activity. In embodiments, the compound has an IC₅₀ value 7 μM or less against p38γ kinase activity. In embodiments, the compound has an IC₅₀ value 6 μM or less against p38γ kinase activity. In embodiments, the compound has an IC₅₀ value 5 μM or less against p38γ kinase activity. In embodiments, the compound has an IC₅₀ value 4 μM or less against p38γ kinase activity. In embodiments, the compound has an IC₅₀ value 3 μM or less against p38γ kinase activity. In embodiments, the compound has an IC₅₀ value 2 μM or less against p38γ kinase activity. In embodiments, the compound has an IC₅₀ value 1 μM or less against p38γ kinase activity. In embodiments, the compound has an IC₅₀ value 900 nM or less against p38γ kinase activity. In embodiments, the compound has an IC₅₀ value 800 nM or less against p38γ kinase activity. In embodiments, the compound has an IC₅₀ value 700 nM or less against p38γ kinase activity. In embodiments, the compound has an IC₅₀ value 600 nM or less against p38γ kinase activity. In embodiments, the compound has an IC₅₀ value 500 nM or less against p38γ kinase activity. In embodiments, the compound has an IC₅₀ value 400 nM or less against p38γ kinase activity. In embodiments, the compound has an IC₅₀ value 300 nM or less against p38γ kinase activity. In embodiments, the compound has an IC₅₀ value 200 nM or less against p38γ kinase activity. In embodiments, the compound has an IC₅₀ value 100 nM or less against p38γ kinase activity. In embodiments, the compound has an IC₅₀ value 50 nM or less against p38γ kinase activity. In embodiments, the compound has an IC₅₀ value 40 nM or less against p38γ kinase activity. In embodiments, the compound has an IC₅₀ value 30 nM or less against p38γ kinase activity. In embodiments, the compound has an IC₅₀ value 20 nM or less against p38γ kinase activity. In embodiments, the compound has an IC₅₀ value 10 nM or less against p38γ kinase activity.

In embodiments, the compound has an IC₅₀ value of about 50 μM or less for cancerous T-cell viability. In embodiments, the compound has an IC₅₀ value of about 40 μM or less against less for cancerous T-cell viability. In embodiments, the compound has an IC₅₀ value of about 30 μM or less against less for cancerous T-cell viability. In embodiments, the compound has an IC₅₀ value of about 25 μM or less against less for cancerous T-cell viability. In embodiments, the compound has an IC₅₀ value of about 20 μM or less against less for cancerous T-cell viability. In embodiments, the compound has an IC₅₀ value of about 15 μM or less against less for cancerous T-cell viability. In embodiments, the compound has an IC₅₀ value of about 10 μM or less against less for cancerous T-cell viability. In embodiments, the compound has an IC₅₀ value of about 9 μM or less against less for cancerous T-cell viability. In embodiments, the compound has an IC₅₀ value of about 8 μM or less against less for cancerous T-cell viability. In embodiments, the compound has an IC₅₀ value of about 7 μM or less against less for cancerous T-cell viability. In embodiments, the compound has an IC₅₀ value of about 6 μM or less against less for cancerous T-cell viability. In embodiments, the compound has an IC₅₀ value of about 5 μM or less against less for cancerous T-cell viability. In embodiments, the compound has an IC₅₀ value of about 4 μM or less against less for cancerous T-cell viability. In embodiments, the compound has an IC₅₀ value of about 3 μM or less against less for cancerous T-cell viability. In embodiments, the compound has an IC₅₀ value of about 2 μM or less against less for cancerous T-cell viability. In embodiments, the compound has an IC₅₀ value of about 1 μM or less against less for cancerous T-cell viability. In embodiments, the compound has an IC₅₀ value of about 900 nM or less against less for cancerous T-cell viability. In embodiments, the compound has an IC₅₀ value of about 800 nM or less against less for cancerous T-cell viability. In embodiments, the compound has an IC₅₀ value of about 700 nM or less against less for cancerous T-cell viability. In embodiments, the compound has an IC₅₀ value of about 600 nM or less against less for cancerous T-cell viability. In embodiments, the compound has an IC₅₀ value of about 500 nM or less against less for cancerous T-cell viability. In embodiments, the compound has an IC₅₀ value of about 400 nM or less against less for cancerous T-cell viability. In embodiments, the compound has an IC₅₀ value of about 300 nM or less against less for cancerous T-cell viability. In embodiments, the compound has an IC₅₀ value of about 200 nM or less against less for cancerous T-cell viability. In embodiments, the compound has an IC₅₀ value of about 100 nM or less against less for cancerous T-cell viability. In embodiments, the compound has an IC₅₀ value of about 50 nM or less against less for cancerous T-cell viability. In embodiments, the compound has an IC₅₀ value of about 40 nM or less against less for cancerous T-cell viability. In embodiments, the compound has an IC₅₀ value of about 30 nM or less against less for cancerous T-cell viability. In embodiments, the compound has an IC₅₀ value of about 20 nM or less against less for cancerous T-cell viability. In embodiments, the compound has an IC₅₀ value of about 10 nM or less against less for cancerous T-cell viability.

In embodiments, the compound has an IC₅₀ value 50 μM or less for cancerous T-cell viability. In embodiments, the compound has an IC₅₀ value 40 μM or less against less for cancerous T-cell viability. In embodiments, the compound has an IC₅₀ value 30 μM or less against less for cancerous T-cell viability. In embodiments, the compound has an IC₅₀ value 25 μM or less against less for cancerous T-cell viability. In embodiments, the compound has an IC₅₀ value 20 μM or less against less for cancerous T-cell viability. In embodiments, the compound has an IC₅₀ value 15 μM or less against less for cancerous T-cell viability. In embodiments, the compound has an IC₅₀ value 10 μM or less against less for cancerous T-cell viability. In embodiments, the compound has an IC₅₀ value 9 μM or less against less for cancerous T-cell viability. In embodiments, the compound has an IC₅₀ value 8 μM or less against less for cancerous T-cell viability. In embodiments, the compound has an IC₅₀ value 7 μM or less against less for cancerous T-cell viability. In embodiments, the compound has an IC₅₀ value 6 μM or less against less for cancerous T-cell viability. In embodiments, the compound has an IC₅₀ value 5 μM or less against less for cancerous T-cell viability. In embodiments, the compound has an IC₅₀ value 4 μM or less against less for cancerous T-cell viability. In embodiments, the compound has an IC₅₀ value 3 μM or less against less for cancerous T-cell viability. In embodiments, the compound has an IC₅₀ value 2 μM or less against less for cancerous T-cell viability. In embodiments, the compound has an IC₅₀ value 1 μM or less against less for cancerous T-cell viability. In embodiments, the compound has an IC₅₀ value 900 nM or less against less for cancerous T-cell viability. In embodiments, the compound has an IC₅₀ value 800 nM or less against less for cancerous T-cell viability. In embodiments, the compound has an IC₅₀ value 700 nM or less against less for cancerous T-cell viability. In embodiments, the compound has an IC₅₀ value 600 nM or less against less for cancerous T-cell viability. In embodiments, the compound has an IC₅₀ value 500 nM or less against less for cancerous T-cell viability. In embodiments, the compound has an IC₅₀ value 400 nM or less against less for cancerous T-cell viability. In embodiments, the compound has an IC₅₀ value 300 nM or less against less for cancerous T-cell viability. In embodiments, the compound has an IC₅₀ value 200 nM or less against less for cancerous T-cell viability. In embodiments, the compound has an IC₅₀ value 100 nM or less against less for cancerous T-cell viability. In embodiments, the compound has an IC₅₀ value 50 nM or less against less for cancerous T-cell viability. In embodiments, the compound has an IC₅₀ value 40 nM or less against less for cancerous T-cell viability. In embodiments, the compound has an IC₅₀ value 30 nM or less against less for cancerous T-cell viability. In embodiments, the compound has an IC₅₀ value 20 nM or less against less for cancerous T-cell viability. In embodiments, the compound has an IC₅₀ value 10 nM or less against less for cancerous T-cell viability.

T cell receptor (TCR) signaling for normal development, activation, and differentiation of T cells typically involves the classical NF-κB signaling pathway [1], which leads to cell proliferation. In malignant T cells, the NF-κB pathway is constitutively activated, although the mechanism remains unknown [1, 4]. NF-kB has been suggested to play a critical role in CTCL development and maintenance [4].

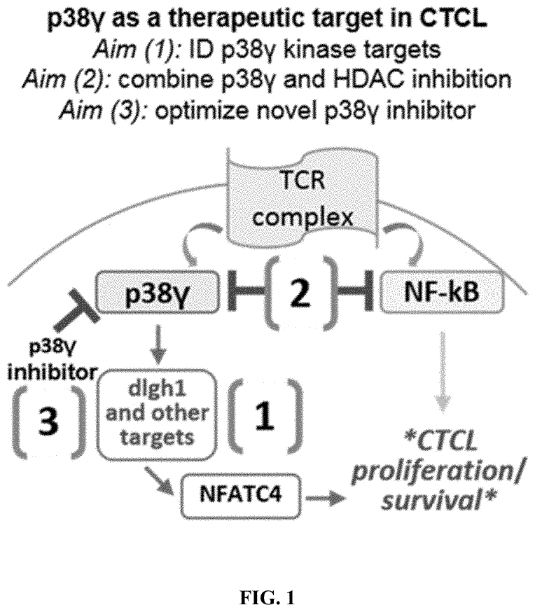

The preliminary data disclosed herein can suggest TCR signaling through the p38 family is also altered in CTCL, targeting NFATC4 instead of NFATC1 (FIG. 1). One candidate molecular regulator is p38 gamma (p38γ), a mitogen-activated protein kinase (MAPK 12), which is a 367-amino acid protein that is highly expressed in muscle, with no detectable expression in normal hematopoietic cells or tissues of the immune system, including lymph nodes and spleen[6,7]. Elevated expression of p38γ has been shown in malignant T cell activity and growth in response to T cell receptor (TCR) signaling. p38γ gene expression is selectively increased in CTCL cell lines and patient samples, but not in healthy T cells, suggesting it may play a key role in CTCL pathogenesis and be an effective target for therapy.

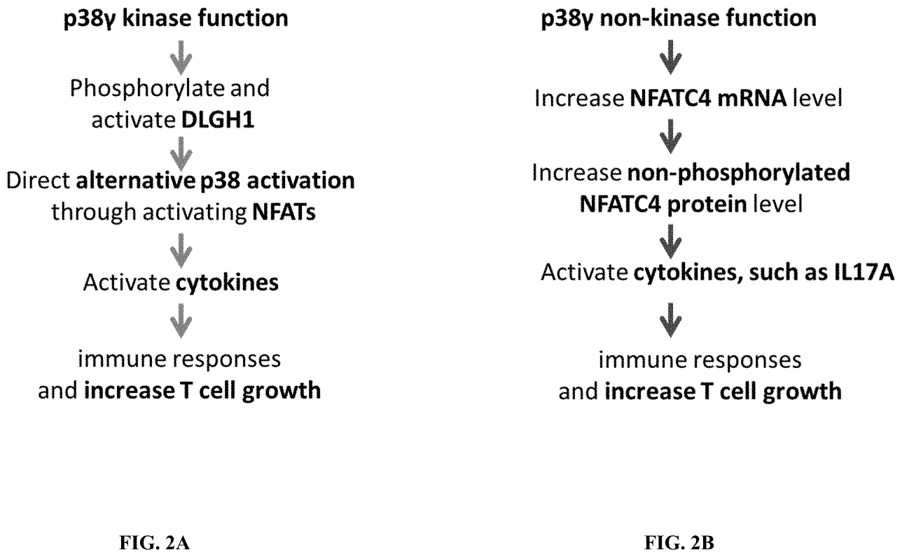

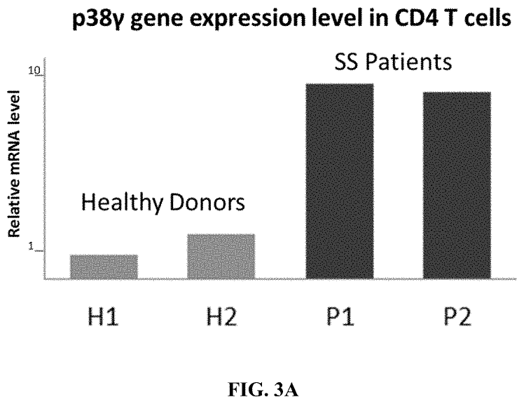

MAPKs, including p38, are typically activated through a classical signal transduction cascade that is triggered by stress stimuli and ultimately impacts intracellular processes such as proliferation and apoptosis [8, 9]. Environmental stress and pro-inflammatory cytokine responses trigger upstream MAPK kinases that facilitate dual phosphorylation of p38, which in turn phosphorylates downstream substrates[8,10] (FIG. 2A). In T cells, there is an additional p38 activation pathway, called the alternative pathway (FIG. 2B). In the alternative p38 pathway, antigen presentation and recognition by the TCR triggers a signaling cascade in which the tyrosine kinase zap70 phosphorylates p38 on Tyr-323, leading to auto-monophosphorylation of Thr-180 of p38 in the activation loop [9, 11, 12]. This alternatively phosphorylated p38 in turn upregulates transcriptional factors NFAT (nuclear factor of activated T cells) and IRF4 (interferon regulatory factor 4) [13]. This may lead to production of pro-inflammatory cytokine IL-17A, which is essential for CD4+ T helper 17 (Th17) cells, and is frequently deregulated in CTCL patients [13].

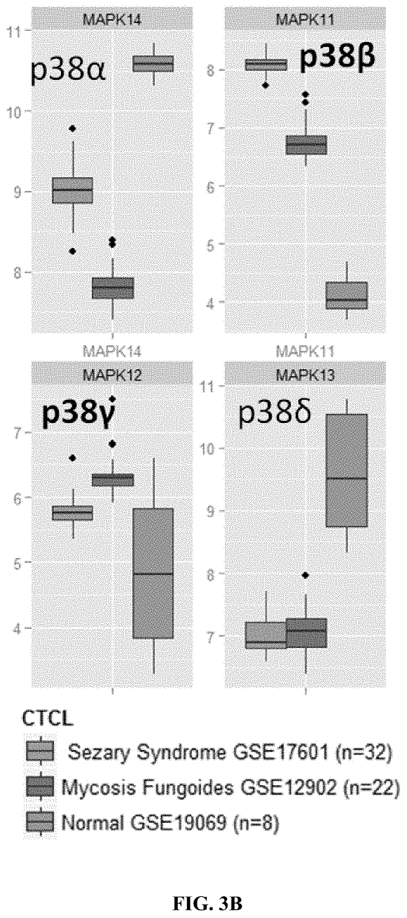

Importantly, although normal healthy T cells do not express p38γ, it has been shown that it is highly expressed in the human CTCL cell line HH, the human SS cell line Hut78, and in primary SS patient samples (FIG. 2A, FIG. 2B, FIG. 3A, FIG. 3B). This suggests that p38γ may be a key driver in CTCL. Overexpression of p38γ affects downstream pathways through its kinase activity. p38γ also affects downstream pathways through protein-protein interactions, due to the presence of a PDZ binding domain[15]. One such target is DLGH1, an important scaffolding protein that directs T cell signaling through the NFAT pathway rather than through the NF-kB pathway[15,16].

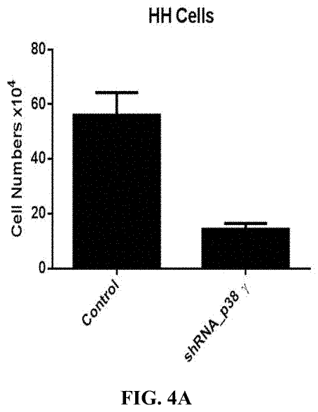

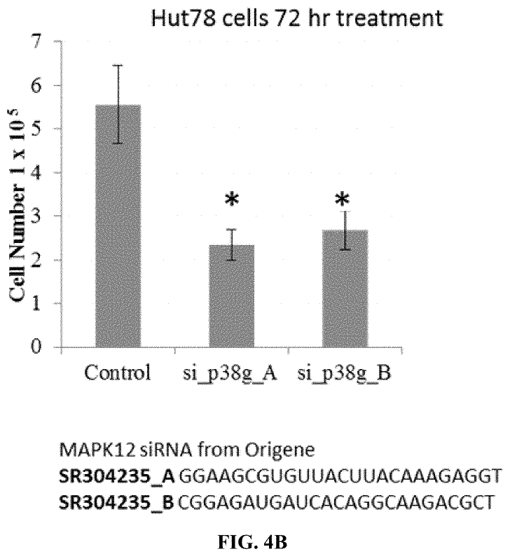

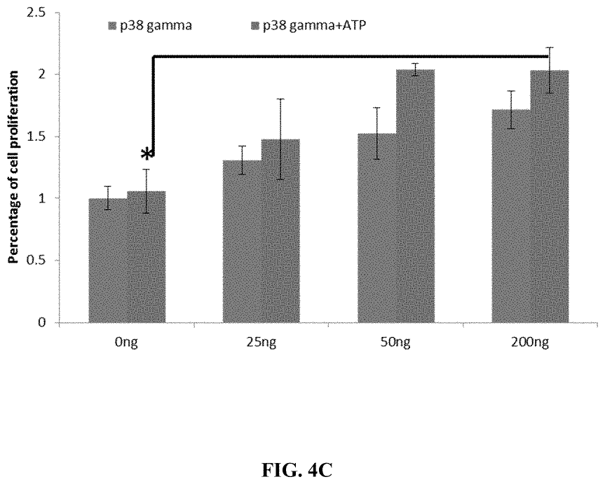

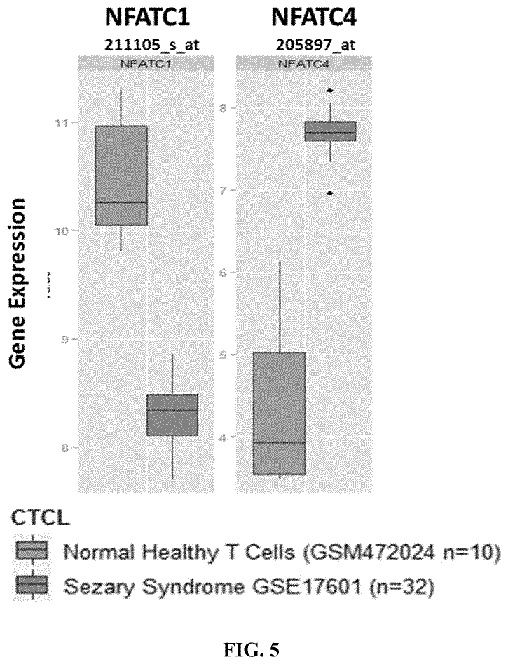

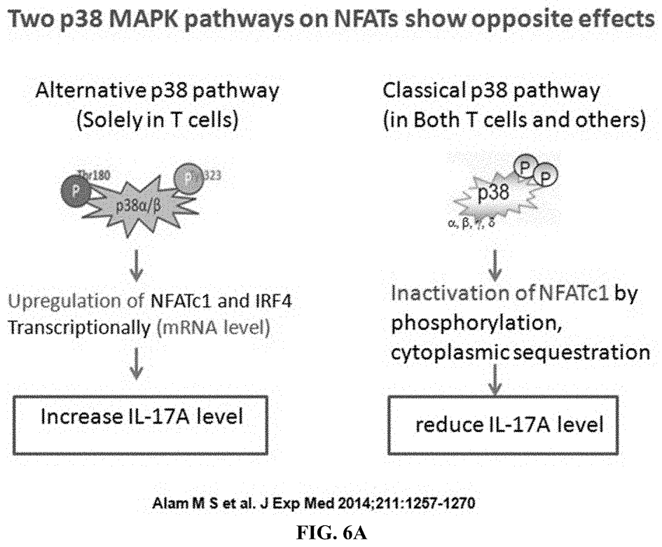

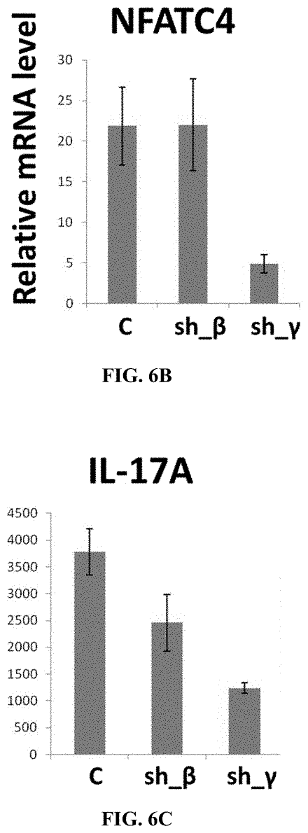

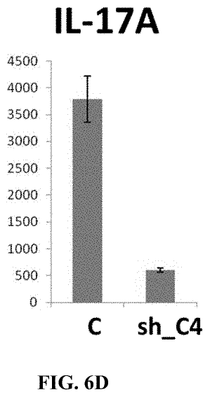

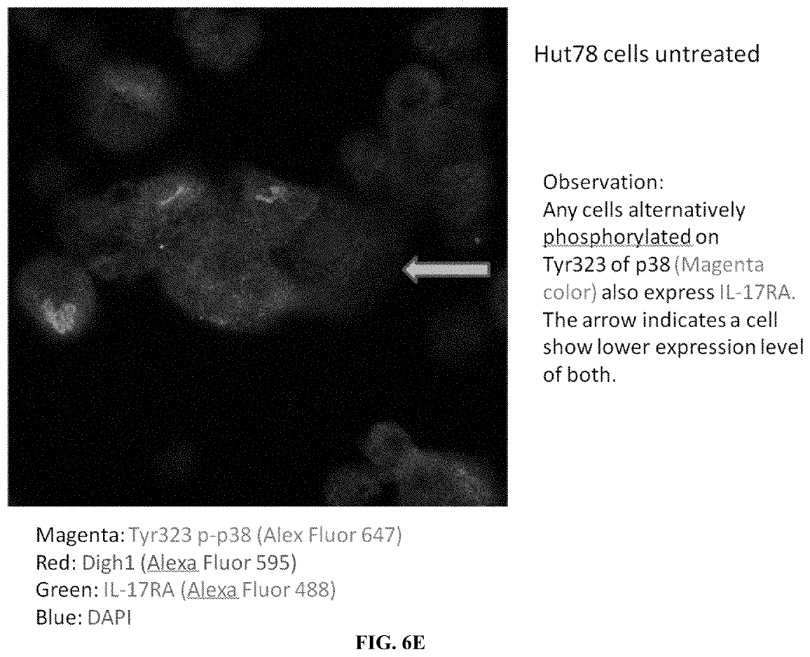

Further, chemical inhibition of p38γ or reduction of p38γ level by shRNA can correlate with significant reduction of cell proliferation in both Hut78 cells and HH cells (FIG. 4A, FIG. 4B). In addition, knockdown of p38γ revealed an unconventional direct role of p38γ on NFATC4 mRNA expression in Hut78 cells: the loss of p38γ ablated mRNA levels of NFATC4 (FIG. 5, FIG. 6B). Moreover, inhibition of NFATC4 by siRNA can reduce the proliferation of CTCL cell lines and significantly reduces cytokine IL-17A mRNA levels in Hut78 cells (FIG. 6C, FIG. 6D).

A compound (Compound 1, F7) in the invention is highly specific and potent p38γ inhibitor with an estimated Kᵢ of 10 nM for inhibiting p38γ enzymatic activity based on IC₅₀, which can suggest that it also binds to phosphorylated p38γ with a dissociation constant in the nM range. Also our NMR data has shown that it binds to both (unphosphorylated) and activated (phosphorylated) form of p38γ, which is unusual for a compound to bind to both forms with high affinity.

Our preliminary data provided herein show that p38γ signaling in CTCL involves nuclear factor of activated T-cells (NFAT), a transcription factor involved in T cell proliferation. In contrast to expression in normal T cells, NFATC4 is elevated in CTCL, while NFATC1 is dramatically reduced.

In addition to p38γ overexpression, constitutive expression and activation of NF-κB is common in CTCL [4, 16]. NF-κB provides a complementary signaling pathway to p38γ, and can be inhibited by histone deacetylase inhibitors (HDACi). Histone deacetylases (HDACs) remove acetyl groups from histone and non-histone targets. The HDAC enzyme family is divided into four major classes each comprised of multiple members. NF-κB interacts with Class I HDACs, to regulate gene expression; thus, inhibition of HDACs offers an opportunity to target NF-κB. We also have found combined application p38γ inhibition by Compound 1 and HDACi SAHA induce synergistic killing and propose that it through the p38γ and NF-kB pathways.

Thus, dissecting the key signaling molecules in the p38γ pathway, particularly in combination with other inhibitors (e.g. HDACi), can predict elements for potential resistance and alternative therapeutic targets for use in CTCL as well as other p38γ-driven malignancies. Multi-disciplinary approach to define a novel TCR-p38γ-NFATs signaling pathway may play an important role in the survival of CTCL. Furthermore, unique biological and clinical relevance of p38γ to develop effective may propose targeted therapies to improve outcome for CTCL patients.

However, without wishing to be bound to the theory, the present invention may provide methods of modulation of CTCL proliferation, for example, by the p38γ pathway influencing NFATs expression. In addition, the efficacy of targeting this pathway can be improved with complementary CTCL drug therapies that intersect with relevant pathways/targets. Inhibition of both p38γ and NF-κB through HDACi can also allow complementary targeting of two critical T cell pathways that are dysregulated in CTCL, thereby increasing therapeutic efficacy with reduced toxicity. In addition, p38γ inhibitor and combination therapy can provide a product that has potential therapeutic benefit for a CTCL and a spectrum of cancers.

### V. Methods of Treating

Accordingly, provided is a method of treating a cutaneous T-cell lymphoma (CTCL) in a subject in need thereof by administering to the subject an effective amount of a p38 gamma (p38γ) kinase inhibitor described herein. In embodiments, the method includes administering to the subject a combined effective amount of a histone deacetylase (HDAC) inhibitor (HDACi) and a compound (e.g. formulae (I), (II), (III), (III′), (III-A), (III-A′), (III-B), (III-C), (III-D), (III-E), (IV), (IV-A), (IV-A′), (IV-A″), (V-A), (V-B), (V-C), (V-D), (VI-A), (VI-B), (VII-A), (VII-B), (VII-C), (VII-D) or (VIII-A)) described above (including all embodiments thereof).

In embodiments, the method further includes co-administering an effective amount of histone deacetylase (HDAC) inhibitor with the p38 gamma (p38γ) kinase inhibitor or the compound described herein. In embodiments, the method further comprises co-administering an effective amount of HDACi with the p38 gamma (p38γ) kinase inhibitor or the compound described herein. Non-limiting examples of HDACi include the compound having the following structure:

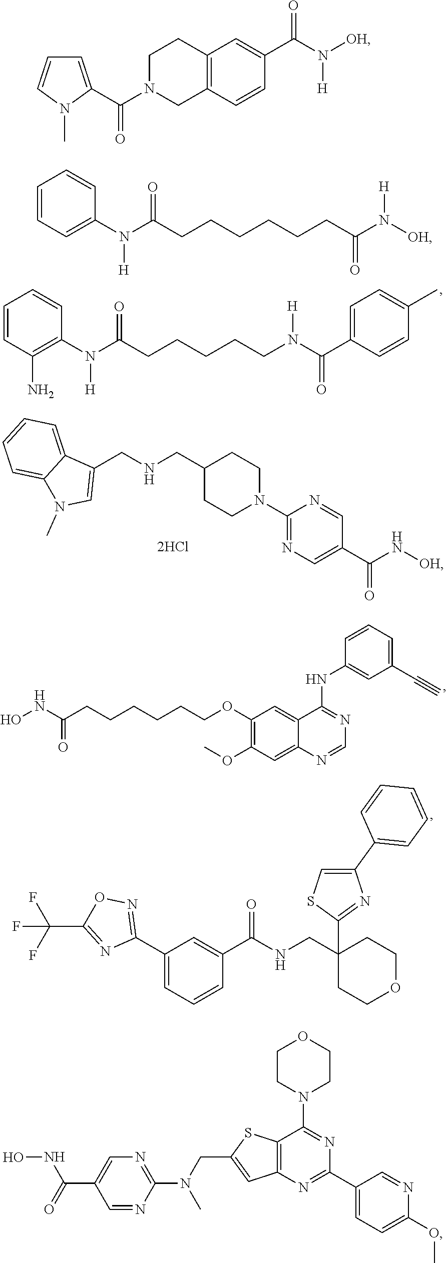

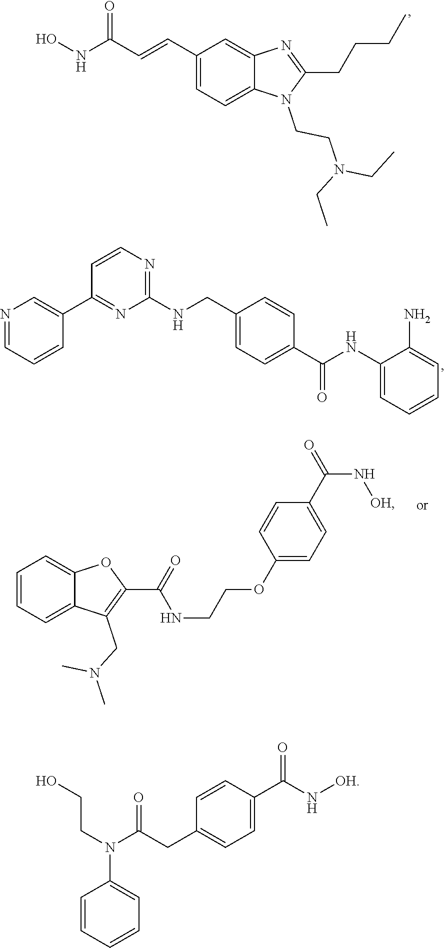 [SMILES: CCCCc1nc2cc(/C=C/C(=O)NO)ccc2n1CCN(CC)CC ; Nc1ccccc1NC(=O)c1ccc(CNc2nccc(-c3cccnc3)n2)cc1 ; CN(C)Cc1c(C(=O)NCCOc2ccc(C(=O)NO)cc2)oc2ccccc12 ; O=C(NO)c1ccc(CC(=O)N(CCO)c2ccccc2)cc1]

In embodiments, the HDACi is Vorinostat (SAHA), Romidepsin, Abexinostat, CI-994, Belinostat, Panobinostat, Givinostat, Entinostat, Mocetinostat, Trichostatin, SRT501, CUDC-101, JNJ-26481585, Quisinostat, RGFP109 or PCI24781.

In embodiments, the method further comprises co-administering an effective amount of the HDAC inhibitor with the p38 gamma (p38γ) kinase inhibitor. In embodiments, the SAHA may be co-administered with the p38 gamma (p38γ) kinase inhibitor. In embodiments, the romedepsin may be co-administered with the p38 gamma (p38γ) kinase inhibitor. In embodiments, the abexinostat may be co-administered with the p38 gamma (p38γ) kinase inhibitor. In embodiments, the entinostat may be co-administered with the p38 gamma (p38γ) kinase inhibitor. In embodiments, the panobinostat may be co-administered with the p38 gamma (p38γ) kinase inhibitor. In embodiments, the trichostatin may be co-administered with the p38 gamma (p38γ) kinase inhibitor. In embodiments, at least two from the HDAC inhibitor may be co-ad mistered with the p38 gamma (p38γ) kinase inhibitor.

In embodiments, the SAHA may be co-administered with the compound described herein. In embodiments, the romedepsin may be co-administered with the compound. In embodiments, the abexinostat may be co-administered with the compound. In embodiments, the entinostat may be co-administered with the compound. In embodiments, the panobinostat may be co-administered with the compound. In embodiments, the trichostatin may be co-administered with the compound. In embodiments, at least two from the HDAC inhibitor may be co-administered with the compound.

In embodiments, the p38 gamma (p38γ) kinase inhibitor or the compound described herein is co-administered with the one or more of HDAC inhibitor at a weight ratio between about 100:0.01 and 0.01:100. In embodiments, the p38 gamma (p38γ) kinase inhibitor or the compound described herein is co-administered with the one or more of HDAC inhibitor at a weight ratio between about 100:0.1 and 0.1:100. In embodiments, the p38 gamma (p38γ) kinase inhibitor or the compound described herein is co-administered with the one or more of HDAC inhibitor at a weight ratio between about 10:0.1 and 0.1:10. In embodiments, the p38 gamma (p38γ) kinase inhibitor or the compound described herein is co-administered with the one or more of HDAC inhibitor at a weight ratio between about 10:1 and 1:10.

In another aspect, provided herein are methods of treating a cancer or proliferative disease in a subject in need thereof. In one aspect is a method of treating a cancer in a subject in need thereof, by administering to the subject a combined effective amount of a histone deacetylase (HDAC) inhibitor and a p38 gamma (p38γ) kinase inhibitor described herein. In embodiments, the method comprises administering to the subject a combined effective amount of a histone deacetylase (HDAC) inhibitor and a compound (e.g. formulae (I), (II), (III), (III′), (III-A), (III-A′), (III-B), (III-C), (III-D), (III-E), (IV), (IV-A), (IV-A′), (IV-A″), (V-A), (V-B), (V-C), (V-D), (VI-A), (VI-B), (VII-A), (VII-B), (VII-C), (VII-D) or (VIII-A)) described above (including all embodiments thereof).

In embodiments, the co-administering of the histone deacetylase (HDAC) inhibitor and p38 gamma (p38γ) kinase inhibitor described herein results in synergestic therapeutic effect.

The terms “synergy”, “synergism” “synergistic” and “synergistic therapeutic effect” are used herein interchangeably and refer to a measured effect of compounds administered in combination where the measured effect is greater than the sum of the individual effects of each of the compounds administered alone as a single agent.

Further provided herein is a method of suppressing proliferation of cancer cell (e.g. breast cancer including triple negative breast cancer, prostate cancer, colon cancer, ovarian cancer or cutaneous T-cell lymphoma cell), by reducing or suppressing a p38 gamma (p38γ) kinase in the cell. In embodiments, the method of suppressing proliferation of cancer cell (e.g. breast cancer including triple negative breast cancer, prostate cancer, colon cancer, ovarian cancer or cutaneous T-cell lymphoma cell) comprises reducing or suppressing a p38 gamma (p38γ) kinase in the cell. In embodiments, the method of suppressing proliferation of the canker cell by reducing or suppressing a p38 gamma (p38γ) kinase in the cell comprises using the p38 gamma (p38γ) kinase inhibitors or the compound described herein. In embodiments, the method of reducing or suppressing a p38 gamma (p38γ) kinase in the cancer cell comprises contacting the cell with the p38 gamma (p38γ) kinase inhibitors or the compound described herein. In embodiments, the method of suppressing proliferation of cutaneous T-cell lymphoma cell by reducing or suppressing a p38 gamma (p38γ) kinase in the cell comprises contacting the cell with the p38 gamma (p38γ) kinase inhibitors or the compound described herein. In embodiments, the method of suppressing proliferation of cutaneous T-cell lymphoma cell comprises contacting the cell with a combined effective amount of a histone deacetylase (HDAC) inhibitor and the p38 gamma (p38γ) kinase inhibitors or the compound described herein.

### VI. Embodiments

Embodiment P1. A compound having a structure of Formula (III):

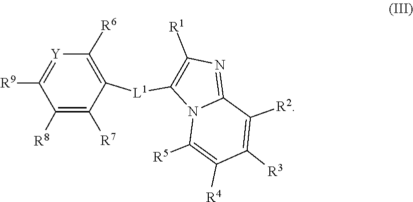

L¹, Y, R¹, R², R³, R⁴, R⁵, R⁶, R⁷, R⁸, and R⁹ are as described herein.

Embodiment P2. The compound according to embodiment P1, the compound is:

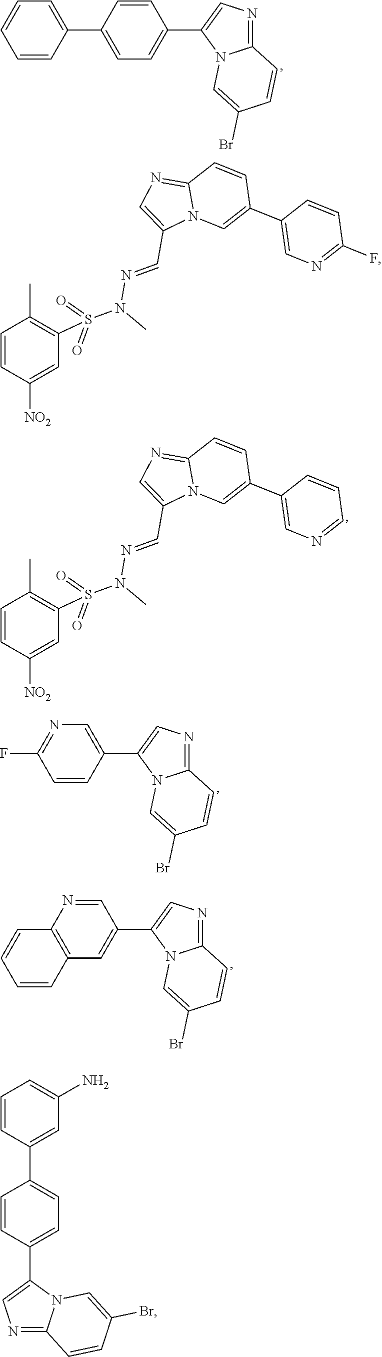

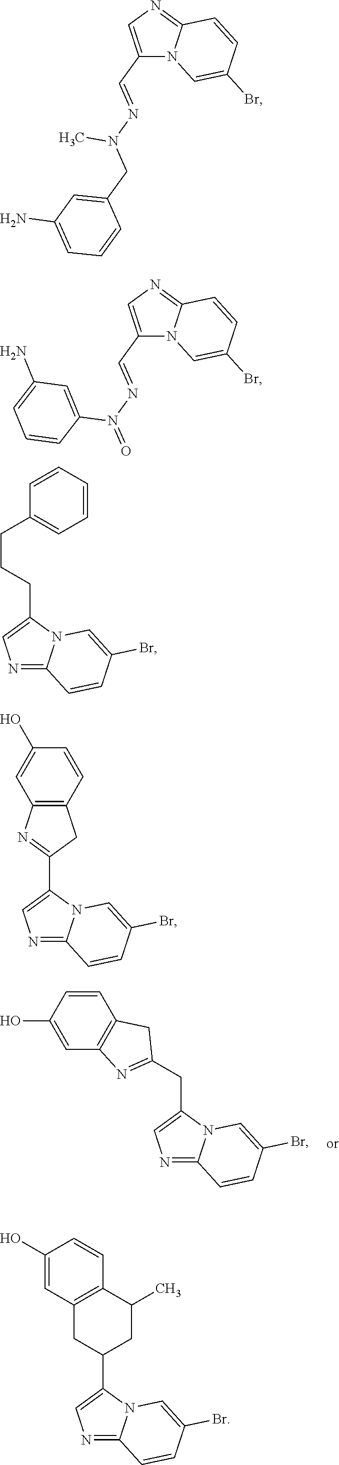

Embodiment P3. A method of treating cutaneous T-cell lymphoma (CTCL) in a subject in need thereof, the method comprising administering an effective amount of a p38 gamma (p38γ) kinase inhibitor to the subject.

Embodiment P4. A method of treating a cancer in a subject in need thereof, the method comprising administering a combined effective amount of a histone deacetylase (HDAC) inhibitor and a p38 gamma (p38γ) kinase inhibitor to said subject.

Embodiment P5. A method of suppressing proliferation of a cutaneous T-cell lymphoma (CTCL) cell, the method comprising contacting the cell with an effective amount of a p38 gamma (p38γ) kinase inhibitor.

Embodiment P6. A compound used for the methods according to the Embodiments P3-P5, the compound is:

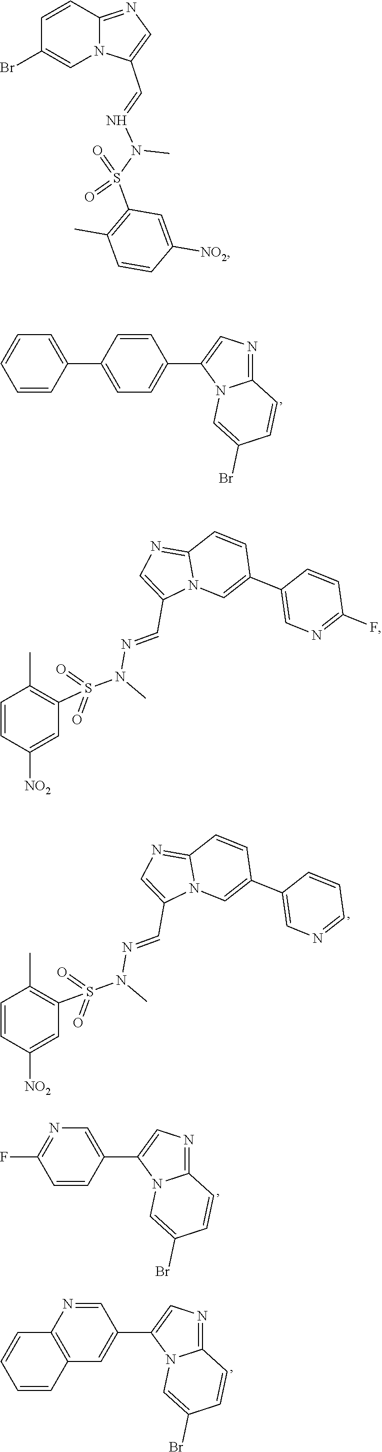

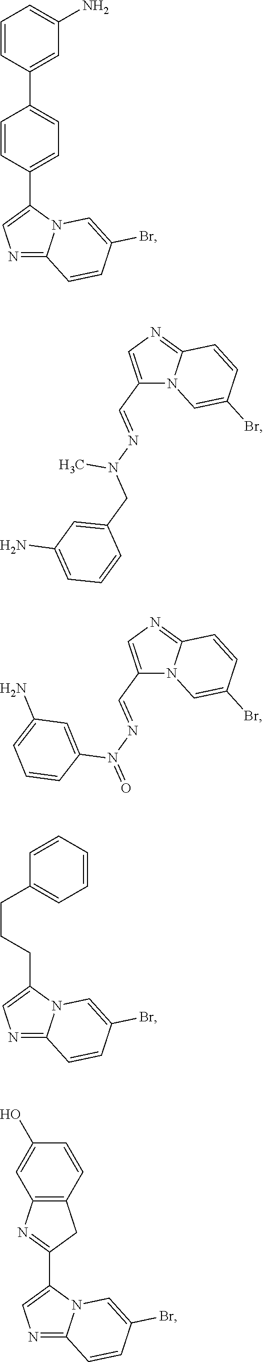

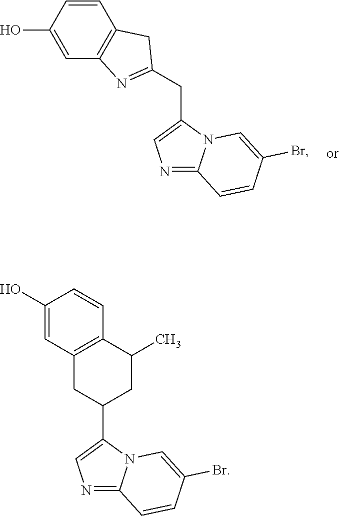 [SMILES: Oc1ccc2c(c1)N=C(Cc1cnc3ccc(Br)cn13)C2 ; CC1CC(c2cnc3ccc(Br)cn23)Cc2cc(O)ccc21]

Embodiment Q1. A method of treating cutaneous T-cell lymphoma (CTCL) in a subject in need thereof, the method comprising administering an effective amount of a p38 gamma (p38γ) kinase inhibitor to said subject.

Embodiment Q2. The method of Embodiment Q1, wherein the p38γ kinase inhibitor is a compound represented by Formula (I):

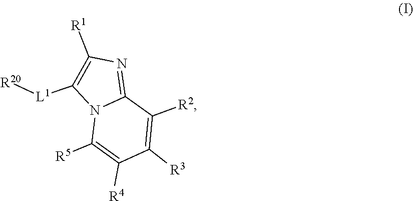

wherein:
L¹ is a bond, —SOₙ₁₁L^{1A}-, —SOᵥ₁₁NR¹¹L^{1A}-, —NHC(O)NR¹¹L^{1A}-, —NR¹¹L^{1A}-, —C(O)L^{1A}-, —C(O)OL^{1A}-, —C(O)NR¹¹L^{1A}-, —OL^{1A}-, —NR¹¹SO₂L^{1A}-, —NR¹¹C(O)L^{1A}-, —NR¹¹C(O)OL^{1A}-, —NR¹¹OL^{1A}-, —SL^{1A}-, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CX¹₃, —CHX¹₂, —CH₂X¹, —OCX¹₃, —OCH₂X¹, —OCHX¹₂, —N₃, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —OCX²₃, —OCH₂X², —OCHX²₂, —N₃, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
   R³ is hydrogen, halogen, —CX³₃, —CHX³₂, —CH₂X³, —OCX³₃, —OCH₂X³, —OCHX³₂, —N₃, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —N₃, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, halogen, —CX⁵₃, —CHX⁵₂, —CH₂X⁵, —OCX⁵₃, —OCH₂X⁵, —OCHX⁵₂, —N₃, —CN, —SOₙ₅R^{5D}, —SOᵥ₅NR^{5A}R^{5B}, —NHC(O)NR^{5A}R^{5B}, —N(O)ₘ₅, —NR^{5A}R^{5B}, —C(O)R^{5C}, —C(O)—OR^{5C}, —C(O)NR^{5A}R^{5B}, —OR^{5D}, —NR^{5A}SO₂R^{5D}, —NR^{5A}C(O)R^{5C}, —NR^{5A}C(O)OR^{5C}, —NR^{5A}OR^{5C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²⁰ is substituted or unsubstituted cycloalkyl, substituted or unsubstituted heterocycloalkyl, substituted or unsubstituted aryl, or substituted or unsubstituted heteroaryl;
wherein R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D} and R¹¹ are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{1A} is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
n1, n2, n3, n4, n5 and n11 are independently an integer from 0 to 4;
m1, m2, m3, m4, m5, v1, v2, v3, v4, v5 and v11 are independently an integer from 1 to 2; and
X, X¹, X², X³, X⁴, and X⁵ are independently —F, —Cl, —Br, or —I.

Embodiment Q3. The method of Embodiment Q2, wherein the p38γ kinase inhibitor is a compound represented by Formula (II):

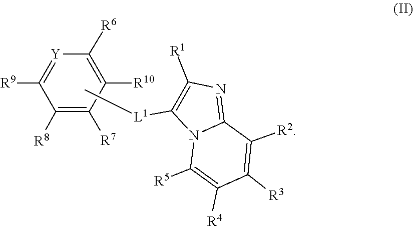

wherein:
L¹ is —SOₙ₁₁L^{1A}-, —SOᵥ₁₁NR¹¹L^{1A}-, —NHC(O)NR¹¹L^{1A}-, —NR¹¹L^{1A}-, —C(O)L^{1A}-, —C(O)OL^{1A}-, —C(O)NR¹¹L^{1A}-, —OL^{1A}-, —NR¹¹SO₂L^{1A}-, —NR¹¹C(O)L^{1A}-, —NR¹¹C(O)OL^{1A}-, —NR¹¹OL^{1A}-, —SL^{1A}-, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
Y is —N═ or —CR¹²═;
R⁶ is a bond (to L¹), hydrogen, halogen, —CX⁶₃, —CHX⁶, —CH₂X⁶, —OCX⁶₃, —OCH₂X⁶, —OCHX⁶₂, —N₃, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is a bond (to L¹), hydrogen, halogen, —CX⁷₃, —CHX⁷₂, —CH₂X⁷, —OCX⁷₃, —OCH₂X⁷, —OCHX⁷₂, —N₃, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is a bond (to L¹), hydrogen, halogen, —CX⁸₃, —CHX⁸₂, —CH₂X⁸, —OCX⁸₃, —OCH₂X⁸, —OCHX⁸₂, —N₃, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —OR^{8D}, —NR^{8A}SO₂R^{8D}, —NR^{8A}C(O)R^{8C}, —NR^{8A}C(O)OR^{8C}, —NR^{8A}OR^{8C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is a bond (to L¹), hydrogen, halogen, —CX⁹₃, —CHX⁹₂, —CH₂X⁹, —OCX⁹₃, —OCH₂X⁹, —OCHX⁹₂, —N₃, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ is a bond (to L¹), hydrogen, halogen, —CX¹⁰₃, —CHX¹⁰₂, —CH₂X¹⁰, —OCX¹⁰₃, —OCH₂X¹⁰, —OCHX¹⁰₂, —N₃, —CN, —SOₙ₁₀R^{10D}, —SOᵥ₁₀NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen, halogen, —CX¹²₃, —CHX¹²₂, —CH₂X¹², —OCX¹²₃, —OCH₂X¹², —OCHX¹²₂, —N₃, —CN, —SOₙ₁₂R^{12D}, —SOᵥ₁₂NR^{12A}R^{12B}, —NHC(O)NR^{12A}R^{12B}, —N(O)ₘ₁₂, —NR^{12A}R^{12B}, —C(O)R^{12C}, —C(O)—OR^{12C}, —C(O)NR^{12A}R^{12B}, —OR^{12D}, —NR^{12A}SO₂R^{12D}, —NR^{12A}C(O)R^{12C}, —NR^{12A}C(O)OR^{12C}, —NR^{12A}OR^{12C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
wherein one of R⁶, R⁷, R⁸, R⁹ are R¹⁰ is a bond to L¹;
wherein R^{6A}, R^{6B}, R^{6C}, R^{6D}, R^{7A}, R^{7B}, R^{7C}, R^{7D}, R^{8A}, R^{8B}, R^{8C}, R^{8D}, R^{9A}, R^{9B}, R^{9C}, R^{9D}, R^{10A}, R^{10B}, R^{10C}, R^{10D}, R^{12A}, R^{12B}, R^{12C}, and R^{12D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ and R⁸ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁸ and R⁹ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁹ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁶ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁶ and R¹⁰ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁷ and R¹⁰ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
n6, n7, n8, n9, n10 and n12 are independently an integer from 0 to 4;
m6, m7, m8, m9, m10, m12, v6, v7, v8, v9, v10 and v12 are independently an integer from 1 to 2; and
X⁶, X⁷, X⁸, X⁹, X¹⁰ and X¹¹ are independently —F, —Cl, —Br, or —I.

Embodiment Q4. The method of Embodiment Q3, wherein the compound is represented by Formula (III),

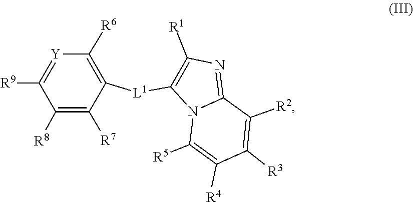

wherein:
Y is —N═ or —CR¹²═;
R⁶ is hydrogen, halogen, —CX⁶₃, —CHX⁶₂, —CH₂X⁶, —OCX⁶₃, —OCH₂X⁶, —OCHX⁶₂, —N₃, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is hydrogen, halogen, —CX⁷₃, —CHX⁷₂, —CH₂X⁷, —OCX⁷₃, —OCH₂X⁷, —OCHX⁷₂, —N₃, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is hydrogen, halogen, —CX⁸₃, —CHX⁸₂, —CH₂X⁸, —OCX⁸₃, —OCH₂X⁸, —OCHX⁸₂, —N₃, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —OR^{8D}, —NR^{8A}SO₂R^{8D}, —NR^{8A}C(O)R^{8C}, —NR^{8A}C(O)OR^{8C}, —NR^{8A}OR^{8C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CX⁹₃, —CHX⁹₂, —CH₂X⁹, —OCX⁹₃, —OCH₂X⁹, —OCHX⁹₂, —N₃, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen, halogen, —CX¹²₃, —CHX¹²₂, —CH₂X¹², —OCX¹²₃, —OCH₂X¹², —OCHX¹²₂, —N₃, —CN, —SOₙ₁₂R^{12D}, —SOᵥ₁₂NR^{12A}R^{12B}, —NHC(O)NR^{12A}R^{12B}, —N(O)ₘ₁₂, —NR^{12A}R^{12B}, —C(O)R^{12C}, —C(O)—OR^{12C}, —C(O)NR^{12A}R^{12B}, —OR^{12D}, —NR^{12A}SO₂R^{12D}, —NR^{12A}C(O)R^{12C}, —NR^{12A}C(O)OR^{12C}, —NR^{12A}OR^{12C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
wherein R^{6A}, R^{6B}, R^{6C}, R^{6D}, R^{7A}, R^{7B}, R^{7C}, R^{7D}, R^{8A}, R^{8B}, R^{8C}, R^{8D}, R^{9A}, R^{9B}, R^{9C}, R^{9D}, R^{12A}, R^{12B}, R^{12C}, and R^{12D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ and R⁸ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁸ and R⁹ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁹ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁶ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
n6, n7, n8, n9 and n12 are independently an integer from 0 to 4;
m6, m7, m8, m9, m12, v6, v7, v8, v9 and v12 are independently an integer from 1 to 2; and
X⁶, X⁷, X⁸, X⁹ and X¹² are independently —F, —Cl, —Br, or —I.

Embodiment Q5. The method of any one of Embodiments Q2-Q4, wherein R¹ is hydrogen.

Embodiment Q6. The method of any one of Embodiments Q3-Q5, wherein:
Y is —CR¹²═.

Embodiment Q7. The method of Embodiment Q6, wherein R¹² is hydrogen, halogen, or unsubstituted C₁-C₄ alkyl.

Embodiment Q8. The method of Embodiment Q6, wherein Y is —CH═.

Embodiment Q9. The method of any one of Embodiments Q3-Q5, wherein Y is —N═.

Embodiment Q10. The method of any one of Embodiments Q2-Q9, wherein:
L¹ is R¹³-substituted or unsubstituted phenylene;
R¹³ is halogen, —CX¹³₃, —CHX¹³₂, —CH₂X¹³, —OCX¹³₃, —OCH₂X¹³, —OCHX¹³₂, —CN, —SOₙ₁₃R^{13D}, —SOᵥ₁₃NR^{13A}R^{13B}, —NHC(O)NR^{13A}R^{13B}, —N(O)ₘ₁₃, —NR^{13A}R^{13B}, —C(O)R^{13C}, —C(O)—OR^{13C}, —C(O)NR^{13A}R^{13B}, —OR^{13D}, —NR^{13A}SO₂R^{13D}, —NR^{13A}C(O)R^{13C}, —NR^{13A}C(O)OR^{13C}, —NR^{13A}OR^{13C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl,
wherein R^{13A}, R^{13B}, R^{13C}, and R^{13D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n13 is independently an integer from 0 to 4, and
m13 and v13 are each independently an integer from 0 to 2, and
   X¹³ is independently —F, —Cl, —Br, or —I.

Embodiment Q11. The method of any one of Embodiments Q2-Q10, wherein L¹ is unsubstituted phenylene.

Embodiment Q12. The method of any one of Embodiments Q2-Q9, wherein:
L¹ is R¹³-substituted or unsubstituted heteroalkylene;
R¹³ is halogen, —CX¹³₃, —CHX¹³₂, —CH₂X¹³, —OCX¹³₃, —OCH₂X¹³, —OCHX¹³₂, —CN, —SOₙ₁₃R^{13D}, —SOᵥ₁₃NR^{13A}R^{13B}, —NHC(O)NR^{13A}R^{13B}, —N(O)ₘ₁₃, —NR^{13A}R^{13B}, —C(O)R^{13C}, —C(O)—OR^{13C}, —C(O)NR^{13A}R^{13B}, —OR^{13D}, —NR^{13A}SO₂R^{13D}, —NR^{13A}C(O)R^{13C}, —N R^{13A}C(O)OR^{13C}, —NR^{13A}OR^{13C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl,
wherein R^{13A}, R^{13B}, R^{13C}, and R^{13D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n13 is independently an integer from 0 to 4,
m13 and v13 are each independently an integer from 0 to 2, and
X¹³ is independently —F, —Cl, —Br, or —I.

Embodiment Q13. The method of any one of Embodiments Q2-Q9, wherein:
L¹ is R¹³-substituted or unsubstituted 2 to 4 membered heteroalkylene; and
R¹³ is halogen, or unsubstituted C₁-C₃ alkyl.

Embodiment Q14. The method of any one of Embodiments Q2-Q9, wherein:
L¹ is —SO₂N(R¹⁴)N═CH—;
wherein R¹⁴ is hydrogen, —CX¹⁴₃, —CHX¹⁴₂, —CH₂X¹⁴, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, and
X¹⁴ is independently —F, —Cl, —Br, or —I.

Embodiment Q15. The method of Embodiment Q14, wherein R¹⁴ is unsubstituted C₁-C₃ alkyl.

Embodiment Q15. The method of any one of Embodiments Q2-Q9, wherein L¹ is —SO₂—N(CH₃)N═CH—.

Embodiment Q17. The method of any one of Embodiments Q2-Q16, wherein at least one of R², R³, R⁴ and R⁵ is halogen.

Embodiment Q18. The method of Embodiment Q17, wherein R², R³, and R⁵ are hydrogen and R⁴ is —F, —Cl or —Br.

Embodiment Q19. The method of any one of Embodiments Q2-Q16, wherein at least one of R², R³, R⁴ and R⁵ is substituted or unsubstituted pyridyl.

Embodiment Q20. The method of Embodiment Q19, wherein R², R³ and R⁵ are hydrogen, and R⁴ is halogen-substituted pyridyl or unsubstituted pyridyl.

Embodiment Q21. The method of any one of Embodiments Q2-Q20, wherein R⁶, R⁷, R⁸ and R⁹ are independently hydrogen, halogen, —NO₂, or unsubstituted C₁-C₃ alkyl.

Embodiment Q22. The method of any one of Embodiments Q2-9, wherein:
L¹ is unsubstituted phenylene;
R¹, R², R³ and R⁵ are hydrogen;
R⁴ is —F, —Cl or —Br;
R⁶, R⁷, R⁸ and R⁹ are independently hydrogen, halogen, —NO₂, or unsubstituted C₁-C₃ alkyl.

Embodiment Q23. The method of any one of Embodiments Q2-Q9, wherein:
L¹ is substituted or unsubstituted C₄-C₆ alkylene, or substituted or unsubstituted 4 to 6 membered heteroalkylene;
R¹, R², R³ and R⁵ are hydrogen;
R⁴ is —Cl or —Br; and
   R⁶, R⁷, R⁸ and R⁹ are independently hydrogen, halogen, NO₂, or unsubstituted C₁-C₃ alkyl.

Embodiment Q24. The method of any one of Embodiments Q2-Q9, wherein:
L¹ is —SO₂—N(CH₃)N═CH—;
R², R³ and R⁵ are hydrogen;
R⁴ is halogen, or substituted or unsubstituted pyridyl;
R⁶ is unsubstituted methyl;
R⁷ is hydrogen;
R⁸ is —NO₂; and
R⁹ is hydrogen.

Embodiment Q25. The method of Embodiment Q24, wherein R⁴ is halogen-substituted pyridyl or unsubstituted pyridyl.

Embodiment Q26. The method of any one of Embodiments Q2-Q25, wherein L¹ has a length of about 4 to 12 Å.

Embodiment Q27. The method of any one of Embodiments Q2-Q26, wherein the compound is:

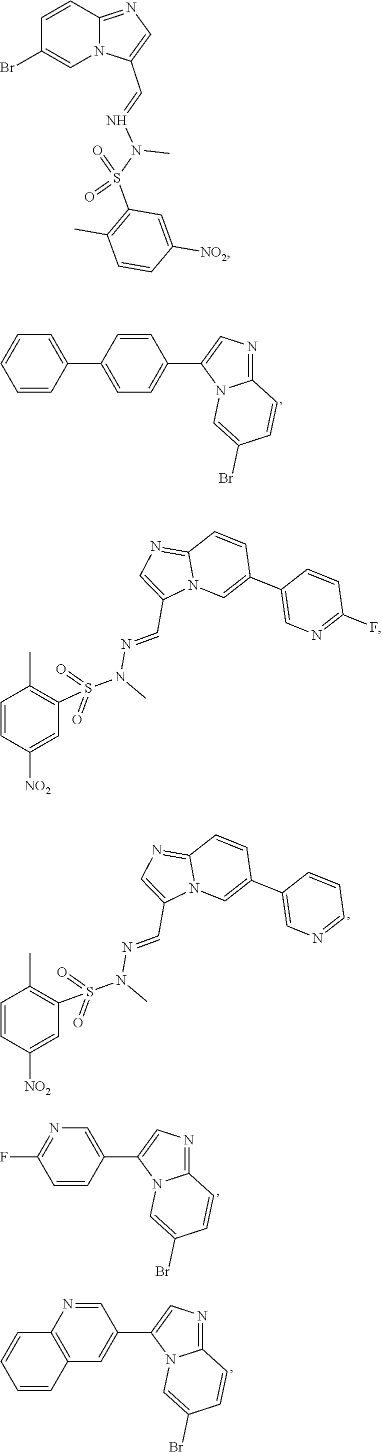

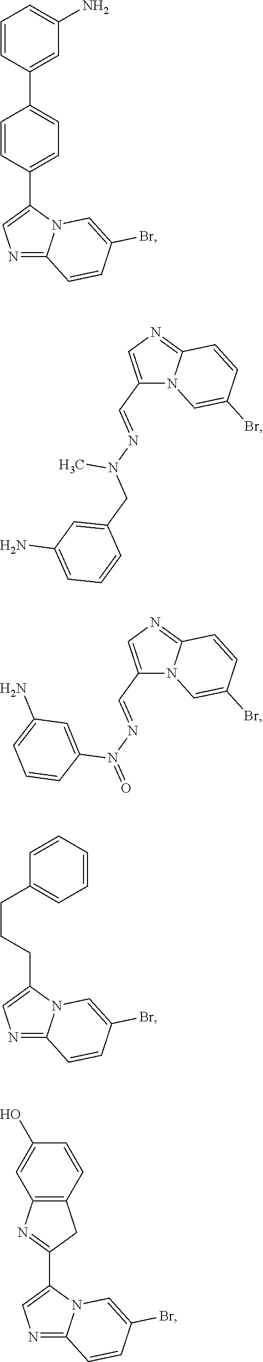

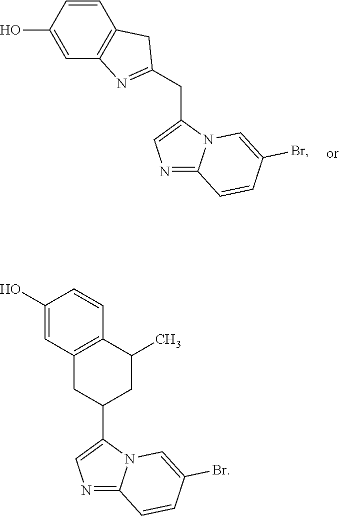 [SMILES: Oc1ccc2c(c1)N=C(Cc1cnc3ccc(Br)cn13)C2 ; CC1CC(c2cnc3ccc(Br)cn23)Cc2cc(O)ccc21]

Embodiment Q28. The method of any one of Embodiments Q2-Q27, wherein the p38γ kinase inhibitor has an IC₅₀ value of about 50 μM or less.

Embodiment Q29. The method of any one of Embodiments Q1-Q28, the method further comprising co-administering an effective amount of histone deacetylase (HDAC) inhibitor.

Embodiment Q30. The method of Embodiment 29, wherein the HDAC inhibitor is a compound selected from SAHA, romedepsin, abexinostat, entinostat, panobinostat and trichostatin A.

Embodiment Q31. A method of treating a cancer in a subject in need thereof, the method comprising administering a combined effective amount of a histone deacetylase (HDAC) inhibitor and a p38 gamma (p38γ) kinase inhibitor to said subject.

Embodiment Q32. The method of Embodiment Q32, wherein the cancer is selected from breast cancer, prostate cancer, colon cancer, lymphoma and ovarian cancer.

Embodiment Q33. The method of Embodiment Q31, wherein the cancer is cutaneous T-cell lymphoma (CTCL).

Embodiment Q33. The method of any one of Embodiments Q32-Q33, wherein the p38γ kinase inhibitor is a compound represented by Formula (I):

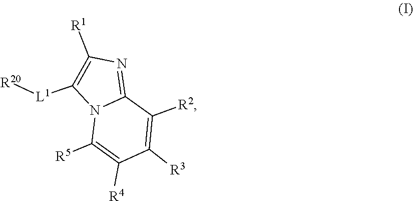

wherein:
L¹ is a bond, —SOₙ₁₁L^{1A}-, —SOᵥ₁₁NR¹¹L^{1A}-, —NHC(O)NR¹¹L^{1A}-, —NR¹¹L^{1A}-, —C(O)L^{1A}-, —C(O)OL^{1A}-, —C(O)NR¹¹L^{1A}-, —OL^{1A}-, —NR¹¹SO₂L^{1A}-, —NR¹¹C(O)L^{1A}-, —NR¹¹C(O)OL^{1A}-, —NR¹¹OL^{1A}-, —SL^{1A}-, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CX¹₃, —CHX¹₂, —CH₂X¹, —OCX¹₃, —OCH₂X¹, —OCHX¹₂, —N₃, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —OCX²₃, —OCH₂X², —OCHX²₂, —N₃, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —N₃, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —N₃, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, halogen, —CX⁵₃, —CHX⁵₂, —CH₂X⁵, —OCX⁵₃, —OCH₂X⁵, —OCHX⁵₂, —N₃, —CN, —SOₙ₅R^{5D}, —SOᵥ₅NR^{5A}R^{5B}, —NHC(O)NR^{5A}R^{5B}, —N(O)ₘ₅, —NR^{5A}R^{5B}, —C(O)R^{5C}, —C(O)—OR^{5C}, —C(O)NR^{5A}R^{5B}, —OR^{5D}, —NR^{5A}SO₂R^{5D}, —NR^{5A}C(O)R^{5C}, —NR^{5A}C(O)OR^{5C}, —NR^{5A}OR^{5C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²⁰ is substituted or unsubstituted cycloalkyl, substituted or unsubstituted heterocycloalkyl, substituted or unsubstituted aryl, or substituted or unsubstituted heteroaryl;
wherein R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D} and R¹¹ are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{1A} is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
n1, n2, n3, n4, n5 and n11 are independently an integer from 0 to 4;
m1, m2, m3, m4, m5, v1, v2, v3, v4, v5 and v11 are independently an integer from 1 to 2;
X, X¹, X², X³, X⁴, and X⁵ are independently —F, —Cl, —Br, or —I.

Embodiment Q35. The method of Embodiment Q34, wherein the p38γ kinase inhibitor is a compound represented by Formula (II),

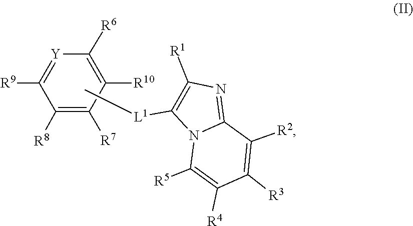

wherein:
L¹ is —SOₙ₁₁L^{1A}-, —SOᵥ₁₁NR¹¹L^{1A}-, —NHC(O)NR¹¹L^{1A}-, —NR¹¹L^{1A}-, —C(O)L^{1A}-, —C(O)OL^{1A}-, —C(O)NR¹¹L^{1A}-, —OL^{1A}-, —NR¹¹SO₂L^{1A}-, —NR C(O)L^{1A}-, —NR¹¹C(O)OL^{1A}-, —NR¹¹OL^{1A}-, —SL^{1A}-, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
Y is —N═ or —CR¹²═;
R⁶ is a bond (to L¹), hydrogen, halogen, —CX⁶₃, —CHX⁶₂, —CH₂X⁶, —OCX⁶₃, —OCH₂X⁶, —OCHX⁶₂, —N₃, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is a bond (to L¹), hydrogen, halogen, —CX⁷₃, —CHX⁷₂, —CH₂X⁷, —OCX⁷₃, —OCH₂X⁷, —OCHX⁷₂, —N₃, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is a bond (to L¹), hydrogen, halogen, —CX⁸₃, —CHX⁸₂, —CH₂X⁸, —OCX⁸₃, —OCH₂X⁸, —OCHX⁸₂, —N₃, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —OR^{8D}, —NR^{8A}SO₂R^{8D}, —NR^{8A}C(O)R^{8C}, —R^{8A}C(O)OR^{8C}, —NR^{8A}OR^{8C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is a bond (to L¹), hydrogen, halogen, —CX⁹₃, —CHX⁹₂, —CH₂X⁹, —OCX⁹₃, —OCH₂X⁹, —OCHX⁹₂, —N₃, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ is a bond (to L¹), hydrogen, halogen, —CX¹⁰₃, —CHX¹⁰₂, —CH₂X¹⁰, —OCX¹⁰₃, —OCH₂X¹⁰, —OCHX¹⁰₂, —N₃, —CN, —SOₙ₁₀R^{10D}SOᵥ₁₀NR^{10A}R^{10B}, —NHC(O)NR^{20A}R^{10B}, —N(O)ₘ₁₀, —NR^{10A}R^{10B}, —C(O)Roc, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen, halogen, —CX¹²₃, —CHX¹²₂, —CH₂X¹², —OCX¹²₃, —OCH₂X¹², —OCHX¹²₂, —N₃, —CN, —SOₙ₁₂R^{12D}, —SOᵥ₁₂NR^{12A}R^{12B}, —NHC(O)NR^{12A}R^{12B}, —N(O)ₘ₁₂, —NR^{12A}R^{12B}, —C(O)R^{12C}, —C(O)—OR^{12C}, —C(O)NR^{12A}R^{12B}, —OR^{12D}, —NR^{12A}SO₂R^{12D}, —NR^{12A}C(O)R^{12C}, —NR^{12A}C(O)OR^{12C}, —NR^{12A}OR^{12C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
wherein one of R⁶, R⁷, R¹, R⁹ are R¹⁰ is a bond to L¹;
wherein R^{6A}, R^{6B}, R^{6C}, R^{6D}, R^{7A}, R^{7B}, R^{7C}, R^{7D}, R^{8A}, R^{8B}, R^{8C}, R^{8D}, R^{9A}, R^{9B}, R^{9C}, R^{9D}, R^{10A}, R^{10B}, R^{10C}, R^{10D}, R^{12A}, R^{12B}, R^{12C}, and R^{12D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ and R⁸ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁸ and R⁹ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁹ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁶ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁶ and R¹⁰ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁷ and R¹⁰ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
n6, n7, n8, n9, n10 and n12 are independently an integer from 0 to 4;
m6, m7, m8, m9, m10, m12, v6, v7, v8, v9, v10 and v12 are independently an integer from 1 to 2; and
X⁶, X⁷, X⁸, X⁹, X¹⁰ and X¹² are independently —F, —Cl, —Br, or —I.

Embodiment Q36. The method of Embodiment Q35, wherein the compound is represented by Formula (III),

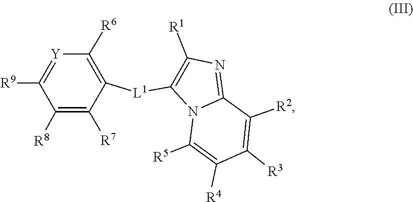

wherein:
Y is —N═ or —CR¹²═;
R⁶ is hydrogen, halogen, —CX⁶₃, —CHX⁶₂, —CH₂X⁶, —OCX⁶₃, —OCH₂X⁶, —OCHX⁶₂, —N₃, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is hydrogen, halogen, —CX⁷₃, —CHX⁷₂, —CH₂X⁷, —OCX⁷₃, —OCH₂X⁷, —OCHX⁷₂, —N₃, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is hydrogen, halogen, —CX⁸₃, —CHX⁸₂, —CH₂X⁸, —OCX⁸₃, —OCH₂X⁸, —OCHX⁸₂, —N₃, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —OR^{8D}, —NR^{8A}SO₂R^{8D}, —NR^{8A}C(O)R^{8C}, —NR^{8A}C(O)OR^{8C}, —NR^{8A}OR^{8C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CX⁹₃, —CHX⁹₂, —CH₂X⁹, —OCX⁹₃, —OCH₂X⁹, —OCHX⁹₂, —N₃, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen, halogen, —CX¹²₃, —CHX¹²₂, —CH₂X¹², —OCX¹²₃, —OCH₂X¹², —OCHX¹²₂, —N₃, —CN, —SOₙ₁₂R^{12D}, —SOᵥ₁₂NR^{12A}R^{12B}, —NHC(O)NR^{12A}R^{12B}, —N(O)ₘ₁₂, —NR^{12A}R^{12B}, —C(O)R^{12C}, —C(O)—OR^{12C}, —C(O)NR^{12A}R^{12B}, —OR^{12D}, —NR^{12A}SO₂R^{12D}, —NR^{12A}C(O)R^{12C}, —NR^{12A}C(O)OR^{12C}, —NR^{12A}OR^{12C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
wherein R^{6A}, R^{6B}, R^{6C}, R^{6D}, R^{7A}, R^{7B}, R^{7C}, R^{7D}, R^{8A}, R^{8B}, R^{8C}, R^{8D}, R^{9A}, R^{9B}, R^{9C}, R^{9D}, R^{12A}, R^{12B}, R^{12C}, and R^{12D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ and R⁸ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁸ and R⁹ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁹ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁶ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
n6, n7, n8, n9 and n12 are independently an integer from 0 to 4;
m6, m7, m8, m9, m12, v6, v7, v8, v9 and v12 are independently an integer from 1 to 2; and
X⁶, X⁷, X⁸, X⁹ and X¹² are independently —F, —Cl, —Br, or —I.

Embodiment Q37. The method of any one of Embodiments Q34-Q36, wherein R¹ is hydrogen.

Embodiment Q38. The method of any one of Embodiments Q35-Q37, wherein: Y is —CR¹²═.

Embodiment Q39. The method of Embodiment Q39, wherein R¹² is hydrogen, halogen, or unsubstituted C₁-C₄ alkyl.

Embodiment Q40. The method of Embodiment Q38, wherein Y is —CH═.

Embodiment Q41. The method of any one of Embodiments Q35-Q37, wherein Y is —N═.

Embodiment Q42. The method of any one of Embodiments Q34-Q41, wherein:
L¹ is R¹³-substituted or unsubstituted phenylene;
R¹³ is halogen, —CX¹³₃, —CHX¹³₂, —CH₂X¹³, —OCX¹³₃, —OCH₂X¹³, —OCHX¹³₂, —CN, —SOₙ₁₃R^{13D}, —SOᵥ₁₃NR^{13A}R^{13B}, —NHC(O)NR^{13A}R^{13B}, —N(O)ₘ₁₃, —NR^{13A}R^{13B}, —C(O)R^{13C}, —C(O)—OR^{13C}, —C(O)NR^{13A}R^{13B}, —OR^{13D}, —NR^{13A}SO₂R^{13D}, —NR^{13A}C(O)R^{13C}, —NR^{13A}C(O)OR^{13C}, —NR^{13A}OR^{13C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl,
wherein R^{13A}, R^{13B}, R^{13C}, and R^{13D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n13 is independently an integer from 0 to 4;
m13 and v13 are each independently an integer from 0 to 2; and
X¹³ is independently —F, —Cl, —Br, or —I.

Embodiment Q43. The method of Embodiment Q42, wherein L¹ is unsubstituted phenylene.

Embodiment Q44. The method of any one of Embodiments Q34-Q41, wherein:
L¹ is R¹³-substituted or unsubstituted heteroalkylene;
R¹³ is halogen, —CX¹³₃, —CHX¹³₂, —CH₂X¹³, —OCX¹³₃, —OCH₂X¹³, —OCHX¹³₂, —CN, —SOₙ₁₃R^{13D}, —SOᵥ₁₃NR^{13A}R^{13B}, —NHC(O)NR^{13A}R^{13B}, —N(O)ₘ₁₃, —NR^{13A}R^{13B}, —C(O)R^{13C}, —C(O)—OR^{13C}, —C(O)NR^{13A}R^{13B}, —OR^{13D}, —NR^{13A}SO₂R^{13D}, —NR^{13A}C(O)R^{13C}, —NR^{13A}C(O)OR^{13C}, —NR^{13A}OR^{13C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl,
wherein R^{13A}, R^{13B}, R^{13C}, and R^{13D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n13 is independently an integer from 0 to 4;
m13 and v13 are each independently an integer from 0 to 2; and
X¹³ is independently —F, —Cl, —Br, or —I.

Embodiment Q45. The method of any one of Embodiments Q34-Q41, wherein:
L¹ is R¹³-substituted or unsubstituted 2 to 4 membered heteroalkylene; and
R¹³ is halogen, or unsubstituted C₁-C₃ alkyl.

Embodiment Q46. The method of any one of Embodiments Q34-Q41, wherein:
L¹ is —SO₂N(R¹⁴)N═CH—;
wherein R¹⁴ is hydrogen, —CX¹⁴₃, —CHX¹⁴₂, —CH₂X¹⁴, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, and
X¹⁴ is independently —F, —Cl, —Br, or —I.

Embodiment Q47. The method of Embodiment Q46, wherein R¹⁴ is unsubstituted C₁-C₃ alkyl.

Embodiment Q47. The method of any one of any one of Embodiments Q34-Q41, wherein L¹ is —SO₂—N(CH₃)N═CH—.

Embodiment Q49. The method of any one of Embodiments Q34-Q48, wherein at least one of R², R³, R⁴ and R⁵ is halogen.

Embodiment Q50. The method of Embodiment Q49, wherein R², R³, and R⁵ are hydrogen and R⁴ is —F, —Cl or —Br.

Embodiment Q51. The method of any one of Embodiments Q34-Q48, wherein at least one of R², R³, R⁴ and R⁵ is substituted or unsubstituted pyridyl.

Embodiment Q52. The method of Embodiment Q51, wherein R², R³ and R⁵ are hydrogen, and R⁴ is halogen-substituted pyridyl or unsubstituted pyridyl.

Embodiment Q53. The method of any one of Embodiments Q34-Q52, wherein R⁶, R⁷, R⁸ and R⁹ are independently hydrogen, halogen, NO₂, or unsubstituted C₁-C₃ alkyl.

Embodiment Q54. The method of any one of Embodiments Q34-Q41, wherein:
L¹ is unsubstituted phenylene;
R¹, R², R³ and R⁵ are hydrogen;
R⁴ is —F, —Cl or —Br;
R⁶, R⁷, R⁸ and R⁹ are independently hydrogen, halogen, NO₂, or unsubstituted C₁-C₃ alkyl.

Embodiment Q55. The method of any one of Embodiments Q34-Q41, wherein:
L¹ is substituted or unsubstituted C₄-C₆ alkylene, or substituted or unsubstituted 4 to 6 membered heteroalkylene;
R¹, R², R³ and R⁵ are hydrogen;
R⁴ is —Cl or —Br; and
R⁶, R⁷, R⁸ and R⁹ are independently hydrogen, halogen, NO₂, or unsubstituted C₁-C₃ alkyl.

Embodiment Q56. The method of any one of Embodiments Q34-Q41, wherein:
L¹ is —SO₂—N(CH₃)N═CH—;
R², R³ and R⁵ are hydrogen;
R⁴ is halogen, or substituted or unsubstituted pyridyl.
R⁶ is unsubstituted methyl;
R⁷ is hydrogen;
R⁸ is —NO₂; and
R⁹ is hydrogen.

Embodiment Q57. The method of Embodiment Q56, wherein R⁴ is halogen-substituted pyridyl or unsubstituted pyridyl.

Embodiment Q58. The method of any one of Embodiments Q34-Q57, wherein L¹ has a length of about 4 to 12 Å.

Embodiment Q59. The method of any one of Embodiments Q34-Q58, wherein the compound is:

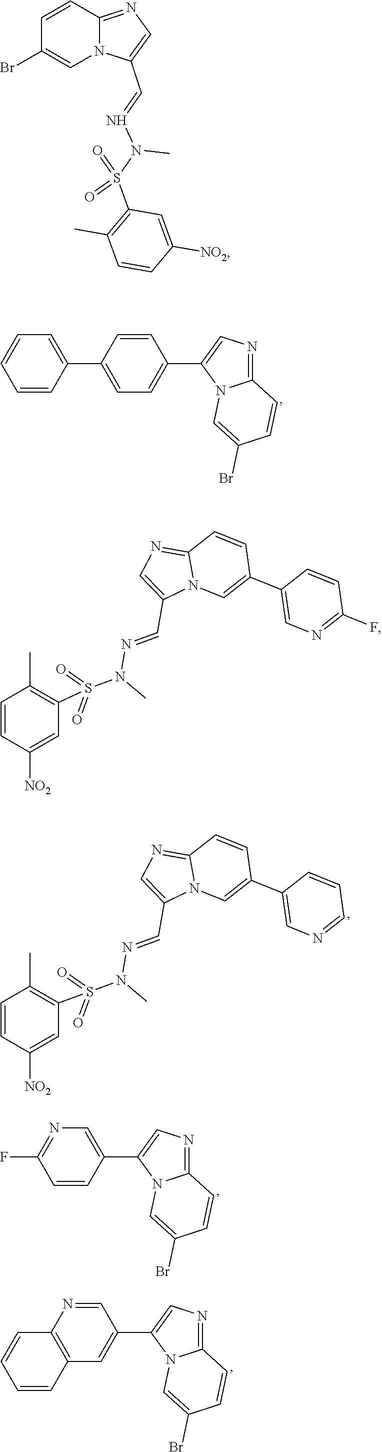

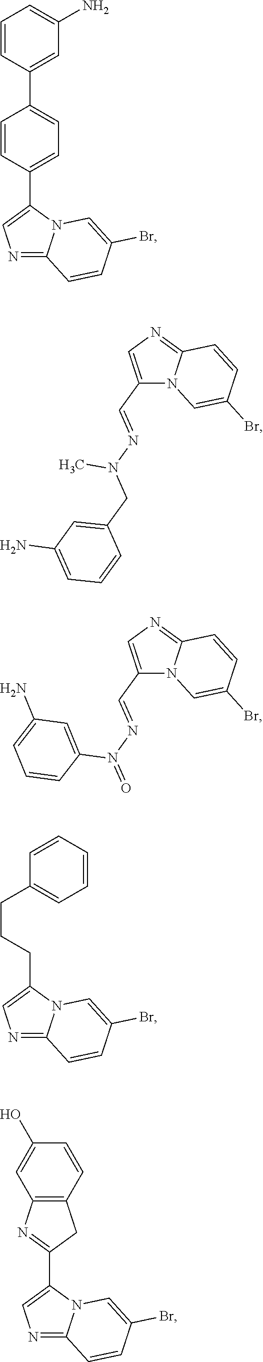

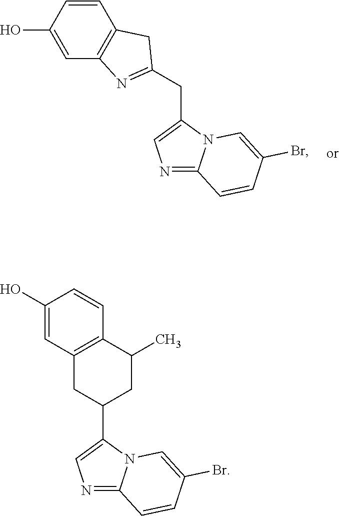 [SMILES: Oc1ccc2c(c1)N=C(Cc1cnc3ccc(Br)cn13)C2 ; CC1CC(c2cnc3ccc(Br)cn23)Cc2cc(O)ccc21]

Embodiment Q60. The method of any one of Embodiments Q34-Q59, wherein the p38γ kinase inhibitor has an IC₅₀ value of about 50 μM or less.

Embodiment Q61. The method of any one of Embodiments Q31-Q60, wherein the HDAC inhibitor is a compound selected from SAHA, romedepsin, abexinostat, entinostat, panobinostat and trichostatin A.

Embodiment Q62. A method of suppressing proliferation of a cutaneous T-cell lymphoma (CTCL) cell, the method comprising contacting the cell with an effective amount of a p38 gamma (p38γ) kinase inhibitor.

Embodiment Q63. The method of Embodiment Q62, wherein the p38γ kinase inhibitor is a compound represented by Formula (I):

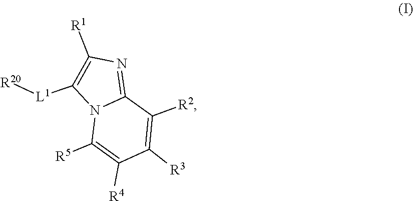

wherein:
L¹ is a bond, —SOₙ₁₁L^{1A}-, —SOᵥ₁₁NR¹¹L^{1A}-, —NHC(O)NR¹¹L^{1A}-, —NR¹¹L^{1A}-, —C(O)L^{1A}-, —C(O)OL^{1A}-, —C(O)NR¹¹L^{1A}-, —OL^{1A}-, —NR¹¹SO₂L^{1A}-, —NR¹¹C(O)L^{1A}-, —NR¹¹C(O)OL^{1A}-, —NR¹¹OL^{1A}-, —SL^{1A}-, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CX¹₃, —CHX¹₂, —CH₂X¹, —OCX¹₃, —OCH₂X¹, —OCHX¹₂, —N₃, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —OCX²₃, —OCH₂X², —OCHX²₂, —N₃, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —N₃, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —N₃, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, halogen, —CX⁵₃, —CHX⁵₂, —CH₂X⁵, —OCX⁵₃, —OCH₂X⁵, —OCHX⁵₂, —N₃, —CN, —SOₙ₅R^{5D}, —SOᵥ₅NR^{5A}R^{5B}, —NHC(O)NR^{5A}R^{5B}, —N(O)ₘ₅, —NR^{5A}R^{5B}, —C(O)R^{5C}, —C(O)—OR^{5C}, —C(O)NR^{5A}R^{5B}, —OR^{5D}, —NR^{5A}SO₂R^{5D}, —NR^{5A}C(O)R^{5C}, —NR^{5A}C(O)OR^{5C}, —NR^{5A}OR^{5C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²⁰ is substituted or unsubstituted cycloalkyl, substituted or unsubstituted heterocycloalkyl, substituted or unsubstituted aryl, or substituted or unsubstituted heteroaryl;
wherein R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D} and R¹¹ are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{1A} is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
n1, n2, n3, n4, n5 and n11 are independently an integer from 0 to 4;
m1, m2, m3, m4, m5, v1, v2, v3, v4, v5 and v11 are independently an integer from 1 to 2; and
X, X¹, X², X³, X⁴, and X⁵ are independently —F, —Cl, —Br, or —I.

Embodiment Q64. The method of Embodiment Q63, wherein the p38γ kinase inhibitor is a compound represented by Formula (II):

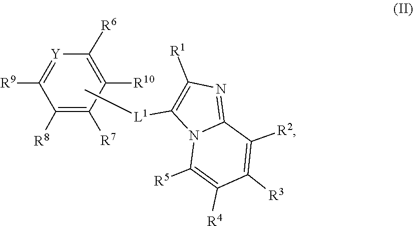

wherein:
L¹ is —SOₙ₁₁L^{1A}-, —SOᵥ₁₁NR¹¹L^{1A}-, —NHC(O)NR¹¹L^{1A}-, —NR¹¹L^{1A}-, —C(O)L^{1A}-, —C(O)OL^{1A}-, —C(O)NR¹¹L^{1A}-, -OL^{1A}-, —NR¹¹SO₂L^{1A}-, —NR¹¹C(O)L^{1A}-, —NR¹¹C(O)OL^{1A}-, —NR¹¹OL^{1A}-, —SL^{1A}-, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
Y is —N═ or —CR¹²═;
R⁶ is a bond (to L¹), hydrogen, halogen, —CX⁶₃, —CHX⁶₂, —CH₂X⁶, —OCX⁶₃, —OCH₂X⁶, —OCHX⁶₂, —N₃, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A} C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is a bond (to L¹), hydrogen, halogen, —CX⁷₃, —CHX⁷₂, —CH₂X⁷, —OCX⁷₃, —OCH₂X⁷, —OCHX⁷₂, —N₃, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A} C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is a bond (to L¹), hydrogen, halogen, —CX⁸₃, —CHX⁸₂, —CH₂X⁸, —OCX⁸₃, —OCH₂X⁸, —OCHX⁸₂, —N₃, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —OR^{8D}, —NR^{8A}SO₂R^{8D}, —NR^{8A}C(O)R^{8C}, —NR^{8A}C(O)OR^{8C}, —NR^{8A}OR^{8C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is a bond (to L¹), hydrogen, halogen, —CX⁹₃, —CHX⁹₂, —CH₂X⁹, —OCX⁹₃, —OCH₂X⁹, —OCHX⁹₂, —N₃, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A} C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ is a bond (to L¹), hydrogen, halogen, —CX¹⁰₃, —CHX¹⁰₂, —CH₂X¹⁰, —OCX¹⁰₃, —OCH₂X¹⁰, —OCHX¹⁰₂, —N₃, —CN, —SOₙ₁R^{10D}, —SOᵥ₁₀NR^{10A}R^{10B}, —NHC(O) NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen, halogen, —CX¹²₃, —CHX¹²₂, —CH₂X¹², —OCX¹²₃, —OCH₂X¹², —OCHX¹²₂, —N₃, —CN, —SOₙ₁₂R^{12D}, —SOᵥ₁₂NR^{12A}R^{12B}, —NHC(O)NR^{12A}R^{12B}, —N(O)ₘ₁₂, —NR^{12A}R^{12B}, —C(O)R^{12C}, —C(O)—OR^{12C}, —C(O)NR^{12A}R^{12B}, —OR^{12D}, —NR^{12A}SO₂R^{12D}, —NR^{12A}C(O)R^{12C}, —NR^{12A}C(O)OR^{12C}, —NR^{12A}OR^{12C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
wherein one of R⁶, R⁷, R⁸, R⁹ are R¹⁰ is a bond to L¹;
wherein R^{6A}, R^{6B}, R^{6C}, R^{6D}, R^{7A}, R^{7B}, R^{7C}, R^{7D}, R^{8A}, R^{8B}, R^{8C}, R^{8D}, R^{9A}, R^{9B}, R^{9C}, R^{9D}, R^{10A}, R^{10B}, R^{10C}, R^{10D}, R^{12A}, R^{12B}, R^{12C}, and R^{12D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ and R⁸ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁸ and R⁹ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁹ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁶ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁶ and R¹⁰ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁷ and R¹⁰ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
n6, n7, n8, n9, n10 and n12 are independently an integer from 0 to 4;
m6, m7, m8, m9, m10, m12, v6, v7, v8, v9, v10 and v12 are independently an integer from 1 to 2; and
X⁶, X⁷, X⁸, X⁹, X¹⁰ and X¹² are independently —F, —Cl, —Br, or —I.

Embodiment Q65. The method of any one of Embodiments Q63-Q64, wherein the compound is represented by Formula (III),

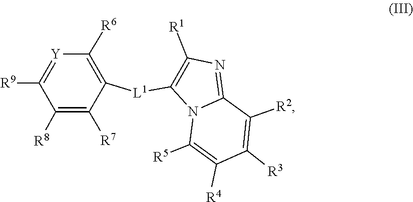

wherein:
Y is —N═ or —CR¹²═;
R⁶ is hydrogen, halogen, —CX⁶₃, —CHX⁶₂, —CH₂X⁶, —OCX⁶₃, —OCH₂X⁶, —OCHX⁶₂, —N₃, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is hydrogen, halogen, —CX⁷₃, —CHX⁷₂, —CH₂X⁷, —OCX⁷₃, —OCH₂X⁷, —OCHX⁷₂, —N₃, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is hydrogen, halogen, —CX⁸₃, —CHX⁸₂, —CH₂X⁸, —OCX⁸₃, —OCH₂X⁸, —OCHX⁸₂, —N₃, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —OR^{8D}, —NR^{8A}SO₂R^{8D}, —NR^{8A}C(O)R^{8C}, —NR^{8A}C(O)OR^{8C}, —NR^{8A}OR^{8C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CX⁹₃, —CHX⁹₂, —CH₂X⁹, —OCX⁹₃, —OCH₂X⁹, —OCHX⁹₂, —N₃, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen, halogen, —CX¹²₃, —CHX¹²₂, —CH₂X¹², —OCX¹²₃, —OCH₂X¹², —OCHX¹²₂, —N₃, —CN, —SOₙ₁₂R^{12D}, —SOᵥ₁₂NR^{12A}R^{12B}, —NHC(O)NR^{12A}R^{12B}, —N(O)ₘ₁₂, —NR^{12A}R^{12B}, —C(O)R^{12C}, —C(O)—OR^{12C}, —C(O)NR^{12A}R^{12B}, —OR^{12D}, —NR^{12A}SO₂R^{12D}, —NR^{12A}C(O)R^{12C}, —NR^{12A}C(O)OR^{12C}, —NR^{12A}OR^{12C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
wherein R^{6A}, R^{6B}, R^{6C}, R^{6D}, R^{7A}, R^{7B}, R^{7C}, R^{7D}, R^{8A}, R^{8B}, R^{8C}, R^{8D}, R^{9A}, R^{9B}, R^{9C}, R^{9D}, R^{12A}, R^{12B}, R^{12C}, and R^{12D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ and R⁸ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁸ and R⁹ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁹ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁶ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
n6, n7, n8, n9 and n12 are independently an integer from 0 to 4;
m6, m7, m8, m9, m12, v6, v7, v8, v9 and v12 are independently an integer from 1 to 2; and
X⁶, X⁷, X⁸, X⁹ and X¹² are independently —F, —Cl, —Br, or —I.

Embodiment Q66. The method of any one of Embodiments Q62-Q65, further comprising contacting the cell with a histone deacetylase (HDAC) inhibitor.

Embodiment Q67. The method of Embodiment Q66, wherein the HDAC inhibitor is a compound selected from SAHA, romedepsin, abexinostat, entinostat, panobinostat and trichostatin A.

Embodiment Q68. A compound represented by Formula (I):

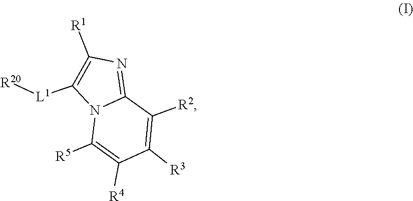

wherein:
L¹ is a bond, —SOₙ₁₁L^{1A}-, —SOᵥ₁₁NR¹¹L^{1A}-, —NHC(O)NR¹¹L^{1A}-, —NR¹¹L^{1A}-, —C(O)L^{1A}-, —C(O)OL^{1A}-, —C(O)NR¹¹L^{1A}-, -OL^{1A}-, —NR¹¹SO₂L^{1A}-, —NR¹¹C(O)L^{1A}-, —NR¹¹C(O)OL^{1A}-, —NR¹¹OL^{1A}-, —SL^{1A}-, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CX¹₃, —CHX¹₂, —CH₂X¹, —OCX¹₃, —OCH₂X¹, —OCHX¹₂, —N₃, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —OCX²₃, —OCH₂X², —OCHX²₂, —N₃, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —N₃, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —N₃, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, halogen, —CX⁵₃, —CHX⁵₂, —CH₂X⁵, —OCX⁵₃, —OCH₂X⁵, —OCHX⁵₂, —N₃, —CN, —SOₙ₅R^{5D}, —SOᵥ₅NR^{5A}R^{5B}, —NHC(O)NR^{5A}R^{5B}, —N(O)ₘ₅, —NR^{5A}R^{5B}, —C(O)R^{5C}, —C(O)—OR^{5C}, —C(O)NR^{5A}R^{5B}, —OR^{5D}, —NR^{5A}SO₂R^{5D}, —NR^{5A}C(O)R^{5C}, —NR^{5A}C(O)OR^{5C}, —NR^{5A}OR^{5C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²⁰ is substituted or unsubstituted cycloalkyl, substituted or unsubstituted heterocycloalkyl, substituted or unsubstituted aryl, or substituted or unsubstituted heteroaryl;
wherein R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D} and R¹¹ are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{1A} is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
n1, n2, n3, n4, n5 and n11 are independently an integer from 0 to 4;
m1, m2, m3, m4, m5, v1, v2, v3, v4, v5 and v11 are independently an integer from 1 to 2; and
X, X¹, X², X³, X⁴, and X⁵ are independently —F, —Cl, —Br, or —I,
with proviso that when Y is —CH═, L¹ is —SO₂—N(CH₃)N═CH— and R⁴ is —Br, then R²⁰ is not 2-methyl-5-nitrophenyl.

Embodiment Q69. The compound of Embodiment Q68, wherein the p38γ kinase inhibitor is a compound represented by Formula (II),

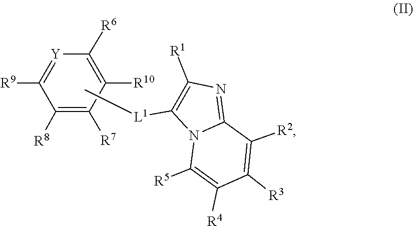

wherein:
L¹ is —SOₙ₁₁L^{1A}-, —SOᵥ₁₁NR¹¹L^{1A}-, —NHC(O)NR¹¹L^{1A}-, —NR¹¹L^{1A}-, —C(O)L^{1A}-, —C(O)OL^{1A}-, —C(O)NR¹¹L^{1A}-, -OL^{1A}-, —NR¹¹SO₂L^{1A}-, —NR¹¹C(O)L^{1A}-, —NR¹¹C(O)OL^{1A}-, —NR¹¹OL^{1A}-, —SL^{1A}-, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
Y is —N═ or —CR¹²═;
R⁶ is a bond (to L¹), hydrogen, halogen, —CX⁶₃, —CHX⁶₂, —CH₂X⁶, —OCX⁶₃, —OCH₂X⁶, —OCHX⁶₂, —N₃, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is a bond (to L¹), hydrogen, halogen, —CX⁷₃, —CHX⁷₂, —CH₂X⁷, —OCX⁷₃, —OCH₂X⁷, —OCHX⁷₂, —N₃, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is a bond (to L¹), hydrogen, halogen, —CX⁸₃, —CHX⁸₂, —CH₂X⁸, —OCX⁸₃, —OCH₂X⁸, —OCHX⁸₂, —N₃, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —OR^{8D}, —NR^{8A}SO₂R^{8D}, —NR^{8A}C(O)R^{8C}, —NR^{8A}C(O)OR^{8C}, —NR^{8A}OR^{8C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is a bond (to L¹), hydrogen, halogen, —CX⁹₃, —CHX⁹₂, —CH₂X⁹, —OCX⁹₃, —OCH₂X⁹, —OCHX⁹₂, —N₃, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ is a bond (to L¹), hydrogen, halogen, —CX¹⁰₃, —CHX¹⁰₂, —CH₂X¹⁰, —OCX¹⁰₃, —OCH₂X¹⁰, —OCHX¹⁰₂, —N₃, —CN, —SOₙ₁₀R^{10D}, —SOᵥ₁₀NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen, halogen, —CX¹²₃, —CHX¹²₂, —CH₂X¹², —OCX¹²₃, —OCH₂X¹², —OCHX¹²₂, —N₃, —CN, —SOₙ₁₂R^{12D}, —SOᵥ₁₂NR^{12A}R^{12B}, —NHC(O)NR^{12A}R^{12B}, —N(O)ₘ₁₂, —NR^{12A}R^{12B}, —C(O)R^{12C}, —C(O)—OR^{12C}, —C(O)NR^{12A}R^{12B}, —OR¹²D, —NR^{12A}SO₂R^{12D}, —NR^{12A}C(O)R^{12C}, —NR^{12A}C(O)OR^{12C}, —NR^{12A}OR^{12C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
wherein one of R⁶, R⁷, R⁸, R⁹ are R¹⁰ is a bond to L¹;
wherein R^{6A}, R^{6B}, R^{6C}, R^{6D}, R^{7A}, R^{7B}, R^{7C}, R^{7D}, R^{8A}, R^{8B}, R^{8C}, R^{8D}, R^{9A}, R^{9B}, R^{9C}, R^{9D}, R^{10A}, R^{10B}, R^{10C}, R^{10D}, R^{12A}, R^{12B}, R^{12C}, and R^{12D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ and R⁸ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁸ and R⁹ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁹ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁶ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁶ and R¹⁰ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁷ and R¹⁰ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
n6, n7, n8, n9, n10 and n12 are independently an integer from 0 to 4;
m6, m7, m8, m9, m10, m12, v6, v7, v8, v9, v10 and v12 are independently an integer from 1 to 2; and
X⁶, X⁷, X⁸, X⁹, X¹⁰ and X¹² are independently —F, —Cl, —Br, or —I,
with proviso that when R¹⁰ is a bond to L¹, Y is —CH═, L¹ is —SO₂—N(CH₃)N═CH—, R⁴ is —Br and R⁶ is unsubstituted methyl, then R⁸ is not —NO₂; or when R⁸ is a bond to L¹, Y is —CH═, L¹ is —SO₂—N(CH₃)N═CH—, R⁴ is —Br and R⁹ is unsubstituted methyl, then R¹⁰ is not —NO₂.

Embodiment Q70. The compound of claim Embodiment Q69, wherein the compound is represented by Formula (III),

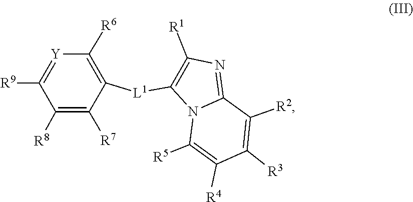

wherein:
Y is —N═ or —CR¹²═;
R⁶ is hydrogen, halogen, —CX⁶₃, —CHX⁶₂, —CH₂X⁶, —OCX⁶₃, —OCH₂X⁶, —OCHX⁶₂, —N₃, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is hydrogen, halogen, —CX⁷₃, —CHX⁷₂, —CH₂X⁷, —OCX⁷₃, —OCH₂X⁷, —OCHX⁷₂, —N₃, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is hydrogen, halogen, —CX⁸₃, —CHX⁸₂, —CH₂X⁸, —OCX⁸₃, —OCH₂X⁸, —OCHX⁸₂, —N₃, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —OR^{8D}, —NR^{8A}SO₂R^{8D}, —NR^{8A}C(O)R^{8C}, —NR^{8A}C(O)OR^{8C}, —NR^{8A}OR^{8C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CX⁹₃, —CHX⁹₂, —CH₂X⁹, —OCX⁹₃, —OCH₂X⁹, —OCHX⁹₂, —N₃, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen, halogen, —CX¹²₃, —CHX¹²₂, —CH₂X¹², —OCX¹²₃, —OCH₂X¹², —OCHX¹²₂, —N₃, —CN, —SOₙ₁₂R^{12D}, —SOᵥ₁₂NR^{12A}R^{12B}, —NHC(O)NR^{12A}R^{12B}, —N(O)ₘ₁₂, —NR^{12A}R^{12B}, —C(O)R^{12C}, —C(O)—OR^{12C}, —C(O)NR^{12A}R^{12B}, —OR^{12D}, —NR^{12A}SO₂R^{12D}, —NR^{12A}C(O)R^{12C}, —NR^{12A}C(O)OR^{12C}, —NR^{12A}OR^{12C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
wherein R^{6A}, R^{6B}, R^{6C}, R^{6D}, R^{7A}, R^{7B}, R^{7C}, R^{7D}, R^{8A}, R^{8B}, R^{8C}, R^{8D}, R^{9A}, R^{9B}, R^{9C}, R^{9D}, R^{12A}, R^{12B}, R^{12C}, and R^{12D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ and R⁸ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁸ and R⁹ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁹ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁶ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
n6, n7, n8, n9 and n12 are independently an integer from 0 to 4;
m6, m7, m8, m9, m12, v6, v7, v8, v9 and v12 are independently an integer from 1 to 2; and
X⁶, X⁷, X⁸, X⁹ and X¹² are independently —F, —Cl, —Br, or —I.

Embodiment Q71. The compound of any one of Embodiments Q68-Q70, wherein R¹ is hydrogen.

Embodiment Q72. The compound of any one of Embodiments Q69-Q71, wherein:
Y is —CR¹²═.

Embodiment Q73. The compound of Embodiment Q72, wherein R¹² is hydrogen, halogen, or unsubstituted C₁-C₄ alkyl.

Embodiment Q74. The compound of Embodiment Q72, wherein Y is —CH═.

Embodiment Q75. The compound of any one of Embodiments Q69-Q71, wherein Y is —N═.

Embodiment Q76. The compound of any one of Embodiments Q68-Q75, wherein:
L¹ is R¹³-substituted or unsubstituted phenylene;
R¹³ is halogen, —CX¹³₃, —CHX¹³₂, —CH₂X¹³, —OCX¹³₃, —OCH₂X¹³, —OCHX¹³₂, —CN, —SOₙ₁₃R^{13D}, —SOᵥ₁₃NR^{13A}R^{13B}, —NHC(O)NR^{13A}R^{13B}, —N(O)ₘ₁₃, —NR^{13A}R^{13B}, —C(O)R^{13C}, —C(O)—OR^{13C}, —C(O)NR^{13A}R^{13B}, —OR^{13D}, —NR^{13A}SO₂R^{13D}, —NR^{13A}C(O)R^{13C}, —NR^{13A}C(O)OR^{13C}, —NR^{13A}OR^{13C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl,
wherein R^{13A}, R^{13B}, R^{13C}, and R^{13D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n13 is independently an integer from 0 to 4;
m13 and v13 are each independently an integer from 0 to 2; and
X¹³ is independently —F, —Cl, —Br, or —I.

Embodiment Q77. The compound of Embodiment Q76, wherein L¹ is unsubstituted phenylene.

Embodiment Q78. The compound of any one of Embodiments Q68-Q75, wherein:
L¹ is R¹³-substituted or unsubstituted heteroalkylene;
R¹³ is halogen, —CX¹³₃, —CHX¹³₂, —CH₂X¹³, —OCX¹³₃, —OCH₂X¹³, —OCHX¹³₂, —CN, —SOₙ₁₃R^{13D}, —SOᵥ₁₃NR^{13A}R^{13B}, —NHC(O)NR^{13A}R^{13B}, —N(O)ₘ₁₃, —NR^{13A}R^{13B}, —C(O)R^{13C}, —C(O)—OR^{13C}, —C(O)NR^{13A}R^{13B}, —OR^{13D}, —NR^{13A}SO₂R^{13D}, —NR^{13A}C(O)R¹³C, —NR^{13A}C(O)OR^{13C}, —NR^{13A}OR^{13C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl,
wherein R^{13A}, R^{13B}, R^{13C}, and R^{13D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n13 is independently an integer from 0 to 4;
m13 and v13 are each independently an integer from 0 to 2; and
X¹³ is independently —F, —Cl, —Br, or —I.

Embodiment Q79. The compound of any one of Embodiments Q68-Q75, wherein:
L¹ is R¹³-substituted or unsubstituted 2 to 4 membered heteroalkylene; and
R¹³ is halogen, or unsubstituted C₁-C₃ alkyl.

Embodiment Q80. The compound of any one of Embodiments Q68-Q75, wherein:
L¹ is —SO₂N(R¹⁴)N═CH—;
wherein R¹⁴ is hydrogen, —CX¹⁴₃, —CHX¹⁴₂, —CH₂X¹⁴, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, and
X¹⁴ is independently —F, —Cl, —Br, or —I.

Embodiment Q81. The compound of Embodiment Q80, wherein R¹⁴ is unsubstituted C₁-C₃ alkyl.

Embodiment Q82. The compound of any one of Embodiments Q68-Q75, wherein L¹ is —SO₂—N(CH₃)N═CH—.

Embodiment Q83. The compound of any one of Embodiments Q68-Q82, wherein at least one of R², R³, R⁴ and R⁵ is halogen.

Embodiment Q84. The compound of Embodiment Q83, wherein R², R³, and R⁵ are hydrogen and R⁴ is —F, —Cl or —Br.

Embodiment Q85. The compound of any one of Embodiments 68-Q82, wherein at least one of R², R³, R⁴ and R⁵ is substituted or unsubstituted pyridyl.

Embodiment Q86. The compound of Embodiment Q85, wherein R², R³ and R⁵ are hydrogen, and R⁴ is halogen-substituted pyridyl or unsubstituted pyridyl.

Embodiment Q87. The compound of any one of Embodiments Q68-Q86, wherein R⁶, R⁷, R⁸ and R⁹ are independently hydrogen, halogen, NO₂, or unsubstituted C₁-C₃ alkyl.

Embodiment Q88. The compound of any one of Embodiments Q68-Q75, wherein:
L¹ is unsubstituted phenylene;
R¹, R², R³ and R⁵ are hydrogen;
R⁴ is —F, —Cl or —Br;
   R⁶, R⁷, R⁸ and R⁹ are independently hydrogen, halogen, NO₂, or unsubstituted C₁-C₃ alkyl.

Embodiment Q89. The compound of any one of Embodiments Q68-Q75, wherein:
L¹ is substituted or unsubstituted C₄-C₆ alkylene, or substituted or unsubstituted 4 to 6 membered heteroalkylene;
R¹, R², R³ and R⁵ are hydrogen;
R⁴ is —Cl; and
R⁶, R⁷, R⁸ and R⁹ are independently hydrogen, halogen, or unsubstituted C₁-C₃ alkyl.

Embodiment Q90. The compound of any one of Embodiments Q68-Q75, wherein:
L¹ is —SO₂—N(CH₃)N═CH—;
R², R³ and R⁵ are hydrogen;
R⁴ is substituted or unsubstituted pyridyl;
R⁶ is unsubstituted methyl;
R⁷ is hydrogen;
R⁸ is —NO₂; and
R⁹ is hydrogen.

Embodiment Q91. The compound of Embodiment Q90, wherein R⁴ is halogen-substituted pyridyl or unsubstituted pyridyl.

Embodiment Q92. The compound of any one of Embodiments Q68-Q91, wherein L¹ has a length of about 4 to 12 Å.

Embodiment Q93. The compound of any one of Embodiments Q68-Q92, wherein the compound is:

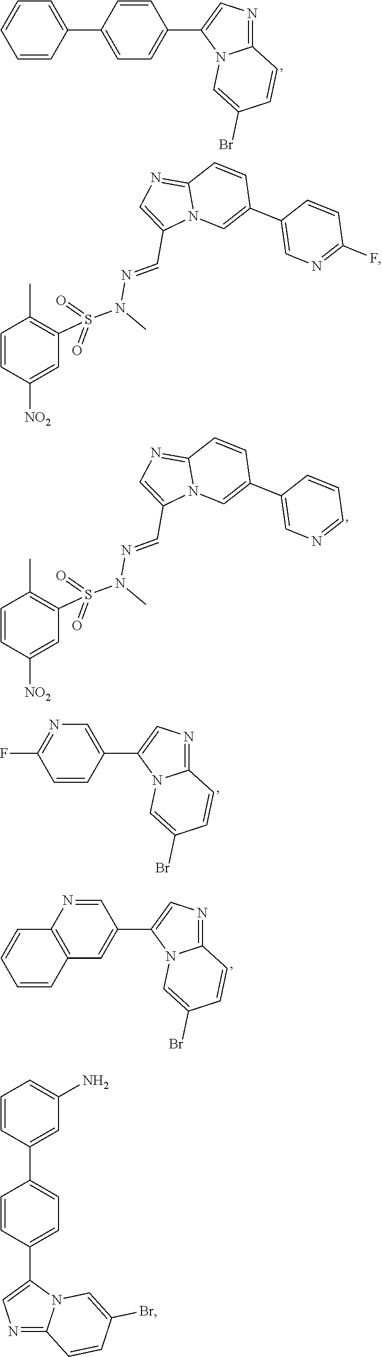

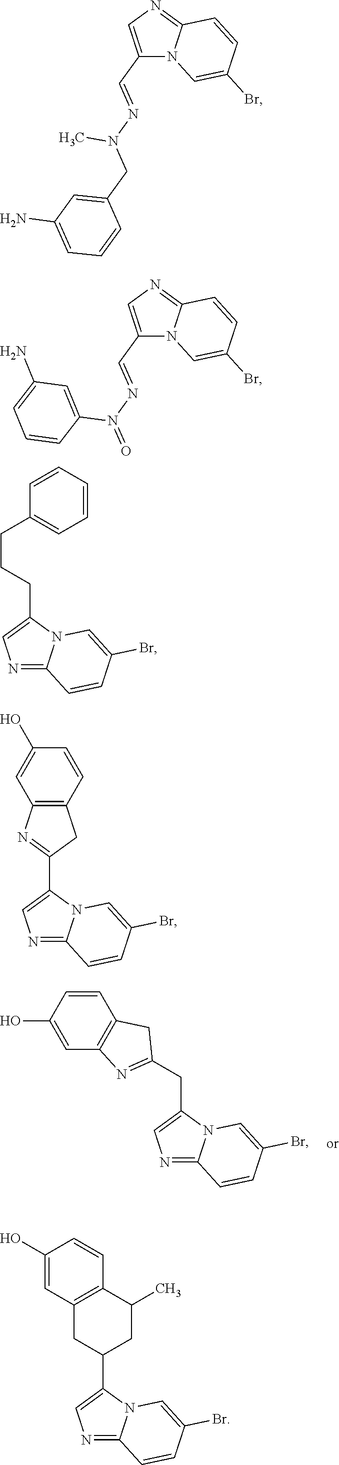

Embodiment Q94. The compound of any one of Embodiments Q68-Q93, wherein the p38γ kinase inhibitor has an IC₅₀ value of about 50 μM or less.

Embodiment Q95. A pharmaceutical composition comprising a compound of any one of Embodiments Q68-Q94, or a pharmaceutically acceptable salt thereof.

### VI. Examples

Although the foregoing section has been described in some detail by way of illustration and example for purposes of clarity of understanding, it is apparent to those skilled in the art that certain minor changes and modifications will be practiced in light of the above teaching. Therefore, the description and examples should not be construed as limiting the scope of any invention described herein.

All references cited herein, including patent applications and publications, are hereby incorporated by reference in their entirety.

### General Materials and Method

Screening a library of kinase inhibitors and the NCI compounds to identify p38γ inhibitors using an in vitro kinase assay: To identify p38γ inhibitors, we screened the EMD Calbiochem kinase inhibitor library and 80 compounds obtained from the NCI Chemicals Repository (results for NCI compounds are not included, provided upon request). The COH in house kinase inhibitor library consists of 244 compounds that are mostly ATP mimics. All compounds are cell-permeable, reversible and well-characterized. For the biochemical screening, kinase assays in vitro were performed using ADP-Glo kit (Promega). The luminescent ADP-Glo kinase assay measures ADP produced from ATP consumption in the reaction.

Kinase assay in vitro: Kinase enzyme assays in vitro were performed using ADP-Glo kit (Promega, Wis.). The luminescent ADP-Glo kinase assay measures ADP produced from ATP consumption in a kinase enzyme reaction. All compounds have been tested and shown no inhibition to luciferase activities. Briefly, in this experiment, human recombinant p38 α, β, γ or δ protein (active full-length) expressed by baculovirus in sf9 insect cells (SignalChem, Vancouver, Canada) was homogenized in wells of white opaque 96-well microplates with the kinase buffer that consisted of 40 mM Tris-HCl (pH 7.5), 20 mM MgCl₂, 0.1 mg/mL BSA and 50 μM DTT. The p38 kinase was preincubated with F7 in a dose-dependent manner for 10 min. Next, synthetic peptide substrates (IPTTPITTTYFFFKKK) were added to the mixture at final concentration of 0.2 μg/μL, followed by adding ATP at various concentrations. The 50 μL mixture was reacted at room temperature for 60 min. Then, ADP-Glo Reagent was incubated in the mixture at room temperature for 40 min, followed by incubation of Kinase Detection Reagent for another 30 min with minor modification as described in the supplier's instruction. Luminescence was measured at an integration time of 0.5 second using an automated BMG PHERAstar plate reader (BMG Labtech). Each experiment was conducted in duplicate or triplicate. We tested if lead compounds inhibit luciferase activities in the assay. IC₅₀ values were determined using CalcuSyn software (Biosoft).

Determination of IC₅₀ values for ATP competitive inhibitor and Ki: (a). An enzymatic kinase assay in vitro was performed with recombinant p38 γ protein and a synthetic peptide substrate at 10 μM, 100 μM and 250 μM concentrations of ATP. Data points are the average of two independent experiments. (b). IC₅₀ values were determined using CalcuSyn software (Biosoft) based on the data of Figure a. Ki=IC₅₀/(S/K_{M}+1) equation was used to determine Ki value of F7 (Cer et al., Nucleic Acids Research, 2009).

Determination of ATP-K_{M} value: Recombinant p38 γ protein was mixed with a synthetic peptide substrate in low volume 384 well microplates, followed with addition of ATP at 2.5 μM to 800 μM final concentrations. The reaction was conducted in 10 μL volume for 10 min. Luminescence was measured using an automated BMG PHERAstar plate reader. Data points are the average of three independent experiments. The data was plotted with Lineweaver-Burk equation. The slope (K_{M}/Vₘₐₓ), Y-intercept (1/Vₘₐₓ) and X-intercept (−1/K_{M}) were determined using GraphPad Prism software.

F7 effects on the kinase activity of p38 isoforms in vitro: An enzymatic kinase assay in vitro was performed with human recombinant p38 α, β, γ or δ protein (active full-length). Data points are the average of two independent experiments. We tested if lead compounds inhibit luciferase activities in the assay. IC₅₀ values were determined using CalcuSyn software (Biosoft).

IC₅₀ value determination of cyto-toxicity of F7 against human NCI-60 cell lines and HDACIs against other cell lines such as H9, and Hut 78: To determine the cytotoxicity of F7 using our in-house NCI-60 cancer cell line panel assay at City of Hope, MTS assays were performed for cell viability as described by the supplier (Promega). Briefly, human NCI-60 cancer cell lines were counted with a Vi-CELL Cell Viability Analyzer (Beckman Coulter), resuspended in RPMI-1640 medium containing 10% FBS and 1% penicillin/streptomycin and dispensed in wells of 96-well microplates (3000 cells per well for solid tumor and 5000 per well for blood tumor cells) using an epMotion 5075 liquid handler (Eppendorf) under a sterile condition, followed by incubation overnight at 37° C. in 5% (v/v) CO₂. Cells were exposed to compounds in a dose-dependent manner for 72 h. An MTS dye (20 uL per well) was added to each well. Cell viability was determined by tetrazolium conversion to its format an dye. Absorbance was monitored at 490 nm using an automated BMG PHERAstar plate reader (BMG Labtech). Each experiment was performed in duplicate. IC₅₀ values were determined using CalcuSyn software (Biosoft). H9 cells are more sensitive to drugs, therefore instead of 72 hours, we choose 48 hour the incubation time for H9 cells.

F7 reduces the tumor size in a dose dependent manner in CTCL xenograft mice (CTCL cell line xenograft mouse model): Female and male 6-week-old NSG mice (NOD-scid IL2Rgammanull) were purchased from Jackson Laboratory. Hut 78 cells were used for developing CTCL xenograft tumors. Male and female 8-12 wk, 20-25 g mice were grouped into 3 (7 mice each group) Group 1, as control group, Group 2 treated with 2 mg/kg, and Group 3 treated with 10 mg/kg. Briefly, 5 million cells in 100 μL (50% Matrigel/50% PBS) were injected subcutaneously into the right flank of each mouse, and tumor development were monitored every other day. Once the tumors are palpable and reach 100 mm³ in volume, treatment will commence. Mice were treated daily. Tumor sizes were measured twice per week using calipers. Experiments were stopped once the tumors reach 30 mm in diameter, or to minimize pain and discomfort as described below. Any mouse that has a body weight loss of more than 20% or exhibits any severe pain/distress signs that match premature euthanasia criteria will be sedated and euthanized.

Immunofluorescence Confocal microscopy: Hut 78 Cells were cytospun unto the slides and fixed with 3.8% paraformaldehyde for 30 min at room temperature (RT) and then permeabilized with 0.25% Triton X-100 in PBS, for 10 min at RT. The cells were washed twice with PBS and blocked in 3% BSA in PBS for 1 h at RT. The cells were then stained for overnight at 4° C. with the primary antibody diluted with the blocking solution, washed three times with PBS and incubated with a secondary antibody coupled to Alexa 546, or 647 or 488 accordingly to the each primary antibodies (used at the dilution 1:1000) for 45 min at RT. Coverslips were permanently mounted with DAPI (life Technologies) for nuclear staining. Observations were carried out using a confocal laser scanning microscope (zeiss LSM 700). Primary antibodies were used at the following dilutions: anti-ß-tubulin (1:200), H3K27 ac (CST, dilution 1:500), p-p38 (CST, dilution 1:500), DLGH1 (abcam) 1:1000.

P38 gamma gene silencing by shRNA: MISSION® shRNA Lentiviral Transduction Particles (Company validated) 5 shRNAs in pLKO.1-puro shRNA vector that targets against 4 exons of the human p38 gamma gene (MAPK12) and a scramble Transduction Particles (pLKO.1-puro shRNA vector only) were purchased from Sigma. Hut78 and H9 cells growing exponentially (70-80% confluent) were seeded into 6-well plates (2×10⁵ cells/well) before transduction. Viral transduction efficiencies were improved by adding polybrene (hexadimethrine bromide) with a final concentration of 8-10 μg/ml. To each well 5 ml medium and hexadimethrine bromide to a final concentration of 8 μg/ml were added. Gently swirl the plate to mix. Multiplicity of Infection (MOI) is the number of transducing lentiviral particles per cell, MOI 2 were selected for the optimal transduction efficiency and knockdown of p38 gamma for Hut78 cells. Hut 78 and H9 Cells were incubated for overnight hours with the medium containing lentiviral particles and polybrene, then the medium containing lentiviral particles were removed from wells and were replaced with fresh medium. Cells are collected on day 5 after transduction before subjecting to RNA extraction and protein isolation.

NANOSTRING NCOUNTER® gene expression quantification and validation: 100 ng of RNA isolated from Hut78 cells that are as suggested by the NanoString protocol, were used in experiment. All samples were validated. Data were analyzed using nSolver 3.0 digital analyzer software and R program.

### Example 1: p38 Kinases Expression Involved in Proliferation of CTCL Cells

In pursuit of key signaling molecules that drive CTCL development, the p38 family had been identified as important molecules for CTCL growth [5]. The p38 family includes four isoforms, α, β, γ, and δ, which share similar protein sequences, but vary in tissue-specific expression, substrate preferences, and downstream effects [17]. In particular, p38γ is considered to be mostly involved in CTCL development. For instance, quantitative RT-PCR showed that p38γ mRNA expression was elevated in both Hut78 cells (SS cell line; data not shown) and in primary CD4+ T cells from SS patients, but not from healthy donors (FIG. 3A). In addition, in order to confirm increased expression of p38γ in CTCL cells, publicly available microarray databases (GSE17601, n=32 for SS) and (GSE12902 [19], n=22 for MF) were analyzed and gene expression level of p38γ was increased in both SS and MF patients, compared to healthy donors/cell lines (GSE19069, n=8) (FIG. 3B) [18, 20]. These observations implicate p38γ as an indispensable driver in CTCL development.

To investigate the effects of loss of p38γ expression on CTCL proliferation, cells with genetically silenced p38γ expression were used. HH cells (CTCL cell line) transduced with lentiviral shRNA against p38γ (FIG. 4A) and Hut78 cells transiently transfected with siRNA (SR304235-A: GGAAGCGUGUUACUUACAAAGAGGT; and SR304235-B CGGAGAUGAUCACAGGCAAGACGC) against p38γ (FIG. 4B). Both results in FIGS. 4A-4B demonstrated significantly reduced cell proliferation.

Stable p38γ depleted cells were generated by lentiviral shRNA specifically against p38γ, and then exogenous expression of p38γ was induced or active p38γ kinase proteins were delivered in the cell. For example, p38γ (active form) was delivered into the p38 γ depleted cells (p38γ knockdown cells) by lipids packaging methods. The incorporation of p38γ proteins in p38 γ depleted Hut 78 cells increased cells proliferation (FIG. 4C).

To investigate how downstream p38γ signaling was affected in CTCL patients, publically available microarray database were analyzed for NFATC1, which is the typical downstream target of the alternative p38 signaling pathway [13]. The results showed that expression of NFATC1 were decreased and expression of NFATC4 were increase in Sézary Syndrome (SS) CTCL patient samples (GSE17601-ENREF_18, n=32) as compared to healthy T cells (GSM472024,_ENREF_20 n=10) (FIG. 5) [18, 20]. Given that NFATC4 is typically expressed at non-detectable levels in normal T cells, the elevated expression of NFATC4 in CTCL suggests a role in cancer development. The mechanism driving increased expression of NFATC4 expression in CTCL remains unknown.

In T cells, two p38 pathways are activated, however, two p38 MAPK pathways on NFATs showed opposite effects (FIG. 6A). According to classic p38 pathway in both T cells and others, NFATC1 may be inactivated by phosphorylation and cytoplasmic sequestration may occur. According to the alternative p38 pathway activation in T cells, NFATC1 and IRF4 may be upregulated during transcription such that RNA level of NFATC1 may increase and IL-17A level may subsequently increase [74].

Moreover, NFATC4 level is modulated by the presence of p38γ, but not by the other p38 isoforms (data not shown). To demonstrate direct connection between p38γ and NFATC4, the mRNA expression level of NFATC4 in p38γ-knockdown Hut78 cells were measured using qRT-PCR. Knockdown of p38γ, but not p38β, led to a reduction in mRNA expression of NFATC4 (FIG. 6B), as well as the downstream target cytokine IL-17A (FIG. 6C). Inhibition of NFATC4 using shRNA also reduced the proliferation of CTCL cell lines (data not shown) and reduced IL-17A mRNA expression (FIG. 6D). These data suggest that malignant T cells express critical components of the p38γ pathway to enhance proliferation in CTCL.

Alternative p38 pathway (p38 phosphorylated at Tyr323) activated in Hut78 cells are shown in FIG. 6E. Cells with phosphorylated-p38^{Tyr323} are indicated in magenta color, and these cells expressed IL-17RA as indicated in green color. It demonstrates that alternatively phosphorylated on Tyr323 of p38 (Magenta color) correlated with IL-17RA level. The arrow indicates a cell show lower expression level of both.

### Example 2: p38γ Inhibitors

Given that p38γ expression is elevated in CTCL cells and that reduction of p38γ protein inhibits CTCL cell proliferation, p38γ is an attractive therapeutic target for CTCL. However, there is no specific p38γ inhibitor available for treatment of CTCL. Pirfenidone, a FDA-approved drug for treatment of pulmonary fibrosis [21], is a known TGF-β inhibitor with off-target activity against p38γ. Pirfenidone has been shown to inhibit p38γ expression in mice, but requires a high concentration (500 mg/kg per day in drinking water) [22], and the mechanism of action is unknown.

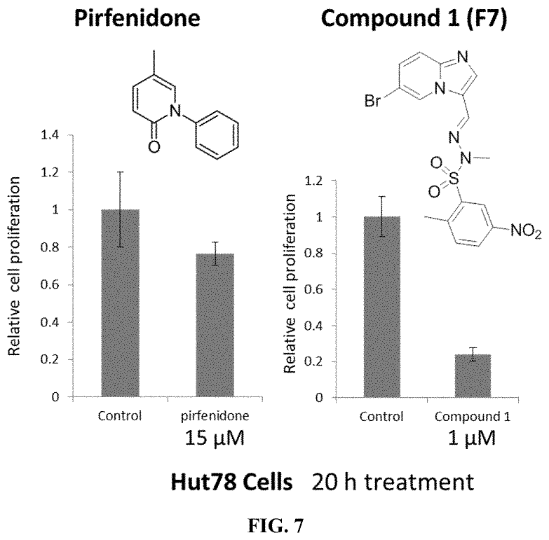

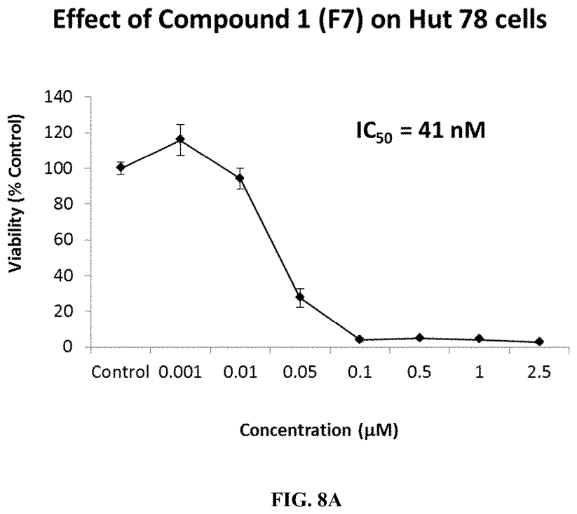

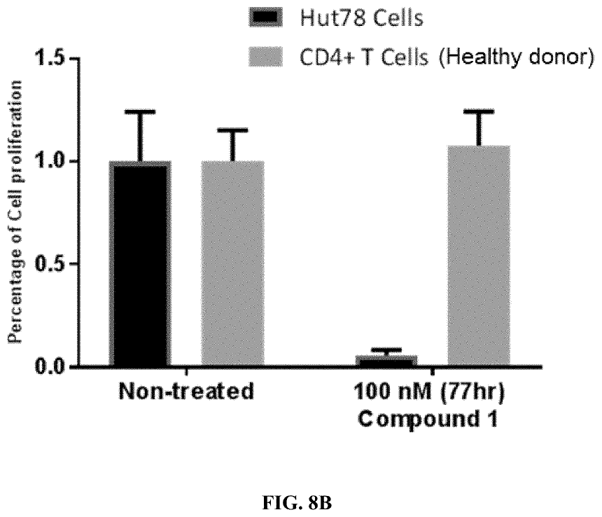

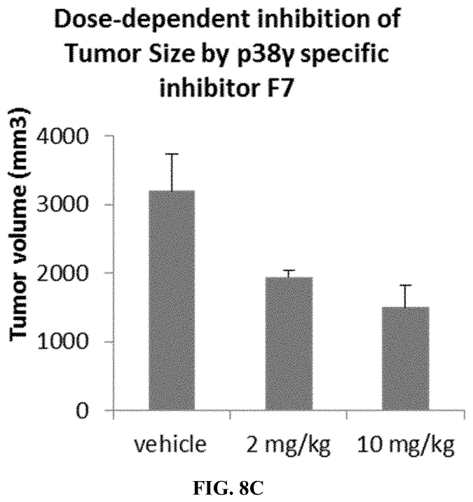

To identify novel, potent, and specific inhibitors of p38γ, molecular modeling and high-throughput screening (HTS) of a commercially-available kinase inhibitor library were performed. Lead Compound 1 (F7; shown) was identified to reduce proliferation of Hut78 cells more effectively than pirfenidone (FIG. 7), and showed a dose-dependent inhibition of cell viability in Hut78 cells (FIG. 8A). Importantly, compound 1 exhibited selective induction of apoptosis (data not shown) and inhibition of growth in the Hut78 CTCL cell line, but not in healthy CD4+ T cells (FIG. 8B). To demonstrate dose-dependent inhibition of tumor growth in an animal model, we treated mice harboring Hut78 xenografts with compound 1 at 2 mg/kg or 10 mg/kg, or with vehicle control. Compound 1 showed statistically significant inhibition of tumor growth at 2 mg/kg (t-test p=0.015), with further significant reduction of tumor size at 10 mg/kg (t-test p=0.025) (FIG. 8C).

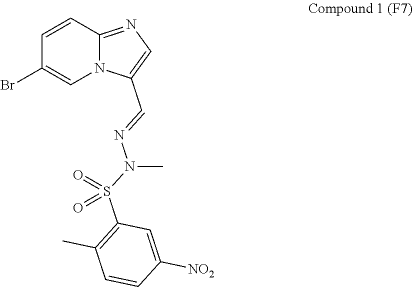 [SMILES: Cc1ccc([N+](=O)[O-])cc1S(=O)(=O)N(C)/N=C/c1cnc2ccc(Br)cn12]

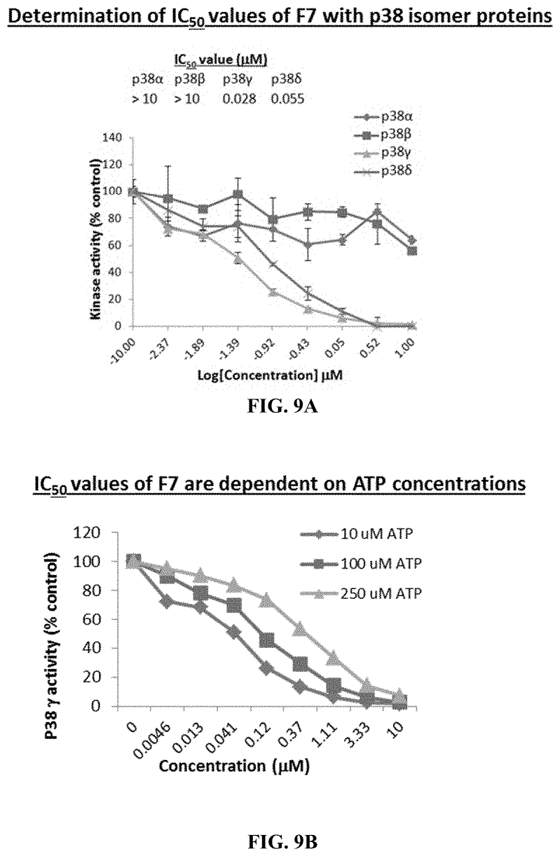

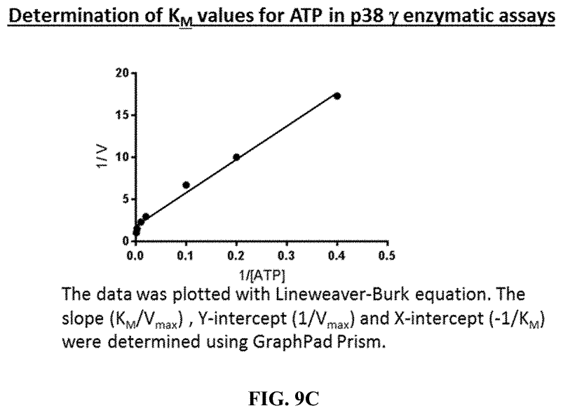

Compound 1 was identified to have specific inhibition against the p38γ isoform in in vitro kinase assays (IC₅₀ in the nanomolar range; FIG. 9A), and inhibited growth of CTCL cells in viability assays (IC₅₀ in the nanomolar to micromolar range).

Compound 1 (F7) was identified as an ATP competitor that can bind to the ATP-binding pocket of p38 gamma and an enzymatic kinase assay in vitro was performed with recombinant p38γ protein and a synthetic peptide substrate at 10 μM, 100 μM and 250 μM concentrations of ATP. Data points are the average of two independent experiments. (FIG. 9B, Table 1). Data points are the average of two independent experiments. Accordingly, ATP-K_{M} value was determined (FIG. 9C). Recombinant p38 γ protein was mixed with a synthetic peptide substrate in low volume 384 well microplates, followed with addition of ATP at 2.5 μM to 800 μM final concentrations. The reaction was conducted in 10 μL volume for 10 min. Luminescence was measured using an automated BMG PHERAstar plate reader. Data points are the average of three independent experiments. The data was plotted with Lineweaver-Burk equation. The slope (K_{M}/Vₘₐₓ), Y-intercept (1/Vₘₐₓ) and X-intercept (−1/K_{M}) were determined using GraphPad Prism software. Compound 1 had a Kᵢ of about 0.019 μM. Kᵢ=IC₅₀/(S/K_{M}+1) equation was used to determine Kᵢ of F7 (Cer et al., Nucleic Acids Research, 2009).

TABLE 1
IC₅₀ value (μM)

| | |
|---|---|
| 10 μM ATP | 0.028 |
| 100 μM ATP | 0.1 |
| 250 μM ATP | 0.34 |

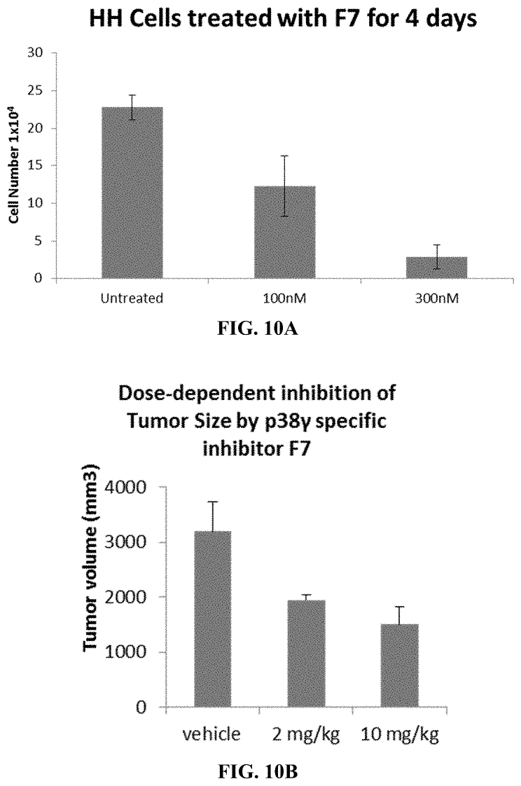

Likewise, Compound 1 sufficiently reduced proliferation of HH cells at 100 nM and 300 nM concentration after treating the cells for four days (FIG. 10A), and exhibited dose-dependent inhibition at 2 mg/kg or 10 mg/kg compared to the non-treated cells (FIG. 10B)

Inhibition of Compound 1 was tested by measuring the mRNA level and protein expression level of p38γ protein. Compound 1 reduces the mRNA level of p38 gamma at 4 hr (FIG. 11A) and Western Blot shown that F7 reduces and eliminates the protein expression of p38 gamma at 24 hr at 500 nM (FIG. 11B). The cytotoxicity effect of F7 is correlated with the disappearance of p38γ protein level which has been confirmed in other experiments. Accordingly, the treatment with Compound 1 can be equal to the knock down of p38 γ protein level at a higher concentration.

In this study, we will elucidate novel targets in the p38γ—NFATC4 signaling pathway that play an important role in the survival or proliferation of CTCL cells. We will determine the extent to which combined application of our lead compound 1 and histone deacetylase inhibitors synergize to enhance cell killing. Finally, we will optimize and validate lead Compound 1 using cell culture, patient samples, mouse models, and eventually clinical trials.

Further, the mechanism by which p38γ affects activation of NFATC4 and a critical downstream molecule for affecting CTCL proliferation and causing the cell death will be investigated. We will build on our preliminary data and the literature to identify additional relevant kinase targets. We will perform global identification of p38γ phosphorylation targets. To validate the interaction of identified targets with p38γ, we will perform site directed mutagenesis and measure the effects on p38γ binding, downstream signaling, and cell proliferation and death.

### Example 3: Determine the Mechanism Through which p38γ Inhibition Induces Cell Death in CTCL

Rationale: We will investigate the mechanism by which p38γ affects activation of NFATC4, a critical downstream molecule for CTCL proliferation, to affect cell proliferation and death. We will build on our preliminary data and the literature to identify additional relevant kinase targets. We will perform global identification of p38γ phosphorylation targets. To validate the interaction of identified targets with p38γ, we will perform site directed mutagenesis and measure the effects on p38γ binding, downstream signaling, and cell proliferation and death.

Identifying downstream targets in the pathway will provide insight into potential mechanisms of resistance and future therapeutic targets. We will work with the COH Mass Spectrometry & Proteomics Core, and do not expect technical difficulties performing proteomic analysis. Though NFATs play a role in many facets of helper T cell function, for this proposal we are focused on the role of NFATC4 on malignant T cell proliferation. We expect to identify numerous potential phosphorylated targets of p38γ signaling that have an effect in CTCL. If one target fails to demonstrate effects on the p38γ pathway, cell killing, or synergy with other inhibitors, we can broaden our screen to other functional categories, such as cellular localization (e.g., p38γ is most abundantly expressed in the mitochondria, and we could examine other mitochondrial proteins identified in the screen). One study indicates that HDAC inhibition prevents NF-κB activation by suppressing proteasome activity [35]; thus, identification of targets involved in proteasome degradation will be exciting, due to the potential synergism with HDACi and NF-κB signaling discussed above. In addition to its kinase function, p38γ also affects downstream pathways through protein-protein interactions, due to the presence of a PDZ binding domain. Lead Compound 1 is also a potent inhibitor of PI3 kinase, p110α (IC₅₀=0.3 nM)[36]. Thus it is possible we may see off-targets effects on this or other kinases discussed above. To rule this out, we will perform a kinase panel, and well as selected knockdown of PI3K and/or its relevant kinases along its pathway such as PTEN in the presence of the Lead Compound 1 (F7) in our CTCL model. If knockdown does not rescue the cytotoxicity effects of CTCL by F7, this will reinforce p38γ as a driver; if it does affect viability, we can investigate its synergy with Compound 1 as well. We do not expect technical difficulties expressing the mutated target proteins in Hut 78 cells as we have successfully performed lentiviral transduction of Hut78 cells. F7 inhibits both p38γ and PI3K110α kinase activity in nanomolar range. Our data exhibited in paragraph [0586] and [0591] in this application that unlike p38γ, blocking PI3K110α enzyme activity either by its specific inhibitor A66 or gene silencing by shRNA transduction does not interfere CTCL cell viability, suggesting that cytotoxicity in CTCL by F7 is due to p38γ, not PI3K110α.

### Example 4: Combined p38γ Inhibition and HDAC Inhibition to Target Complementary Pathways and Induce Synergistic Therapeutic Effects

Rational: Our preliminary data show that a therapeutic combination of our lead p38γ inhibitor compound 1 and the pan-HDACi SAHA induce synergistic killing of CTCL cells. This suggests that the p38γ/NFATC4 and NF-κB pathways, which are both overexpressed in CTCL, are complementary. Thus, we hypothesize that the synergistic toxicity we demonstrated is a result of effects on two key proliferation pathways in normal and malignant T cells, NFATC4 and NF-κB. Accordingly, we will study the effects of combination p38γ and HDAC inhibition on CTCL response and downstream targets of both pathways. In addition to an improved understanding of pan-HDACi effects, we will dissect which HDAC subsets are critical targets for mediating synergistic killing. Our goal is to identify the most effective combination of p38γ and HDAC inhibition that enhances therapeutic efficacy while minimizing toxicity.

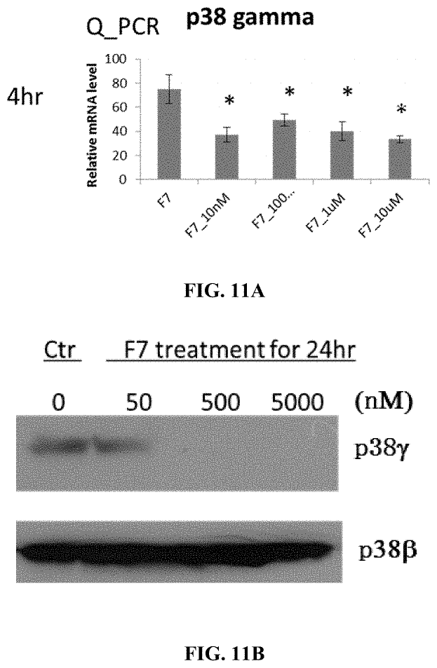

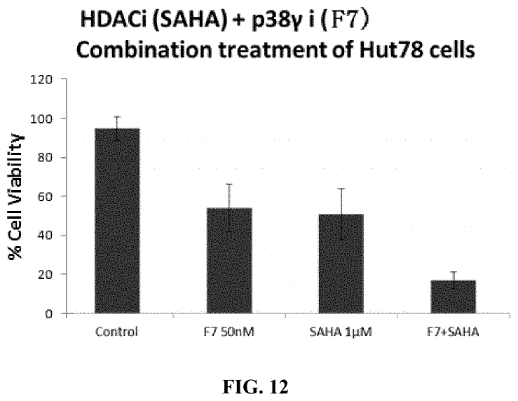

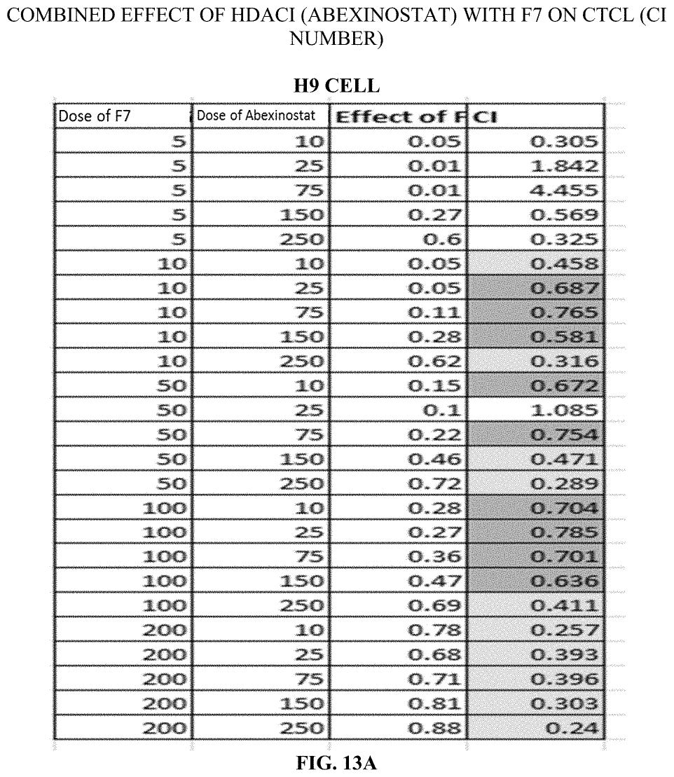

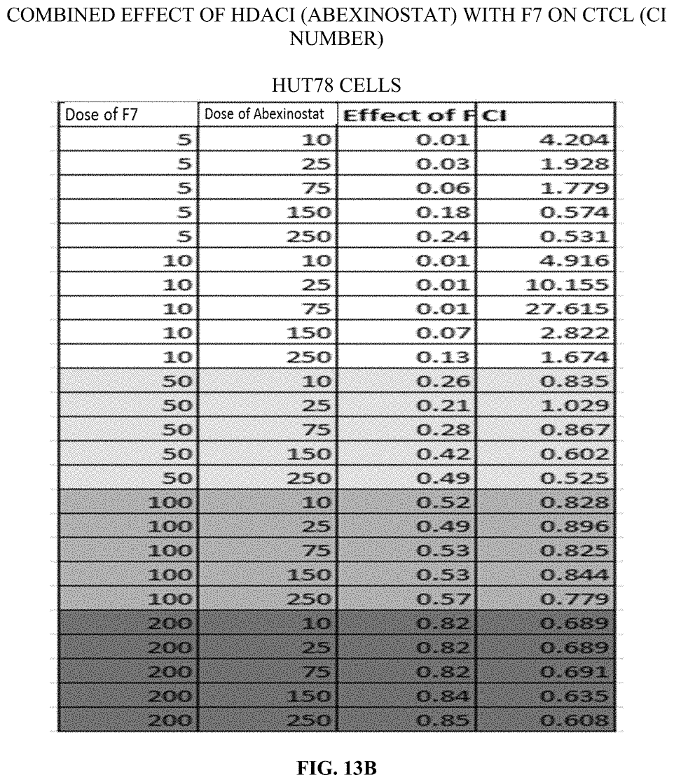

Background and Preliminary Studies: Opposing activities of histone acetyltransferases (HATs) and histone deacetylases (HDACs) tightly regulate gene expression through chromatin modification, and are often dysregulated in cancer. HDACs catalyze the removal of histone acetyl groups, leading to chromatin condensation and transcriptional repression. Additionally, HDACs can target non-histone substrates, and non-transcriptional HDAC mechanisms can regulate cellular processes that are disrupted in cancer, making HDACs an even more attractive therapeutic target[37]. HDAC inhibitors (HDACi) can limit cancer cell growth through cell cycle arrest and promotion of apoptosis, and have also been shown to modulate the immune response [38]. The HDACi vorinostat (SAHA) and romidepsin are FDA-approved for treatment of CTCL[37]. These HDACi are among the most effective agents in CTCL and are well-tolerated, with modest toxicity; side effects are predominantly fatigue and transient suppression of hematopoiesis[1]. However, drug resistance to vorinostat and romidepsin are universally observed in clinical studies of CTCL, emphasizing the need to develop synergistic therapies to overcome resistance [37,39-41]. Preclinical studies and early clinical trials have shown that HDACi can be used effectively in combination with other drugs to induce synergistic anti-cancer effects[41]. Combinational targeting of the B cell receptor (BCR) and NF-κB in B-cell malignancies have also been demonstrated, so we expect a similar approach targeting TCR and NF-κB signaling to be successful in CTCL[42]. Our preliminary data demonstrate that the combination of our p38γ inhibitor, lead compound 1, with the pan-HDACi, SAHA, shows synergistic inhibition of proliferation in CTCL Hut78 cells (FIG. 12). Our preliminary data also demonstrate that the combination of our p38γ inhibitor, lead compound 1, with Abexinostat, shows synergistic inhibition of proliferation in H9 cells and Hut78 cells (FIGS. 13A-13B). Compound 1 can sensitized T malignant cell and its combination treatment with HDAC inhibitors can be an alternative regimen for HDAC drug-resistance of CTCL patients. We used calcuSyn software to calculate a combination index (CI) of 0.79; this CI value indicates the synergistic effect of the two inhibitors. HDACi target other types of cancers in part through effects on NF-κB signaling. The NF-κB transcription factor is known as a “master regulator,” and is an important mediator of immune and inflammatory responses[43]. It also plays critical roles in a number of cellular pathways, including promotion of cell cycle progression and inhibition of apoptosis [44]. NF-κB is frequently overexpressed in cancer, leading to dysregulation of these pathways and promotion of tumorigenesis, invasion and metastasis, and resistance to therapy [43,45]. Upon activation, NFκB rapidly enhances the expression of proinflammatory genes such as cytokines and cell adhesion molecules, as well as genes that promote cell proliferation, angiogenesis, and cell survival of many cancer types[46]. The ability of many HDAC inhibitors to modulate NF-κB activity coincides with its cancer suppressing and anti-inflammatory activities in several models of cancer[35]. A detailed understanding of the underlying functions of HDACs, and a corresponding understanding of the mechanism of HDACi effects remain poorly understood[37]. In CTCL, whether HDACi target NF-κB or not, and the mechanism through which HDACi target NF-κB, are still unclear. However, mycosis fungoides and Sézary syndrome are associated with constitutive activation of the NF-κB pathway, a well-known feature of CTCL and other haematopoetic malignanices[16,47], suggesting that the success of HDACi in CTCL may be in part through effects on NF-κB. Nuclear NF-kB expression has been demonstrated in Hut78 and HH cell lines, and inhibition of NF-kB in Hut78 and HH cells using chemical inhibitors or a proteasome inhibitor induced apoptosis and reduced proliferation, respectively [4,16]. Furthermore, small molecule inhibition of NF-kB inhibited tumor growth and metastasis in a CTCL xenograft mouse model[48].

Combined pan-HDACi and p38γ inhibition: We will apply the p38γ inhibitor compound 1 and/or optimized analogs singly and in combination with pan-HDACi (SAHA) to CTCL cell lines Hut78 and HH. We will base dose escalation and timing regimens on our preliminary data and the literature. We will measure CTCL response to single and combined inhibitor treatment using proliferation, cell cycle, viability, and apoptosis assays. To confirm cell line results in CTCL patient tissues, we will use primary patient samples from the COH CTCL bank as available; if we see an effect, we will increase patient samples to achieve statistically significance. Vehicle-treated CTCL cells will be used as a control for drug treatment. CD4+ T cells from healthy donors will be used as controls for CTCL-specific effects. To determine the effects of combined treatment on downstream pathways, we will collect whole cell lysates and nuclear extracts from the treated cells and measure a number of downstream targets in the p38γ/NFAT and NF-κB pathways. For example, to measure effects on p38γ activation, i) p38γ kinase assays, ii) qRT-PCR and iii) western blot can be performed to quantitate mRNA and protein expression, respectively, of p38γ and NFATC4. Other targets of p38γ signaling will include those identified in above. We will measure effects on DLGH1 as described in above; importantly, DLGH1 can be used as a marker for inhibition of p38γ/NFAT versus NF-κB TCR signaling [15]. We will confirm HDACi specific effects by measuring expression of HR23B, also known as RAD23B. This protein is involved in protein targeting to the proteasome, predicts sensitivity to HDACi, and furthermore, is considered a potential biomarker for CTCL [49-51]. We will perform qRT-PCR and western blot to confirm reduced expression of NF-κB mRNA and protein, respectively. To measure the downstream effects of combination treatment on the transcriptional activity of NF-κB, we will perform an ELISA assay. The NF-κB (p65) Transcription Factor Assay (Rockland) is a non-radioactive, sensitive method for detecting specific transcription factor DNA binding activity in nuclear extracts and whole cell lysates. NF-κB specifically binds to the NF-κB response element and is detected by addition of a specific primary antibody. We will also measure NF-κB protein level and phosphorylation and acetylation status using Western blot. Nuclear extracts and whole cells lysates will be separated using SDS-PAGE and Western blot for quantification of protein level. We will measure the phosphorylation status of NF-κB p65 with anti-phosphop65 (Ser276) and anti-phospho p65 (Ser536) (EMD Millipore). We will measure the acetylation status of NF-κB p65 with anti-acetyl-p65 (Lys310) (Cell Signaling Technology).

Specific HDAC inhibition and p38γ inhibition: HDACs affect a variety of histone and non-histone targets; despite some redundancy, unique HDACs can cause substrate-specific effects[37]. For example, HDAC1 [52], HDAC2 [53], and HDAC3 [54] directly interact with subunits of NF-κB. HDAC isoform expression also varies in CTCL, and can have prognostic significance; HDAC2 and HDAC6 expression is elevated in CTCL [55], and elevated expression of HDAC2, as well as acetylated histone H4, is associated with aggressive CTCL, while HDAC6 expression is associated with a favorable prognosis in any CTCL subtype [56]. Thus, we will use siRNA and small molecule inhibitors against HDAC family members (HDAC1, 2, 3, 6) to determine the effects of inhibiting individual HDACs versus HDAC subsets, and to identify the most efficacious combination of HDACi and p38γ inhibition. We will treat cell lines, primary patient samples, and controls as describe above and use assays described to measure the downstream effects of specific HDACi inhibition in combination with p38γ inhibitor compound 1. The most effective combinations identified above will be tested in the mouse models subsequently. In response to p38γ inhibition, we expect to see reduced NFATC4. Potential off-targets effects of lead compound 1 will be considered as described above. If we see reduction of both NFAT and NF-κB expression, we expect see cell killing. In response to HDACi, we expect to see inhibition of NF-κB activity, reduced phosphorylation of NF-κB p65 at Ser276 and Ser536, and reduced acetylation of NF-κB p65 at Lys310. If Western blot shows too many non-specific bands for accurate detection of phosphorylation or acetylation status, we will consider using immunoprecipitation or T7-tag IP method_ENREF_54 [54] to eliminate non-specific bands. If we do not detect any reduction of NF-κB activity in response to HDACi, this will indicate that NF-κB is not an HDACi target in CTCL, although we expect the chance of this to be limited, given evidence in the primary literature. However, if NF-κB is not an HDACi target, or is induced by HDACi, as demonstrated in another study [57], we will explore alternative targets that may explain the efficacy of HDACi in CTCL [56,58]. For example, HDAC6 and HDAC10 inhibitors cause VEGF reduction in CTCL [59,60], and HDACi are well known for effects on the proteasome degradation pathway[35,51]. If we do see reduced activity of NF-κB, but not reduced phospho-p65, we will continue to decipher the possible mechanism of action, such as finding co-repressors of NF-κB that bind to HDACs by the proteomics profiling methodology described. If we do not see reduced acetylation of p65, this will indicate the mechanism is not through HDAC3. For the combination treatment, we expect to see both NFAT and NF-κB reduction and cell killing effects. If we do not see any reduction or cell killing, we will screen alternative inhibitors of p38γ and/or alternative HDACi.

### Example 5: Optimize and Validate a Candidate p38γ Inhibitor

Rational: Our inhibitor development will employ two strategies: characterize and optimize the current lead compound 1 identified from our preliminary screening studies, which likely inhibits the enzyme by a novel mechanism; and structure-based design of inhibitors of phosphorylated p38γ, leveraging on the extensive knowledge of kinase inhibitor discovery. In addition to improving potency and selectivity, we will optimize compounds to improve drug metabolism and pharmacokinetic properties (DMPK), and achieve oral bioavailability. Our preliminary data and extensive publications demonstrate that we have the appropriate expertise in lead optimization, bioassays, and structural characterization to develop and evaluate candidate compounds as p38γ inhibitors for preclinical studies.

Preliminary Studies: Understanding the mechanism of inhibition is necessary to guide rational lead optimization. Preliminary effort in co-crystalizing p38γ-lead compound 1 complex was unsuccessful, but we have obtained high quality NMR data for p38γ and its complex with Compound 1 (not shown) that demonstrates the feasibility of studying p38γ-inhibitor interactions by solution NMR methods. Significant chemical shift changes were observed for residues on the top of the ATP-binding site of p38γ upon addition of 1 to the solution. Compound 1 has slow off-rate on the NMR chemical shift timescale, as indicated by distinct resonances of the free and bound population. Such slow off-rate corresponds to nM dissociation constant. This result is unexpected. 1 has an estimated Kᵢ of 10 nM for inhibiting p38γ enzymatic activity based on IC₅₀ and enzyme concentration used, suggesting that it also binds to phosphorylated p38γ with a dissociation constant in the nM range. Because inactive (unphosphorylated) and activated (phosphorylated) kinases have different conformations, it is unusual for a compound to bind to both forms with high affinity.

Optimization of the HTS lead compound: As discussed in Preliminary studies, the mechanism of action of the lead compound 1 is unique in that it binds to both activated and inactive p38γ with high affinity. To understand the mechanism of inhibition of 1, we will characterize the structure of the compound bound to the phosphorylated and unphosphorylated forms of p38γ Although we will continue to pursue crystallization experiments to obtain a co-crystal structure of the enzyme-inhibitor complex, we have also shown that it is feasible to determine the structure by NMR spectroscopy. To obtain structural information, we will measure the distances between the inhibitor and p38γ using 3D-NOESY for structural calculations of the protein-inhibitor complexes using the HADDOCK program [61], as we have shown previously [62]. For example, protein samples can be labeled by culturing in M9 medium containing deuterated-glucose, 100% D₂O, [¹³CH₃; 3,3-D₂]-alpha-ketobutyric acid, and [3-¹³CH₃; 3,4,4,4-D₄]-alpha-ketoisovaleric acid to produce Ileδ1-[¹³CH₃], Leu, Val-[¹³CH₃, ¹²CD₃] p38γ samples for NMR experiments, as described previously[64, 65]. ¹³C NOESY-HMQC experiments will be carried out from non-uniform sampling (NUS) in order to maximize sensitivity. The precise binding affinities of 1 for the activated and inactive forms of p38γ will be determined using surface plasma resonance and isothermal titration calorimetry. Lead optimization will be guided by structural insights of how Compound 1 interacts with p38γ. To improve efficacy in vivo and reduce potential liability, our lead optimization strategy will apply medicinal chemistry to improve the solubility, potency, specificity, and other DMPK properties of 1. The general synthetic strategy[36] is outlined in Scheme 1, and will be guided by structural insights gained above. Many commercial analogs exist, with various R₁ to R₄, which can be incorporated as described in Scheme 1.

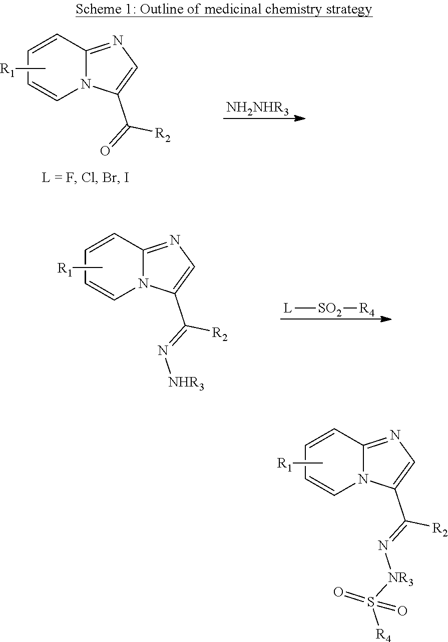

To improve potency and specificity, we will choose R₁ to R₄-groups that fully fill the binding pocket, based on structural studies. To improve solubility, we will introduce solubilizing groups. For example, we will add solubilizing groups that will not interfere with enzyme binding. We will focus on smaller alkyl and heterocyloalkyl substituents known to impart solubility in other kinase projects (e.g., ethylpiperdine, ethylmorpholine) to minimize molecular weight and interference with enzyme binding. We will also investigate by structure-activity relationship the need/role for the metabolically labile aromatic nitro group.

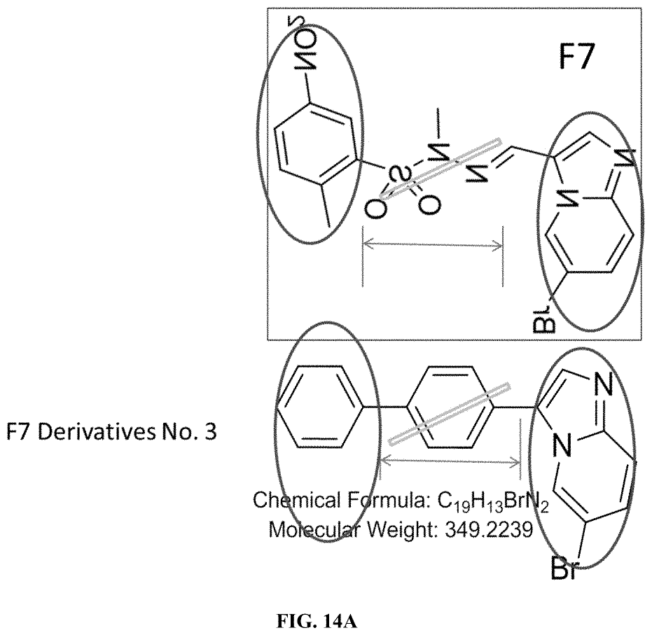

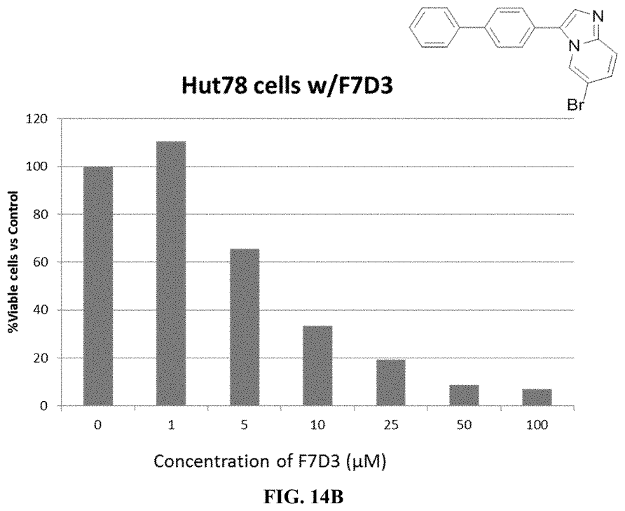

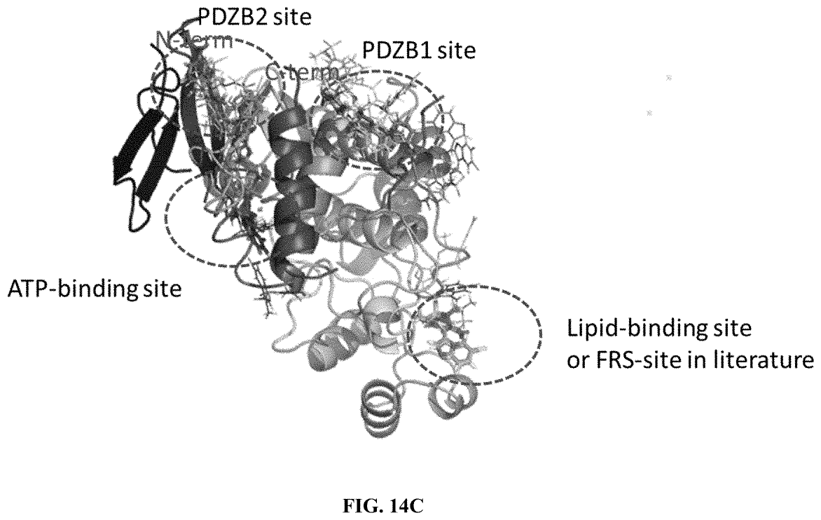

Structure-guided drug design: We will also design inhibitors that target the active form of p38γ, based on the extensive knowledge of the structure-activity relationship of kinase inhibitors to inform structure-based design as we did for F7D3. FIG. 14A shown novel approach for designing compounds based on the L¹ length of F7D3. For instance, the spatial distance between two (circled) aromatic groups in Compound 1 range from 5.0 to 5.9 Å. Plausible length may range from about 4 to 12 Å or preferably from about 4 to 10 Å. The spatial distance between two circled groups in F7 may be important for the potency of the drug and anew plan to generate F7 derivatives may be based on that distance (FIG. 14A). F7D3 was designed to have a length of L¹ of about 5.8 Å and was shown to suppress viability of Hut 78 cells in dose dependent manner (FIG. 14B). In other approach, possible docking model can pose analogues of Compound Ion p38γ (FIG. 14C). For instance, first choice can be made when an analogue likes ATP-binding site a little better with docking score −5.5, second choice can be made when lipid-binding site/FRS-site has docking score −4.51; third choice can be made when PDZB1 site has docking score −3.6; or the fourth choice can be made when PDZB2 site (between N-term and C-term) has docking score −2.9. However, our approach does not exclude the possibility when analogues may bind to other sites of p38γ.

Biochemical and cellular assays, structural characterization, and drug metabolism and pharmacokinetic (DMPK) properties studies: The new analogs can be quickly screened to evaluate their inhibitory effect on and selectivity for p38γ in biochemical and cellular assays. In addition to directly measuring effects on p38γ, we can also measure protein level of p38 gamma as an efficient way to evaluate downstream effects of cell viability. Because our recent result indicates the direct correlation of disappearance of p38 gamma protein itself with cancer cell death. Western blot will be our very first validation step as some compounds do not inhibit the p38gamma kinase activity but can stop it protein expression such as shRNA gene silence approach. The specificity of the new lead compounds for p38γ can be confirmed by examining the effects on activity of other p38 isoforms. The compounds that reach the desired potency and specificity according to biochemical and cellular assays can be further analyzed for drug metabolism and pharmacokinetic (DMPK) properties. Solubility, plasma protein binding, plasma stability, and hepatocyte stability can be measured using commercial services, such as Quintara Discovery (San Francisco, Calif.). For compounds that lack stability, the metabolites can be identified by a commercial service (such as Ricerca LLC) to feed back to medicinal chemistry design. Metabolic “hot spots” can be modified to improve the compound profile. In addition, data from potency, solubility assessment, plasma protein binding, and hepatocyte stability can also direct medicinal chemistry efforts. The iterative approach can act as a feedback loop that moves compounds down the screening cascade. Medicinal chemistry, biochemical, cellular studies, and DMPK analysis can be coupled with iterative structural studies of protein-ligand interactions. We can treat cells (e.g., Hut78) with the candidate inhibitor and determine the extent to which effects (e.g., proliferation and apoptosis) phenocopy that of p38γ knockdown. In addition, genome-wide gene expression analysis can be conducted on inhibitor-treated cells and compared to that of cells with p38γ knockdown.

Animal Studies: Determine the maximum tolerated dose (MTD) of the lead inhibitors in vivo. We can conduct mouse model studies to determine the MTD of the lead compound. Male and female 8-12 wk, 20-25 g mice (up to 40 mice, 20 male and 20 female) can be used for the MTD. To begin, one mouse can receive a presumed toxic dose of the inhibitor through intravenous (IV) tail vein injection, intraperitoneal injection (IP), or oral gavage. Toxicity can then be monitored for 15 days to ensure there are no delayed toxic side effects. Mice can be weighed and monitored daily for signs of toxicity, such as loss of appetite, hunched posture, coat ruffling, eye crustiness, and changes in activity level.

Perform PK studies of candidate inhibitors. Both single and multi-dose PK studies of the candidate compound can be conducted. The effects of dose and route of administration on PK can be investigated to yield key PK parameters, such as maximum achievable plasma concentration (Cₘₐₓ), plasma half-life (T_{1/2}), clearance, volume of distribution, and bioavailability (F_{IP/oral}), as well as to determine inhibitor concentrations achievable in normal and tumor tissues. PK studies can be conducted for at least two dose levels and can determine the optimal route of administration. The optimal route can be defined as the one that results in the highest plasma area-under-the-curve (AUC). We hope to achieve oral bioavailability greater than 50%, so that the oral route can be chosen for further investigation. Male and female 8-12 wk, 20-25 g mice (70 mice, 35 male and 35 female) can be used for PK studies. Groups of three mice can be euthanized at each time point (2 min, 15 min, 30 min, 1 h, 2 h, 6 h, 12 h, and 24 h) to obtain intracardiac blood and tissue. The sampling time points can be chosen to capture prolonged drug elimination as well as the initial absorption, distribution, and elimination of the drug. Mice can be euthanized by CO₂ asphyxiation, after which blood can be immediately collected by cardiac puncture, transferred into heparinized tubes, and centrifuged for 5 min. The resulting plasma can be stored at −70° C. for subsequent analysis using a validated LC-MS/MS assay that can be developed in the COH Analytical Pharmacology Core Facility (APCF).

Determine therapeutic efficacy in cell line and patient-derived xenograft models in mice: The lead compounds can be examined for therapeutic efficacy in xenograft mouse models developed from CTCL cell lines as in preliminary data. Experiments can be performed on equal numbers of female and male NSG mice (NOD-scid IL2Rg^{null}; Jackson Labs #005557), age 8-15 weeks. Briefly, 5-10 million tumor cells in 100 μL (50% Matrigel/50% PBS) can be injected subcutaneously into the flanks of the animals. Once the tumors are palpable and reach 100 mm³ in volume, mice can be administered drugs by oral gavage, or intraperitoneal or intravenous injection, based on PK data. Through lead optimization, we can likely obtain compounds with oral bioavailability. Tumor size can be measured twice per week using calipers. Experiments can be stopped once the tumors reach 15 mm in diameter. Any mouse that has a body weight loss of more than 20% or exhibits any severe pain/distress signs that match premature euthanasia criteria can be sedated and euthanized. Mice can be monitored for tumor regression for 4 weeks post-treatment. All mice that survive the 4-week monitoring period can be sedated and euthanized. The autopsy can include visual examination, weight, and histological examination of plasma, tumor, and tissues, including but not limited to liver, kidney, heart, lung, and spleen. The efficacy studies can be split into two categories: 1) a dose finding mono-therapy, and 2) a combination study. For mono-therapy study, we can use 10 mice (5 male and 5 female) per experimental group, 10 groups total for control vehicle and three analogs at three dose-levels each. To compare an analog to control vehicle, the sample size of 10 per group can provide 85% power for an effect size of 1.5 at 1-sided significance level of 0.017 (Bonferroni adjustment for three doses at an overall significance level 0.05) in a two-sample t-test, where effect size is the difference in the mean tumor volume between the treated group and the control group divided by the standard deviation of tumor volume. In the subsequent 2-drug combination study, we can use 10 mice per group with four groups total for control vehicle, monotherapy for p38γ, monotherapy for HDACi, and 2-drug combination. A sample size of 10 per group can provide 82% power for an effect size of 1.2 at 1-sided significance level of 0.05 in a two-sample t-test. Pharmacodynamic (PD) effects can be analyzed to establish a PK-PD correlation. NFATC4 and IL-17A expression in tumor tissues of sacrificed mice can be measured to confirm p38γ inhibition. Furthermore, cell proliferation (Ki67 staining) and apoptosis (TUNEL assay) can be examined. In addition, if preliminary data supports further preclinical (PDX) investigation of combination therapies, we can compare single agent activity of HDAC inhibitors, the optimized p38γ inhibitor, and their combination in PDX models. We can acquire a pre-existing CTCL SS PDX mouse model (DFTL-90501-V3) from Dr. David M. Weinstock's Leukemia and Lymphoma Xenograft (LLX) public repository[69] to verify the efficacy of our lead compounds and combination with HDACi treatment, as described for xenograft models above. We can use 10 mice per group (5 male, 5 female) for with four groups total for control vehicle, monotherapy for p38γ, monotherapy for HDACi, and 2-drug combination. Preclinical activity data from these PDX studies can determine the optimal combination and can support the integration of p38γ inhibitors in combination with HDACi in future clinical trials.

Expected Results, Potential Pitfalls, and Alternative Approaches: We expect to validate p38γ as a good target for developing anti-cancer therapeutics in CTCL and possibly other cancers that are dependent on p38γ alternate signaling. We expect to obtain a p38γ inhibitor that does not have compound-related toxicity, have improved solubility and other DMPK properties, and is at least as potent as 1, with minimal off-target effects and feasible for oral administration. Because p38γ is not expressed in normal T-cells and not essential because knockout mouse are viable[15], we do not expect significant mechanism-based toxicity. Compound-based toxicity can be addressed by the lead optimization effort. We can perform kinase screening to determine effects on targets besides p38γ; in particular, compound 1 is known to be a PI3K inhibitor. However, even if off-target kinase effects are identified, and cannot be addressed through additional optimization, this may not preclude the value of the inhibitor, given that several of the most effective FDA-approved kinase inhibitors (e.g., Gleevec) fall into this category. Because of our extensive experience with the proposed studies, we do not anticipate technical difficulties. We expect our results to have immediate relevance for CTCL therapy. Furthermore, p38γ protein is highly expressed in several human malignant cell lines with a spectrum of histologies, including CTCL, melanoma[70], colon cancer, and breast cancer [71,72], indicating a possible role in tumorigenesis of multiple cancers. For example, human p38γ is overexpressed in both ER+ and ER-breast cancer cells[73], as well as triple-negative breast cancer. This suggests that our study results can be relevant to understanding the biology of other p38γ-driven cancers, as well as identifying therapeutic targets.

### Example 6: Gene Expression Profiling

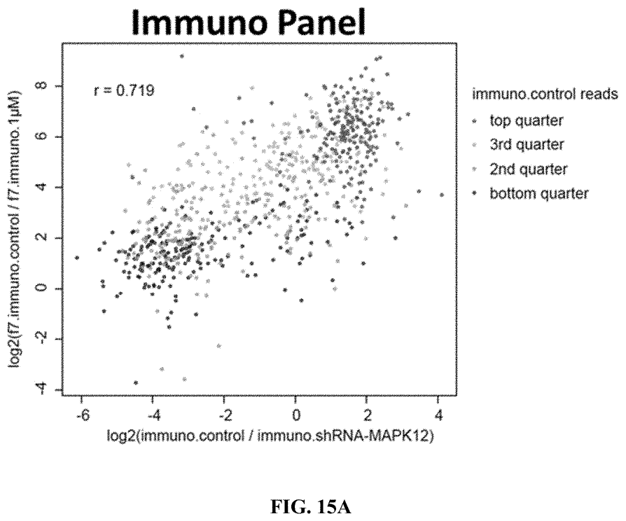

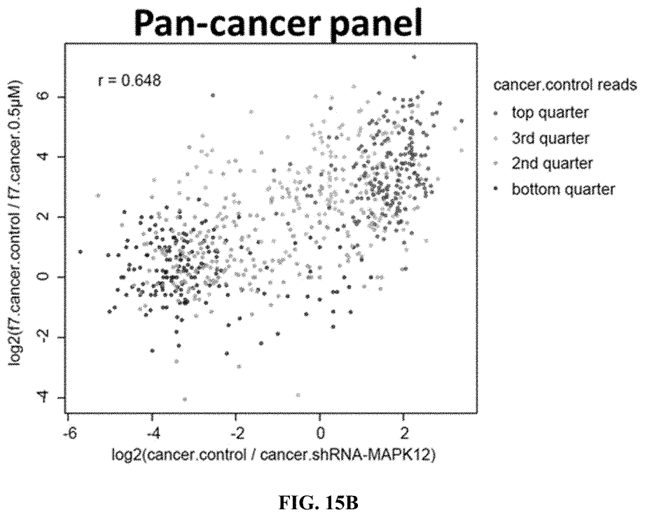

The Gene expression profiling of treatment with Compound 1 (0.5 mM) shows highly positive correlation to that of the shRNA-p38 gamma (treatments vs control of Hut 78 cells) in both Immuno (FIG. 15A) and Pan-cancer panel (FIG. 15B). The dataset of Compound 1 resembles that of the shRNA-p38 gamma expression. It exhibits high positive correlations between seemingly unrelated two gene expression profiling datasets by Nanostring RNA analysis (Compound 1 treatment and sh_p38 gamma expression in the Hut78 cells). The correlation coefficient are 0.719 and 0.648 for NS Immuno panel and Pan Cancer panel, respectively. The result indicates both sh-p38 gamma expression and Compound 1 treatment have the same target—p38 gamma gene expression.

In addition, the Nanostring RNA analysis data shows that there is internal upheavals within CTCL cells upon the loss of p38 gamma by either Compound 1 or shRNA p38 gamma knock down treatment (Table 2), which has revealed through its gene expression profiling that many genes that are originally low expressed (in control untreated cells) become highly expressed, and many genes that are originally high expressed (in control untreated cells) become low expressed. A group of housekeeping genes which supposed to be unchanged upon drugs treatment has reduced their expression level significantly, which raises the possibility that MAPK12 has some global effect on transcription or transcript half-life.

TABLE 2

| | | | | | |
|---|---|---|---|---|---|
| Min. | 1st Qu. | Median | Mean | 3rd Qu. | Max |

summary (in R) for the H9 control NS immuno-Panel data

| | | | | | |
|---|---|---|---|---|---|
| 1.00 | 8.00 | 24.25 | 582.50 | 375.50 | 33790.00 |

summary for the H9 shRNA_p38 gamma NS immuno-Panel data:

| | | | | | |
|---|---|---|---|---|---|
| 1.00 | 60.33 | 113.00 | 270.60 | 211.60 | 29000.00 |

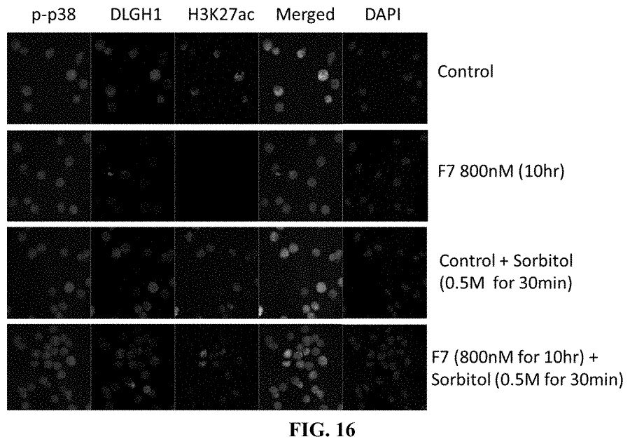

Our housekeeping gene list hits H3K27 ac in database mining, which can suggest that acetylation H3K27 reduced by Compound 1. Compound 1 at a concentration of 800 nM for 10 hr treatment blocked H3K27 acetylation and the blockage can be released by sorbitol, a p38 gamma inducer/activator (FIG. 16). Sorbitol is known to activate p38 gamma.

### Example 7: Analogues

Synthesis

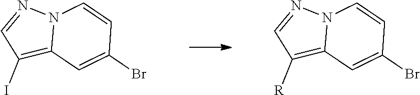

To a solution of 6-bromo-3-iodoimidazo[1,2-a]pyridine (33 mg, 0.1 mmol) were added arylboronic acid (0.11 mmol), K₂CO₃ (55 mg, 0.4 mmol), and PdCl₂(dppf)·CH₂Cl₂ (4 mg, 0.005 mmol). The resulting mixture was stirred at 80° c. for 4 h and then cooled to rt. After removal of the solvent, the residue was purified by flash column chromatography (MeOH:CH₂Cl₂, 1:50) to give products.

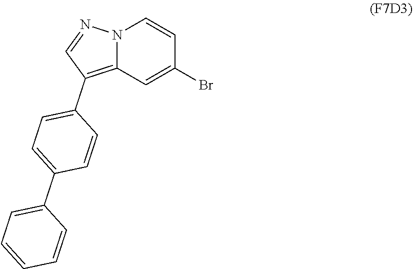 [SMILES: Brc1ccn2ncc(-c3ccc(-c4ccccc4)cc3)c2c1]

¹H NMR (CDCl3, 400 MHz) δ 7.56-7.68 (m, 5H), 7.72-7.80 (m, 3H), 7.56-7.68 (m, 5H), 7.46-7.52 (m, 2H), 7.36-7.42 (m, 1H), 7.24-7.30 (m, 1H).

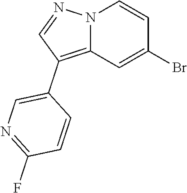 [SMILES: Fc1ccc(-c2cnn3ccc(Br)cc23)cn1]

¹H NMR (CDCl3, 400 MHz) δ 8.42 (s, 1H), 8.29 (s, 1H), 7.97 (dt, 1H, J=2.0, 8.0 Hz), 7.74 (s, 1H), 7.62 (d, 1H, J=8.6 Hz), 7.33 (d, 1H, J=8.4 Hz), 7.15 (dd, 1H, J=2.8, 8.4 Hz).

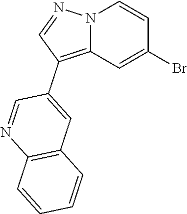 [SMILES: Brc1ccn2ncc(-c3cnc4ccccc4c3)c2c1]

¹H NMR (CDCl3, 400 MHz) δ 9.11 (d, 1H, J=2.4 Hz), 8.49 (s, 1H), 8.35 (d, 1H, J=2.0 Hz), 8.21 (d, 1H, J=8.4 Hz), 7.94 (d, 1H, J=8.0 Hz), 7.88 (s, 1H), 7.81-7.86 (m, 1H), (d, 1H, J=8.8 Hz), 7.67 (dt, 1H, J=1.2, 8.0 Hz), 7.40 (d, 1H, J=8.8 Hz).

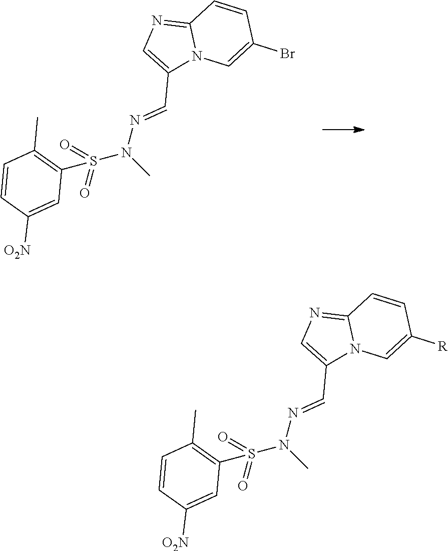

To a solution of starting material (23 mg, 0.05 mmol) were added arylboronic acid (0.12 mmol), K₂CO₃ (27 mg, 0.2 mmol), and PdCl₂(dppf)CH₂Cl₂ (3 mg, 0.004 mmol). The resulting mixture was stirred at 100° C. for 6 h and then cooled to rt. After removal of the solvent, the residue was purified by flash column chromatography (MeOH:CH₂Cl₂, 1:20) to give products.

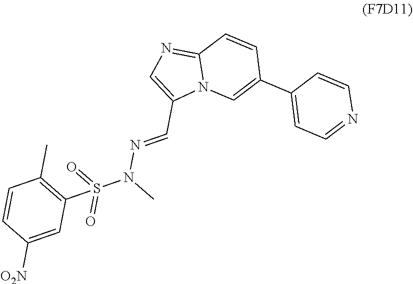 [SMILES: Cc1ccc([N+](=O)[O-])cc1S(=O)(=O)N(C)/N=C/c1cnc2ccc(-c3ccncc3)cn12]

¹H NMR (CDCl3, 400 MHz) δ 9.88 (s, 1H), 8.72-8.85 (m, 2H), 8.71 (d, 1H, J=2.4 Hz), 8.19 (dd, 1H, J=2.4, 8.4 Hz), 8.00 (s, 1H), 7.94 (s, 1H), 7.79 (d, 1H, J=9.6 Hz), 7.68 (dd, 1H, J=1.2, 9.6 Hz), 7.62-7.66 (m, 2H), 7.43 (d, 1H, J=8.0 Hz), 3.46 (s, 3H), 2.72 (s, 3H).

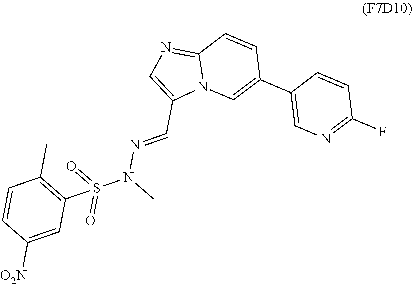 [SMILES: Cc1ccc([N+](=O)[O-])cc1S(=O)(=O)N(C)/N=C/c1cnc2ccc(-c3ccc(F)nc3)cn12]

¹H NMR (CDCl3, 400 MHz) δ 9.65 (s, 1H), 8.71 (d, 1H, J=2.4 Hz), 8.42 (d, 1H, J=2.0 Hz), 8.22 (dd, 1H, J=2.4, 8.0 Hz), 8.11 (dt, 1H, J=2.4, 8.0 Hz), 8.02 (s, 1H), 7.96 (s, 1H), 7.85 (d, 1H, J=9.2 Hz), 7.62 (dd, 1H, J=1.6, 9.2 Hz), 7.47 (d, 1H, J=8.8 Hz), 7.14 (dd, 1H, J=3.2, 8.8 Hz), 3.46 (s, 3H), 2.72 (s, 3H).

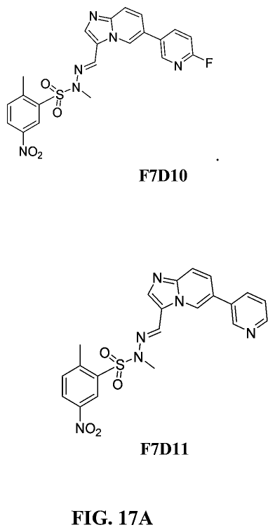

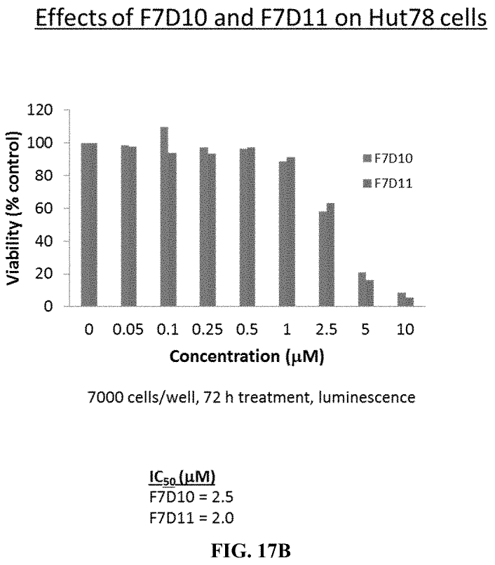

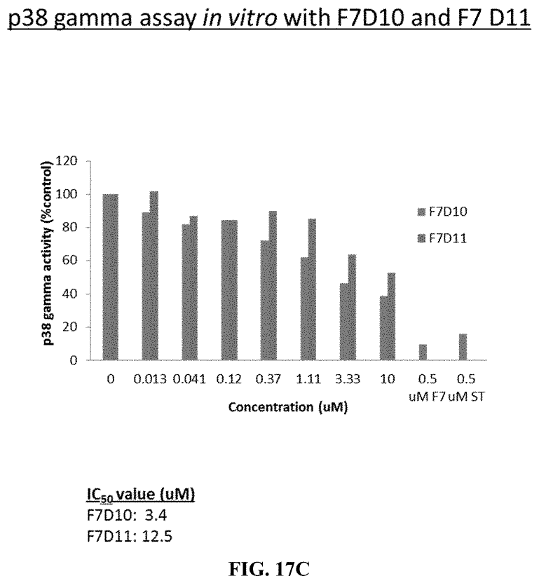

Results. Two analogues (F7D10 and F7D11) as shown in FIG. 17A were identified to have potency of suppressing or cell viability of Hut78 Cells upon 73 hours of treatments at micro-molar concentration. IC₅₀ value of F7D10 and F7D11 by cell viability assays with Hut 38 cells is respectively, about 2.5 μM and about 2.0 μM (FIG. 17B). IC₅₀ value of F7D10 and F7D11 by in vitro inhibition assays against p38γ was respectively, about 3.4 μM and about 12.5 μM (FIG. 17C).

### Example 8: Multi-Kinase Inhibitor with Anti-p38γ Activity in Cutaneous T Cell Lymphoma

Abbreviations: CTCL, Cutaneous T cell lymphoma; MAPK, mitogen-activated protein kinase; DLGH1, Discs large MAGUK scaffold protein 1; NFAT, Nuclear factor of activated T-cells; NF-kB, Nuclear factor Kappa B subunit 1; NMR, Nuclear magnetic resonance; All-around docking (AAD); TCR, T cell receptor signaling; SAHA, vorinostat; PI3K, phosphatidylinositol-3-kinase

Current cutaneous T cell lymphoma (CTCL) therapies are marked by an abbreviated response, subsequent drug resistance, and poor prognosis for patients with advanced disease. An understanding of molecular regulators involved in CTCL is needed to develop effective targeted therapies. One candidate regulator is p38γ, a mitogen-activated protein kinase crucial for malignant T cell activity and growth. p38γ gene expression is selectively increased in CTCL patient samples as well as cell lines, but not in healthy T cells. In addition, gene silencing of p38γ reduced CTCL cell viability, demonstrating a key role in CTCL pathogenesis. Screening p38γ inhibitors is critical for understanding the mechanism of CTCL tumorigenesis and developing therapeutic applications. We prioritized a potent p38γ inhibitor (F7, also known as PIK75) through a high-throughput kinase inhibitor screen. At nanomolar concentrations, PIK75, a multiple kinase inhibitor, selectively killed CD4+ malignant CTCL cells, but spared healthy CD4+ cells; induced significant reduction of tumor size in mouse xenografts; and effectively inhibited p38γ enzymatic activity and phosphorylation of its substrate, DLGH1, in CTCL cells and mouse xenografts. Here we report that PIK75 has a potential clinical application to serve as a scaffold molecule for the development of a more selective p38γ inhibitor.

Cutaneous T cell lymphoma (CTCL) is a severe, disfiguring, and incurable malignancy with a poor prognosis for patients with advanced disease. Current therapies are associated with an abbreviated response and subsequent drug resistance [1, 2]. Unlike many cancers, CTCL pathogenesis remains poorly understood. Until recently, no molecular drivers had been identified, prohibiting the development of driver-based targeted therapies. Thus, identifying critical pathways and molecular drivers of CTCL is essential to understanding progression of the disease and developing effective therapies that improve quality of life and outcome for CTCL patients.

We recently demonstrated enrichment of transcripts involved in the T-cell receptor (TCR) and mitogen-activated protein kinase (MAPK) pathways in CTCL, and identified the MAPK p38β isoform as a potential therapeutic target for CTCL [5]. That study prompted us to characterize the potential of other isoforms of p38 to be therapeutic targets in CTCL. One candidate is p38γ, a 367-amino acid protein that is highly expressed in skeletal muscle, with no detectable expression in normal hematopoietic cells or tissues of the immune system, including lymph nodes and spleen [6, 7].

It is noteworthy that DLGH1, an important scaffolding protein that directs T cell signaling through the NFAT pathway rather than through the NF-kB pathway [14, 15], is a substrate of p38γ. In HeLa cells, p38γ phosphorylates DLGH1 on serine 158 and the phosphorylation can release DLGH1 (SAP97) from its co-factors GKAP for further related function in HeLa cells [15]. Thus, DLGH1, which signals early in the TCR signaling pathway, may influence the TCR pathway through its phosphorylation by the unexpected presence of p38γ kinase in cancerous T cells, such as CTCL cells.

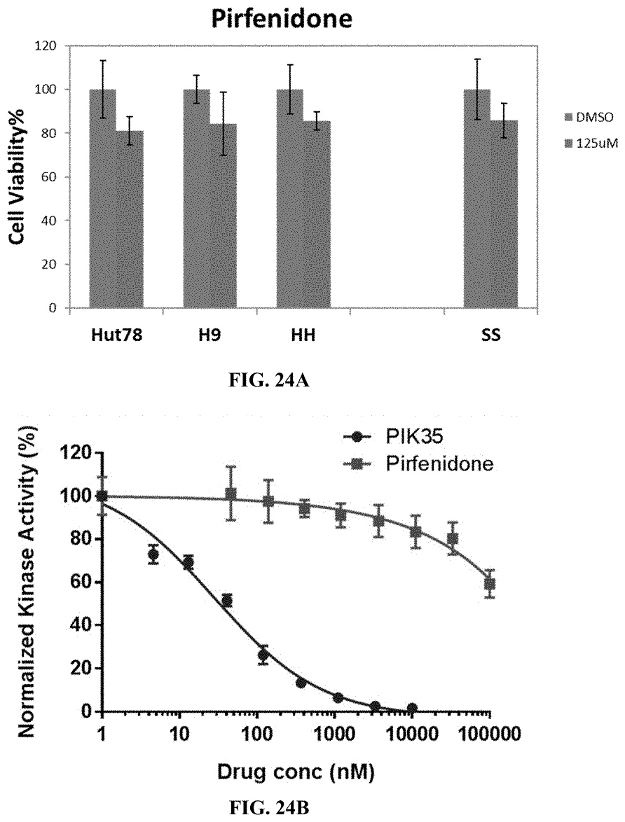

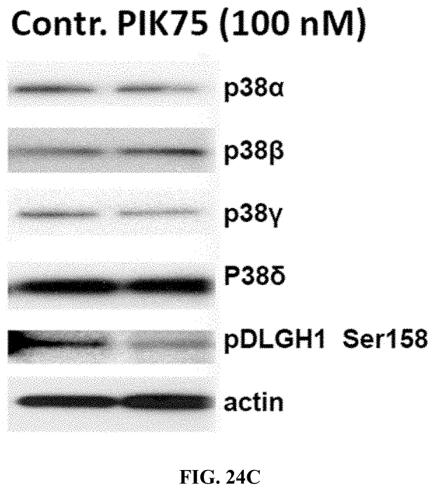

To date, there is only one p38γ inhibitor in clinical practice. Pirfenidone (the orphan drug Esbriet), an FDA-approved drug for treatment of pulmonary fibrosis, is a p38γ inhibitor that blocks TGF-β synthesis [75]. It inhibits p38γ expression in mice, but requires a very high daily dosage (500 mg/kg per day in drinking water) [22], which raises questions about its clinical value against cancer. Therefore, more potent p38γ inhibitors may be of benefit for the therapy of this disease. In this study, we demonstrate a potential role for p38γ in malignant T cell activity and growth, identify a potent small molecule inhibitor (F7, also known as PIK75) through high-throughput screening of a kinase inhibitor library, and describe the unique effects of F7/PIK75 on CTCL and p38γ along with other kinases. However, our data indicated that pirfenidone doesn't inhibit p38γ kinase activity (IC₅₀ over 125 μM) and not inhibit CTCL cell growth (IC₅₀ over 125 μM) (FIGS. 24A-24B).

Results

p38γ is Elevated in CTCL and is Important for Cell Viability

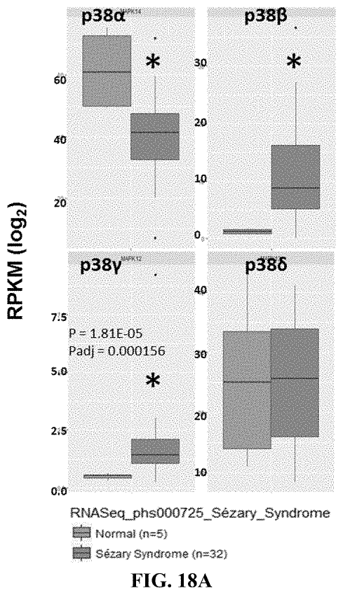

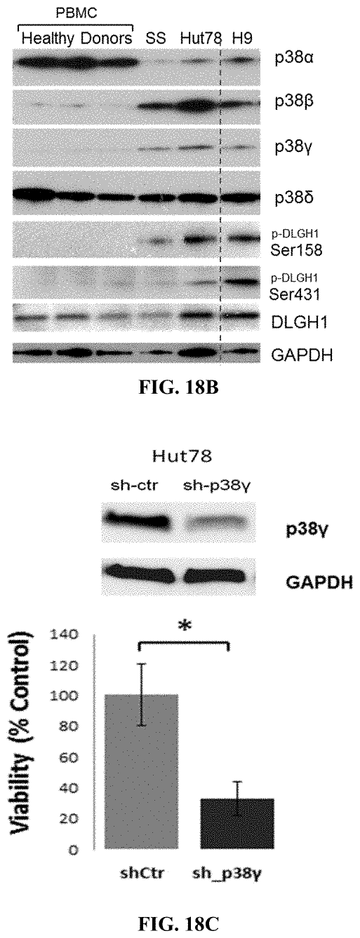

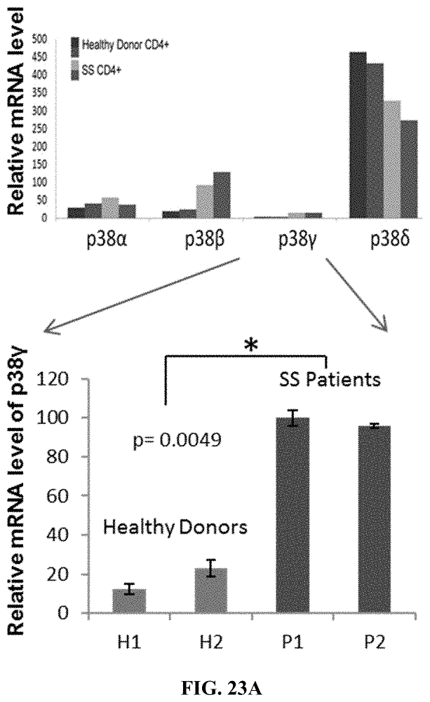

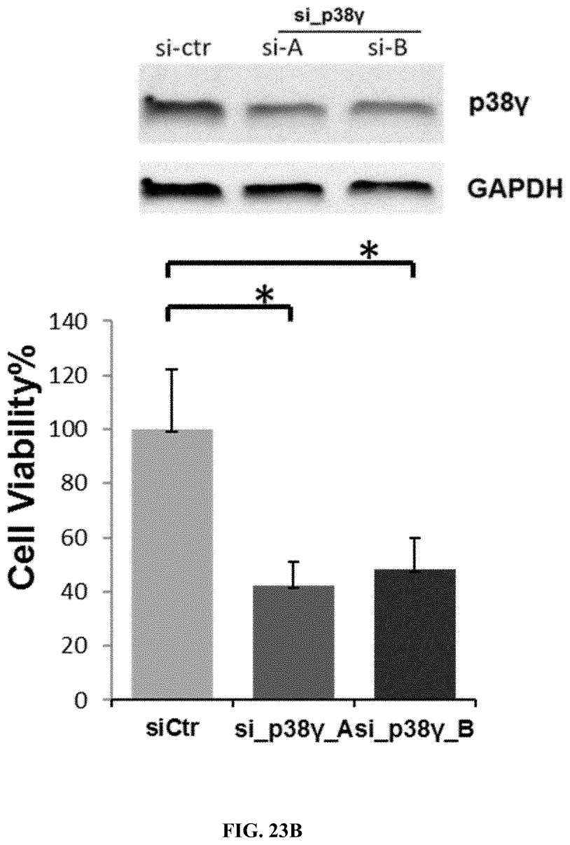

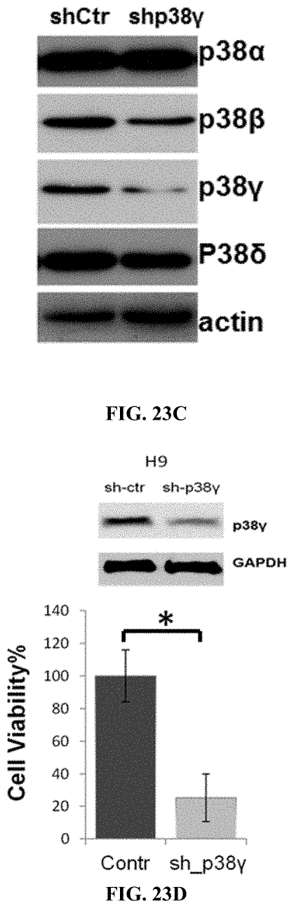

Given that we previously described a role for p38β in CTCL [5], we evaluated the role of other p38 isoforms in CTCL. To examine expression of the p38 isoforms in CTCL, we first analyzed a publicly available RNA-seq database [phs000725] for Sézary Syndrome (SS) [26] and microarray databases [GSE17601, n=32 for SS; GSE12902, n=22 for mycosis fungoides (MF)] [20]. We found that mRNA expression of p38γ as well as p38s from both RNA-seq (FIG. 18A) and microarray analysis (FIG. 3B) was significantly increased while that of p38a was significantly reduced in both CTCL SS patients, compared to healthy donors and primary CD4⁺ T cells [GSE19069, n=8]. It is known that although p38α, p38β, and p38δ are expressed in normal healthy T cells, p38γ is undetectable [76]. We used qRT-PCR to show that p38γ mRNA level, despite being lowest among other p38 isoforms (FIG. 23), was significantly elevated in CD4⁺ T cells from two SS patients compared to that of two healthy donors (Two-sample T-test, p value=0.0049, FIG. 3A).

We used western blot to evaluate protein expression of p38γ in a CTCL SS patient sample and CTCL cell lines (Hut78, H9, and HH). We showed elevated protein expression of p38γ in one SS patient PBMC sample and two CTCL cell lines compared to (PBMC) cells from three normal healthy donors, who did not express p38γ (FIG. 18B). p38α protein levels were reduced, and those of p38δ remain unchanged. p38β protein expression (FIG. 18B), as well as mRNA level (FIG. 3B, FIG. 23), were elevated in CTCL cells compared to healthy donor cells, which is consistent with our previous finding [5].

Given that p38γ is elevated in CTCL cells, we investigated the effects of p38γ expression on CTCL viability by silencing p38γ via siRNA or shRNA, using Hut78 cells as a model cell line for CTCL. We showed that both transient transfection with two validated p38γ siRNAs (FIG. 23B) as well as transduction with lentiviral shRNA against p38γ significantly reduced viability compared to the scrambled control (p<0.05, FIG. 18C). We used western blot to verify the reduced protein expression of p38γ (FIG. 18C), and that knockdown is specific to p38γ compared to other p38 isoforms (FIG. 23C). We also confirmed that shRNA silencing of p38γ significantly reduced viability in another SS cell line, H9 with similar effects (FIG. 23D).

Identification of a p38γ Inhibitor with Unique Features in CTCL

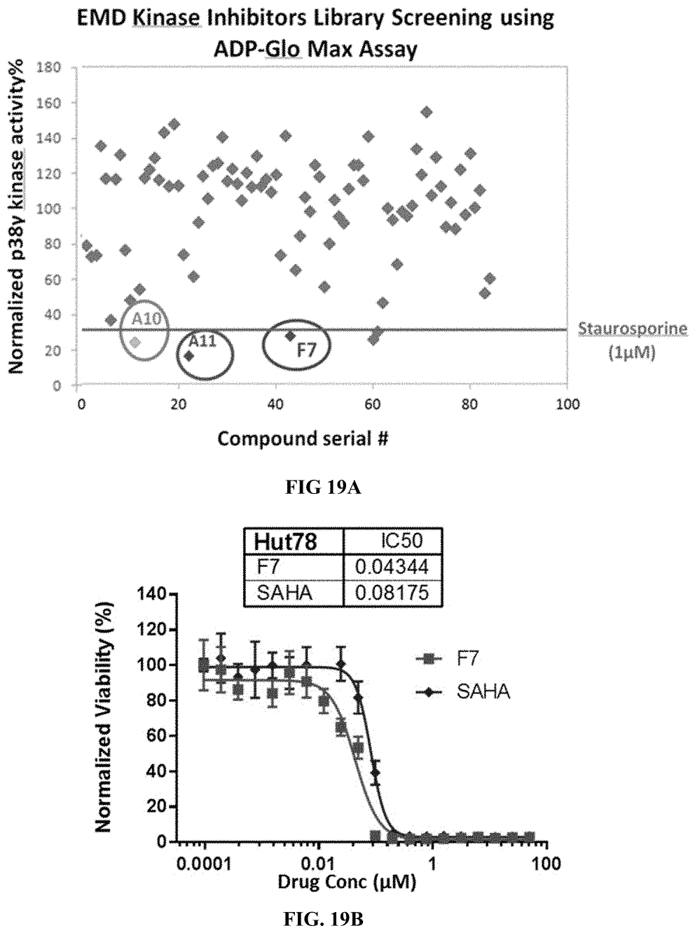

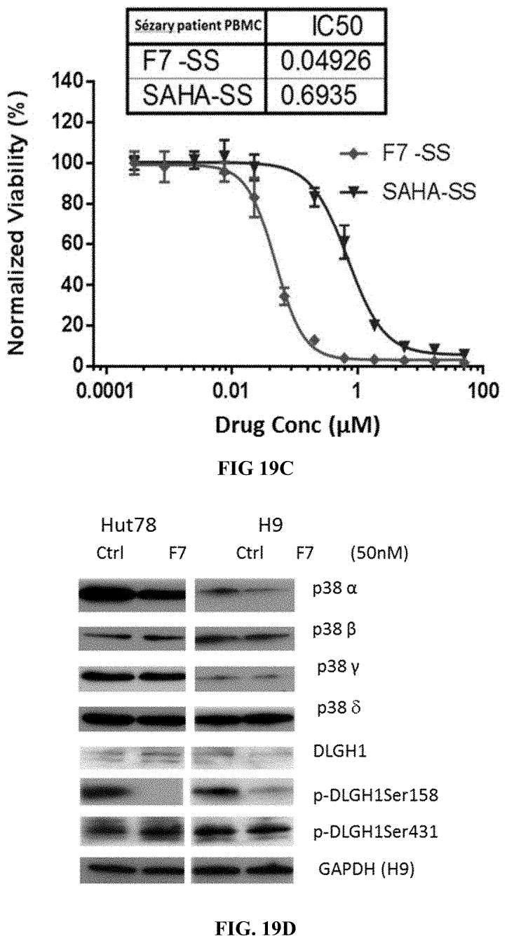

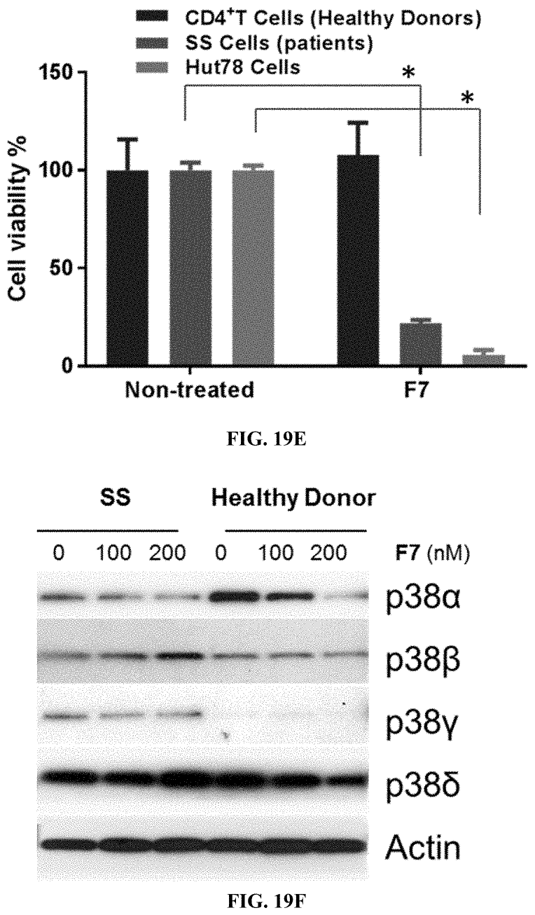

Given that gene silencing of p38γ reduced viability of CTCL cells and few p38γ inhibitors are presently known, identifying novel, potent, and specific inhibitors of p38γ has great potential for clinical application in CTCL. We used high-throughput screening (HTS) of a commercially available kinase inhibitor library (EMD Biosciences), and identified three candidates with activity against p38γ: A10 (also known as Ro3306), A11 (known as PHA-767491), and F7/PIK75 (FIG. 19A).

To test whether the selected compounds caused death in cancer cell lines, we first profiled these three hits across the NCI60 cell line collection. In general, PIK75 was far more potent than A10 or A11 in many types of cancer cells, including solid tumor and hematopoietic cell lines (Table 3). Table 3 is IC₅₀ (μM) of p38γ inhibitors F7, A10, and A11 in NCI60 cell line panel. CELLTITERGLO Cell Viability Assay was used to measure viability and IC₅₀ was determined in NCI 60 tumor cell lines. Therefore, we centered our subsequent studies on PIK75. PIK75 also showed a dose-dependent reduction of proliferation in CTCL cell lines (Hut78, HH, and H9) and SS cells from patient PBMCs, with a similar IC₅₀ range (FIGS. 19B-19C, Table 4). Table 4 shows determination of IC₅₀ (μM) in Hut78, H9, and HH cells treated with F7/PIK75, Pirfenidone (Pir), or SAHA for 72 h. *p<0.05. We compared the potency of PIK75 and SAHA (Vorinostat, as a hydroxymate HDAC inhibitor), a FDA-approved drug for CTCL. PIK75 exceeded SAHA in the efficacy of killing CTCL cells (FIGS. 19B-19C, Table 4). PIK75 also exceeds pirfenidone greatly in both p38γ kinase activity and cell toxicity to the CTCL cells. The IC₅₀ of pirfenidone against p38γ kinase activity is over 125 μM and the IC₅₀ of pirfenidone in Hut78 cells is over 125 μM (FIGS. 24A-24B, and Table 4).

TABLE 3
F7 effects on the NCI 60 cells

| Cells | F7/PIK75 | A10 | A11 | Disease | Cells | F7/PIK75 | A10 | A11 | Disease |
|---|---|---|---|---|---|---|---|---|---|
| K-562 | 0.416 | >10 | >10 | CML | UACC257 | 2.32 | >10 | >10 | Melanoma |
| RPMI-8226 | 0.077 | 9 | >10 | MM | LOX IMVI | 0.073 | 4.4 | 3.58 | Melanoma |
| SR | <0.001 | 0.372 | 0.286 | Leukemia | SK-MEL-5 | >10 | >10 | >10 | Melanoma |
| HL60 | 0.005 | 1.14 | 0.247 | AML | MALME-3M | 0.116 | >10 | 3.05 | Melanoma |
| MOLT-4 | 0.527 | 5.2 | 6.98 | ALL | UACC-62 | 0.362 | >10 | 6.12 | Melanoma |
| CCRF-CEM | 3.72 | 2.5 | 2.5 | Leukemia | SK-MEL-2 | 0.317 | >10 | >10 | Melanoma |
| NCI-H226 | 8.39 | >10 | >10 | Lung | M14 | 0.195 | 10 | 3.33 | Melanoma |
| NCI-H460 | 0.01 | >10 | 10 | Lung | MDA-MB-435 | >10 | 6.6 | >10 | Melanoma |
| HOP-92 | 0.094 | 6.04 | 6.4 | Lung | SK-MEL-28 | 1.69 | >10 | 9.6 | Melanoma |
| NCI-H552 | >10 | >10 | >10 | Lung | T-47D | >10 | 6.19 | >10 | Breast |
| NCI-H322M | 1.5 | >10 | >10 | Lung | BT-S49 | <0.001 | 4.78 | 0.705 | Breast |
| NCI-H23 | >10 | >10 | >10 | Lung | MDA-MB-231 | 0.466 | >10 | >10 | Breast |
| HOP-62 | 0.008 | >10 | 9.94 | Lung | MCF7 | 0.238 | 10 | 3.77 | Breast |
| EKVX | 1.22 | >10 | >10 | Lung | HS 578T | 0.586 | 5.83 | >10 | Breast |
| A549 | 2.66 | >10 | >10 | Lung | MDA-MB-468 | 0.103 | 6.97 | 1.77 | Breast |
| HCT-15 | 0.008 | 3.84 | 2.54 | Colon | U251 | 0.253 | 1.92 | 4.57 | CNS |
| KM12 | 0.006 | >10 | 2.28 | Colon | SF-539 | 0.177 | 2.55 | 3.68 | CNS |
| HCT-116 | 0.001 | 3.2 | 1.26 | Colon | SF-268 | 2.23 | >10 | >10 | CNS |
| HCC-2998 | >10 | >10 | >10 | Colon | SF-295 | 0.141 | >10 | 7.64 | CNS |
| Colo 205 | 2.18 | >10 | >10 | Colon | SNB-19 | 0.385 | >10 | >10 | CNS |
| SW-620 | 0.074 | >10 | 3.93 | Colon | SNB-75 | 0.435 | >10 | 6.14 | CNS |
| HT29 | 0.087 | >10 | 5.04 | Colon | UO31 | 0.115 | >10 | 4.03 | Renal |
| NCI/ADR-RES | 0.024 | 3.67 | 1.44 | Ovarian | 786-0 | 0.008 | 7.77 | 9.3 | Renal |
| OVCAR-3 | 0.071 | >10 | 2.52 | Ovarian | RXF 393 | 0.08 | >10 | 3.47 | Renal |
| SKOV3 | 0.291 | >10 | >10 | Ovarian | ACHN | 0.092 | 4.46 | >10 | Renal |
| IGR-OV1 | 0.251 | >10 | >10 | Ovarian | A498 | 0.027 | >10 | >10 | Renal |
| OVCAR-8 | 0.059 | >10 | 3.6 | Ovarian | CAKI-1 | 0.35 | >10 | >10 | Renal |
| OVCAR-4 | 0.403 | >10 | >10 | Ovarian | SN12C | 0.468 | >10 | 10 | Renal |
| OVCAR-5 | 0.04 | >10 | 5.31 | Ovarian | TK-10 | 0.805 | >10 | >10 | Renal |
| PC-3 | 0.02 | >10 | 9.18 | Prostate | DU-145 | 0.001 | 1.38 | 2.26 | Prostate |

TABLE 4
IC₅₀ (μM)

| | Hut78 | H9 | HH |
|---|---|---|---|
| F7 | 0.042 +/− 0.003 | 0.046 +/− 0.02 | 0.034 +/− 0.005 |
| Pir | >125 | >125 | >125 |
| SAHA | 0.097 +/− 0.018 | 0.255 +/− 0.081 | 0.369 +/− 0.08 |

To determine whether F7/PIK75 was selective for CTCL over healthy cells, we tested its toxicity in SS patient cells (n=2) and CTCL cell lines, as well as normal CD4⁺ T cells from healthy donors. We showed that 100 nM F7/PIK75 significantly reduced viability of patient SS and Hut78 cells, but spared healthy CD4⁺ T cells (Two-sample T-test, p<0.05, FIG. 19E). Cell death caused by F7/PIK75 was via apoptosis in both CTCL cell lines and the SS patient sample (Table 5). Table 5 indicates that F7/PIK75 caused apoptosis in an SS patient sample and Hut78 cells. Annexin V/PI staining followed by flow cytometry analysis in PBMCs isolated from an SS patient or Hut78 CTCL cell line treated with either DMSO control or 200 nM F7/PIK75 for 24 h. Data presented are one experiment in triplicate. Furthermore, western blot results of CD4⁺ cells from both healthy donors and SS patients indicated that p38γ remained undetectable in healthy donor cells, but elevated in SS (FIG. 19F). F7/PIK75 inhibited phosphorylation of DLGH1 at Ser158 (pDLGH1-Ser158, the specific site of p38γ phosphorylation) [15]; in SS cells (FIG. 24C) and in Hut78 and H9 cells (FIG. 19D). This suggests that F7/PIK75 inhibits the ability of the p38γ kinase to phosphorylate its substrates (e.g., DLGH1) in CTCL cells.

TABLE 5

| | | Live | | PI |
|---|---|---|---|---|
| PBMC | Percentage | cells | AnnexV_PI | Stained |
| SS | Control | 87.3 | 4.26 | 4.78 |
| | F7 (200 nM) | 42.797 | 17.8 | 35.2 |
| Hut78 | Control | 82.9 | 4.81 | 5.38 |
| | F7 (200 nM) | 44.48 | 25.5 | 29.53 |

F7/PIK75 Inhibits p38γ Kinase Activity In Vitro and is ATP Dose-Dependent

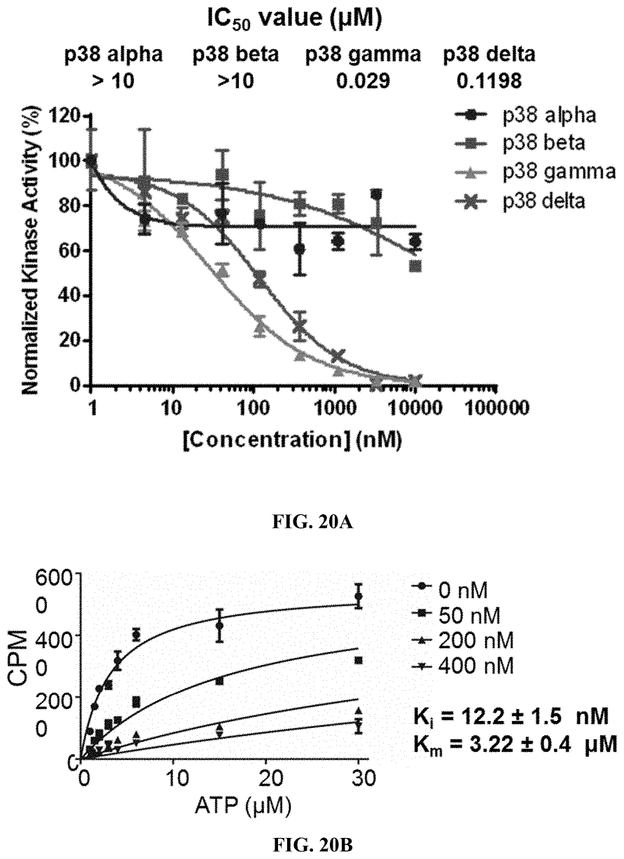

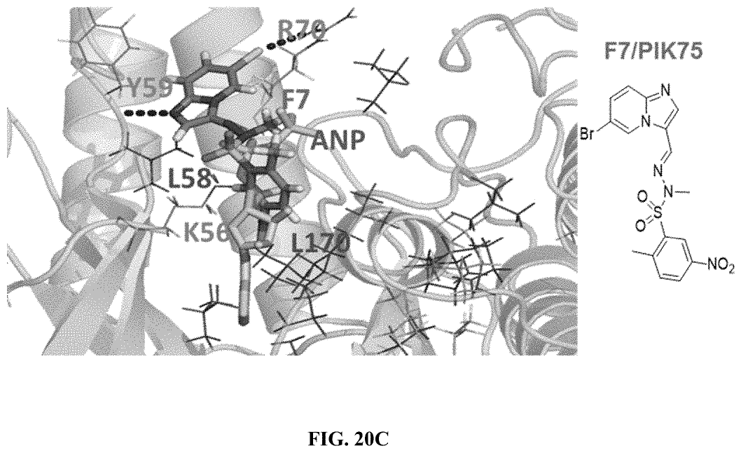

To demonstrate that F7/PIK75 specifically inhibits the kinase activity of p38γ among other p38 isoforms, we performed in vitro ADP-Glo kinase assays. We observed a dose-dependent inhibition of p38γ as well as p38δ kinase activity, but not p38α and p38β, by F7/PIK75 (FIG. 20A). We tested the F7/PIK75 compound from different sources (data not shown) and observed a consistent effect with the calculated IC₅₀ values ranging from 4.1-29 nM for p38γ and 32.1 nM-119.98 nM for p38δ, with p38γ being ˜5-fold more sensitive than p38δ. We further confirmed these data using radiometric kinase assays. At 50 nM and 200 nM, F7/PIK75 showed a 22.5% and 59% inhibitory effect, respectively, on p38γ, compared to 5.4% and 34.9% on p38δ; neither concentration inhibited p38α or β (Table 6). Table 6 shows effect of compound F7/PIK75 on p38 isoforms. Four isoforms of p38 kinase were measured by SignalChem (Canada) using radiometric in vitro kinase assays in two concentrations of F7/PIK75, 50 nM or 200 nM respectively. Both assays suggest that the specific effects of F7/PIK75 on p38γ relative to p38δ are more pronounced at lower doses.

TABLE 6

| | % Inhib. | % Inhib. |
|---|---|---|
| p38 | 50 nM | 200 nM |
| p38 α | −1.92% | −0.48685% |
| p38 β | −21.26% | −24.845% |
| p38 δ | 5.35% | 34.91% |
| p38 γ | 22.45% | 58.98% |

To confirm that F7/PIK75 binds to the ATP-binding pocket of p38γ, we performed an in vitro kinase assay using increasing concentrations of ATP in the presence of p38γ and a synthetic peptide substrate. We performed time-resolved fluorescence energy transfer (TR-FRET) using a peptide substrate, ULight™-4E-BP1, derived from the p38γ phosphorylation site of 4E-binding protein 1 (4E-BP1) [91]. We determined the ATP Kₘ and inhibitor Kᵢ to be 3.2±0.4 μM and 12.21±1.5 nM, respectively (FIG. 20B). The ATP Kₘ for p38γ is nearly identical to that described in the product manual for the peptide substrate. The Kᵢ value indicates strong binding and inhibition of p38γ by F7/PIK75. Together, the enzyme kinetics indicate that F7/PIK75 inhibits p38γ by a competitive mechanism, i.e., competing with ATP binding to the enzyme.

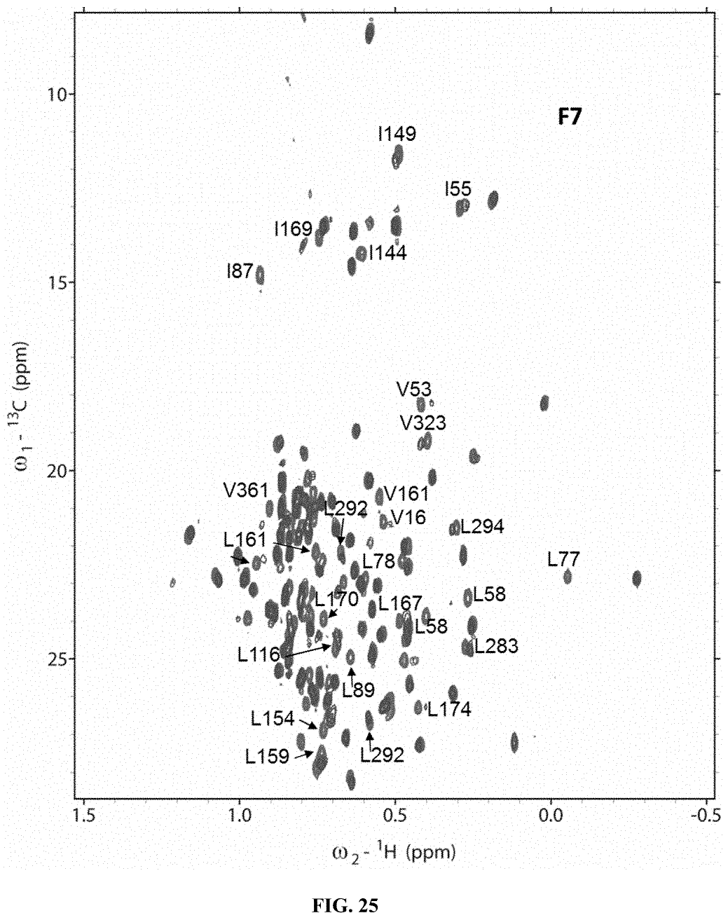

We used NMR methods to identify the F7/PIK75 binding site on p38γ. F7/PIK75 induced extensive NMR chemical shift perturbations (CSPs, calculated as described in Methods) and line broadening in the 1H-13C HMQC spectrum (FIG. 25). We used our in-house-developed All-Around Docking (AAD) methodology [92] to model PIK75 binding to p38γ. The 3-D structural binding model predicted that PIK75 binds to the ATP-binding pocket of p38γ protein through three hydrogen bonds at K56, Y59, and R70 in the ATP pocket (displayed as dots in FIG. 20C) based on the binding score calculation (Glide XP docking score of −8.0 kcal/mol). The residues that showed line broadening effects of NMR data are indicated in red in the structure of p38γ (FIG. 20C); residues that showed significant NMR CSPs are indicated in green. Because the most significant CSP occurs around the ATP-binding pocket, these results, combined with the enzyme kinetic studies, indicate that PIK75 binds to the ATP-binding pocket of p38γ.

F7/PIK75 Targets p38γ Kinase Activity In Vivo and Reduces Xenograft Tumors

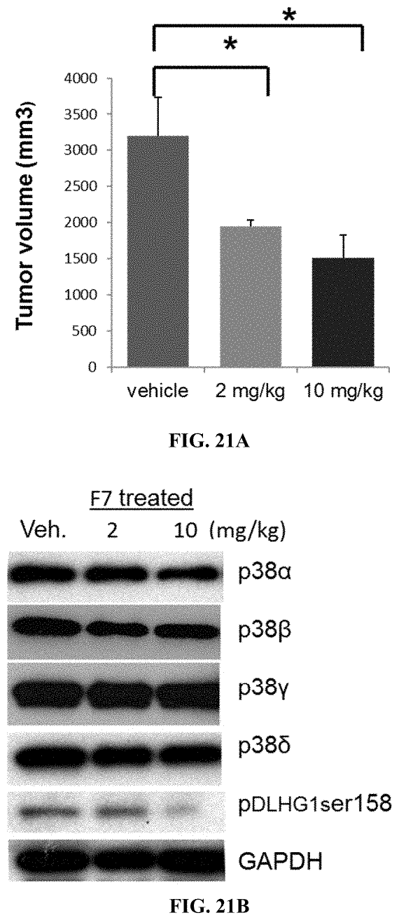

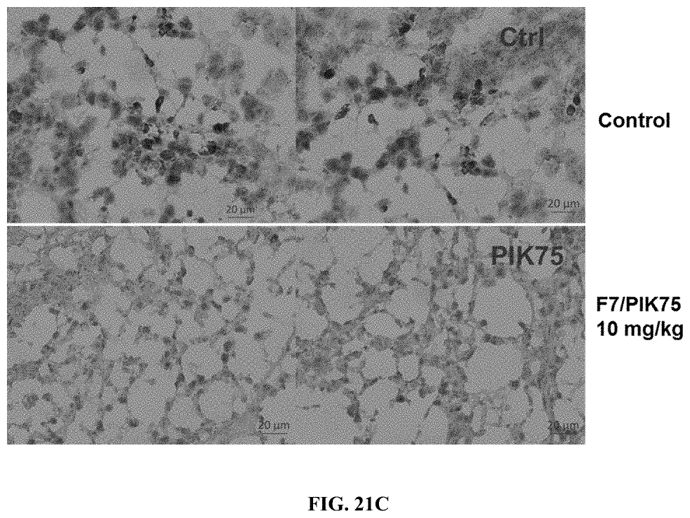

Because the effects of kinase inhibition in vitro can differ from kinase inhibition in vivo due to a number of factors, such as retention in the cytosol (or other compartment of the cells) due to phosphorylation of kinase substrates [13], we further investigated the effects of PIK75 in a CTCL xenograft model. We treated mice harboring Hut78 xenografts with PIK75 or vehicle control by intraperitoneal injection, and harvested the tumors. We showed that PIK75 significantly inhibited tumor growth at 2 mg/kg (p=0.015) and 10 mg/kg (p=0.025) (FIG. 21A). We then monitored p38γ activity using pDLGH1-Ser158. We demonstrated that, pDLGH1-Ser158 was significantly reduced in xenografts when mice were treated with PIK75 at 10 mg/kg; in contrast, untreated control mice xenografts expressed pDLGH1-Ser158 (FIG. 21B).

To confirm that PIK75 inhibited p38γ kinase activity in vivo, we also performed immunohistochemistry on tumor tissues from xenografted mice, and demonstrated that pDLGH1-Ser158 staining was dramatically reduced in PIK75-treated tumors compared to controls (FIG. 21C). Taken together, our data indicate that PIK75 reduces cell growth and inhibits p38γ kinase activity both in vitro and in vivo.

PIK75 Inhibits Multiple Kinases Including p38γ and PI3K p110a

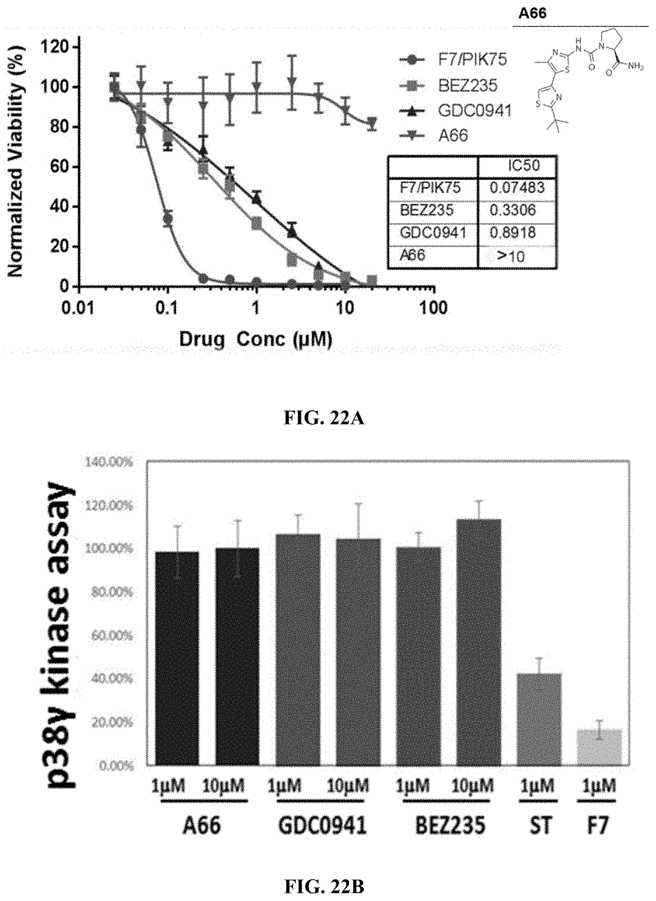

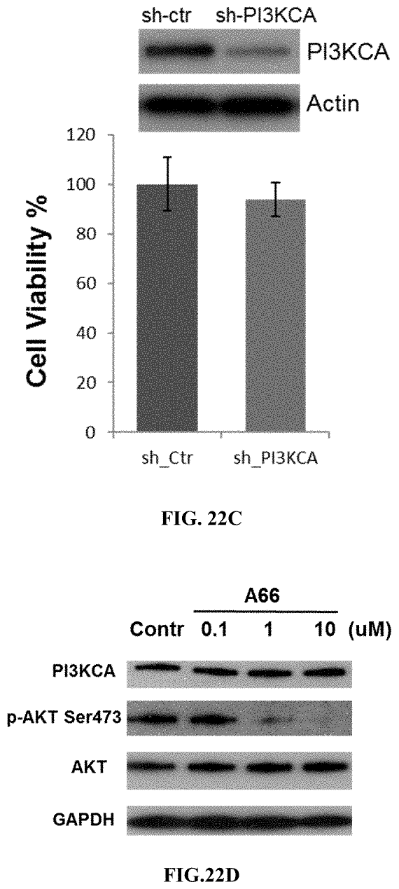

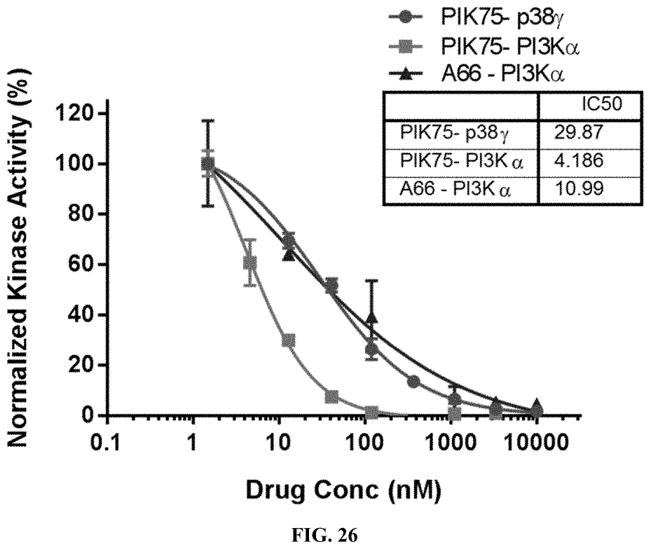

Because PIK75 was originally identified as a PI3K p110α inhibitor with an IC₅₀ of 5.8 nM [79], we measured the IC₅₀ of both PI3K p110α and p38γ; we showed IC₅₀s of 4.1 and 30 nM respectively (FIG. 26). To determine whether PI3K p110α targeting by F7/PIK75 contributes to cell death in CTCL, we first used lentiviral shRNA to silence PI3K p110α in Hut78 cells. To our surprise, unlike knockdown of p38γ (FIG. 18C, and FIGS. 23D-23E), cell viability was not affected by gene silencing of PI3K p110α isoform (FIG. 22C). This suggests that in CTCL, F7/PIK75 causes cell death by targeting p38γ rather than targeting PI3K p110α. To further confirm this, we evaluated cell toxicity effects in CTCL cells with three other potent PI3K p110α specific inhibitors, A66, GDC0941, and BEZ235. GDC-0941 (Pictilisib, which also targets mTOR) is a Pan-PI3K inhibitor [93] and BEZ 235 (Dactolisib, which also targets ATR)) is a dual inhibitor of PI3K and mTOR inhibitor, which blocks the dysfunctional activation of the PI3K pathway [94]. A66 is a unique p110α-specific inhibitor, which has over 100-fold selectivity over other class-I PI3K isoforms (IC₅₀ of 11 nM, FIG. 26). These inhibitors have low IC₅₀s (Table 7) against PI3K p110α in cell-free assays but no inhibitory kinase activities against p38γ (FIG. 22B). Table 7 shows comparison of IC₅₀ for inhibition of PI3K kinase activity by three PI3K-specific inhibitors (BEZ235, GDC-0941, or A66) and F7/PIK75. This table is summarized from publically available databases (summarized from the companies of Selleck and Apexbio). F7/PIK75 had the most potent inhibitory effect on Hut78 cells compared with the other three PI3K inhibitors (FIG. 22A). GDC0941 and BEZ235 showed only modest reduction of cell viability. Western blot analysis indicated that A66 has dose-dependent inhibition of phosphorylation of ATK at ser473 in Hut78 cells (4 hr treatment, FIG. 22D), but has no influence to the Hut78 cells viability up to ˜10 μM in 72 hr (FIG. 22A). Consistent with our shRNA results, PI3K p110α inhibition alone does not contribute to CTCL cell toxicity, whereas p38γ inhibition causes cell death in CTCL.

TABLE 7

| | |
|---|---|
| PI3K | IC₅₀ of PI3K Isoforms* |

| inhibitors | p110α | p110β | p110γ | p110δ |
|---|---|---|---|---|
| BEZ235 | 4 nM | 75 nM | 5 nM | 7 nM |
| GDC-0941 | 3 nM | 33 nM | 3 nM | 75 nM |
| F7(PIK75) | 5.8 nM | 1.3 μM | 76 nM | 0.51 μM |
| A66 | 32 nM | | | |

*summarized from Selleck.com and Apexbic.com

Discussion

Several studies have identified p38γ inhibitors as promising therapeutic agents for many tumor types; such as colon [22], prostate [77], esophageal [78] and breast cancers [81]. In addition to activity against CTCL, the p38γ inhibitor we identified, F7/PIK75, also shows activity against cells derived from several types of cancers, including prostate, ovarian, and breast cancers (Table 3). In comparison, the known p38γ inhibitor pirfenidone (FIGS. 24A-24B) had an IC50 for cell killing>125 μM on the CTCL cells and for p38γ kinase activity>125 μM, which is minimal compared to the effects of PIK75 on CTCL cells. Our data further revealed many unique features of PIK75, which warrant further study of PIK75 as a potential anticancer treatment, particularly for CTCL.

Here, we report that F7/PIK75, a multiple kinase inhibitor, works in part through p38γ inhibition, either directly or indirectly, the mechanism of which may be through targeting the TCR signaling pathway in CTCL. It binds and inhibits p38γ at nanomolar concentrations (FIGS. 20A-20C); more importantly, it selectively killed CTCL patient sample cells but spared healthy CD4⁺ T cells (FIGS. 19E-19F). A likely explanation for this is that CTCL cancer cells are more vulnerable to p38γ inhibitors, as p38γ is elevated in CTCL, but is not expressed in normal T cells.

PIK75 also targets other kinases [83] including PI3K p110α. Our data show it inhibits in vitro activity of p38γ at a nanomolar range (IC₅₀ values of 29 nM, FIG. 20A) which is the same magnitude as that of PI3K p110α (4.1 nM, FIG. 26). A66 shows potent inhibitory effects on in vitro kinase activity of PI3K p110α (IC₅₀ values of 11 nM, FIG. 26), but doesn't inhibit in vitro activity of p38γ (FIG. 22B). Surprisingly, silencing of PI3K p110α via shRNA or kinase inhibition via the specific inhibitor A66 does not affect cell viability in CTCL (FIG. 22A), suggesting that p38γ plays a more critical role than PI3K p110α in CTCL, and that cell death caused by PIK75 was due to targeting p38γ rather than PI3K p110a. Yet, mechanism of which specifically inhibition of PI3Kp110a by A66 results in blocking phosphorylation of Akt at ser473 (FIG. 22D) remains further investigation. It is worth noting that the p110δ isoform of PI3K not the p110α isoform predominantly expressed in T-cells and that p110δ plays more important roles in T-cell proliferation [84]. However, p110δ is less sensitive to PIK75 (IC₅₀=510 nM) than p110α isoforms of PI3K (IC₅₀=5.8 nM) (Table 7). The PI3K pathway is known to be one of the most frequently mutated pathways in human cancer and is critical for driving cancer cell progression [85]. A recent study [111] illustrated that a PI3K-dependent but AKT-independent pathway is just as important as the PI3K/AKT-dependent pathway in promoting carcinogenesis and progression. PI3K/AKT dependence provides a therapeutic target for cancers [86] which is also true in CTCL [87]. Our NanoString RNA analysis data followed by IPA analysis demonstrates that the activation Z score of PI3K signaling (both AKT-dependent and AKT-independent) is −3.108, whereas that of AKT-dependent PI3K signaling is −0.63, implicating that the AKT-independent PI3K pathway is much more affected by F7/PIK75 (50 nM) in CTCL cells than that of AKT-dependent PI3K pathway (Table 8). Table 8 is NanoString RNA and IPA data analysis indicated top 9 pathways in Hut78 cells altered by F7/PIK75 treatment (50 nM for 10 h) including 2 upregulated pathways and 7 downregulated pathways.

TABLE 8

| | Exp fold | Activation | |
|---|---|---|---|
| Pathway Name | change | z-score | p-value |
| NF-kB signaling | −0.633 | −3.623 | 5.76E−90 |
| Th2 pathway | −0.687 | −2.236 | 7.96E−87 |
| Th1 pathway | −0.719 | −3.533 | 6.95E−85 |
| IL-6 Signaling | −0.717 | −3.109 | 1.80E−79 |
| Role of NFAT in regulation | −0.53 | −3.962 | 7.96E−67 |
| of the immune response | | | |
| PI3K signaling | −0.586 | −3.108 | 1.04E−55 |
| PTEN | 0.588 | 1.25 | 3.21E_52 |
| PI3KI/AKT signaling | −0.54 | −0.63 | 1.11E−46 |
| Wnt/B-catenin signaling | 0.426 | 1.152 | 5.70E−41 |

F7/PIK75 targets other kinases in vitro as well, including GSK3 and PKCβ [83], potential therapeutic targets we have previously studied in CTCL cells. The combined inhibition of GSK3 and PKCβ synergistically kills CTCL cells through the p38-TCR pathway [5], although the mechanism remains unclear. However, we observed reduction of both p38γ and p38β at transcriptional level in this combined inhibition, which may contribute to the cell death. In addition, p38γ has many substrates, which may result in different signaling pathways in cancers, as our NanoString RNA analysis has revealed (Table 8).

F7/PIK75 is known to have inhibitory activity against other kinases such as the CMGC kinase family, an evolutionarily conserved kinase group across all eukaryotes, including cyclin-dependent kinases (CDKs), mitogen-activated protein kinases (MAP kinases), glycogen synthase kinases (GSK) and CDK-like kinases [83]. Therefore, we do not rule out possibility of other kinase involvement in cell killing in CTCL by F7/PIK75. However, we argue that its multi-targeting nature does not diminish the importance of its activities on p38γ in CTCL. In fact, PI3K p110α is more sensitive to F7/PIK75 than that of p38γ in cell free based analysis (FIG. 26), our shRNA data indicate it is p38γ inhibition (FIG. 18C), but not PI3K p110α inhibition (FIGS. 22A and 22C) contributes to the Hut78 cell death in CTCL. Also, we have previously demonstrated that PKCβ, another target of F7/PIK75, its inhibition in CTCL increases apoptosis in vitro [87], but it was not effective in in vivo patient's preclinical and clinical studies [89]. In contrast, F7/PIK75 exhibits a greater clinical potential for drug development in CTCL preclinical studies, as shown through its in vivo mouse studies reducing the size of Hut78 cells xenografts and targeting p38γ kinase activity.

In addition, in our animal studies we did not observe any toxic effects, especially notable given the much lower dosages required in comparison to Pirfenidone which requires 500 mg/kg dosages [81, 82]. A dosage as low as 10 mg/kg of F7/PIK75 every two days, we showed significantly inhibited tumor growth in xenograft mice within 8 days (FIG. 21A), and reduced phosphorylation of the p38γ substrate DLGH1 (FIG. 21C). This suggests that F7/PIK75 inhibition of p38γ kinase activity in CTCL plays a role in the reduction of tumor size in xenograft mice, and marks F7/PIK75 as a potential lead compound for optimizing p38γ-specific inhibition in the future. Nevertheless, there are potential precautions that clinical application of a p38γ inhibitor could have cytotoxic effects on skeletal muscles, where p38γ is highly expressed [88]. However, we argue that in other cell types, the mechanistic role of p38γ, e.g., non-kinase activities [80] differs dramatically from its role that in CTCL in which unique TCR signaling pathway is being targeted. Further studies are needed to address F7/PIK75 synergistic effects on p38γ with other kinases or its indirect inhibition upstream of the MAPK pathway in CTCL.

Materials & Methods

Compound, Samples, and Cell Culture: PIKP110a inhibitors, PIK75, A66, GDC0941, and BEZ235 are from Selleck. Isolation of PBMCs or CD4⁺T cells from both patients and healthy donors and culture of CTCL cell lines (Hut78, HH, and H9) were performed as previously described [5].

qRT-PCR (real-time quantitative reverse transcription PCR): Total RNA was generated using TRIZOL reagent (Life Technologies, Carlsbad, Calif.) followed by double DNase treatment and column purification using Qiagen RNeasy Clean-up (Qiagen, Germantown, Md.). The Invitrogen SUPERSCRIPT III First-Strand Synthesis System was used for cDNA synthesis and TaqMan 5700 Sequence Detection System (Applied Biosystems, Foster City, Calif.) were used for qRT-PCR. All PCR reactions were run in triplicate. The amplified transcripts were quantified using the comparative CT method as described [90]. cDNA and primers for MAPK11, 12, 13, and 14 and GAPDH (Table 9) were added to SYBR GREEN PCR Master Mix (SYBR Green I Dye, AMPLITAQ® DNA polymerase, dNTPs with dUTP and optimal buffer components; Applied Biosystems) and subjected to PCR amplification (1 cycle at 95° C. for 3 min; 35 cycles at 95° C. for 15 s and 55° C. for 30 s; 1 cycle at 50° C. for 2 min).

TABLE 9
Primers for four p38 isoforms and GAPDH genes
Primers used for the real time qRT-PCR experiments

| Genes | Sequences | SEQ ID NO |
|---|---|---|
| MAPK11 | ForwardCCCGGACATATATCCAGTCC | SEQ ID NO: 1 |
| | ReverseTCACTGCTCAATCTCCAGG | SEQ ID NO: 2 |
| MAPK12 | FOrwardGCCCATCCCTACTTCGAGTC | SEQ ID NO: 3 |
| | ReverseCTTCACAGAGGCGTCTCCTT | SEQ ID NO: 4 |
| MAPK13 | ForwardGGCAGTTTAACGTGGCCTGTTA | SEQ ID NO: 5 |
| | ReverseACAGTGGATGAATGGAAGCAGC | SEQ ID NO: 5 |
| MAPK14 | ForwardGCCGAGCTGTTGACTGGAAG | SEQ ID NO: 7 |
| | ReverseGGAGGTCCCTGCTTTCAAAGG | SEQ ID NO; 8 |
| GAPDH | ForwardCCCGGACATATATCCAGTCC | SEQ ID NO: 9 |
| | ReverseTCACTGCTCAATCTCCAGG | SEQ ID NO: 10 |

Viability assays using trypan blue exclusion and CellTiterGlo Cell Viability Assay: Cell viability was calculated by diluting cell suspensions 1:1 in 0.4% TRYPAN BLUE solution (Sigma, St. Louis, Mo.) and counting the number of viable cells using a TC20™ automated cell counter (Bio-Rad, Irvine, Calif.) that automatically excludes the number of non-viable cells stained with trypan blue per total cells. CELLTITERGLO CELL Viability Assay (Promega, Fitchburg, Wis.) method was used as described previously. All data points are an average of triplicate experiments.

siRNA: For p38γ silencing, Hut78 cells were transfected using X-TREMEGENE transfection reagent (Roche, Indianapolis, Ind.) and 100 μmol/L of specific human siRNAs against p38γ (GenScript, Piscataway, N.J.): si_p38gA (Cat #SR304235A) sequences-5′ GGAAGCGUGUUACUUACAAAG-AGGT3′-; si_p38gB (Cat #SR304235B) sequences-5′ CGGAGAUGAUCACAGGCAAGAC-GCT3′-.

shRNA: MISSION® shRNA Lentiviral Transduction Particles (company-validated) expressing 5 shRNAs in pLKO.1-puro shRNA vector that targets 4 exons of the human p38γ gene (MAPK12) and scrambled Transduction Particles (pLKO.1-puro shRNA vector-only) were purchased from Sigma. Four shRNA lentiviral particles each contain unique 29mer target Human PIK3CA-specific shRNA (Cat. #TL310428V) and one Lenti shRNA scramble control particles (Cat. #TR30021V) are from OnGene Technologies, Inc. Hut78 and H9 cells growing exponentially (70-80% confluent) were seeded into 6-well plates (2×10⁵ cells/well) before transduction. Viral transduction efficiencies were improved by adding polybrene (hexadimethrine bromide; Sigma) to a final concentration of 8-10 μg/ml. Hut78 and H9 cells were transduced at a multiplicity of infection of 2. Hut78 and H9 cells were incubated overnight with the medium containing lentiviral particles and polybrene; then the medium containing lentiviral particles was removed from wells and replaced with fresh medium. Cells were collected on day 5 after transduction before RNA extraction and protein isolation.

In vitro kinase assay (ADP-GLO): We identified p38γ inhibitors using in vitro kinase assay by screening a library of kinase inhibitors: The library consists of 244 compounds on three plates (EMD Cat #539744, #539745, and #539746) that are mostly ATP mimics. All compounds are cell-permeable, reversible, and well-characterized. For the biochemical screening, kinase assays in vitro were performed using ADP-GLO kit (Promega) following manufactures instructions. All data points are average of triplicate experiments unless stated otherwise, and all compounds were tested and show no inhibition of luciferase activities when using the ADP-Glo kit. Briefly, for in vitro assay experiments, human recombinant p38 α, β, γ or δ proteins (active full-length) were from SIGNALCHEM, and followed the protocol of the company. The p38 kinase was preincubated with compound F7 in a dose-dependent manner for 10 min before synthetic peptide substrates (IPTTPITTTYFFFKKK) were added at final concentration of 0.2 μg/μL, followed by addition of ATP. Then, ADP-GLO REAGENT was incubated in the mixture at room temperature for 40 min, followed by incubation of Kinase Detection Reagent for another 30 min. IC₅₀ values were determined using CALCUSYN software (Biosoft, Cambridge, United Kingdom).

Radiometric in vitro kinase assay: Radiometric in vitro kinase assay was performed by SIGNALCHEM (Richmond, BC, Canada) using F7 at two concentrations (50 nM and 200 nM) according to the company's proprietary research methodologies.

Enzyme kinetics: The inhibition mechanism of the compound was measured using the TR-FRET method [117]. Assays were conducted in a 384-well black round bottom plate in kinase reaction buffer (50 mM HEPES, pH 7.5; 10 mM MgCl₂; 1 mM EGTA, 100 μM Na₃VO₄; 0.01% Tween-20; 0.5 mM DTT). p38γ kinase (700 ng/ml) was mixed with ULIGHT™-4E-BP1 peptide (50 nM, PerkinElmer, Waltham, Mass.) and varying concentrations of ATP (1, 1.5, 2, 3, 4, 6, 15, 30 μM) and F7 (0, 50, 200, 400 nM). Time course data were collected by stopping the kinase reaction at different times by adding detection buffer containing Europium-anti-phospho-4E-BP1 antibody (4 nM, PerkinElmer). Fluorescence signals were measured at 665 nm with a 50 μs delay after excitation at 320 nm using a CLARIOSTAR microplate reader (BMG LABTECH, Ortenberg, Germany). The signal ratio at 665/620 nM was used for data analysis. The inhibition mechanism and kinetic rate constants were analyzed using GRAPHPAD PRISM 7 software (GraphPad, La Jolla, Calif.).

NMR Studies: ²D; Ileδ1-[¹³CH₃]; Leu, Val-[¹³CH₃, ¹²CD₃]-labeled p38γ sample was purified and prepared for NMR studies. The sample contained 20 μM protein in buffer (20 mM sodium phosphate, pH 7.3; 100 mM NaCl; 0.03% NaN₃; and 10% D₂O). The compound was added to a final concentration of 200 μM. The same volume of dimethyl sulfoxide (DMSO) was used as a control. ¹H-¹³C HMQC spectra were collected with 2048×128 complex points at 25° C. for 5 h on a BRUKER ASCEND 700 spectrometer equipped with a cryoprobe. The spectra were processed and analyzed with the software NMRPIPE [112] and SPARKY (UCSF, San Francisco, Calif.), respectively. NMR CSPs were calculated as

CSP=\sqrt{Δδ_{H}²+(0.341·Δδ_{C})²}

where Δδ_{H} and Δδ_{C} are the chemical shift differences between the free and bound states in the proton and carbon dimensions, respectively. For p38γ resonance assignments, NMR experiments were acquired using ¹⁵N; ¹³C; ²D; Ileδ1-[¹³CH₃]; Leu, Val-[¹³CH₃, ¹²CD₃]-labeled p38γ sample. Triple-resonance spectra, including HNCA, HNCOCA, HNCACB, HNCOCACB, HNCO, and HNCACO, were used for the backbone assignments as we described previously [62]. Methyl groups of Ile, Val, and Leu were assigned using ¹³C NOESY and methyl-backbone correlation experiments [113]. The additional assignments of backbone amides and methyls were transferred from the deposited assignments (BMRB entry: 26732 and 26733) [114].

Modeling of F7/PIK75 binding to p38γ protein by using All-Around Docking (AAD) methodology: We implemented our in-house All-Around-Docking (AAD) methodology to predict the best binding site and binding pose of F7 on p38γ protein. Based on the GLIDE software (Schrödinger, New York, N.Y.) for docking, AAD allows a small molecule to search the whole surface of a target protein for the best docking site with the lowest docking score. The structure of the p38γ protein in complex with the ANP molecule phosphoaminophosphonic acid-adenylate ester was used as the docking target (RCSB protein databank ID: 1 cm8) as shown in FIG. 26C.

IC₅₀ value determination of cytotoxicity of F7/PIK75 against human NCI-60 cell lines and CTCL cells: To determine the cytotoxicity of F7/PIK75 using our in-house NCI-60 cancer cell line panel assay at City of Hope, MTS assays (Promega) were performed and cell viability was determined as described previously [115]. For determination in CTCL cells, CELLTITERGLO Cell Viability Assay (Promega) was used to measure viability of Hut78, HH, and H9 cells, and SS patient samples. Absorbance was monitored at 490 nm using an automated BMG PHERAstar plate reader (BMG Labtech). IC₅₀ values were determined using CALCUSYN software (Biosoft). All experiments are repeated in three independent experiments and data represented are the average of triplicate experiments.

CTCL xenograft tumor model: All animals were housed and handled in accordance with the guidelines of City of Hope Institutional Animal Care and Use Committee (IACUC). The experiments described here were specifically approved by IACUC protocols #07049 and #17118. Female and male 6-week-old NSG mice (NOD-scid IL2Rγ null) were purchased from Jackson Laboratory (Sacramento, Calif.). Hut78 cells were used for developing CTCL xenograft tumors. Briefly, 5 million cells in 100 μL (50% Matrigel/50% PBS) were injected subcutaneously into the right flank of each mouse, and tumor development was monitored every other day. Once the tumors are were palpable and reached 100 mm³ in volume, treatment was commenced. Male and female 8-12 wk, 20-25 g mice were divided into 3 groups (7 mice each group): control Group 1 received saline containing 2% DMSO; Group 2 were treated with 2 mg/kg F7/PIK75; and Group 3 were treated with 10 mg/kg F7/PIK75. Mice were treated every two days via intraperitoneal injection. Tumors were measured twice per week using calipers. Animals were sedated and euthanized once tumors reached 30 mm in diameter, or if any animal lost more than 20% body weight or exhibited any severe pain/distress signs that matched premature euthanasia criteria.

Western blot: Western blots were performed as described previously [5]. Rabbit primary antibodies (Cell Signaling Technology (CST), Danvers, Mass.) were used at the following dilutions: anti-p38α, -p38β, -p38γ, and -p38δ (1:1000), anti-B-tubulin (1:2000), anti-p38 GAPDH 1:1000. Anti-β-Actin (8H10D10) mouse mAb (1:2000, CST), anti-DLGH1 total protein and anti-pDLGH1 at serine 158 and 431 (total SAP97, S285B SAP97 phospho-Ser158 and 431, affinity purified sheep polyclonal antibody, University of Dundee, Scotland, 1:1000). HRP-conjugated goat anti-rabbit antibody #7074 and anti-mouse IgG HRP-linked antibody #7076 (CST, 1:2000) were used as secondary antibodies. For detection of p38γ protein level in SS patient samples we used a monoclonal antibody that close to N-terminus of human p38γ MAPK (SAPK3) from Abcam ((ab205926).

Immunohistochemistry: Xenografts tumors were prepared in paraffin, and representative paraffin blocks were chosen for immunohistochemical analysis, performed by City of Hope Pathology Core (Solid Tumor) as described previously [116], using primary anti-pDLGH1 phospho-Ser158 polyclonal antibody (S285B SAP97, 1:500) and secondary anti-sheep-HRP antibodies 1:2000.

NANOSTRING NCOUNTER® gene expression quantification and validation: 100 ng of RNA isolated from Hut78 cells, as suggested by the NANOSTRING protocol, was used in the experiment. All samples were validated. Data were analyzed using nSolver 3.0 digital analyzer software and R program.

Statistical analysis: All experimental data are shown as mean±SEM unless indicated otherwise. The statistical significance of differences, i.e., in cell viability assays and mRNA expression of target genes, were assessed by student T-test (SPSS, IBM, Armonk, N.Y.) or one-way ANOVA (GraphPad PRISM v. 3.0, GraphPad). Differences were considered significant if P<0.05. Sign Test, a nonparametric test was used for inhibitory IC₅₀ of NCI 60 cells by p38 γ candidates, significant if P<0.005.

### Example 9: Targeting p38 Gamma Signaling to Advance Cutaneous T Cell Lymphoma Therapy

Cutaneous T cell lymphoma (CTCL) is a disfiguring and incurable malignancy. Current CTCL therapies are marked by an abbreviated response and subsequent drug resistance, and prognosis for patients with advanced disease is poor. SAHA (Vorinostat) and romidepsin are histone deacetylase (HDAC) inhibitors that are FDA-approved as an effective option for treating CTCL, but are often limited by drug resistance when used as single agents. Thus, combination regimens that target complementary pathways are urgently needed. An inadequate understanding of molecular regulators involved in CTCL has limited the development of effective targeted therapies. One candidate molecular regulator is p38γ, a mitogen-activated protein kinase crucial for malignant T cell activity and growth in response to T cell receptor (TCR) signaling. p38γ gene expression is selectively increased in CTCL cell lines and patient samples, but not in healthy T cells, suggesting it may play a key role in CTCL pathogenesis and be an effective target for therapy. Furthermore, chemical inhibition or gene silencing of p38γ inhibits proliferation and induces CTCL cell death. An improved mechanistic understanding of the p38γ pathway(s) in CTCL will inform the development of novel targeted therapies that fulfill the critical need to advance treatment and improve outcomes for CTCL patients. Our long-term research goal is to develop effective, targeted therapies for T cell lymphoma by identifying targets in critical signaling pathways that drive pathogenesis. Our objective in this proposal is to understand and exploit the p38γ pathway in CTCL, using a combination of molecular, chemical, and genetic approaches. Our central hypothesis is that p38γ is indispensable for CTCL tumorigenesis, and that inhibition of p38γ has great potential for clinical application in CTCL, especially when combined with HDAC inhibitors. The research team includes an experienced lymphoma clinician-researcher and drug discovery expert; together with our extensive preliminary data, we are well positioned to complete the proposed research.

Determine the mechanisms through which p38γ inhibition induces cell death in CTCL. Dissect the p38γ pathway in CTCL. To investigate the mechanism by which p38γ induces cell killing, we will compare wild-type and p38γ knockdown in CTCL cell lines. We will define the kinase cascade involved in p38γ inhibition-induced CTCL cell killing and identify phosphorylation targets of p38γ signaling. Identify pathways that are complementary to p38γ inhibition. We will use RNAi screens in CTCL cell lines to identify signaling proteins that cause cell death upon depletion in the presence of sub-lethal p38γ inhibition. We will also determine the extent to which combined inhibition of p38γ and HDACs target complementary pathways and induce synergistic therapeutic effects in CTCL cell lines and patient samples. Validate targets for the ability to affect downstream signaling and cellular responses in vitro and in vivo. We will use CTCL cell lines, and cell line xenograft and patient-derived xenograft (PDX) mouse models.

Develop novel p38γ inhibitors for potential therapeutic application. Develop selective p38γ inhibitors for clinical application, using computational models of candidate inhibitor F7 as the scaffold molecule: We identified a candidate p38γ inhibitor (F7) with efficacy in CTCL cells; however, F7 is known to target other kinases, such as PI3K. To improve F7 selectivity for p38γ, we will use both ligand-based and structure-based methodologies. Identify novel functional domains of p38γ, using CRISPR-based screening, as targets for next-generation therapeutics: In addition to its role as a kinase, p38γ mediates multiple signal transduction pathways through protein-protein interactions. Thus, we expect that additional functional elements in p38γ mediate its unique role in CTCL. We will use CRISPR-based screening to identify novel functional domains of p38γ, and perform computational screening to identify novel small molecules that target these domains. Validate hits for CTCL cytotoxicity and p38γ-specific inhibition in vitro and in vivo: We will synthesize small molecule inhibitors and validate them in CTCL cell lines, and cell line xenograft and PDX mouse models. This work is innovative in that it will illuminate critical signaling pathways with clinical significance in CTCL, which to date remain enigmatic. We expect that successful completion of this proposal will yield mechanistic information about the unique biological and clinical relevance of p38γ signaling and complementary pathways in CTCL. This will advance the field by providing new targets for the development of more effective therapeutic treatments for CTCL and lead to future studies investigating the function of p38γ and its downstream targets as predictors of CTCL progression. Importantly, validation of a specific p38γ inhibitor with efficacy in CTCL animal models will have immediate relevance for CTCL therapy. Our work also holds potential for application in a spectrum of other cancers affected by p38γ dysregulation, and associated with poor prognosis and limited treatment options.

Research Strategy

Cutaneous T cell lymphoma (CTCL) develops from clonal expansion of effector/central memory CD4+ T cells [1]. It most commonly presents on the skin as mycosis fungoides (MF) or the leukemic variant, Sézary syndrome (SS), and may involve the blood, lymph nodes, or other organs [2]. In the US, approximately 3,000 cases are diagnosed each year, and 60,000 patients live with this chronic, relapsing disease. If skin-directed therapy is inadequate or disease is advanced, the most effective systemic therapies are biologic response modifiers including interferons, rexinoids/retinoids, and selective histone deacetylase inhibitors (HDACi) [1, 3]. However, current therapies are associated with an abbreviated response and subsequent drug resistance, and prognosis for patients with advanced disease is poor [1, 2]. Until recently, no molecular drivers of CTCL had been identified, prohibiting the development of driver-based targeted therapies. Thus, identifying critical pathways and molecular drivers of CTCL is essential to understanding progression of the disease and developing effective therapies that improve quality of life and outcome for CTCL patients. We identified the mitogen-activated protein kinase (MAPK) p38 family of isoforms as a potential therapeutic target for CTCL [5]. p38 MAPKs are typically activated through a classical signal transduction cascade via dual phosphorylation of p38, which in turn phosphorylates downstream substrates that is triggered by stress stimuli and ultimately impacts intracellular processes such as proliferation and apoptosis [8, 9]. In contrast, the alternative p38 activation pathway exists solely in T cells. Instead of signaling through the classical MAPK cascade, upregulates NFAT (nuclear factor of activated T cells) transcription factors and interferon regulatory factor 4 (IRF4) [13], followed by production of the pro-inflammatory cytokine IL-17A by Th17 cells, which is frequently deregulated in CTCL patients [13]. Our preliminary data narrowed our focus to the p38γ isoform as a promising new target for CTCL. This 367-aa p38γ protein is highly expressed in muscle, with no detectable expression in normal hematopoietic cells or tissues of the immune system including lymph nodes and spleen [6, 7]. Importantly, although normal healthy T cells do not express p38γ, our preliminary data show it is highly expressed in the human CTCL cell line HH, the human SS cell line Hut78, and primary SS patient samples. This suggests that p38γ may be a key driver in CTCL. Overexpression of p38γ affects downstream pathways both through its kinase activity and through protein-protein interactions mediated by the presence of a PDZ binding domain [14]. PDZ domains share a common 80-90-aa structural domain that plays a key role in anchoring receptor proteins in the membrane to cytoskeletal elements. One such target is human discs large homolog 1 (DLGH1; also known as synapse-associated protein 97, or SAP97), a mammalian MAGUK-family member protein. DLGH1 is a direct substrate of p38γ kinase activity, and can be phosphorylated by p38γ at serine 158. DLGH1 also has three PDZ domains, is an important scaffolding protein that is required for the alternative pathway of p38 activation, and directs T cell signaling through the NFAT pathway rather than through the NF-kB pathway [14, 15]. Whether p38γ modulates alternative p38 activation via DLGH1 in CTCL remains unclear. T cell receptor (TCR) signaling for normal development, activation, and differentiation of T cells typically involves the classical NFAT (nuclear factor of activated T-cells) and NF-κB signaling pathways [1], which leads to cell proliferation. We demonstrated that chemical inhibition of p38γ or reduction of p38γ level by shRNA correlated with significant reduction of cell proliferation in both Hut78 cells and HH cells, and that loss of p38γ correlates with reduction of NFATC4 and its downstream target IL-17A. In malignant T cells, the NF-κB pathway is constitutively activated, although the mechanism remains unknown [1, 14]. NF-kB has been suggested to play a critical role in CTCL development and maintenance [14, 95]. Histone deacetylases (HDACs) remove acetyl groups from histone and non-histone targets. Opposing activities of histone acetyltransferases (HATs) and HDACs tightly regulate gene expression through chromatin modification, and are often dysregulated in cancer, making HDACs an even more attractive therapeutic target [37]. The HDAC enzyme family is divided into four major classes, each comprised of multiple members. NF-κB interacts with Class I HDACs, to regulate gene expression. Therefore, combined use of p38γ inhibitors and HDACi offers an opportunity to synergistically target both NFAT and NF-κB pathways. We show that combined p38γ inhibition and HDACi inhibition reduces expression of at least two critical modulators of TCR signaling: inducible T cell kinase (ITK), a Tec family tyrosine kinase; and p38δ, whose specific role in TCR signaling remains elusive. Our objective is to use a combination of molecular, chemical, and genetic approaches to understand and exploit the p38γ pathway in CTCL as a unique therapeutic target. Dissecting the key signaling molecules in the p38γ pathway, particularly in combination with other CTCL therapies (e.g., HDACi), will predict elements for potential resistance and alternative therapeutic targets for use in CTCL therapy. We expect our results to have immediate relevance for CTCL therapy. Furthermore, p38γ protein is highly expressed in several additional human malignant cell lines with a spectrum of histologies, including melanoma [70], colon cancer, and breast cancer [71-73], indicating a possible role in tumorigenesis of multiple cancers. This suggests that our study results will be relevant to understanding the biology of other p38γ-driven cancers with poor prognosis, as 81 well as identifying therapeutic targets to improve treatment options.

The proposed work will use a multi-disciplinary approach to illuminate critical signaling pathways with clinical significance in CTCL, which to date remain enigmatic. We expect successful completion of this proposal to: i) Yield mechanistic information about the unique biological and clinical relevance of p38γ signaling and complementary pathways in CTCL, which will advance the field by providing new targets for the development of more effective treatments for CTCL and lead to future studies investigating p38γ and its downstream targets/function as predictors of CTCL progression; ii) improve the efficacy of targeting the p38γ pathway with complementary therapies that intersect relevant pathways/targets. Inhibition of both p38γ and NF-κB through HDACi will allow complementary targeting of two critical T cell pathways that are dysregulated in CTCL, for increased efficacy with reduced toxicity iii) develop and validate a more selective p38γ inhibitor and combination therapy in CTCL animal models for immediate relevance in CTCL therapy. Our work also holds potential for application in a spectrum of other cancers affected by p38γ dysregulation, and associated with poor prognosis and limited treatment options. City of Hope (COH) is unique in providing in-house access to an FDA-licensed current good manufacturing practice-compliant facility for production of clinical-grade small molecules, and the regulatory expertise to advance therapeutics without involving pharmaceutical companies.

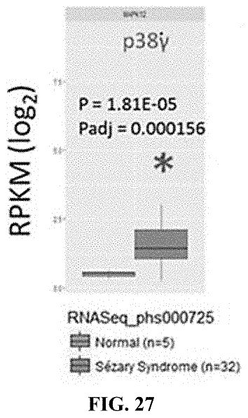

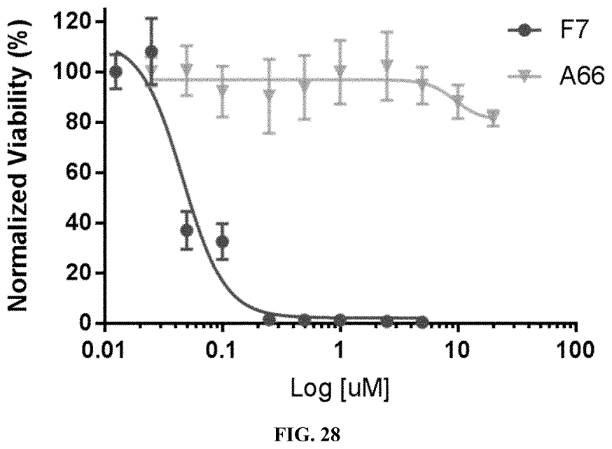

p38γ is elevated in CTCL and is important for cell viability: In pursuit of key signaling molecules that drive CTCL, we identified the p38 family as important for CTCL growth [5]. The p38 family includes α, β, γ, and δ isoforms, which share similar protein sequences, but vary in tissue-specific expression, substrate preference, and downstream effects [6]. We narrowed our focus to p38γ as the most appropriate target: quantitative RT-PCR showed that p38γ mRNA expression is elevated in Hut78 cells (SS cell line), and in primary CD4+ T cells from SS patients, but not healthy donors (data not shown). To confirm increased expression of p38γ in CTCL, we analyzed a publicly available microarray database (GSE176017, n=32 for SS; GSE129028, n=22 for MF; GSE190699, n=8 for healthy donors and cell lines) and RNA seq database (phs_000725). Both databases showed that gene expression of p38γ is increased in CTCL patients compared to healthy donors/cell lines (FIG. 3B, FIG. 27). To demonstrate the effect of loss of p38γ expression on CTCL proliferation, we genetically silenced p38γ expression using siRNA in HH cells (CTCL cell line; data not shown) or shRNA in Hut78 cells (FIG. 18C); both demonstrated significantly reduced cell proliferation. These data implicate p38γ as an indispensable driver in CTCL development. F7 kinase inhibitor shows unique features in CTCL Given our data, p38γ is an attractive therapeutic target for CTCL. However, no specific p38γ inhibitor is available for CTCL. Pirfenidone, a TGF-β inhibitor with off-target activity against p38γ, is FDA-approved for treatment of pulmonary fibrosis [41, 75]. It inhibits p38γ in mice, but requires a high dose [96], and the mechanism of action is unknown. To identify novel, potent, specific inhibitors of p38γ, we used molecular modeling and high-throughput screening of a commercial kinase inhibitor library. We identified F7 (also known as PIK75) as a candidate inhibitor that reduced proliferation of Hut78 cells more effectively than Pirfenidone (FIG. 7). F7 showed a selective, dose-dependent inhibition of p38γ and 6 but not p38a and p kinase activity (FIG. 20A) and reduced cell viability in Hut78 CTCL cells (FIG. 19B). Importantly, F7 exhibited selective induction of apoptosis (data not shown) and inhibition of growth in SS patient cells and Hut78 CTCL cells, but not healthy CD4+ T cells (FIG. 19E). In addition, although F7 was originally identified as a potent PI3K 110α inhibitor, we showed it was more effective against CTCL cells than a PI3K-specific inhibitor (A66; FIG. 28). We further confirmed that cell viability was not affected by gene silencing of PI3K p110α (FIG. 22C), in contrast to knockdown of p38γ (FIG. 18C). This suggests that in CTCL, F7 causes cell death by targeting p38γ rather than targeting PI3K p110α. To demonstrate inhibition of tumor growth in vivo, we treated mice harboring Hut78 xenografts with vehicle control or F7. F7 showed a statistically significant dose-dependent inhibition of tumor growth at 2 mg/kg (p=0.015) and 10 mg/kg (p=0.025) (FIGS. 21A-21B). We also showed that F7 affected p38γ kinase activity (i.e., reduced phosphorylation of p38γ substrate DLGH1 at Ser158) but not p38γ protein level using western blot of Hut78 cells (FIGS. 21A-21B, inset) and immunohistochemistry in F7-treated CTCL xenograft mouse tissue (data not shown). Together, our data show that F7 specifically and potently targets p38γ in vitro and in vivo, despite targeting multiple kinases.

F7 is an ATP-competitive p38γ inhibitor: To locate where F7 binds to p38γ, we performed time-resolved fluorescence energy transfer using a peptide substrate derived from the p38γ phosphorylation site of 4E-binding protein 1 [91], which was labeled with a fluorescent tag at the N-terminus. Enzyme kinetic analysis indicates that F7 inhibits p38γ via a competitive mechanism, i.e., competing with ATP binding to the enzyme. We determined the ATP Km to be 3.2±0.4 μM and inhibitor Ki to be 12.2±1.5 nM (FIG. 20B, left). Consistent with this, NMR data showed that F7 induced extensive chemical shift perturbations (CSPs) and line broadening in the ₁H-₁₃C HMQC spectrum (data not shown) for residues at or near the ATP-binding pocket, which indicates it binds to the ATP-binding pocket of p38γ. We used our in-house-developed All-Around Docking (AAD) methodology [92] to model F7 binding to p38γ protein. The 3-D structural binding model predicted that F7 binds the p38γ ATP-binding pocket (FIG. 20C, right).

F7 targets NFAT pathways: Our preliminary data indicate that NFATC4 expression is modulated by the presence of p38γ. To demonstrate a direct connection between p38γ and NFATC4, we used qRT-PCR to measure the expression of NFATC4 in p38γ-knockdown Hut78 cells. Knockdown of p38γ, but not p38β, induced a reduction in mRNA expression of NFATC4 (FIG. 6B), as well as its downstream target, IL-17A (FIG. 6C). Inhibition of NFATC4 using shRNA also reduced the proliferation of CTCL cell lines (data not shown) and reduced IL-17A mRNA expression (FIG. 6D). Because ITK signaling regulates IL-17A production through NFAT activation [97], our data suggest an interaction between p38γ and ITK. Altogether, these data suggest that malignant T cells express critical components of the p38γ pathway to enhance proliferation in CTCL.

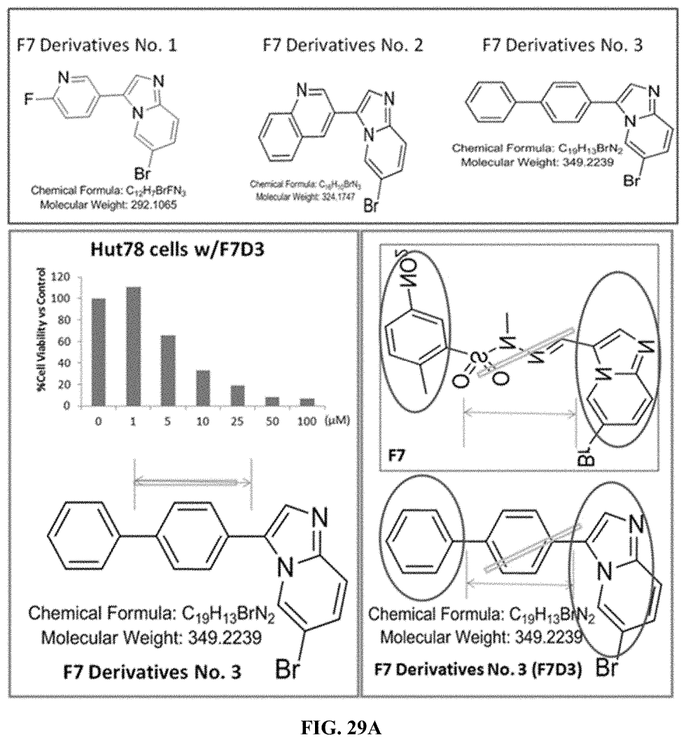

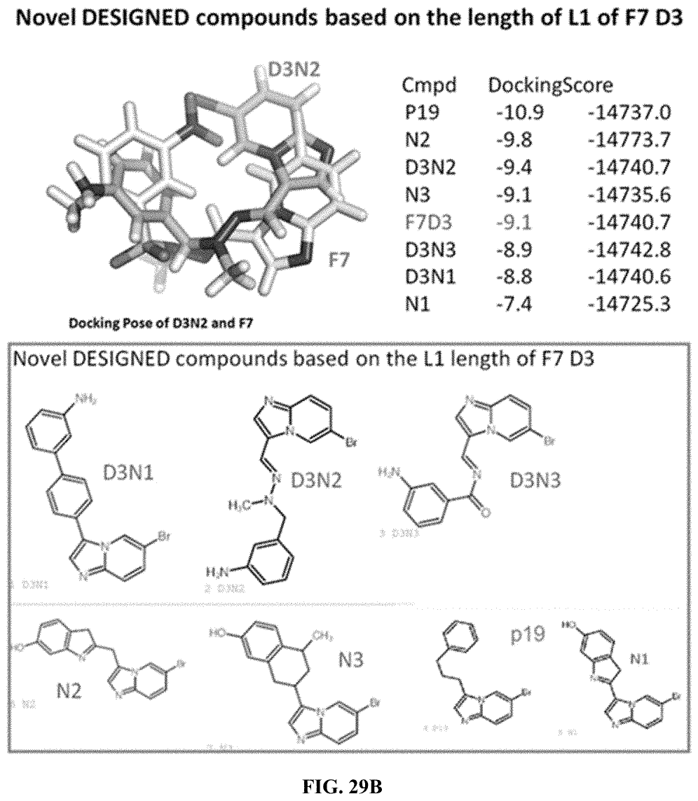

Structure-guided drug design of F7 analogs: Our initial investigations into the specificity of F7 focused on targeting the active form of p38γ, based on our extensive knowledge of the structure-activity relationship (SAR) of kinase inhibitors to inform structure-based design. Three small molecule derivatives of F7 (F7D1-3) were synthesized; among these, only F7D3 reduced cell viability in Hut78 cells (FIG. 29A). Therefore, in addition to the sulfonamide moiety, the spatial distance between the two circled groups (aromatic rings) in F7 may be important for binding of the drug, and new F7 derivatives will in part conserve that feature (FIG. 29A). F7D3 was designed to have a length of L₁ of ˜5.8 Å and was shown to suppress viability of Hut 78 cells in a dose-dependent manner (FIG. 29A, bottom left). This shows a novel approach for designing compounds based on the L₁ length of F7D3 (FIG. 29A, bottom right); the spatial distance between two (circled) aromatic groups in F7 range from 5.0-5.9 Å. With this scheme based on L¹ length of F7D3, we used a docking pose to generate 7 compounds (D3N1-N3, N1-N3, and p19; FIG. 29B, bottom). We showed that 4 compounds (P19, N2, D3N2, and N3 FIG. 29B, top) exceed F7D3 in docking score of predicted binding to p38γ. We will further validate these compounds, in part, using cell-based analyses discussed above as well as describe future analogs based on modeling studies.

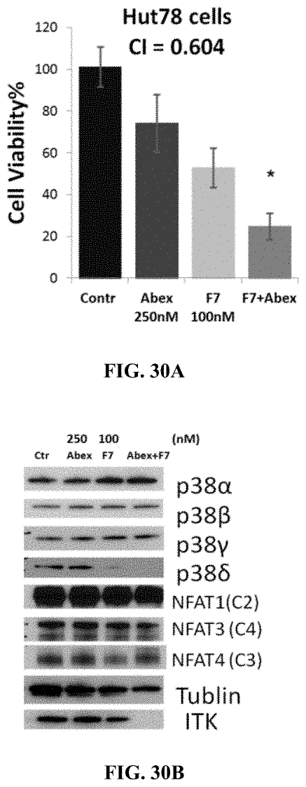

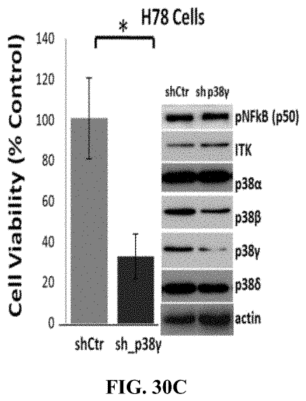

F7 and HDACi induce synergistic therapeutic effects: We have shown that F7 is a more potent inhibitor of CTCL cell growth than the FDA-approved HDACi Vorinostat (SAHA; FIG. 20A, FIG. 19B, FIG. 19E); we also showed that combination of F7 and SAHA induced synergistic killing (FIG. 12). To better understand the mechanism of synergy between F7 and HDACi in CTCL cells, we used western blot to show that ITK, a downstream biomarker of the T cell signaling pathway, is significantly reduced upon combination treatment with F7 and the pan-HDACi Abex (FIGS. 30A-30C), but not F7 alone, suggesting the reduction is due to HDAC inhibition. Therefore, we expect that the mechanism of synergy between F7 and HDACi occurs in part via reducing ITK protein level, which in turn decreases NFAT and IL-17A.

Research Methods: Determine the mechanisms through which p38γ inhibition induces cell death in CTCL.

Dissect the p38γ pathway in CTCL: Fully define the kinase cascade involved in p38γ inhibitor-induced CTCL cell killing. To identify novel critical components in the p38γ kinase cascade related to several known signaling factors in CTCL (i.e., NFATs, ITK, or p38δ), we will compare and validate them in both wild-type and p38γ-knockdown cells. We will transduce human CTCL Hut78 cells with shRNA lentivirus to create p38γ-knockdown cells as described in preliminary data. We will use inducible shRNA constructs (ptripz-GFP vector with tet operator) to overcome the otherwise lethal effect of p38γ knockdown. We will use qRT-PCR and western blot with protein- and phospho-protein specific antibodies to determine mRNA expression, protein expression, and phosphorylation status of NFATs, ITK, and p38δ in both p38γ wild-type vs. knockdown cells. In detail, we will investigate if p38γ signaling occurs via ITK modulation with different isoforms of NFATs (e.g., NFATC2 or NFATC4). In addition, we will further characterize the expression and phosphorylation status of these proteins in CTCL patient samples. We have access to primary CTCL cells through a COH IRB-approved CTCL banking protocol, and they will be authenticated using flow cytometry for CTCL phenotyping [23]. As described above, the DLGH1/SAP97 substrate of p38γ kinase activity(11), is a scaffolding protein involved in T cell signaling [14]. It associates with p38γ and directs activation of NFATC4. The phosphorylation status of NFATs is critical because it determines the subcellular localization of this type of transcription factors. Phosphorylation of NFATs results in accumulation in the cytosol (preventing their entry to the nucleus) [13], thereby interrupting the proliferation of T cells by blocking transcriptional activity via subcellular sequestration. Phosphorylation of DLGH1 is also functionally important because this determines if it can be released from its binding partner protein for other functions, such as anchoring receptors proteins in the cytoskeleton [15]. We demonstrated that p38γ inhibition correlated with the phosphorylation of DLGH1-ser158 (FIGS. 21A-21B). In addition, we showed p38γ inhibition reduced p38δ protein level (FIG. 30B), suggesting that p38γ is upstream of p38δ. We will identify and validate direct downstream targets of p38δ, such as T50 on Tau protein (the direct downstream targets of p38γ such as S205 on Tau protein [98]). We will further identify direct targets/substrates of p38δ kinase activity together with p38γ inhibition for additional biomarker identification. We will use western blot and microscopy with relevant phospho-specific antibodies against the targets above to quantify their expression and phosphorylation status, in CTCL cell lines and patient samples. To determine the extent to which p38δ is required for p38γ inhibitor efficacy in CTCL cells, we will create p38δ knockout cells and p38γ/p38δ double-knockout cells. We will express wild-type or mutated DLGH1 (depleted of aa ser158), or knock down DLGH1, in p38γ and p38δ single- and p38γ/p38δ double-knockout cells and control Hut 78 cells. We will measure if CTCL cells can survive upon p38γ inhibition (i.e., F7 treatment) when its downstream substrate is depleted. If we identify a group of targets that are differentially expressed in double-knockout compared to single-knockout cells, then we will compare them with targets identified in cells treated with F7 and/or an optimized analog, respectively.

Identify phosphorylation targets of p38γ signaling. In addition to the specific targets we will study discussed above, we will use proteomic analysis [31] to identify global phosphorylation targets of p38γ signaling. We will stably transduce Hut78 and HH human CTCL cell lines with a p38γ-targeting shRNA lentivirus (Sigma) or scrambled control. Cells will be lysed, cleared by centrifugation, reduced, and alkylated, and at least 200 μg of protein from each sample will be digested overnight by trypsin. We will enrich for phosphopeptides using TiO₂ columns, and the enriched phosphopeptides will be eluted and analyzed using LC-MS/MS. We will search the resulting MS/MS data using Mascot and SEQUEST search algorithms against a Human RefSeq database for phosphorylation (+79.96633 Da) of serine, threonine, and tyrosine residues. The probability of phosphorylation for each Ser/Thr/Tyr site on each peptide will be calculated by dividing the intensity of each phosphorylated peptide over the intensity of the corresponding peptide. Through comparison of phosphorylated peptides from cells with or without p38γ knockdown, we will not only identify proteins that are differentially expressed in a p38γ-dependent manner, but will also identify the specific phosphorylation sites affected by p38γ signaling. We will then perform a multi-step screening and validation of the candidate p38γ targets. We will measure downstream cellular responses to p38γ and/or p38δ inhibition using cell proliferation, cell cycle, viability, and apoptosis assays. To identify other targets that differ between wild-type and p38γ knockdown cells, we will measure expression of targets known to be important in TCR and MAPK signaling in CTCL and related lymphomas [25, 26, 95]. These may include: PRKG1 (protein kinase, cGMP-dependent, type I) [95] and PLCG1 (phospholipase C gamma 1) [26, 27] which activate NFAT signaling; IL-32, a cytokine upregulated in CTCL [26, 28], which modulates p38 signaling in esophageal cancer [29]; and CD28 [25, 26], a co-stimulatory receptor that can activate p38a [30]. Several of these targets are also associated with NF-kB, which may have relevance the above. We will use approaches similar to those described for DLGH1 above to determine the extent to which these proteins are critical for p38γ signaling in CTCL.

Identify pathways that are complementary to p38γ inhibition. Additional Background: Preclinical studies and early clinical trials have shown that HDACi can be used effectively in combination with other drugs to induce synergistic anti-cancer effects [41]. Based on homology with yeast counterparts, HDACis can be divided into 4 classes. Class I includes HDACs 1, 2, 3 and 8, which are located in the nucleus; Class IIa HDACs includes 4, 5, 7 and 9; Class IIb includes 6 and 8, which are located in both nucleus and cytoplasm; Class III are yeast Sir2 protein (sirtuin) homologues1-7, which are NAD+−dependent and have mono-ADP-ribosyltransferase activity; Class V has only one member, HDAC11. Based on mechanisms of removing the acetyl group, HDACs can also be divided into two distinct groups: the first one, the “classical family” are Zn2+ dependent HDACs; the second group are NAD+ dependent, and as a result of acetyl transfer, O-acetyl-ADP-ribose and nicotinamide are formed [99]. Thus, various HDACs affect a variety of histone and non-histone targets; despite some redundancy, unique HDACs can cause substrate-specific effects [37]. For example, HDAC1 [52], HDAC2 [53], and HDAC3 [53] directly interact with subunits of NF-κB. Furthermore, different HDACs are relevant in different cancers; e.g., HDAC3 is overexpressed in gastric, prostate, and colorectal cancer [100]; HDAC6 is highly expressed in breast cancer; and HDAC1, 2 and 6 overexpressed in CTCL [101] and PTCL [102]. HDAC isoform expression also varies in CTCL, and can have prognostic significance; HDAC2 and HDAC6 expression is elevated in CTCL [55], and elevated expression of HDAC2, as well as acetylated histone H4, is associated with aggressive CTCL, while HDAC6 expression is associated with a favorable prognosis in any CTCL subtype [56]. A detailed understanding of the underlying functions of HDACs, and a corresponding understanding of the mechanism of HDACi effects are currently lacking [37]. In CTCL, whether HDACi target NFκB or not, and the mechanism through which HDACi target NF-κB, is still unclear. However, mycosis fungoides and Sézary syndrome are associated with constitutive activation of the NF-κB pathway, a well-known feature of CTCL and other hematopoietic malignancies [47, 95], suggesting that the success of HDACi in CTCL may be in part through effects on NF-κB. Nuclear NF-kB expression has been demonstrated in Hut78 and HH cell lines, and inhibition of NF-kB in Hut78 and HH cells using chemical inhibitors or a proteasome inhibitor induced apoptosis and reduced proliferation, respectively [95]. Furthermore, small molecule inhibition of NF-kB inhibited tumor growth and metastasis in a CTCL xenograft mouse model [48]. Thus, as we dissect the p38γ pathway and identify pathways that are complementary to p38γ inhibition, we will look for synergy of the p38γ with HDACs.

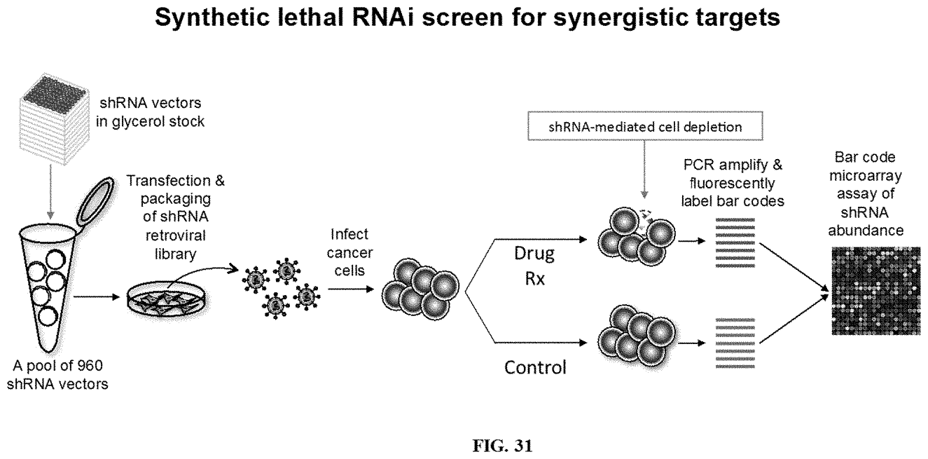

Synthetic lethal RNAi screen. We will use a synthetic lethal RNAi screen in the presence of sub-lethal p38γ inhibition to identify critical downstream signaling components in CTCL (FIG. 31). We will transduce control and p38γ inhibitor-treated Hut78 CTCL cells with a pooled retroviral RNAi library that targets genes involved in human cancers. Because loss of p38γ alone causes CTCL cell death, we will titrate the dose and/or length of exposure to the p38γ inhibitor to ensure a sub-lethal effect. The target gene of an shRNA that causes death in cells treated with sub-lethal p38γ inhibitor but not control cells will be identified as synthetic lethal/synergistic. Based on our preliminary data showing synergy between F7 and HDACi, we are expecting to identify HDAC family members in the RNAi screen. We will further narrow down the corresponding HDACi for each HDAC member identified, and study potential synergistic effects to achieve of killing CTCL cells. For example, our preliminary data has shown using an RNAi library plus the p38 inhibitor Ly2228820, we have successfully selected loss of HDAC3 as a synergistically lethal element to p38 pathway inhibition, including α, β, and γ isoforms of p38 by LY2228820 (10 uM) [67] (the concentration we chose caused p38γ inhibition, data not shown). We will further test the combination of p38γ inhibition with several HDAC3 inhibitors (e.g., RGFP966) for further mechanistic study in Hut78 cells.

Combined p38γ inhibition and HDACi: We will use siRNA and small molecule inhibitors against HDAC family members (HDAC1, 2, 3, 6) to determine the effects of inhibiting individual HDACs versus HDAC subsets, and to identify the most efficacious combination of HDACi and p38γ inhibition. We will apply vehicle control or F7 and/or optimized analogs singly and in combination with various HDACi in CTCL cell lines (e.g., Hut78, HH); we will base dose escalation and timing on our preliminary data and the literature. We will assess effects on cells as above. We will confirm cell line results in CTCL primary patient samples from the COH CTCL bank as available; if we see an effect, we will increase samples to achieve statistical significance. CD4+ T cells from healthy donors will be used as controls for CTCL-specific effects. The most effective combinations identified from cell base analysis will further be tested in mouse models discussed above. Validate targets for ability to affect downstream signaling and cellular responses in vitro and in vivo. Validate and confirm p38γ targets. We will validate selected shRNA clones (targets) by transducing them into CTCL cells combined with/or without p38γ inhibitors (e.g., F7). To circumvent the issue of multi-kinase targets of F7, we will perform further validation via genetic approaches such as gene silencing of p38γ by shRNA approach with those selected shRNA targets. NFATs and NFkB play a role in many facets of helper T cell function; for this proposal, we are focused on the role of ITK in synergistic p38γ and HDAC inhibition, which may modulate both the NFAT and NFkB pathways, when combined with p38γ inhibition. We will generate ITK knockout cells and treat them with p38γ inhibitor and HDACi. The cell lines in this proposal are Hut78 cells and H9 cells. To validate the selected differential proteins and their phosphorylation sites, we will first perform functional categorization using pathway analysis software. The identified phosphoproteins will be categorized based on known association with biological processes, molecular functions, and cellular components based on gene ontology. Phosphorylated proteins involved in processes such as signal transduction, transcription, and cell cycle progression, especially those involved in (dysregulated) MAPK [8, 9, 17] and TCR [25, 26] signaling pathways and/or transcriptional activation of NFATs [32], as well as proteins identified as mutated in CTCL [25, 26, 95] will be selected for further study. We will use the ProteoConnections bioinformatics platform to facilitate peptide and protein identification and inform biological validation of the phosphorylated targets [33]. In vitro validation. We will validate identified proteins for the ability to affect downstream signaling and cellular responses; i.e., proliferation, cell cycle, cell viability, apoptosis, sub-cellular localization, and phosphorylation of p38γ target proteins related to p38γ inhibition or HDAC inhibition alone, and combination treatments (e.g., using western blot, RT-PCR, and flow cytometry). We will perform a screen in CTCL cell lines using FDA-approved pan-HDACi (i.e. SAHA, Romidepsin (targets HDAC1 and 2), Belinostat) to confirm cytotoxic effects in CTCL and that the specific HDACs of interest are effectively targeted; depending on which HDACs are identified, we will also screen more specific HDACi for efficacy (e.g., Santacruzamate A (CAY10683, Sellekchem) targets HDAC2; Tubastatin A (S8049, Selleckchem) targets HDAC6; RGFP966 (S7229, Selleckchem) targets HDAC3). In vivo CTCL xenograft and PDX models. Two specific HDAC targets will be selected as the top targets from the in vitro studies (i.e., greatest effects on cell cytotoxicity, apoptosis, and kinase activity of p38γ). Based on in vitro results, we will examine pan-HDACi or HDACi that target the identified HDAC proteins for therapeutic efficacy in xenograft mouse models developed from 2 CTCL cell lines (Hut78 and HH); the most potent inhibitor will then be tested in 2 PDX models as in preliminary data. To determine the optimal dose and route for the candidate HDAC inhibitors in xenograft and PDX models, we will first obtain drug metabolism and pharmacokinetic (DMPK) data from Pharmaron, who are our preferred R&D vendor, and whom we have used previously with good results. For CTCL cell line xenograft studies, female and male, 8-15-week NSG mice (NOD-scid IL2Rgnull; Jackson Labs #005557) will be injected with 5-10 million CTCL cells in 100 μL (50% Matrigel/50% PBS). Mice will be weighed and monitored daily for signs of toxicity for 14 days. Once the tumors reach 100 mm₃ in volume, the lead compounds will be administered (route based on PK data); we will compare 10 groups total, with 7 mice per group: Group 1=control vehicle; Group 2=monotherapy for p38γ inhibition (i.e., F7 or optimized analog), 1 dose level; Group 3-4=monotherapy for HDACi #1, at 2 dose levels; Group 5-6=monotherapy for HDACi #2, at 2 dose levels; Group 7-8=2-drug combination, 1 dose level for p38γ inhibition, 2 dose levels per HDACi #1; and Group 9-10=2-drug combination, 1 dose level for p38γ inhibition, 2 dose levels per HDACi #1. Tumor size will be measured twice per week, and mice will be monitored for tumor regression for 4 weeks post-treatment. 87 Experiments will be stopped once tumors>reach 15 mm in in diameter; any mouse with body weight loss>20%, exhibiting any severe pain/distress, signs, or surviving the 4-week monitoring period will be sedated and euthanized. Autopsy will include visual examination, weight, and histological examination of plasma, tumor, and tissues. We will validate the efficacy of the single agents and combination by measuring downstream targets of p38γ signaling. We will then compare the single agent activity of p38γ inhibition, the best HDACi validated in CTCL xenograft models, and their combination in PDX models. CTCL SS PDX mouse models (DFTL-90501-V3) will be commercially obtained from Dr. David M. Weinstock's Leukemia and Lymphoma Xenograft (LLX) public repository [69], and maintained according to their guidelines. Female and male 8-12-week-old mice will be divided into four groups (7 mice per group): Group 1=control vehicle; Group 2=monotherapy for p38γ inhibition (i.e., F7 or optimized analog), 1 dose level; Group 3=monotherapy for HDACi, 1 dose level; group 4=2-drug combination, 1 dose level per agent. We will measure endpoints as described for CTCL xenograft models. Statistical analysis: We will perform standard statistical measures. Seven mice per group are sufficient for achieving statistical significance based on power analysis and preliminary studies. We will analyze animal model data separately for females and males to rule out any sex differences.

Expected Results, Potential Pitfalls, and Alternative Approaches: Identifying downstream targets in the p38γ pathway will provide valuable insights into potential mechanisms of resistance and future therapeutic targets. We expect loss of ITK resulting from the combination treatment of HDACi+p38γ inhibition. As an alternative approach, we will investigate the role of p38δ for its reduction in the combined regimen with each inhibitor that targets individual HDACs (not pan-HADCi), and define the extent to which p38δ expression level correlates with ITK using western blots of cell lysates treated with combined F7/HDACi compared to F7 alone and HDACi alone. If we do not identify HDAC family members (i e., HDAC1-11) by RNAi screen, as we expected, we will subject CTCL cells to combination treatment (p38γ inhibition+HDACi) and perform RNA expression analysis followed by IPA analysis to screen the pathways, and compare with that of p38γ inhibition or HDAC inhibition alone. We will include additional pathways; i.e., apoptosis pathway that can be targeted by HDACi, using FAS or TRAIL as downstream targets. Downstream of HDAC inhibition covers a spectrum of targets, and thus generates difficulties in proposing which will be the hit(s); however, there are three major downstream targets of HDACs in immunity & inflammation pathways: NF-κB, STAT3, and TNFa [103]. Inhibition of NF-κB activity is marked by reduced phosphorylation of NF-κB p65 at Ser276 and Ser536, and reduced acetylation of NF-κB p65 at Lys310. In response to HDACi, we expect to see inhibition these p65 post-translation modification forms by western blot. If western blot shows too many non-specific bands for accurate detection of phosphorylation or acetylation status, we will consider using immunoprecipitation or T7-tag IP method [53] to eliminate non-specific bands. If we do not detect any reduction of NF-κB activity in response to HDACi, this will indicate that NF-κB is not an HDACi target in CTCL, although we expect the chance of this to be limited, given evidence in the primary literature. However, if NF-κB is not an HDACi target, or is induced by HDACi, as demonstrated in another study [57], we will explore alternative targets such as STAT3 and TNFα that may explain the efficacy of HDACi in CTCL [56, 58]. HDACi are well known for effects on the proteasome degradation pathway [35, 51]. If we do see reduced activity of NF-κB, but not reduced phospho-p65, we will continue to decipher the possible mechanism of action, such as finding co-repressors of NF-κB that bind to HDACs by the proteomics profiling methodology described above. We will work with the COH Mass Spectrometry & Proteomics Core, and do not expect technical difficulties performing proteomic analysis.

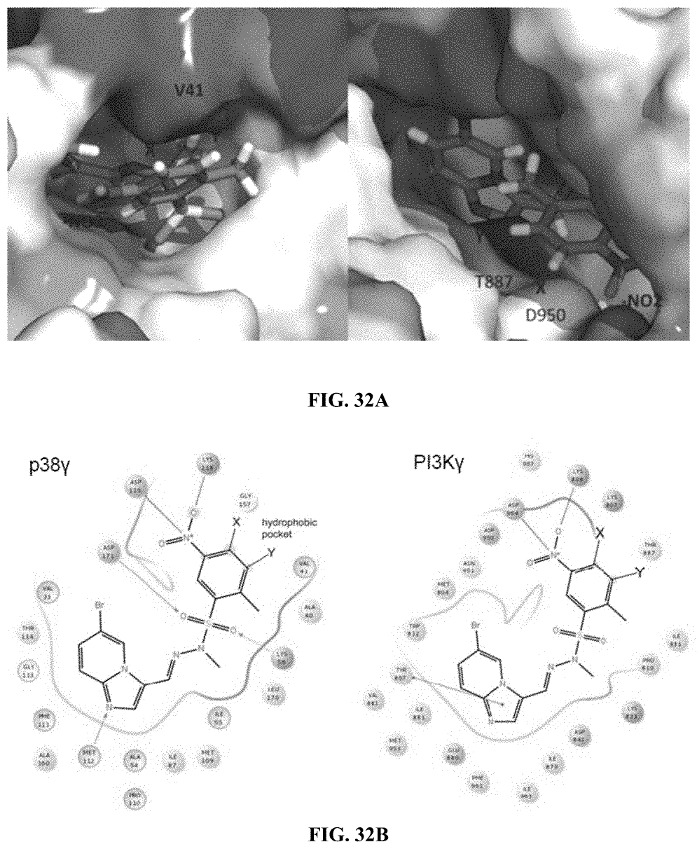

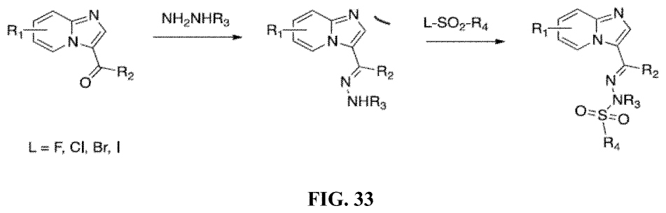

Develop novel p38γ inhibitors for potential therapeutic application. Additional Background and Preliminary Studies: We previously identified a candidate p38γ inhibitor, lead compound F7, which selectively kills CTCL cells at nanomolar concentrations. Although F7 also targets PI3K, as shown in our Preliminary Data, the cell killing effects of F7 in CTCL cells are due to targeting p38γ rather than PI3K 110α (a target associated with significant clinical autoimmune cytotoxicity). Because of its multi-targeting nature, we cannot rule out involvement of other kinases in the cell death induced by this compound; however, we are confident that p38γ inhibition is critical. To study the effects of p38γ gene silencing on expression of other genes, we performed NanoString RNA analysis of H9 cells whose p38γ mRNA was silenced by shRNA, using a scrambled shRNA as control, as well as H9 cells treated with F7 (500 nM) for 24 h compared to vehicle control. We found there is a high correlation between gene expression alterations (i.e. upregulation or downregulation) upon knockdown of p38γ or F7 treatment, with correlation coefficients of 0.7 and 0.6 in the two panels of NanoString analysis (PanCancer Pathway and PanCancer Immune panels), respectively (data not shown), in two merged panels of H9 cells, correlation coefficient=0.628) (FIG. 15A). This indicates that F7 inhibits CTCL through the function of p38γ. We have similar results using Hut78 cells (data not shown). Therefore, we will use F7 as a scaffold p38γ inhibitor for lead optimization studies. Our primary goal is to develop a more selective p38γ inhibitor over PI3Kγ, which should ameliorate the undesirable autoimmune cytotoxicity seen in the clinic with PI3K inhibitors. We chose the PI3Kγ isoform as the 88 model protein because it is relative sensitive to F7 (IC50=76 nM) than other isoforms (i.e., PIK3δ, IC₅₀=510 nM). We will use both ligand-based and structure-based methodologies. For ligand-based lead optimization, we will search for F7 analogs using our in-house developed TTCS (Three-dimensional and Two-dimensional Compound structure Search online tools). For structure-based lead optimization, we will use our in-house developed SAG (Side-chain Auto-Grow) method to improve the side-chains of F7 to have higher binding affinity for p38γ than PI3K (FIG. 32A). Based on these results we will synthesize the various analogs and validate hits for CTCL cytotoxicity and p38γ-specific kinase inhibition.

Develop selective p38γ inhibitors for clinical application, using computational models of candidate inhibitor F7 as the scaffold molecule. To improve F7 selectivity for p38γ, we will combine molecular dynamics simulations (MD) starting from the crystal structures of the target p38γ, and PI3Kγ with ligand docking and optimization methods. Based on molecular modeling there is an opportunity to explore structural analogs that would distinguish between these two kinases. Structural opportunities to discriminate between these two kinases appear to be at positions X (ortho to nitro) and Y (meta to nitro) on the benzene ring. The F7-protein interaction diagrams below are illustrative (FIG. 32B). One difference between the ATP binding sites of p38γ and PI3Kγ is that the former has a hydrophobic pocket near V41, whereas PI3Kγ does not. Therefore, by introducing larger structural substituents at X and Y on the benzene ring such as a methyl or ethyl moiety, we predict a gain in hydrophobic interactions in p38γ while simultaneously introducing a negative interaction (steric hindrance) with residues D950/T887 in PI3Kγ. In addition, introduction of the guanidium group at position X of F7 is predicted to produce a disfavored Columbic interaction involving cationic repulsion with residue Lys807 of PI3Kγ. The general synthetic strategy is outlined in Scheme 1 [36], and is relatively straightforward. Based on literature precedence, we do not anticipate any difficulties in the synthesis. In addition, many commercial analogs exist, with various R₁ to R₄ substituents, which can be incorporated for testing as well. We do not believe that complete selectivity between p38γ and PI3Kγ is necessary to achieve a clinical benefit since therapeutic blood concentrations can be adjusted to achieve the desired effect. In this regard, our goal is to achieve a 100-fold selectivity between p38γ and PI3Kγ. Hits will be structurally characterized by high-resolution NMR spectroscopy and the structural information gleaned will be used as parameter-setting inputs for additional molecular modeling refinements. In addition to the studies described above, we will also investigate by SAR the need/role for an aromatic nitro group and determine if a more metabolically stable sulfonamide moiety can be substituted. We have assembled an inter-disciplinary team of experts in molecular modeling (Vaidehi), NMR structural characterization (Chen), and medicinal chemistry (Home) to maximize our chances of success in developing a more selective p38γ inhibitor.

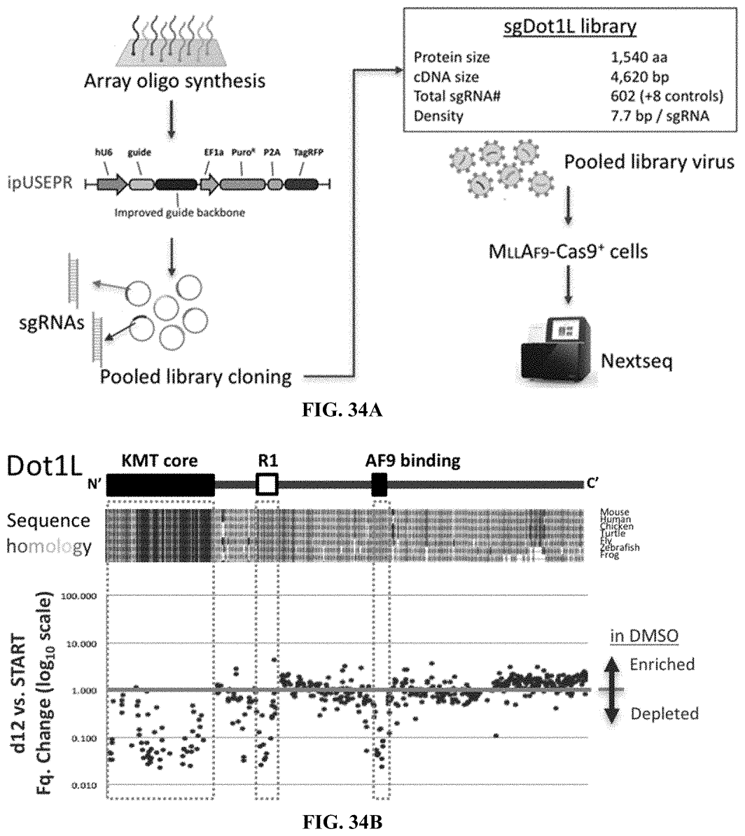

Identify novel functional domains of p38γ, using CRISPR-based screening, as targets for developing allosteric next-generation therapeutics. The goal may be to discover novel elements and potential therapeutic targets in p38γ, which will provide valuable insight into the molecular mechanisms of p38γ signaling pathways. Completion of this study will also create a new research avenue for more advanced p38γ targeted therapies. Additional Background and Preliminary Studies: To identify the functional domains in p38γ (including previously unknown elements), based on a recent report “CRISPR-Cas9 screening of protein domains” [104], we developed a novel genetic screen approach called “high-density CRISPR protein scan.” This powerful methodology enables discovery of the functional elements within a protein by saturation mutagenesis achieved through CRISPR (Clustered Regularly Interspaced Short Palindromic Repeats)-mediated genome editing. For this, we established a custom screen pipeline including microarray oligo synthesis (90K oligos ordered from CustomArray Inc.), pooled sgRNA library cloning, lentiviral production/infection, and high-throughput CRISPR guide sequencing (FIG. 34A). We conducted a proof-of-principal experiment in an acute myeloid leukemia model harboring an MLL1 gene translocation (MLL-AF9 leukemia), which is known to be highly dependent on the enzymatic activity of the histone H3K79 methyltransferase DOT1L for continuous proliferation [105, 106]. To perform the screen, we first generated MLL-AF9 leukemia cells that stably express a Cas9 nuclease (MLL-AF9-Cas9+) via lentiviral transduction. We also constructed a pooled lentiviral library composed of 602 sgRNA that target every “NGG” protospacer adjacent motif (PAM) within the endogenous Dot1l coding regions. This allowed us to perform a saturation mutagenesis screen by high-density CRISPR targeting in mouse MLL-AF9-Cas9₊ leukemia with an average density 13.0 sgRNA per 100 bp exon (FIG. 34B). We then compared the frequencies of the integrated sgRNA sequences before vs. after a 12-day culture using high-throughput sequencing (Illumina NextSeq). Of note, clusters of sgRNA targeting the known functional regions including the lysine methyltransferase (KNIT) core and AF9-binding were significantly more depleted in the screen compared to sgRNA targeting most of the other regions (FIG. 34C). Importantly, this screen identifies a previously undescribed functional element that is essential to DOT1L's function (FIG. 34B, region R1), thus demonstrating the utility of our high-density CRISPR protein scan for de novo functional element discovery.

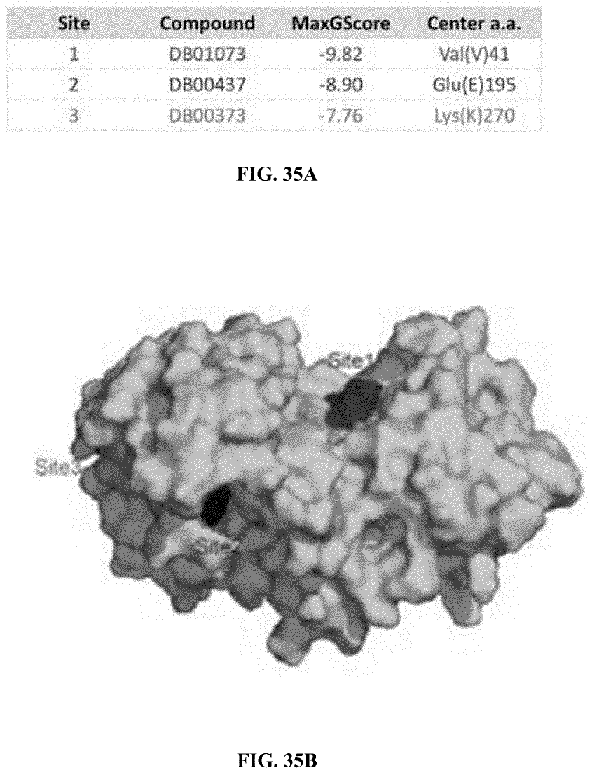

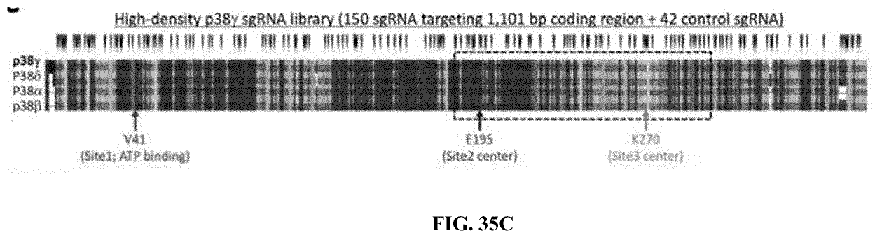

High-density CRISPR protein scan of p38γ. Based on our preliminary studies, we will perform a high-density CRISPR protein scan of p38γ in the CTCL cell line Hut78. First, we will transduce Hut78 cells with a lentiviral construct LentiCas9-Blast (Addgene) to stably express the Cas9 nuclease. Next, we will use microarray (90K oligos from CustomArray, Inc) to synthesis sgRNA oligos and conduct a pooled sgRNA library targeting every “NGG” PAM position in the coding regions of human p38γ. This design will allow us to examine the potential functional elements in p38γ with an average density 13.6 sgRNA per 100 bp exon (total 150 sgRNA in 1,101 bp exon region; FIGS. 35A-35C). These p38γ-targeting sgRNA, together with a panel of control sgRNA (total 30 constructs targeting non-essential sequences in mammalian cells including LacZ, Firefly luciferase, and Renilla luciferase), will be cloned into a CRISPR vector (ipUSEPR; see FIGS. 34A-34C) that expresses sgRNA with an improved guide backbone (for increased stability and efficacy), and co-expresses both a puromycin resistance gene (Puro_{R}) and a red fluorescent protein (tagRFP). We will deliver these sgRNA libraries into Hut78-Cas9₊ cells using lentiviral transduction (MOI=0.5; about 10-15% infection measured by flow cytometry of RFP), and select cells harboring the sgRNA constructs using puromycin. We will calculate to obtain at least 180,000 independently infected Hut78-Cas9₊ cells per replicate (total n=6) and maintain at least 1000× sgRNA library coverage across the course of the 12-day culture during the screen. We will then assess the integrated sgRNA sequences using high-throughput sequencing (Illumina NextSeq). We have developed analysis pipelines to align the functional genetic screen results (i.e. fold enrichment or depletion compared to initial time point) of individual sgRNAs to the corresponding amino acid position in the targeted proteins. We will then focus on the peptide regions in p38γ that possess multiple sgRNAs depleted in the screen. These regions represent potential functional elements that when mutated (i.e. loss-of-function) will suppress the proliferation of Hut78 cells, thus provide candidate therapeutic targets for future CTCL treatments.

Validation of the novel functional domains in p38γ protein. To validate the results from the high-density CRISPR protein screen, we will clone the individual sgRNA that targets the newly identified functional elements in p38γ into the ipUSEPR vector. We will then individually transduce the Hut78-Cas9₊ cells with these p38γ domain-targeting sgRNAs and select cells harboring the sgRNA constructs using puromycin. Constructs of sgRNA targeting non-essential DNA sequences in mammalian cells (sgLacZ, sgLuc, and sgRen; two constructs each) will be used as controls. Based on our preliminary studies using shRNA targeting p38γ (FIGS. 35A-35C), we will examine the effect of the relevant p38γ domain-targeting sgRNAs on suppressing the proliferation of CTCL cells (i.e., Hut 78, HH). We will also perform RT-qPCR and western blot to monitor reduced NF-κB expression (mRNA and protein, respectively) in these cells. We will also test if there are any NF-κB signaling changes by detecting the phosphorylation of Ser276 and Ser536, as well as the acetylation of Lys310 positions in NF-κB p65 (methods described above). Finally, we will measure the phosphorylation of DLGH1 at ser158. After validation of the high-density CRISPR screen by individual sgRNAs, we will confirm the role of the newly identified functional elements using a cDNA rescue strategy. We will clone the wild-type human p38γ cDNA (NM_002969.5; 1,101 bp) into the MSCV-ires-GFP retroviral vector. Based on this, we will use wild-type p38γ cDNA construct to clone the individual functional element-deleted constructs via inverse PCR cloning. These p38γ cDNA constructs (both wild-type and domain mutants) will be retrovirally transduced into Hut78 cells with shRNA silencing the endogenous p38γ. This design will allow us to examine the requirement of individual elements in p38γ cDNA to rescue the effects induced by p38γ shRNA in Hut78 cells. We will measure suppressed cell proliferation, reduced expression of NFATC4 and IL17, as well as attenuated NF-κB signaling activation as described above.

Validate hits for CTCL cytotoxicity and p38γ-specific inhibition in vitro and in vivo. We will evaluate the new analogs for inhibitory effect on and selectivity for p38γ in biochemical and cellular in vitro assays, as described above. We will measure effects on p38γ and its downstream targets. The specificity of the new lead compounds for p38γ will be confirmed by examining the effects on activity of other p38 isoforms and other kinases (e.g., PI3K). We will treat CTCL cells (e.g., Hut78, HH) with the candidate inhibitor(s) and determine the extent to which effects (e.g., proliferation and apoptosis) phenocopy that of p38γ knockdown. The compounds that reach the desired potency and specificity will be further analyzed for DMPK properties (via Pharmaron above), coupled with iterative structural studies of protein-ligand interactions. In vivo CTCL xenograft and PDX models. Two lead inhibitors will be selected as the top targets from the in vitro and DMPK studies and evaluated for therapeutic efficacy in 2 CTCL xenograft mouse models developed from 2 CTCL cell lines (Hut78 and HH); the most potent inhibitor will then be tested in 2 PDX models. We will evaluate the top two lead analogs for single agent tumor efficacy in CTCL xenograft models above. Female and male, 8-15-week NSG xenograft mice will be divided into 7 groups (7 mice per group): Group 1=vehicle control; Group 2-4=analog #1 at 3 dose levels; and Group 5-7=analog #2 at 3 dose levels. We will measure and analyze endpoints as described above. We will then compare the single agent activity of the lead p38γ inhibitor, pan-HDACi, and their combination in PDX models as described above. Female and male 8-12-week-old mice will be divided into four groups (7 mice per group): Group 1=vehicle control; Group 2=monotherapy for lead p38γ inhibitor, 1 dose level; Group 3=monotherapy for panHDACi, 1 dose level; group 4=2-drug combination, 1 dose level per agent. We will measure and analyze endpoints as described above.

Expected Results, Potential Pitfalls, and Alternative Approaches: We expect to validate p38γ as a good target for developing anti-cancer therapeutics in CTCL and possibly other cancers that are dependent on p38γ alternate signaling. We expect to obtain a p38γ inhibitor that does not have compound-related toxicity; has improved solubility and other DMPK properties; is at least as potent as F7, with minimal off-target effects; and is feasible for oral administration. Because p38γ is not expressed in normal T-cells and not essential, given that knockout mice are viable (11), we do not expect significant mechanism-based toxicity. Compound-based toxicity will be addressed by the lead optimization effort. We will perform kinase screening to determine effects on targets besides p38γ; in particular, compound F7 is known to be a PI3K inhibitor. However, even if off-target kinase effects are identified, and cannot be addressed through additional optimization, this may not preclude the value of the inhibitor, given that several of the most effective FDA-approved kinase inhibitors (e.g., Gleevec) fall into this category. Because of our extensive experience with the proposed studies, we do not anticipate technical difficulties.

Based on our preliminary experiments using high-density CRISPR protein scan to identify novel functional elements in Dot1l, we anticipate depletion of multiple sgRNAs in select regions of p38γ. These elements represent novel therapeutic targets that when mutated (i.e. loss-of-function) will impair the function of p38γ in supporting CTCL cells. In addition to the known functional elements, including the ATP-binding pocket (Site1 shown in FIGS. 35A-35C), we expect to detect novel elements required for p38γ function. In particular, we will determine the function of two potential drugable sites (Sites 2 and 3 shown in FIGS. 35A-35C) that we identified through our in-house virtual ligand screen pipeline [107]. These two sites belong to a lipid-binding domain that was previously described in a p38 family protein (Ref Diskin 2008, PBD_2NPQ). We have total 60 sgRNA targeting this predicted lipid-binding domain (Gly184-Glu297; FIG. 35C; dotted box) with an average targeting density˜1.9 a.a per sgRNA (FIGS. 35A-35C). We expect our combined efforts merging computational compound docking and the high-density CRISPR genetic screen will reveal novel therapeutic pockets on p38γ amenable to pharmaceutical targeting. We also expect that expression of individual sgRNA targeting these putative functional elements in p38γ will phenocopy the p38γ silencing (by shRNA) and kinase inhibition (by p38γ inhibitor F7). Importantly, we foresee that expression of mutant p38γ with individual domain deletion will not be able to (or have attenuated ability to) rescue p38γ knockdown in CTCL cells. Although we have pre-tested the utility of using high-density CRISPR protein scan to discover novel functional domains, we are aware that the CRISPR/Cas9 system has noticeable off-targeting noises. In addition to the CRISPR/Cas9 from Streptococcus pyogenes that we are currently using, we are now developing the high-density CRISPR protein screen using the CRISPR/Cpf1 system derived from the bacterium Francisella novicida [108-110]. This Cpf1 nuclease uses a “T-rich” PAM sequence, which is distinct from the “G-rich” or “NGG” PAM sequence used by the Cas9 nuclease. This new CRISPR/Cpf1 tool will allow us to conduct “orthogonal validation screens” using sgRNA libraries with distinct sequences to the CRISPR/Cas9 system, thereby minimizing the off-targeting and sequence bias of the single CRISPR system. We also understand that the functional regions suggested by the high-density CRISPR protein scan requires further investigations such as crystallization of these newly identified domains for X-ray structural analyses, as well as investigation of the binding co-effectors to these domains by mass spectrometric studies.

### Example 10: Use of Targeting p38 Gamma Signaling to Advance Cutaneous T Cell Lymphoma Therapy

We have made a very interesting observation regarding gene expression of p38γ in peripheral blood mononuclear cells (PBMC) of both healthy donor and SS patient samples with two p38γ antibodies that targeting two different regions of p38γ (N-terminal or C-terminal). N-terminal antibody is a monoclonal antibody from Abcam (ab205926) that recognizes a region closer to the N-terminus (aa 50-150) of human p38γ MAPK (SAPK3), and C-terminus antibody is from Cell Signaling Technologies (CST) (#2307), a polyclonal antibody that targets the C-terminal residues of human p38γ MAPK.

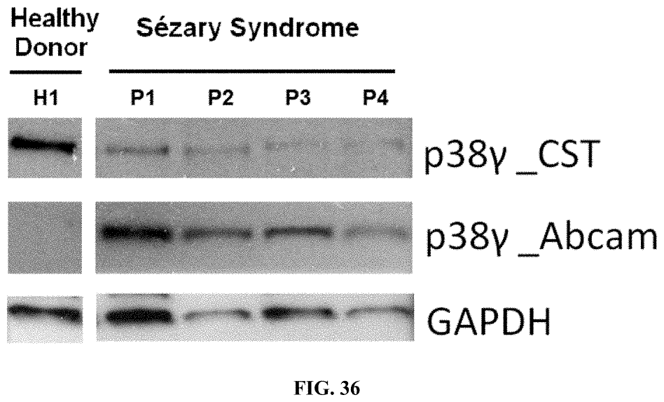

Consistent with our previous result, p38γ MAPK signals in 4 SS patients are not recognized when using the C-terminal antibody (CST) in our western blot analysis; a signal is barely detectable, as reported previously [5]. However, using the Abcam p38γ MAPK antibody, the signals in SS patients are overall stronger in comparison to the healthy normal controls, after normalizing to the loading control of GAPDH (FIG. 36). Although the mechanism in this observation needs further investigation, one possible explanation is that p38γ MAPK maybe truncated (or mutated) at the C-terminus in SS patient samples. We plan to further analyze if there is any mutations near C-terminal in those 32 SS patient Samples by using RNA seq rawdata set that are downloaded from Nature Genetics [26].

This also answers the confusion it may have because the antibody picks up signals that in Healthy donors where the known fact that p38 gamma level is not expressed in normal healthy T cells but only expressed in T-malignant cells. This may due to the possible non-specific binding of the antibody (CST) among MAPKs.

### REFERENCES

1. Wilcox R A. Cutaneous T-cell lymphoma: 2016 update on diagnosis, risk-stratification, and management. American journal of hematology 2016; 91:151-65.
2. Hwang S T, Janik J E, Jaffe E S, Wilson W H. Mycosis fungoides and Sézary syndrome. The Lancet; 371:945-57.
3. Jawed S I, Myskowski P L, Horwitz S, Moskowitz A, Querfeld C. Primary cutaneous T-cell lymphoma (mycosis fungoides and Sézary syndrome): part II. Prognosis, management, and future directions. Journal of the American Academy of Dermatology 2015; 70:223 e1-17; quiz 40-2.
4. Izban K F, Ergin M, Qin J-Z, et al. Constitutive expression of NF-kB is a characteristic feature of mycosis fungoides: Implications for apoptosis resistance and pathogenesis. Human pathology; 31:1582-90.
5. Bliss-Moreau M, Coarfa C, Gunaratne P H, Guitart J, Krett N L, Rosen S T. Identification of p38beta as a therapeutic target for the treatment of Sézary syndrome. The Journal of investigative dermatology 2015; 135:599-608.
6. Li Z, Jiang Y, Ulevitch R J, Han J. The Primary Structure of p38γ: A New Member of p38 Group of MAP Kinases. Biochemical and biophysical research communications 1996; 228:334-40.
7. Wang X S, Diener K, Manthey C L, et al. Molecular cloning and characterization of a novel p38 mitogen-activated protein kinase. The Journal of biological chemistry 1997; 272:23668-74.
8. Pearson G, Robinson F, Beers Gibson T, et al. Mitogen-activated protein (MAP) kinase pathways: regulation and physiological functions. Endocrine reviews 2001; 22:153-83.
9. Mittelstadt P R, Salvador J M, Fornace A J, Jr., Ashwell J D. Activating p38 MAPK: new tricks for an old kinase. Cell cycle 2005; 4:1189-92.
10. Wu C C, Hsu S C, Shih H M, Lai M4. Nuclear factor of activated T cells c is a target of p38 mitogen-activated protein kinase in T cells. Molecular and cellular biology 2003; 23:6442-54.
11. Salvador J M, Mittelstadt P R, Guszczynski T, et al. Alternative p38 activation pathway mediated by T cell receptor-proximal tyrosine kinases. Nature immunology 2005; 6:390-5.
12. Mittelstadt P R, Yamaguchi H, Appella E, Ashwell J D. T cell receptor-mediated activation of p38{alpha} by mono-phosphorylation of the activation loop results in altered substrate specificity. The Journal of biological chemistry 2009; 284:15469-74.
13. Alam M S, Gaida M M, Ogawa Y, Kolios A G, Lasitschka F, Ashwell J D. Counter-regulation of T cell effector function by differentially activated p38. The Journal of experimental medicine 2015; 211:1257-70.
14. Round J L, Humphries L A, Tomassian T, Mittelstadt P, Zhang M, Miceli M C. Scaffold protein Dlgh1 coordinates alternative p38 kinase activation, directing T cell receptor signals toward NFAT but not NF-kappaB transcription factors. Nature immunology 2007; 8:154-61.
15. Sabio G, Arthur J S C, Kuma Y, et al. p38γ regulates the localisation of SAP97 in the cytoskeleton by modulating its interaction with GKAP. The EMBO journal 2005; 24:1134-45.
16. da Silva Almeida A C, Abate F, Khiabanian H, et al. The mutational landscape of cutaneous T cell lymphoma and Sézary syndrome. Nature genetics 2015; 47:1565-70.
17. Risco A, Cuenda A. New Insights into the p38γ and p38δ MAPK Pathways. Journal of Signal Transduction 2012; 2012:8.
18. Caprini E, Cristofoletti C, Arcelli D, et al. Identification of key regions and genes important in the pathogenesis of Sézary syndrome by combining genomic and expression microarrays. Cancer research 2009; 69:8438-46.
19. van Doorn R, van Kester M S, Dijkman R, et al. Oncogenomic analysis of mycosis fungoides reveals major differences with Sézary syndrome. Blood 2009; 113:127-36.
20. Iqbal J, Weisenburger D D, Greiner T C, et al. Molecular signatures to improve diagnosis in peripheral T-cell lymphoma and prognostication in angioimmunoblastic T-cell lymphoma. Blood 2010; 115:1026-36.
21. Ozes O N, Blatt L M, Seiwert S D. Use of pirfenidone in therapeutic regimens. Google Patents; 2008.
22. Yin N, Qi X, Tsai S, et al. p38gamma MAPK is required for inflammation-associated colon tumorigenesis. Oncogene 2016; 35:1039-48.
23. Querfeld C, Curran S A, Leung S, et al. T Cells in CTCL Have an Exhausted Phenotype While Cutaneous Dendritic Cells Display a Normally Activated Mature Phenotype. Blood 2015; 124:1695-.
24. Ichida M, Finkel T. Ras regulates NFAT3 activity in cardiac myocytes. The Journal of biological chemistry 2001; 276:3524-30.
25. Vallois D, Dobay M P, Morin R D, et al. Activating mutations in genes related to TCR signaling in angioimmunoblastic and other follicular helper T-cell-derived lymphomas. Blood 2016; 128:1590-502.
26. Wang L, Ni X, Covington K R, et al. Genomic profiling of Sézary syndrome identifies alterations of key T cell signaling and differentiation genes. Nature genetics 2015; 47:1426-34.
27. Vaque J P, Gomez-Lopez G, Monsalvez V, et al. PLCG1 mutations in cutaneous T-cell lymphomas. Blood 2015; 123:2034-43.
28. Suga H, Sugaya M, Miyagaki T, et al. The role of IL-32 in cutaneous T-cell lymphoma. The Journal of investigative dermatology 2015; 134:1528-35.
29. Yousif N G, Al-Amran F G, Hadi N, Lee J, Adrienne J. Expression of IL-32 modulates NF-kappaB and p38 MAP kinase pathways in human esophageal cancer. Cytokine 2013; 61:223-7.
30. Schafer P H, Wadsworth S A, Wang L, Siekierka J J. p38 alpha mitogen-activated protein kinase is activated by CD28-mediated signaling and is required for IL-4 production by human CD4+CD45RO+ T cells and Th2 effector cells. Journal of immunology 1999; 162:7110-9.
31. Ficarro S B, Zhang Y, Carrasco-Alfonso M J, et al. Online nanoflow multidimensional fractionation for high efficiency phosphopeptide analysis. Molecular & cellular proteomics: MCP 2011; 10:O111 011064.
32. Yang T T, Xiong Q, Enslen H, Davis R J, Chow C W. Phosphorylation of NFATc4 by p38 mitogen-activated protein kinases. Molecular and cellular biology 2002; 22:3892-904.
33. Courcelles M, Lemieux S, Voisin L, Meloche S, Thibault P. ProteoConnections: a bioinformatics platform to facilitate proteome and phosphoproteome analyses. Proteomics 2011; 11:2654-71.
34. Tycko J, Myer V E, Hsu P D. Methods for Optimizing CRISPR-Cas9 Genome Editing Specificity. Molecular cell 2016; 63:355-70.
35. Place R F, Noonan E J, Giardina C. HDAC inhibition prevents NF-κB activation by suppressing proteasome activity: Down-regulation of proteasome subunit expression stabilizes IκBα. Biochemical Pharmacology 2005; 70:394-406.
36. Hayakawa M, Kawaguchi K, Kai4awa H, et al. Synthesis and biological evaluation of sulfonylhydrazone-substituted imidazo[1,2-a]pyridines as novel PI3 kinase p110alpha inhibitors. Bioorganic & medicinal chemistry 2007; 15:5837-44.
37. New M, Olzscha H, La Thangue N B. HDAC inhibitor-based therapies: Can we interpret the code? Molecular Oncology 2012; 6:637-56.
38. Brogdon J L, Xu Y, Szabo S J, et al. Histone deacetylase activities are required for innate immune cell control of Th1 but not Th2 effector cell function. Blood 2007; 109:1123-30.
39. Fantin V R, Loboda A, Pawelet4 C P, et al. Constitutive activation of signal transducers and activators of transcription predicts vorinostat resistance in cutaneous T-cell lymphoma. Cancer research 2008; 68:3785-94.
40. Bates S E, Eisch R, Ling A, et al. Romidepsin in peripheral and cutaneous T-cell lymphoma: mechanistic implications from clinical and correlative data. British journal of haematology 2015; 170:96-109.
41. Nolan L, Johnson P W, Ganesan A, Packham G, Crabb S J. Will histone deacetylase inhibitors require combination with other agents to fulfil their therapeutic potential? British journal of cancer 2008; 99:689-94.
42. Saba N S, Liu D, Herman S E, et al. Pathogenic role of B-cell receptor signaling and canonical NF-kappaB activation in mantle cell lymphoma. Blood 2016; 128:82-92.
43. Greten F R, Karin M. The IKK/NF-kappaB activation pathway—a target for prevention and treatment of cancer. Cancer Lett 2004; 206:193-9.
44. Karin M, Lin A. NF-[kappa]B at the crossroads of life and death. Nature immunology 2002; 3:221-7.
45. Covassin L, Jangalwe S, Jouvet N, et al. Human immune system development and survival of non-obese diabetic (NOD)-scid IL2rgamma(null) (NSG) mice engrafted with human thymus and autologous haematopoietic stem cells. Clinical and experimental immunology 2013; 174:372-88.
46. Baldwin A S, Jr. The NF-kappa B and I kappa B proteins: new discoveries and insights. Annual review of immunology 1996; 15:649-83.
47. Sors A, Jean-Louis F, Begue E, et al. Inhibition of IkappaB kinase subunit 2 in cutaneous T-cell lymphoma down-regulates nuclear factor-kappaB constitutive activation, induces cell death, and potentiates the apoptotic response to antineoplastic chemotherapeutic agents. Clinical cancer research: an official journal of the American Association for Cancer Research 2008; 15:901-11.
48. Nicolay J P, Muller-Decker K, Schroeder A, et al. Dimethyl fumarate restores apoptosis sensitivity and inhibits tumor growth and metastasis in CTCL by targeting NF-kappaB. Blood 2016; 128:805-15.
49. Khan O, Fotheringham S, Wood V, et al. HR23B is a biomarker for tumor sensitivity to HDAC inhibitor-based therapy. Proceedings of the National Academy of Sciences of the United States of America 2010; 107:6532-7.
50. New M, Olzscha H, Liu G, et al. A regulatory circuit that involves HR23B and HDAC6 governs the biological response to HDAC inhibitors. Cell Death and Differentiation 2013; 20:1306-16.
51. Fotheringham S, Epping M T, Stimson L, et al. Genome-wide loss-of-function screen reveals an important role for the proteasome in HDAC inhibitor-induced apoptosis. Cancer cell 2009; 15:57-66.
52. Zhong H, May M J, Jimi E, Ghosh S. The phosphorylation status of nuclear NF-kappa B determines its association with CBP/p300 or HDAC-1. Molecular cell 2002; 9:625-36.
53. Ito K, Barnes P J, Adcock I M. Glucocorticoid receptor recruitment of histone deacetylase 2 inhibits interleukin-1beta-induced histone H4 acetylation on lysines 8 and 12. Molecular and cellular biology 2000; 20:6891-903.
54. Chen L, Fischle W, Verdin E, Greene W C. Duration of nuclear NF-kappaB action regulated by reversible acetylation. Science 2001; 293:1653-7.
55. West A C, Johnstone R W. New and emerging HDAC inhibitors for cancer treatment. The Journal of clinical investigation 2015; 124:30-9.
56. Marquard L, Gjerdrum L M, Christensen I J, Jensen P B, Sehested M, Ralfkiaer E. Prognostic significance of the therapeutic targets histone deacetylase 1, 2, 6 and acetylated histone H4 in cutaneous T-cell lymphoma. Histopathology 2008; 53:267-77.
57. Dai Y, Rahmani M, Dent P, Grant S. Blockade of histone deacetylase inhibitor-induced RelA/p65 acetylation and NF-kappaB activation potentiates apoptosis in leukemia cells through a process mediated by oxidative damage, XIAP downregulation, and c-Jun N-terminal kinase 1 activation. Molecular and cellular biology 2005; 25:5429-44.
58. Zhang Z, Yamashita H, Toyama T, et al. HDAC6 expression is correlated with better survival in breast cancer. Clinical cancer research: an official journal of the American Association for Cancer Research 2004; 10:6962-8.
59. Kotian S, Liyanarachchi S, zelent A, Parvin J D. Histone deacetylases 9 and 10 are required for homologous recombination. The Journal of biological chemistry 2011; 286:7722-6.
60. Park J H, Kim S H, Choi M C, et al. Class II histone deacetylases play pivotal roles in heat shock protein 90-mediated proteasomal degradation of vascular endothelial growth factor receptors. Biochemical and biophysical research communications 2008; 368:318-22.
61. Domingue4 C, Boelens R, Bonvin A M. HADDOCK: a protein-protein docking approach based on biochemical or biophysical information. Journal of the American Chemical Society 2003; 125:1731-7.
62. Wang J, Hu W, Cai S, Lee B, Song J, Chen Y. The intrinsic affinity between E2 and the Cys domain of El in ubiquitin-like modifications. Molecular cell 2007; 27:228-37.
63. Lee W, Tonelli M, Markley J L. NMRFAM-SPARKY: enhanced software for biomolecular NMR spectroscopy. Bioinformatics 2015; 31:1325-7.
64. Goto N K, Gardner K H, Mueller G A, Willis R C, Kay L E. A robust and cost-effective method for the production of Val, Leu, Ile (delta 1) methyl-protonated 15N-, 13C-, 2H-labeled proteins. J Biomol NMR 1999; 13:369-74.
65. Hu W, Namanja A T, Wong S, Chen Y. Selective editing of Val and Leu methyl groups in high molecular weight protein NMR. J Biomol NMR 2012; 53:113-24.
66. Wurz R P, Pettus L H, Henkle B, et al. Part 2: Structure-activity relationship (SAR) investigations of fused pyrazoles as potent, selective and orally available inhibitors of p38alpha mitogen-activated protein kinase. Bioorganic & medicinal chemistry letters 2010; 20:1680-4.
67. Campbell R M, Anderson B D, Brooks N A, et al. Characterization of LY2228820 dimesylate, a potent and selective inhibitor of p38 MAPK with antitumor activity. Mol Cancer Ther 2015; 13:364-74.
68. Pellecchia M, Meininger D, Dong Q, Chang E, Jack R, Sem D S. NMR-based structural characterization of large protein-ligand interactions. J Biomol NMR 2002; 22:165-73.
69. Townsend E C, Murakami M A, Christodoulou A, et al. The Public Repository of Xenografts Enables Discovery and Randomized Phase II-like Trials in Mice. Cancer cell 2016; 29:574-86.
70. Pillaire M-J, Nebreda A R, Darbon J-M. Cisplatin and UV Radiation Induce Activation of the Stress-Activated Protein Kinase p38γ in Human Melanoma Cells. Biochemical and biophysical research communications 2000; 278:724-8.
71. Wang X, McGowan C H, 4hao M, et al. Involvement of the MKK6-p38gamma cascade in gamma-radiation-induced cell cycle arrest. Molecular and cellular biology 2000; 20:4543-52.
72. Chen L, Mayer J A, Krisko T I, et al. Inhibition of the p38 kinase suppresses the proliferation of human ER-negative breast cancer cells. Cancer research 2009; 69:8853-61.
73. Qi X, Tang J, Loesch M, Pohl N, Alkan S, Chen G. p38γ Mitogen-Activated Protein Kinase Integrates Signaling Crosstalk between Ras and Estrogen Receptor to Increase Breast Cancer Invasion. Cancer research 2006; 66:7540-7.
74. Alam M S et al., J Exp Med 2015; 211: 1257-1270.
75. Moran N. p38 kinase inhibitor approved for idiopathic pulmonary fibrosis. Nature biotechnology 2011; 29(4):301.
76. Hale K K, Trollinger D, Rihanek M, Manthey C L. Differential expression and activation of p38 mitogen-activated protein kinase alpha, beta, gamma, and delta in inflammatory cell lineages. Journal of immunology 1999; 162(7):4246-52.
77. Browne A J, Gobel A, Thiele S, Hofbauer L C, Rauner M, Rachner T D. p38 MAPK regulates the Wnt inhibitor Dickkopf-1 in osteotropic prostate cancer cells. Cell death & disease 2016; 7:e2119.
78. Zheng S, Yang C, Liu T, Liu Q, Dai F, Sheyhidin I, et al. Clinicopathological significance of p38beta, p38gamma, and p38delta and its biological roles in esophageal squamous cell carcinoma. Tumour biology: the journal of the International Society for Oncodevelopmental Biology and Medicine 2016; 37(6):7255-66.
79. Zheng Z, Amran S I, Thompson P E, Jennings I G. Isoform-selective inhibition of phosphoinositide 3-kinase: identification of a new region of nonconserved amino acids critical for p110alpha inhibition. Molecular pharmacology 2011; 80(4):657-64.
80. Qi X, Tang J, Pramanik R, Schultz R M, Shirasawa S, Sasazuki T, et al. p38 MAPK activation selectively induces cell death in K-ras-mutated human colon cancer cells through regulation of vitamin D receptor. The Journal of biological chemistry 2004; 279(21):22138-44.
81. Qi X, Yin N, Ma S, Lepp A, Tang J, Jing W, et al. p38gamma MAPK Is a Therapeutic Target for Triple-Negative Breast Cancer by Stimulation of Cancer Stem-Like Cell Expansion. Stem cells 2015; 33(9):2738-47.
82. Kozono S, Ohuchida K, Eguchi D, Ikenaga N, Fujiwara K, Cui L, et al. Pirfenidone inhibits pancreatic cancer desmoplasia by regulating stellate cells. Cancer research 2013; 73(7):2345-56.
83. Lemke J, von Karstedt S, Abd El Hay M, Conti A, Arce F, Montinaro A, et al. Selective CDK9 inhibition overcomes TRAIL resistance by concomitant suppression of cFlip and Mcl-1. Cell Death Differ 2014; 21(3):491-502.
84. Soond D R, Bjorgo E, Moltu K, Dale V Q, Patton D T, Torgersen K M, et al. PI3K p110delta regulates T-cell cytokine production during primary and secondary immune responses in mice and humans. Blood 2010; 115(11):2203-13.
85. Hanahan D, Weinberg R A. Hallmarks of cancer: the next generation. Cell 2011; 144(5):646-74.
86. Courtney K D, Corcoran R B, Engelman J A. The PI3K pathway as drug target in human cancer. J Clin Oncol 2010; 28(6):1075-83.
87. Querfeld C, Rizvi M A, Kuzel T M. The selective protein kinase C beta inhibitor enzastaurin induces apoptosis in cutaneous T-cell lymphoma cell lines through the AKT pathway. The Journal of investigative dermatology 2006; 126.
88. Ho R C, Alcazar O, Fujii N, Hirshman M F, Goodyear U. p38gamma MAPK regulation of glucose transporter expression and glucose uptake in L6 myotubes and mouse skeletal muscle. American journal of physiology Regulatory, integrative and comparative physiology 2004; 286(2):R342-9.
89. Querfeld C, Kuzel T M, Kim Y H, Porcu P, Duvic M, Musiek A, Rook A H, Mark L A, Pinter-Brown L, Hamid O, Lin B, Bian Y, Boye M, Day J M, Rosen S T. Multicenter phase II trial of enzastaurin in patients with relapsed or refractory advanced cutaneous T-cell lymphoma. Leuk Lymphoma. 2011 August; 52(8):1474-80. PubMed PMID: 21649541
90. Zhang X H, Zhao C, Ma Z A. The increase of cell-membranous phosphatidylcholines containing polyunsaturated fatty acid residues induces phosphorylation of p53 through activation of ATR. J Cell Sci. 2007 Dec. 1; 120(Pt 23):4134-43. PubMed PMID: 18032786; PubMed Central PMCID: PMC2915541.
91. Pons B, Armengol G, Livingstone M, Lopez L, Coch L, Sonenberg N, et al. Association between LRRK2 and 4E-BP1 protein levels in normal and malignant cells. Oncology reports 2012; 27(1):225-31.
92. Nam S, Wen W, Schroeder A, Herrmann A, Yu H, Cheng X, et al. Dual inhibition of Janus and Src family kinases by novel indirubin derivative blocks constitutively-activated Stat3 signaling associated with apoptosis of human pancreatic cancer cells. Molecular oncology 2013; 7(3):369-78.
93. Wallin J J, Guan J, Prior W W, Lee L B, Berry L, Belmont L D, et al. GDC-0941, a novel class I selective PI3K inhibitor, enhances the efficacy of docetaxel in human breast cancer models by increasing cell death in vitro and in vivo. Clin Cancer Res 2012; 18(14):3901-11.
94. Maira S M, Stauffer F, Brueggen J, Furet P, Schnell C, Fritsch C, et al. Identification and characterization of NVP-BEZ235, a new orally available dual phosphatidylinositol 3-kinase/mammalian target of rapamycin inhibitor with potent in vivo antitumor activity. Molecular cancer therapeutics 2008; 7(7):1851-63.
95. da Silva Almeida A C, Abate F, Khiabanian H, Martinez-Escala E, Guitart J, Tensen C P, et al. The mutational landscape of cutaneous T cell lymphoma and Sézary syndrome. Nature genetics. 2015; 47(12):1465-70. Epub 2015/11/10. doi: 10.1038/ng.3442. PubMed PMID: 26551667; PubMed Central PMCID: PMCPMC4878831.
96. Yin N, Qi X, Tsai S, Lu Y, Basir Z, Oshima K, et al. p38gamma MAPK is required for inflammation-associated colon tumorigenesis. Oncogene. 2015. doi: 10.1038/onc.2015.158. PubMed PMID: 25961922.
97. Berg L J. Strength of T cell receptor signaling strikes again. Immunity. 2009; 31(4):529-31. doi: 10.1016/j.immuni.2009.09.008. PubMed PMID: 19833081.
98. Cuenda A, Sanz-Ezquerro J J. p38gamma and p38delta: From Spectators to Key Physiological Players. Trends in biochemical sciences. 2017; 42(6):431-42. doi: 10.1016/j.tibs.2017.02.008. PubMed PMID: 28473179.
99. Trapp J, Jochum A, Meier R, Saunders L, Marshall B, Kunick C, et al. Adenosine mimetics as inhibitors of NAD+-dependent histone deacetylases, from kinase to sirtuin inhibition. Journal of medicinal chemistry. 2006; 49(25):7307-16. doi: 10.1021/jm060118b. PubMed PMID: 17149860.
100. Wilson A J, Byun D S, Popova N, Murray L B, L'Italien K, Sowa Y, et al. Histone deacetylase 3 (HDAC3) and other class I HDACs regulate colon cell maturation and p21 expression and are deregulated in human colon cancer. The Journal of biological chemistry. 2006; 281(19):13548-58. doi: 10.1074/jbc.M510023200. PubMed PMID: 16533812.
101. Marquard L, Gjerdrum L M, Christensen I J. Prognostic significance of the therapeutic targets histone deacetylase 1, 2, 6 and acetylated histone H4 in cutaneous T-cell lymphoma. Histopathology. 2008; 53.
102. Marquard L, Poulsen C B, Gjerdrum L M, de Nully Brown P, Christensen I J, Jensen P B, et al. Histone deacetylase 1, 2, 6 and acetylated histone H4 in B- and T-cell lymphomas. Histopathology. 2009; 54(6):688-98. doi: 10.1111/j.1365-2559.2009.03290.x. PubMed PMID: 19438744.
103. Hrabeta J, Stiborova M, Adam V, Kizek R, Eckschlager T. Histone deacetylase inhibitors in cancer therapy. A review. Biomedical papers of the Medical Faculty of the University Palacky, Olomouc, Czechoslovakia. 2014; 158(2):161-9. doi: 10.5507/bp.2013.085. PubMed PMID: 24263215.
104. Shi J, Wang E, Milazzo J P, Wang Z, Kinney J B, Vakoc C R. Discovery of cancer drug targets by CRISPR-Cas9 screening of protein domains. Nature biotechnology. 2015; 33(6):661-7. doi: 10.1038/nbt.3235. PubMed PMID: PMC4529991.
105. Bernt K M, Zhu N, Sinha A U, Vempati S, Faber J, Krivtsov A V, et al. MLL-rearranged leukemia is dependent on aberrant H3K79 methylation by DOT1L. Cancer cell. 2011; 20(1):66-78. doi: 10.1016/j.ccr.2011.06.010. PubMed PMID: 21741597; PubMed Central PMCID: PMC3329803.
106. Chen C W, Koche R P, Sinha A U, Deshpande A J, Zhu N, Eng R, et al. DOT1L inhibits SIRT1-mediated epigenetic silencing to maintain leukemic gene expression in MLL-rearranged leukemia. Nature medicine. 2015; 21(4):335-43. doi: 10.1038/nm.3832. PubMed PMID: 25822366; PubMed Central PMCID: PMC4390532.
107. Li H, Kasam V, Tautermann C S, Seeliger D, Vaidehi N. Computational method to identify druggable binding sites that target protein-protein interactions. Journal of chemical information and modeling. 2014; 54(5):1391-400. Epub 2014/04/26. doi: 10.1021/ci400750x. PubMed PMID: 24762202.
108. Zetsche B, Gootenberg J S, Abudayyeh O O, Slaymaker I M, Makarova K S, Essletzbichler P, et al. Cpf1 is a single RNA-guided endonuclease of a class 2 CRISPR-Cas system. Cell. 2015; 163(3):759-71. doi: 10.1016/j.cell.2015.09.038. PubMed PMID: 26422227; PubMed Central PMCID: PMC4638220.
109. Dong D, Ren K, Qiu X, Zheng J, Guo M, Guan X, et al. The crystal structure of Cpf1 in complex with CRISPR RNA. Nature. 2016; 532(7600):522-6. doi: 10.1038/nature17944. PubMed PMID: 27096363.
110. Li B, Zhao W, Luo X, Zhang X, Li C, Zeng C, et al. Engineering CRISPR-Cpf1 crRNAs and mRNAs to maximize genome editing efficiency. Nature biomedical engineering. 2017; 1(5). doi: 10.1038/s41551-017-0066. PubMed PMID: 28840077; PubMed Central PMCID: PMC5562407.
111. Lien E C, Dibble C C, Toker A. PI3K signaling in cancer: beyond AKT. Current Opinion in Cell Biology. 2017; 45:62-71.
112. Delaglio F, Grzesiek S, Vuister G W, Zhu G, Pfeifer J, Bax A. NMRPipe: a multidimensional spectral processing system based on UNIX pipes. Journal of biomolecular NMR. 1995; 6(3):277-93. PubMed PMID: 8520220.
113. Tugarinov V, Kay L E. Ile, Leu, and Val methyl assignments of the 723-residue malate synthase G using a new labeling strategy and novel NMR methods. Journal of the American Chemical Society. 2003; 125(45):13868-78. doi: 10.1021/ja030345s. PubMed PMID: 14599227.
114. Aoto P C, Martin B T, Wright P E. NMR Characterization of Information Flow and Allosteric Communities in the MAP Kinase p38gamma. Scientific reports. 2016; 6:28655. doi: 10.1038/srep28655. PubMed PMID: 27353957; PubMed Central PMCID: PMC4926091.
115. Yang F, Nam S, Brown C E, Zhao R, Starr R, Home D A, et al. A Novel Berbamine Derivative Inhibits Cell Viability and Induces Apoptosis in Cancer Stem-Like Cells of Human Glioblastoma, via Up-Regulation of miRNA-4284 and JNK/AP-1 Signaling. PloS one. 2014; 9(4):e94443.
116. Chu P G, Chung L, Weiss L M, Lau S K. Determining the site of origin of mucinous adenocarcinoma: an immunohistochemical study of 175 cases. The American journal of surgical pathology. 2011; 35(12):1830-6.
117. Ergin E, Dogan A, Parmaksiz M, Elcin A E, Elcin Y M. Time-Resolved Fluorescence Resonance Energy Transfer [TR-FRET] Assays for Biochemical Processes. Current pharmaceutical biotechnology 2016; 17(14):1222-30.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of treating cutaneous T-cell lymphoma (CTCL) in a subject in need thereof, the method comprising administering an effective amount of a p38 gamma (p38γ) kinase inhibitor to said subject.

2. The method of claim 1, wherein the p38γ kinase inhibitor is a compound represented by Formula (I): 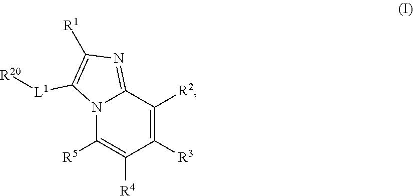
wherein:
L¹ is a bond, —SOₙ₁₁L^{1A}-, —SOₙ₁₁NR¹¹L^{1A}-, —NHC(O)NR¹¹L^{1A}-, —NR¹¹L^{1A}-, —C(O)L^{1A}-, —C(O)OL^{1A}-, —C(O)NR¹¹L^{1A}-, —OL^{1A}-, —NR¹¹SO₂L^{1A}-, —NR¹¹C(O)L^{1A}-, —NR¹¹C(O)OL^{1A}-, —NR¹¹OL^{1A}-, —SL^{1A}-, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CX¹₃, —CHX¹₂, —CH₂X¹, —OCX¹₃, —OCH₂X¹, —OCHX¹₂, —N₃, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —OCX²₃, —OCH₂X², —OCHX²₂, —N₃, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —N₃, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —N₃, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, halogen, —CX⁵₃, —CHX⁵₂, —CH₂X⁵, —OCX⁵₃, —OCH₂X⁵, —OCHX⁵₂, —N₃, —CN, —SOₙ₅R^{5D}, —SOᵥ₅NR^{5A}R^{5B}, —NHC(O)NR^{5A}R^{5B}, —N(O)ₘ₅, —NR^{5A}R^{5B}, —C(O)R_{5C}, —C(O)—OR^{5C}, —C(O)NR^{5A}R^{5B}, —OR^{5D}, —NR^{5A}SO₂R^{5D}, —NR^{5A}C(O)R^{5C}, —NR^{5A}C(O)OR^{5C}, —NR^{5A}OR^{5C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²⁰ is substituted or unsubstituted cycloalkyl, substituted or unsubstituted heterocycloalkyl, substituted or unsubstituted aryl, or substituted or unsubstituted heteroaryl;
wherein R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D} and R¹¹ are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{1A} is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
n1, n2, n3, n4, n5 and n11 are independently an integer from 0 to 4;
m1, m2, m3, m4, m5, v1, v2, v3, v4, v5 and v11 are independently an integer from 1 to 2; and
X, X¹, X², X³, X⁴, and X⁵ are independently —F, —Cl, —Br, or —I.

3. The method of claim 2, wherein the p38γ kinase inhibitor is a compound represented by Formula (III): 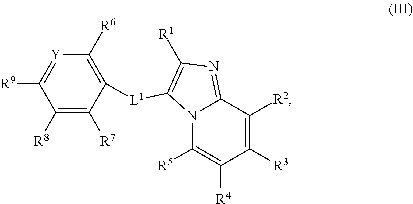
wherein:
Y is —N═ or —CR¹²═;
R⁶ is hydrogen, halogen, —CX⁶₃, —CHX⁶₂, —CH₂X⁶, —OCX⁶₃, —OCH₂X⁶, —OCHX⁶₂, —N₃, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is hydrogen, halogen, —CX⁷₃, —CHX⁷₂, —CH₂X⁷, —OCX⁷₃, —OCH₂X⁷, —OCHX⁷₂, —N₃, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is hydrogen, halogen, —CX⁸₃, —CHX⁸₂, —CH₂X⁸, —OCX⁸₃, —OCH₂X⁸, —OCHX⁸₂, —N₃, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —OR^{8D}, —NR^{8A}SO₂R^{8D}, —NR^{8A}C(O)R^{8C}, —NR^{8A}C(O)OR^{8C}, —NR^{8A}OR^{8C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CX⁹₃, —CHX⁹₂, —CH₂X⁹, —OCX⁹₃, —OCH₂X⁹, —OCHX⁹₂, —N₃, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen, halogen, —CX¹²₃, —CHX¹²₂, —CH₂X¹², —OCX¹²₃, —OCH₂X¹², —OCHX¹²₂, —N₃, —CN, —SOₙ₁₂R^{12D}, —SOᵥ₁₂NR^{12A}R^{12B}, —NHC(O)NR^{12A}R^{12B}, —N(O)ₘ₁₂, —NR^{12A}R^{12B}, —C(O)R^{12C}, —C(O)—OR^{12C}, —C(O)NR^{12A}R^{12B}, —OR^{12D}, —NR^{12A}SO₂R^{12D}, —NR^{12A}C(O)R^{12C}, —NR^{12A}C(O)OR^{12C}, —NR^{12A}OR^{12C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
wherein R^{6A}, R^{6B}, R^{6C}, R^{6D}, R^{7A}, R^{7B}, R^{7C}, R^{7D}, R^{8A}, R^{8B}, R^{8C}, R^{8D}, R^{9A}, R^{9B}, R^{9C}, R^{9D}, R^{12A}, R^{12B}, R^{12C}, and R^{12D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ and R⁸ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁸ and R⁹ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁹ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁶ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
n6, n7, n8, n9 and n12 are independently an integer from 0 to 4;
m6, m7, m8, m9, m12, v6, v7, v8, v9 and v12 are independently an integer from 1 to 2; and
X⁶, X⁷, X⁸, X⁹ and X¹² are independently —F, —Cl, —Br, or —I.

4. The method of claim 2, wherein R¹ is hydrogen.

5. The method of claim 3, wherein Y is —CR¹²═.

6. The method of claim 5, wherein R¹² is hydrogen, halogen, or unsubstituted C₁-C₄ alkyl.

7. The method of claim 3, wherein Y is —N═.

8. The method of claim 2, wherein:
L¹ is R¹³-substituted or unsubstituted phenylene;
R¹³ is halogen, —CX¹³₃, —CHX¹³₂, —CH₂X¹³, —OCX¹³₃, —OCH₂X¹³, —OCHX¹³₂, —CN, —SOₙ₁₃R^{13D}, —SOᵥ₁₃NR^{13A}R^{13B}, —NHC(O)NR^{13A}R^{13B}, —N(O)ₘ₁₃, —NR^{13A}R^{13B}, —C(O)R^{13C}, —C(O)—OR^{13C}, —C(O)NR^{13A}R^{13B}, —OR^{13D}, —NR^{13A}SO₂R^{13D}, —NR^{13A}C(O)R^{13C}, —NR^{13A}C(O)OR^{13C}, —NR^{13A}OR^{13C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl,
wherein R^{13A}, R^{13B}, R^{13C}, and R^{13D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n13 is independently an integer from 0 to 4, and
m13 and v13 are each independently an integer from 0 to 2, and
X¹³ is independently —F, —Cl, —Br, or —I.

9. The method of claim 2, wherein:
L¹ is R¹³-substituted or unsubstituted heteroalkylene;
R¹³ is halogen, —CX¹³₃, —CHX¹³₂, —CH₂X¹³, —OCX¹³₃, —OCH₂X¹³, —OCHX¹³₂, —CN, —SO₁₃R^{13D}, —SOᵥ₁₃NR^{13A}R^{13B}, —NHC(O)NR^{13A}R^{13B}, —N(O)ₘ₁₃, —N R^{13A}R^{13B}, —C(O)R^{13C}, —C(O)—OR^{13C}, —C(O)NR^{13A}R^{13B}, —OR^{13D}, —NR^{13A}SO₂R^{13D}, —NR^{13A}C(O)R^{13C}, —NR^{13A}C(O)OR^{13C}, —NR^{13A}OR^{13C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl,
wherein R^{13A}, R^{13B}, R^{13C}, and R^{13D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n13 is independently an integer from 0 to 4,
m13 and v13 are each independently an integer from 0 to 2, and
X¹³ is independently —F, —Cl, —Br, or —I.

10. The method of claim 2, wherein:
L¹ is R¹³-substituted or unsubstituted 2 to 4 membered heteroalkylene; and
R¹³ is halogen, or unsubstituted C₁-C₃ alkyl.

11. The method of claim 2, wherein:
L¹ is —SO₂N(R¹⁴)N═CH—;
wherein R¹⁴ is hydrogen, —CX¹⁴₃, —CHX¹⁴₂, —CH₂X¹⁴, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, and
X¹⁴ is independently —F, —Cl, —Br, or —I.

12. The method of claim 11, wherein R¹⁴ is unsubstituted C₁-C₃ alkyl.

13. The method of claim 2, wherein L¹ is —SO₂—N(CH₃)N═CH—.

14. The method of claim 2, wherein at least one of R², R³, R⁴ and R⁵ is halogen.

15. The method of claim 14, wherein R², R³, and R⁵ are hydrogen and R⁴ is —F, —Cl or —Br.

16. The method of claim 2, wherein at least one of R², R³, R⁴ and R⁵ is halogen-substituted or unsubstituted pyridyl.

17. The method of claim 3, wherein R⁶, R⁷, R⁸ and R⁹ are independently hydrogen, halogen, —NO₂, or unsubstituted C₁-C₃ alkyl.

18. The method of claim 3, wherein:
L¹ is unsubstituted phenylene;
R¹, R², R³ and R⁵ are hydrogen;
R⁴ is —F, —Cl or —Br;
R⁶, R⁷, R⁸ and R⁹ are independently hydrogen, halogen, NO₂, or unsubstituted C₁-C₃ alkyl.

19. The method of claim 3, wherein:
L¹ is substituted or unsubstituted C₄-C₆ alkylene, or substituted or unsubstituted 4 to 6 membered heteroalkylene;
R¹, R², R³ and R⁵ are hydrogen;
R⁴ is —Cl or —Br; and
R⁶, R⁷, R⁸ and R⁹ are independently hydrogen, halogen, NO₂, or unsubstituted C₁-C₃ alkyl.

20. The method of claim 3, wherein:
L¹ is —SO₂—N(CH₃)N═CH—;
R², R³ and R⁵ are hydrogen;
R⁴ is halogen, or halogen-substituted or unsubstituted pyridyl;
R⁶ is unsubstituted methyl;
R⁷ is hydrogen;
R⁸ is —NO₂; and
R⁹ is hydrogen.

21. The method of claim 2, wherein L¹ has a length of about 4 to 12 Å.

22. The method of claim 1, wherein the p38 gamma (p38γ) kinase inhibitor is:

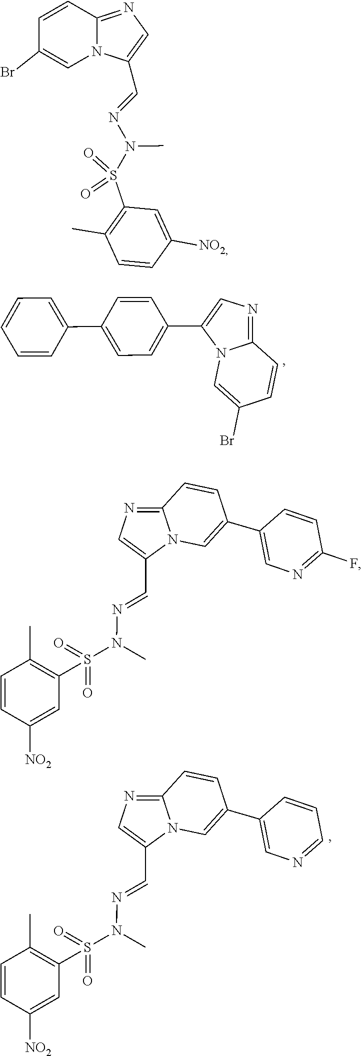

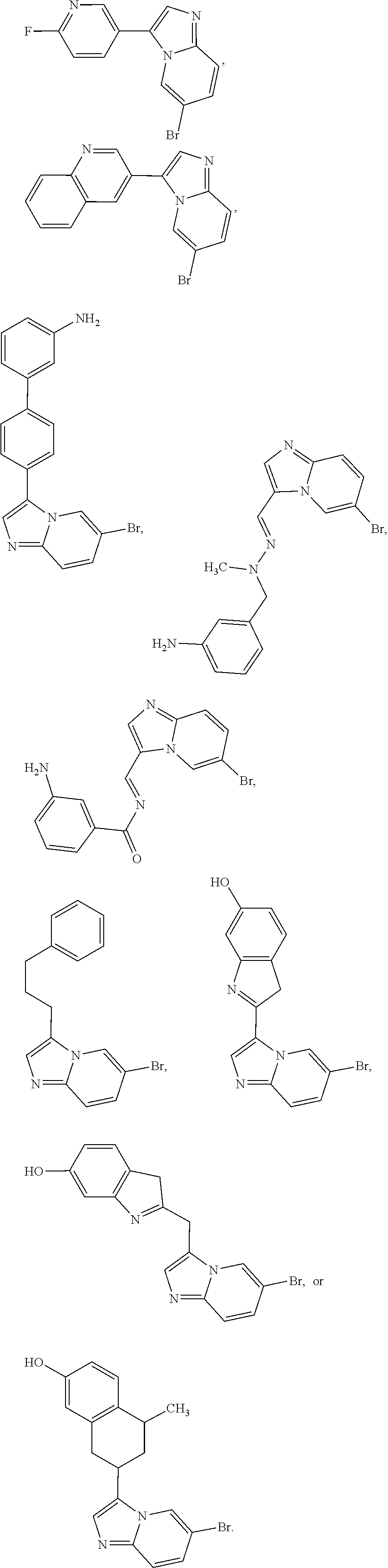

23. The method of claim 1, the method further comprising co-administering an effective amount of histone deacetylase (HDAC) inhibitor.

24. The method of claim 23, wherein the HDAC inhibitor is a compound selected from SAHA, romedepsin, abexinostat, entinostat, panobinostat and trichostatin A.

25. A method of treating a cancer in a subject in need thereof, the method comprising administering a combined effective amount of a histone deacetylase (HDAC) inhibitor and a p38 gamma (p38γ) kinase inhibitor to said subject.

26. The method of claim 25, wherein the cancer is selected from breast cancer, prostate cancer, colon cancer, lymphoma and ovarian cancer.

27. The method of claim 25, wherein the cancer is cutaneous T-cell lymphoma (CTCL).

28. The method of claim 25, wherein the p38γ kinase inhibitor is a compound represented by Formula (I): 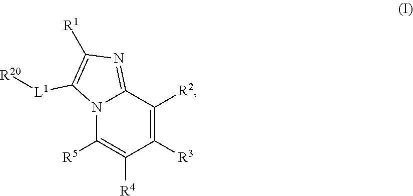
wherein:
L¹ is a bond, —SOₙ₁₁L^{1A}-, —SOᵥ₁₁NR¹¹L^{1A}-, —NHC(O)NR¹¹L^{1A}-, —NR¹¹L^{1A}-, —C(O)L^{1A}, —C(O)OL^{1A}, —C(O)NR¹¹L^{1A}-, —OL^{1A}-, —NR¹¹SO₂L^{1A}-, —NR¹¹C(O)L^{1A}-, —NR¹¹C(O)OL^{1A}-, —NR¹¹OL^{1A}-, —SL^{1A}-, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CX¹₃, —CHX¹₂, —CH₂X¹, —OCX¹₃, —OCH₂X¹, —OCHX¹₂, —N₃, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —OCX²₃, —OCH₂X², —OCHX²₂, —N₃, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —N₃, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —N₃, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, halogen, —CX⁵₃, —CHX⁵₂, —CH₂X⁵, —OCX⁵₃, —OCH₂X⁵, —OCHX⁵₂, —N₃, —CN, —SOₙ₅R^{5D}, —SOᵥ₅NR^{5A}R^{5B}, —NHC(O)NR^{5A}R^{5B}, —N(O)ₘ₅, —NR^{5A}R^{5B}, —C(O)R^{5C}, —C(O)—OR^{5C}, —C(O)NR^{5A}R^{5B}, —OR^{5D}, —NR^{5A}SO₂R^{5D}, —NR^{5A}C(O)R^{5C}, —NR^{5A}C(O)OR^{5C}, —NR^{5A}OR^{5C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²⁰ is substituted or unsubstituted cycloalkyl, substituted or unsubstituted heterocycloalkyl, substituted or unsubstituted aryl, or substituted or unsubstituted heteroaryl;
wherein R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D} and R¹¹ are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{1A} is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
n1, n2, n3, n4, n5 and n11 are independently an integer from 0 to 4;
m1, m2, m3, m4, m5, v1, v2, v3, v4, v5 and v11 are independently an integer from 1 to 2;
X, X¹, X², X³, X⁴, and X⁵ are independently —F, —Cl, —Br, or —I.

29. The method of claim 28, wherein the p38γ kinase inhibitor is a compound represented by Formula (III), 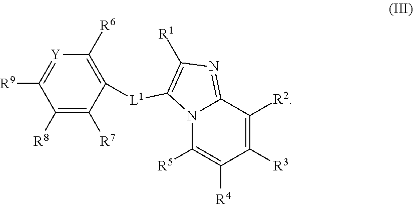
wherein:
Y is —N═ or —CR¹²═;
R⁶ is hydrogen, halogen, —CX⁶₃, —CHX⁶₂, —CH₂X⁶, —OCX⁶₃, —OCH₂X⁶, —OCHX⁶₂, —N₃, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR c, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is hydrogen, halogen, —CX⁷₃, —CHX⁷₂, —CH₂X⁷, —OCX⁷₃, —OCH₂X⁷, —OCHX⁷₂, —N₃, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is hydrogen, halogen, —CX⁸₃, —CHX⁸₂, —CH₂X⁸, —OCX⁸₃, —OCH₂X⁸, —OCHX⁸₂, —N₃, —CN, —SOₙ₈R^{8D}, —SOᵥ₈NR^{8A}R^{8B}, —NHC(O)NR⁸R^{8B}, —N(O)ₘ₈, —NR^{8A}R^{8B}, —C(O)R^{8C}, —C(O)—OR^{8C}, —C(O)NR^{8A}R^{8B}, —OR^{8D}, —NR^{8A}SO₂R^{8D}, —NR^{8A}C(O)R^{8C}, —NR^{8A}C(O)OR^{8C}, —NR^{8A}OR^{8C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CX⁹₃, —CHX⁹₂, —CH₂X⁹, —OCX⁹₃, —OCH₂X⁹, —OCHX⁹₂, —N₃, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen, halogen, —CX¹²₃, —CHX¹²₂, —CH₂X¹², —OCX¹²₃, —OCH₂X¹², —OCHX¹²₂, —N₃, —CN, —SOₙ₁₂R^{12D}, —SOᵥ₁₂NR^{12A}R^{12B}, —NHC(O)NR^{12A}R^{12B}, —N(O)ₘ₁₂, —NR^{12A}R^{12B}, —C(O)R^{12C}, —C(O)—OR^{12C}, —C(O)NR^{12A}R^{12B}, —OR^{12D}, —NR^{12A}SO₂R^{12D}, —NR^{12A}C(O)R^{12C}, —NR^{12A}C(O)OR^{12C}, —NR^{12A}OR^{12C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
wherein R^{6A}, R^{6B}, R^{6C}, R^{6D}, R^{7A}, R^{7B}, R^{7C}, R^{7D}, R^{8A}, R^{8B}, R^{8C}, R^{8D}, R^{9A}, R^{9B}, R^{9C}, R^{9D}, R^{12A}, R^{12B}, R^{12C}, and R^{12D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ and R⁸ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁸ and R⁹ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁹ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁶ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
n6, n7, n8, n9 and n12 are independently an integer from 0 to 4;
m6, m7, m8, m9, m12, v6, v7, v8, v9 and v12 are independently an integer from 1 to 2; and
X⁶, X⁷, X⁸, X⁹, and X¹² are independently —F, —Cl, —Br, or —I.

30. The method of claim 28, wherein R¹ is hydrogen.

31. The method of claim 29, wherein Y is —CR¹²═.

32. The method of claim 31, wherein R¹² is hydrogen, halogen, or unsubstituted C₁-C₄ alkyl.

33. The method of claim 29, wherein Y is —N═.

34. The method of claim 28, wherein:
L¹ is R¹³-substituted or unsubstituted phenylene;
R¹³ is halogen, —CX¹³₃, —CHX¹³₂, —CH₂X¹³, —OCX¹³₃, —OCH₂X¹³, —OCHX¹³₂, —CN, —SOₙ₁₃R^{13D}, —SOᵥ₁₃NR^{13A}R^{13B}, —NHC(O)NR^{13A}R^{13B}, —N(O)ₘ₁₃, —NR^{13A}R^{13B}, —C(O)R^{13C}, —C(O)—OR^{13C}, —C(O)NR^{13A}R^{13B}, —OR^{13D}, —NR^{13A}SO₂R^{13D}, —NR^{13A}C(O)R^{13C}, —NR^{13A}C(O)OR^{13C}, —NR^{13A}OR^{13C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl,
wherein R^{13A}, R^{13B}, R^{13C}, and R^{13D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n13 is independently an integer from 0 to 4;
m13 and v13 are each independently an integer from 0 to 2; and
X¹³ is independently —F, —Cl, —Br, or —I.

35. The method of claim 28, wherein:
L¹ is R¹³-substituted or unsubstituted heteroalkylene;
R¹³ is halogen, —CX¹³₃, —CHX¹³₂, —CH₂X¹³, —OCX¹³₃, —OCH₂X¹³, —OCHX¹³₂, —CN, —SO₁₃R^{13D}, —SOᵥ₁₃NR^{13A}R^{13B}, —NHC(O)NR^{13A}R^{13B}, —N(O)ₘ₁₃, —NR^{13A}R^{13B}, —C(O)R^{13C}, —C(O)—OR^{13C}, —C(O)NR^{13A}R^{13B}, —OR^{13D}, —NR^{13A}SO₂R^{13D}, —NR^{13A}C(O)R^{13C}, —NR^{13A}C(O)OR^{13C}, —NR^{13A}OR^{13C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl,
wherein R^{13A}, R^{13B}, R^{13C}, and R^{13D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n13 is independently an integer from 0 to 4;
m13 and v13 are each independently an integer from 0 to 2; and
X¹³ is independently —F, —Cl, —Br, or —I.

36. The method of claim 28, wherein:
L¹ is R¹³-substituted or unsubstituted 2 to 4 membered heteroalkylene; and
R¹³ is halogen, or unsubstituted C₁-C₃ alkyl.

37. The method of any one of claims 28-33, wherein:
L¹ is —SO₂N(R¹⁴)N═CH—;
wherein R¹⁴ is hydrogen, —CX¹⁴₃, —CHX¹⁴₂, —CH₂X¹⁴, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, and
X¹⁴ is independently —F, —Cl, —Br, or —I.

38. The method of claim 37, wherein R¹⁴ is unsubstituted C₁-C₃ alkyl.

39. The method of claim 28, wherein L¹ is —SO₂—N(CH₃)N═CH—.

40. The method of claim 28, wherein at least one of R², R³, R⁴ and R⁵ is halogen.

41. The method of claim 40, wherein R², R³, and R⁵ are hydrogen and R⁴ is —F, —Cl or —Br.

42. The method of claim 28, wherein at least one of R², R³, R⁴ and R⁵ is halogen-substituted or unsubstituted pyridyl.

43. The method of claim 29, wherein R⁶, R⁷, R⁸ and R⁹ are independently hydrogen, halogen, NO₂, or unsubstituted C₁-C₃ alkyl.

44. The method of claim 29, wherein:
L¹ is unsubstituted phenylene;
R¹, R², R³ and R⁵ are hydrogen;
R⁴ is —F, —Cl or —Br;
R⁶, R⁷, R⁸ and R⁹ are independently hydrogen, halogen, NO₂, or unsubstituted C₁-C₃ alkyl.

45. The method of claim 29, wherein:
L¹ is substituted or unsubstituted C₄-C₆ alkylene, or substituted or unsubstituted 4 to 6 membered heteroalkylene;
R¹, R², R³ and R⁵ are hydrogen;
R⁴ is —Cl or —Br; and
R⁶, R⁷, R⁸ and R⁹ are independently hydrogen, halogen, NO₂, or unsubstituted C₁-C₃ alkyl.

46. The method of claim 29, wherein:
L¹ is —SO₂—N(CH₃)N═CH—;
R², R³ and R⁵ are hydrogen;
R⁴ is halogen, or halogen-substituted or unsubstituted pyridyl;
R⁶ is unsubstituted methyl;
R⁷ is hydrogen;
R⁸ is —NO₂; and
R⁹ is hydrogen.

47. The method of claim 28, wherein L¹ has a length of about 4 to 12 Å.

48. The method of any one of claims 28-47, wherein the p38 gamma (p38γ) kinase inhibitor is:

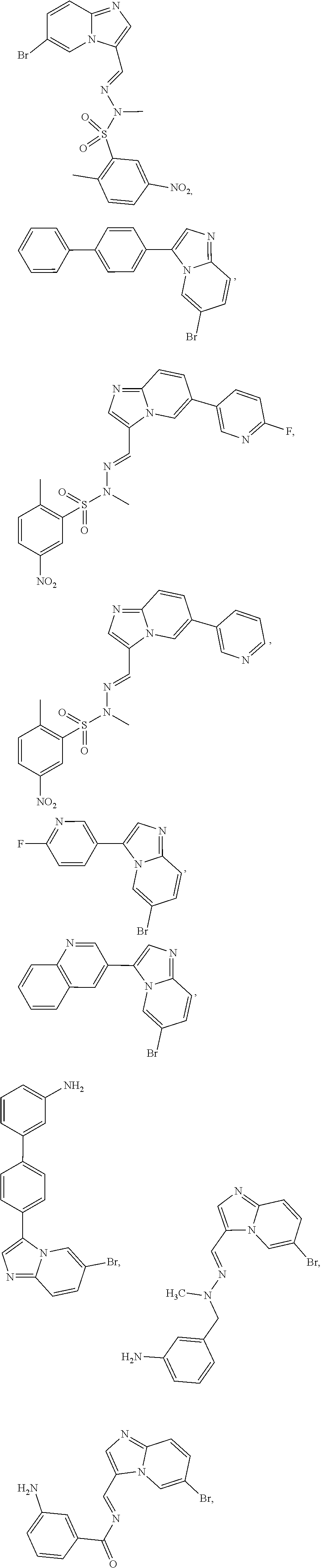

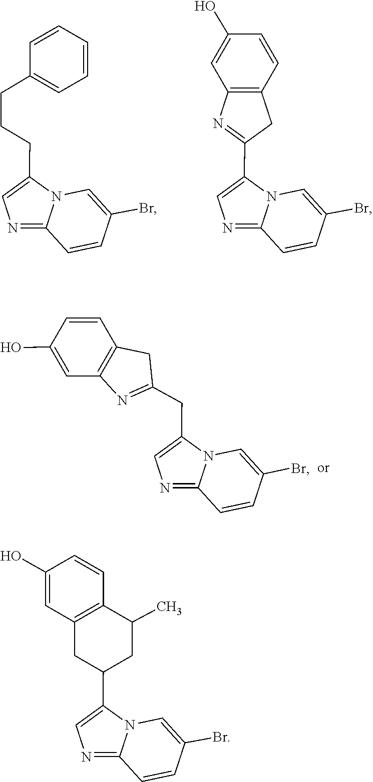 [SMILES: Brc1ccc2ncc(CCCc3ccccc3)n2c1 ; Oc1ccc2c(c1)N=C(c1cnc3ccc(Br)cn13)C2 ; Oc1ccc2c(c1)N=C(Cc1cnc3ccc(Br)cn13)C2 ; CC1CC(c2cnc3ccc(Br)cn23)Cc2cc(O)ccc21]

49. The method of claim 25, wherein the HDAC inhibitor is a compound selected from SAHA, romedepsin, abexinostat, entinostat, panobinostat and trichostatin A.

50. A method of suppressing proliferation of a cutaneous T-cell lymphoma (CTCL) cell, the method comprising contacting the cell with an effective amount of a p38 gamma (p38γ) kinase inhibitor.

51. A compound represented by Formula (I): 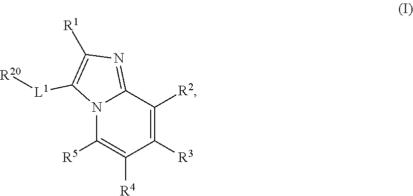
wherein:
L¹ is a bond, —SOₙ₁₁L^{1A}-, —SOᵥ₁₁NR¹L^{1A}-, —NHC(O)NR¹L^{1A}-, —NR¹¹L^{1A}-, —C(O)L^{1A}-, —C(O)OL^{1A}-, —C(O)NR¹¹L^{1A}-, —OL^{1A}-, —NR¹¹SO₂L^{1A}-, —NR¹¹C(O)L^{1A}-, —NR¹¹C(O)OL^{1A}-, —NR¹¹OL^{1A}-, —SL^{1A}-, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is hydrogen, halogen, —CX¹₃, —CHX¹₂, —CH₂X¹, —OCX¹₃, —OCH₂X¹, —OCHX¹₂, —N₃, —CN, —SOₙ₁R^{1D}, —SOᵥ₁NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —OCX²₃, —OCH₂X², —OCHX²₂, —N₃, —CN, —SOₙ₂R^{2D}, —SOᵥ₂NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, halogen, —CX³₃, —CHX³₂, —CH₂X³, —OCX³₃, —OCH₂X³, —OCHX³₂, —N₃, —CN, —SOₙ₃R^{3D}, —SOᵥ₃NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁴ is hydrogen, halogen, —CX⁴₃, —CHX⁴₂, —CH₂X⁴, —OCX⁴₃, —OCH₂X⁴, —OCHX⁴₂, —N₃, —CN, —SOₙ₄R^{4D}, —SOᵥ₄NR^{4A}R^{4B}, —NHC(O)NR^{4A}R^{4B}, —N(O)ₘ₄, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, halogen, —CX⁵₃, —CHX⁵₂, —CH₂X⁵, —OCX⁵₃, —OCH₂X⁵, —OCHX⁵₂, —N₃, —CN, —SOₙ₅R^{5D}, —SOᵥ₅NR^{5A}R^{5B}, —NHC(O)NR^{5A}R^{5B}, —N(O)ₘ₅, —NR^{5A}R^{5B}, —C(O)R^{5C}, —C(O)—OR^{5C}, —C(O)NR^{5A}R^{5B}, —OR^{5D}, —NR^{5A}SO₂R^{5D}, —NR^{5A}C(O)R^{5C}, —NR^{5A}C(O)OR^{5C}, —NR^{5A}OR^{5C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R²⁰ is substituted or unsubstituted cycloalkyl, substituted or unsubstituted heterocycloalkyl, substituted or unsubstituted aryl, or substituted or unsubstituted heteroaryl;
wherein R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4C}, R^{4D}, R^{5A}, R^{5B}, R^{5C}, R^{5D} and R¹¹ are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{1A} is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
n1, n2, n3, n4, n5 and n11 are independently an integer from 0 to 4;
m1, m2, m3, m4, m5, v1, v2, v3, v4, v5 and v11 are independently an integer from 1 to 2; and
X, X¹, X², X³, X⁴, and X⁵ are independently —F, —Cl, —Br, or —I, with proviso that when Y is —CH═, L¹ is —SO₂—N(CH₃)N═CH— and R⁴ is —Br, then R²⁰ is not 2-methyl-5-nitrophenyl.

52. The compound of claim 51, wherein the p38γ kinase inhibitor is a compound represented by Formula (III), 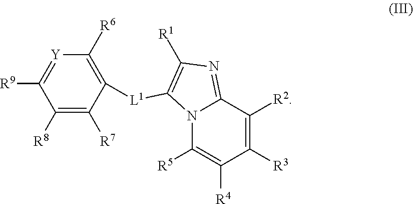
wherein:
Y is —N═ or —CR¹²═;
R⁶ is hydrogen, halogen, —CX⁶₃, —CHX⁶₂, —CH₂X⁶, —OCX⁶₃, —OCH₂X⁶, —OCHX⁶₂, —N₃, —CN, —SOₙ₆R^{6D}, —SOᵥ₆NR^{6A}R^{6B}, —NHC(O)NR^{6A}R^{6B}, —N(O)ₘ₆, —NR^{6A}R^{6B}, —C(O)R^{6C}, —C(O)—OR^{6C}, —C(O)NR^{6A}R^{6B}, —OR^{6D}, —NR^{6A}SO₂R^{6D}, —NR^{6A}C(O)R^{6C}, —NR^{6A}C(O)OR^{6C}, —NR^{6A}OR^{6C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ is hydrogen, halogen, —CX⁷₃, —CHX⁷₂, —CH₂X⁷, —OCX⁷₃, —OCH₂X⁷, —OCHX⁷₂, —N₃, —CN, —SOₙ₇R^{7D}, —SOᵥ₇NR^{7A}R^{7B}, —NHC(O)NR^{7A}R^{7B}, —N(O)ₘ₇, —NR^{7A}R^{7B}, —C(O)R^{7C}, —C(O)—OR^{7C}, —C(O)NR^{7A}R^{7B}, —OR^{7D}, —NR^{7A}SO₂R^{7D}, —NR^{7A}C(O)R^{7C}, —NR^{7A}C(O)OR^{7C}, —NR^{7A}OR^{7C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is hydrogen, halogen, —CX⁸₃, —CHX⁸₂, —CH₂X⁸, —OCX⁸₃, —OCH₂X⁸, —OCHX⁸₂, —N₃, —CN, —SOₙ₈R^{8D}, —SOᵥ₈NR^{8A}R^{8B}, —NHC(O)NR^{8A}R^{8B}, —N(O)ₘ₈, —NR^{8A}R^{8B}, —C(O)R^{8C}, —C(O)—OR^{8C}, —C(O)NR^{8A}R^{8B}, —OR^{8D}, —NR^{8A}SO₂R^{8D}, —NR^{8A}C(O)R^{8C}, —NR^{8A}C(O)OR^{8C}, —NR^{8A}OR^{8C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is hydrogen, halogen, —CX⁹₃, —CHX⁹₂, —CH₂X⁹, —OCX⁹₃, —OCH₂X⁹, —OCHX⁹₂, —N₃, —CN, —SOₙ₉R^{9D}, —SOᵥ₉NR^{9A}R^{9B}, —NHC(O)NR^{9A}R^{9B}, —N(O)ₘ₉, —NR^{9A}R^{9B}, —C(O)R^{9C}, —C(O)—OR^{9C}, —C(O)NR^{9A}R^{9B}, —OR^{9D}, —NR^{9A}SO₂R^{9D}, —NR^{9A}C(O)R^{9C}, —NR^{9A}C(O)OR^{9C}, —NR^{9A}OR^{9C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen, halogen, —CX¹²₃, —CHX¹²₂, —CH₂X¹², —OCX¹²₃, —OCH₂X¹², —OCHX¹²₂, —N₃, —CN, —SOₙ₁₂R^{12D}, —SOᵥ₁₂NR^{12A}R^{12B}, —NHC(O)NR^{12A}R^{12B}, —N(O)ₘ₁₂, —NR^{12A}R^{12B}, —C(O)R^{12C}, —C(O)—OR^{12C}, —C(O)NR^{12A}R^{12B}, —OR^{12D}, —NR^{12A}SO₂R^{12D}, —NR^{12A}C(O)R^{12C}, —NR^{12A}C(O)OR^{12C}, —NR^{12A}OR^{12C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
wherein R^{6A}, R^{6B}, R^{6C}, R^{6D}, R^{7A}, R^{7B}, R^{7C}, R^{7D}, R^{8A}, R^{8B}, R^{8C}, R^{8D}, R^{9A}, R^{9B}, R^{9C}, R^{9D}, R^{12A}, R^{12B}, R^{12C}, and R^{12D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁷ and R⁸ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁸ and R⁹ together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁹ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
R⁶ and R¹² together with atoms attached thereto may optionally be joined to form substituted or unsubstituted cycloalkyl, substituted or unsubstituted heterocycloalkyl, substituted or unsubstituted aryl, or substituted or unsubstituted heteroaryl;
n6, n7, n8, n9 and n12 are independently an integer from 0 to 4;
m6, m7, m8, m9, m12, v6, v7, v8, v9 and v12 are independently an integer from 1 to 2; and
X⁶, X⁷, X⁸, X⁹ and X¹² are independently —F, —Cl, —Br, or —I.

53. The compound of claim 51, wherein R¹ is hydrogen.

54. The compound of claim 52, wherein Y is —CR¹²═.

55. The compound of claim 54, wherein R¹² is hydrogen, halogen, or unsubstituted C₁-C₄ alkyl.

56. The compound of claim 52, wherein Y is —N═.

57. The compound of claim 51, wherein:
L¹ is R¹³-substituted or unsubstituted phenylene;
R¹³ is halogen, —CX¹³₃, —CHX¹³₂, —CH₂X¹³, —OCX¹³₃, —OCH₂X¹³, —OCHX¹³₂, —CN, —SOₙ₁₃R^{13D}, —SOᵥ₁₃NR^{13A}R^{13B}, —NHC(O)NR^{13A}R^{13B}, —N(O)ₘ₁₃, —N R^{13A}R^{13B}, —C(O)R^{13C}, —C(O)—OR^{13C}, —C(O)NR^{13A}R^{13B}, —OR^{13D}, —NR^{13A}SO₂R^{13D}, —NR^{13A}C(O)R^{13C}, —NR^{13A}C(O)OR^{13C}, —NR^{13A}OR^{13C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl,
wherein R^{13A}, R^{13B}, R^{13C}, and R^{13D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n13 is independently an integer from 0 to 4;
m13 and v13 are each independently an integer from 0 to 2; and
X¹³ is independently —F, —Cl, —Br, or —I.

58. The compound of claim 51, wherein:
L¹ is R¹³-substituted or unsubstituted heteroalkylene;
R¹³ is halogen, —CX¹³₃, —CHX¹³₂, —CH₂X¹³, —OCX¹³₃, —OCH₂X¹³, —OCHX¹³₂, —CN, —SOₙ₁₃R^{13D}, —SOᵥ₁₃NR^{13A}R^{13B}, —NHC(O)NR^{13A}R^{13B}, —N(O)ₘ₁₃, —NR^{13A}R^{13B}, —C(O)R^{13C}, —C(O)—OR^{13C}, —C(O)NR^{13A}R^{13B}, —OR^{13D}, —NR^{13A}SO₂R^{13D}, —NR^{13A}C(O)R^{13C}, —NR^{13A}C(O)OR^{13C}, —NR^{13A}OR^{13C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl,
wherein R^{13A}, R^{13B}, R^{13C}, and R^{13D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n13 is independently an integer from 0 to 4;
m13 and v13 are each independently an integer from 0 to 2; and
X¹³ is independently —F, —Cl, —Br, or —I.

59. The compound of claim 51, wherein:
L¹ is R¹³-substituted or unsubstituted 2 to 4 membered heteroalkylene; and
R¹³ is halogen, or unsubstituted C₁-C₃ alkyl.

60. The compound of claim 51, wherein:
L¹ is —SO₂N(R¹⁴)N═CH—;
wherein R¹⁴ is hydrogen, —CX¹⁴₃, —CHX¹⁴₂, —CH₂X¹⁴, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, and
X¹⁴ is independently —F, —Cl, —Br, or —I.

61. The compound of claim 60, wherein R¹⁴ is unsubstituted C₁-C₃ alkyl.

62. The compound of claim 51, wherein L¹ is —SO₂—N(CH₃)N═CH—.

63. The compound of claim 51, wherein at least one of R², R³, R⁴ and R⁵ is halogen.

64. The compound of claim 63, wherein R², R³, and R⁵ are hydrogen and R⁴ is —F, —Cl or —Br.

65. The compound of claim 51, wherein at least one of R², R³, R⁴ and R⁵ is halogen-substituted or unsubstituted pyridyl.

66. The compound of claim 51, wherein R⁶, R⁷, R⁸ and R⁹ are independently hydrogen, halogen, NO₂, or unsubstituted C₁-C₃ alkyl.

67. The compound of claim 52, wherein:
L¹ is unsubstituted phenylene;
R¹, R², R³ and R⁵ are hydrogen;
R⁴ is —F, —Cl or —Br;
R⁶, R⁷, R⁸ and R⁹ are independently hydrogen, halogen, NO₂, or unsubstituted C₁-C₃ alkyl.

68. The compound of claim 52, wherein:
L¹ is substituted or unsubstituted C₄-C₆ alkylene, or substituted or unsubstituted 4 to 6 membered heteroalkylene;
R¹, R², R³ and R⁵ are hydrogen;
R⁴ is —Cl; and
R⁶, R⁷, R⁸ and R⁹ are independently hydrogen, halogen, or unsubstituted C₁-C₃ alkyl.

69. The compound of claim 52, wherein:
L¹ is —SO₂—N(CH₃)N═CH—;
R², R³ and R⁵ are hydrogen;
R⁴ is halogen-substituted or unsubstituted pyridyl;
R⁶ is unsubstituted methyl;
R⁷ is hydrogen;
R⁸ is —NO₂; and
R⁹ is hydrogen.

70. The compound of claim 51, wherein L¹ has a length of about 4 to 12 Å.

71. The compound of claim 51, wherein the compound is:

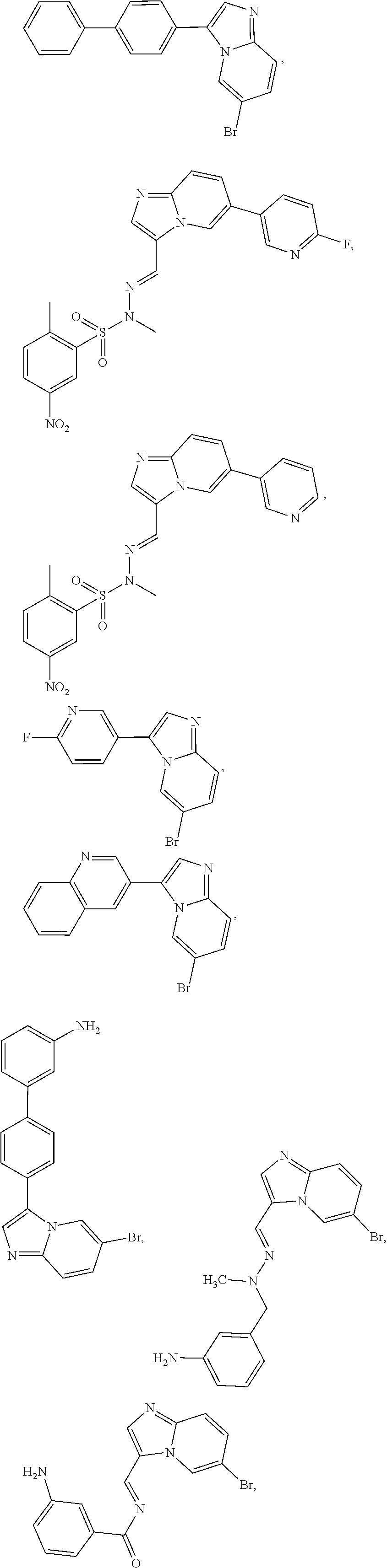

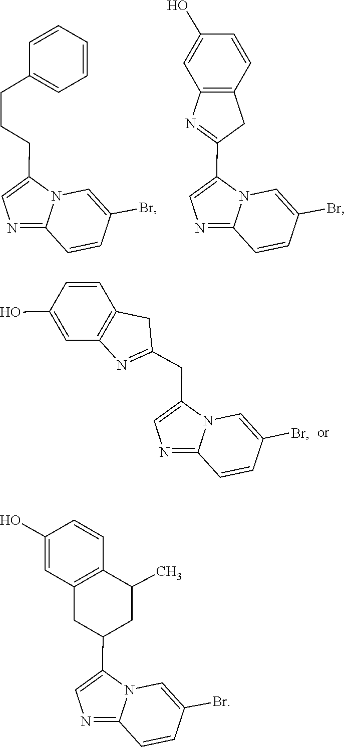

72. A pharmaceutical composition comprising a compound of 51, or a pharmaceutically acceptable salt thereof.


SEQUENCE LISTING (16 sequences)

SEQ ID NO: 1 - DNA, 20 bp, Artificial Sequence
  Synthetic polynucleotide
cccggacatatatccagtcc

SEQ ID NO: 2 - DNA, 19 bp, Artificial Sequence
  Synthetic polynucleotide
tcactgctcaatctccagg

SEQ ID NO: 3 - DNA, 20 bp, Artificial Sequence
  Synthetic polynucleotide
gcccatccctacttcgagtc

SEQ ID NO: 4 - DNA, 20 bp, Artificial Sequence
  Synthetic polynucleotide
cttcacagaggcgtctcctt

SEQ ID NO: 5 - DNA, 22 bp, Artificial Sequence
  Synthetic polynucleotide
ggcagtttaacgtggcctgtta

SEQ ID NO: 6 - DNA, 22 bp, Artificial Sequence
  Synthetic polynucleotide
acagtggatgaatggaagcagc

SEQ ID NO: 7 - DNA, 20 bp, Artificial Sequence
  Synthetic polynucleotide
gccgagctgttgactggaag

SEQ ID NO: 8 - DNA, 21 bp, Artificial Sequence
  Synthetic polynucleotide
ggaggtccctgctttcaaagg

SEQ ID NO: 9 - DNA, 20 bp, Artificial Sequence
  Synthetic polynucleotide
cccggacatatatccagtcc

SEQ ID NO: 10 - DNA, 19 bp, Artificial Sequence
  Synthetic polynucleotide
tcactgctcaatctccagg

SEQ ID NO: 11 - protein, 360 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 12 - protein, 364 aa, Homo sapiens
MSGPRAGFYRQELNKTVWEVPQRLQGLRPVGSGAYGSVCSAYDARLRQKVAVKKLSRPFQSLIHARRTYRELRLLKHLKHENVIGLLDVFTPATSIEDFSEVYLVTTLMGADLNNIVKCQALSDEHVQFLVYQLLRGLKYIHSAGIIHRDLKPSNVAVNEDCELRILDFGLARQADEEMTGYVATRWYRAPEIMLNWMHYNQTVDIWSVGCIMAELLQGKALFPGSDYIDQLKRIMEVVGTPSPEVLAKISSEHARTYIQSLPPMPQKDLSSIFRGANPLAIDLLGRMLVLDSDQRVSAAEALAHAYFSQYHDPEDEPEAEPYDESVEAKERTLEEWKELTYQEVLSFKPPEPPKPPGSLEIEQ

SEQ ID NO: 13 - protein, 367 aa, Homo sapiens
MSSPPPARSGFYRQEVTKTAWEVRAVYRDLQPVGSGAYGAVCSAVDGRTGAKVAIKKLYRPFQSELFAKRAYRELRLLKHMRHENVIGLLDVFTPDETLDDFTDFYLVMPFMGTDLGKLMKHEKLGEDRIQFLVYQMLKGLRYIHAAGIIHRDLKPGNLAVNEDCELKILDFGLARQADSEMTGYVVTRWYRAPEVILNWMRYTQTVDIWSVGCIMAEMITGKTLFKGSDHLDQLKEIMKVTGTPPAEFVQRLQSDEAKNYMKGLPELEKKDFASILTNASPLAVNLLEKMLVLDAEQRVTAGEALAHPYFESLHDTEDEPQVQKYDDSFDDVDRTLDEWKRVTYKEVLSFKPPRQLGARVSKETPL

SEQ ID NO: 14 - protein, 365 aa, Homo sapiens
MSLIRKKGFYKQDVNKTAWELPKTYVSPTHVGSGAYGSVCSAIDKRSGEKVAIKKLSRPFQSEIFAKRAYRELLLLKHMQHENVIGLLDVFTPASSLRNFYDFYLVMPFMQTDLQKIMGMEFSEEKIQYLVYQMLKGLKYIHSAGVVHRDLKPGNLAVNEDCELKILDFGLARHADAEMTGYVVTRWYRAPEVILSWMHYNQTVDIWSVGCIMAEMLTGKTLFKGKDYLDQLTQILKVTGVPGTEFVQKLNDKAAKSYIQSLPQTPRKDFTQLFPRASPQAADLLEKMLELDVDKRLTAAQALTHPFFEPFRDPEEETEAQQPFDDSLEHEKLTVDEWKQHIYKEIVNFSPIARKDSRRRSGMKL

SEQ ID NO: 15 - RNA, 24 nt, Artificial Sequence
  Synthetic polynucleotide
ggaagcguguuacuuacaaagagg

SEQ ID NO: 16 - RNA, 24 nt, Artificial Sequence
  Synthetic polynucleotide
cggagaugaucacaggcaagacgc